(19) United States
(12) Patent Application Publication
(10) Pub. No.: US 2026/0263544 A1
(43) Pub. Date: Sep. 10, 2026

(54) PHAGE THERAPY UTILIZING A UNIQUE COCKTAIL OF PSEUDOMONAS AERUGINOSA PHAGES

(71) Applicant: UNIVERSITY OF FLORIDA RESEARCH FOUNDATION, INCORPORATED, Gainesville, FL (US)

(72) Inventors: Mehdi S. MIRSAEIDI, Gainesville, FL (US); Abdolrazagh HASHEMI SHAHRAKI, Gainesville, FL (US)

(21) Appl. No.: 19/489,147

(22) PCT Filed: Jun. 3, 2024
(86) PCT No.: PCT/US2024/032290

§ 371 Date: Dec. 1, 2025

Related U.S. Application Data
(60) Provisional application No. 63/470,216, filed on Jun. 1, 2023.

(51) Int. Cl. A61K 35/76; A61K 9/08; A61K 45/06; A61L 2/18; A61L 15/44; A61P 31/04; C12Q 1/02

(52) U.S. Cl. CPC A61K 35/76; A61K 9/08; A61K 45/06; A61L 2/18; A61L 15/44; A61P 31/04; C12Q 1/025

(57) ABSTRACT

In this disclosure, the genomes of five newly isolated lytic phages, which infect Pseudomonas aeruginosa, UF_RH1, UF_RH5, UF_RH6, UF_RH7, and UF_RH9 were sequenced, and a new phage cocktail comprising those phages was designed. When the anti P. aeruginosa activity of the phage cocktail was compared with an antibiotic, it was proved that the phage cocktail was at least as effective as the antibiotic. With an in vivo model of wax worm larvae, the phage cocktail increased the survival rate of the worms infected by bacteria. Based on these results, pharmaceutical compositions comprising any of the newly discovered phages or phage cocktail thereof is introduced as a novel antibacterial approach, and use methods thereof are also presented.

### REFERENCE TO ELECTRONIC SEQUENCE LISTING

The application contains a Sequence Listing which has been submitted electronically in. XML format and is hereby incorporated by reference in its entirety. Said .XML copy, created on Jun. 3, 2024, is named “10457556PC_seq-listing.xml” and is 286,096 bytes in size. The sequence listing contained in this .XML file is part of the specification and is hereby incorporated by reference herein in its entirety.

### BACKGROUND

### 1. Field of the Invention

The invention relates to the general field of phage treatment of multidrug resistant (MDR) bacteria, and in particular to a pharmaceutical composition comprising at least one phage disclosed herein for the treatment of MDR Pseudomonas aeruginosa.

### 2. Background of the Invention

Pseudomonas infections, caused by the bacterium Pseudomonas aeruginosa, can have a significant financial impact in the United States. This is because these infections can lead to prolonged hospital stays, increased healthcare costs, and a higher risk of mortality (De Soyza et al., 2013; Lyczak et al., 2000).

According to a study published in the journal Clinical Microbiology and Infection, the cost of treating Pseudomonas infections in the United States is estimated to be around $1.5 billion per year (Jernigan et al., 2020). This includes both direct medical costs, such as hospitalization and medication, as well as indirect costs, such as lost productivity and decreased quality of life.

One of the main factors contributing to the financial impact of Pseudomonas infections is their resistance to antibiotics (Lister et al., 2009; Tacconelli et al., 2018). This means that patients may require more expensive and complex treatments, such as combination therapy or intravenous antibiotics. In addition, patients with Pseudomonas infections often require longer hospital stays and more intensive care, which can increase healthcare costs.

Another factor is the high mortality rate associated with Pseudomonas infections, particularly in vulnerable populations such as the elderly and immunocompromised individuals (Pappas et al., 2009). In addition to the emotional toll on families and communities, these deaths can result in lost productivity and increased healthcare costs.

Bacteriophages, or simply phages, arc viruses that specifically infect bacteria (Wittebolc et al., 2014; Friedberg et al., 1975). These viruses are ubiquitous in nature and can be found in various environments, such as soil, water, and the human body. Phages have been extensively studied as potential therapeutic agents in the field of phage therapy (Au & Assavarittirong, 2022; Cesta et al., 2020; Hashemi & Mirsaeidi, 2021).

Phages have a complex structure that allows them to target and infect specific types of bacteria. They typically consist of a head or capsid that contains the viral DNA, and a tail that facilitates attachment to the bacterial host (David Hou et al., 2022; Folmer et al., 1995). Phages also have unique mechanisms for entering and replicating within the bacterial cell, which can vary depending on the specific phage and host (Reyes-Cortes et al., 2012).

One of the advantages of phages as therapeutic agents is their ability to target specific bacterial strains, including those that are resistant to antibiotics. This specificity is due to the fact that different phages have different host ranges and can only infect certain types of bacteria (Koskella & Meaden, 2013). In addition, phages have been shown to be effective against biofilms, which are communities of bacteria that are often more resistant to antibiotics.

Phage therapy involves the use of phages to treat bacterial infections, either alone or in combination with antibiotics (Torres-Barcelo & Hochberg, 2016). While the use of phages as therapeutic agents has been around for over a century, it fell out of favor in the West with the advent of antibiotics.

However, with the rise of antibiotic resistance, phage therapy has regained interest as a potential alternative or complementary treatment option (Chan et al., 2013).

Phage therapy is a promising approach for the treatment of Pseudomonas aeruginosa infections, which can be difficult to treat with antibiotics due to the emergence of antibiotic-resistant strains (Van Nieuwenhuyse et al., 2022; Chen et al., 2022). Several studies have investigated the use of phages to treat Pseudomonas aeruginosa infections, with promising results.

One study by Krylov et al. (2018) investigated the use of anti-pseudomonal bacteriophages to prevent ventilator-associated pneumonia in an intensive care unit. The study found that phage therapy reduced the incidence of pneumonia and improved clinical outcomes (Maddocks et al., 2019).

Another study in Jault et al. (2019) conducted a randomized, controlled, double-blind phase 1/2 trial to evaluate the efficacy and tolerability of a cocktail of bacteriophages to treat burn wounds infected by Pseudomonas aeruginosa. The study found that phage therapy was safe and effective in treating burn wounds infected with Pseudomonas aeruginosa.

These studies, along with others, demonstrate the potential of phage therapy as a promising alternative to antibiotics for the treatment of Pseudomonas aeruginosa infections. However, more research is needed to fully understand the efficacy, safety, and feasibility of phage therapy in clinical practice (Tamma et al., 2022; Lyon et al., 2022; Gavric & Knezevic, 2022).

### SUMMARY

The treatment of Pseudomonas aeruginosa infections using antibiotics poses a significant challenge (Shariati et al., 2018). Phage therapy has emerged as a highly effective alternative approach, particularly with lytic phages (Chegini et al., 2020).

Bacteriophage therapy employs bacteriophages (viruses that infect bacteria) to combat bacterial pathogens. These natural predators of bacteria offer a highly specific approach to target and eradicate bacterial infections, potentially circumventing the pitfalls associated with broad-spectrum antibiotics, such as disruption of the host microbiome and the promotion of resistance.

Bacteriophages are abundant in different environments that are conducive to bacterial survival, such as soil, water, and wastewater, as well as animal and human tissues, and can be used for elimination of harmful bacteria (Clokie et al., 2011; Alexander et al., 2020).

In this disclosure, five newly discovered and purified phages and phage cocktails comprising them are provided for the treatment of P. aeruginosa infection.

Phage UF_RH1, which was isolated from wastewater, belongs to the Siphoviridae family and the Septimatrevirus genus, and has a 42,567 bp genome, encoding 53 proteins, UF_RH1 shares genetic similarities with Stenotrophomonas phage vB_SmaS-DLP_2.

Phage UF_RH5, which was isolated from wastewater belongs to the Siphovirus family and Septimatrevirus genus, and has a 42,566-bp genome with a GC content of 53.60%, encoding 58 proteins. Under electron microscopy, UF_RH5 exhibits a length of 121 nm and a capsid size of 45 nm.

Phage UF_RH6, which was isolated from a respiratory secretion sample from a patient with pulmonary P. aeruginosa infection, belongs to the Caudoviricetes family and the Samunavirus genus, and has a 94,715 bp genome encoding 130 proteins.

Phage UF_RH7 belongs to Casjensviridae family and has a genome length of 58,217 bp encoding 82 proteins.

Phage UF_RH9 belongs to Caudoviricetes class and has a genome length of 42,609 bp encoding 55 proteins.

For the development of a pharmaceutical composition comprising at least one of those phages, preferably as a phage cocktail, which may minimize phage-resistance in the bacteria, phage cocktails comprising three phages or all five phages were tested against an MDR pathogen, and both phage cocktails significantly reduced bacterial growth (FIG. 5).

Next, the antibacterial effect of the phage cocktail on quinolone-resistant P. aeruginosa was compared with high dose of Cipro, and the phage cocktail was as effective as the antibiotic (FIG. 7)

In addition, the antibacterial effect of the phage cocktail was tested using an in vivo model of bacteria-infected wax worm larvae. The phage cocktail injection reduced bacterial growth in the larvae and increased larvae survival rate, which proved therapeutic potential of the phage cocktail for the treatment of bacterial infection.

Furthermore, the effect of the phage cocktail on bacterial growth in the presence of human immune cells was tested. The addition of phages to peripheral blood mononuclear cell (PBMC) cultures containing bacteria resulted in a marked reduction in bacterial growth. These findings underscore the efficacy of phages in suppressing bacterial populations, even in the presence of host immune cells like PBMCs. Phages alone did not adversely affect PBMC health.

Based on these results, compositions comprising at least one phage selected from a group comprising UF_RH1 (SEQ ID NO:1), UF_RH5 (SEQ ID NO:2), UF_RH6 (SEQ ID NO:3), UF_RH7 (SEQ ID NO:4), and UF_RH9 (SEQ ID NO:5) are presented, and any variants of the nucleic acid sequence having at least 97% sequence identity therewith, or a fragment thereof can also be included in the composition.

In preferred embodiments, the composition comprises at least three phages, and optionally all five phages of UF_RH1, UF_RH5, UF_RH6, UF_RH7, and UF_RH9. In another embodiment, the composition may further comprise other phages that can infect Pseudomonas aeruginosa in addition to one or more or two or more of these phages. Further, the composition optionally comprises pharmaceutically acceptable excipients for parenteral, nasal, or topical administration. In a specific embodiment, provided is a wound dressing comprising an absorbent pad or gauze that comprises a phage composition described herein and optionally an adhesive for holding the wound dressing on the skin of a patient.

For the administration of the composition, the composition may be formulated into a solution for parenteral administration, a solution for nebulizer, a solution or aerosol for nasal spray, aerosol or dry powder for inhalation, or a lotion, cream, gel, an emulsion, ointment, or dry powder for topical administration.

In addition, a method of treating a subject having an antibiotic-susceptible- and/or antibiotic resistant P. aeruginosa infection by administering an effective amount of the composition through parenteral, nasal, or topical route is provided. If need be, the composition may co-administered with an antibiotic or antibiotics.

The target subject of such phage therapy or prevention is a subject who has P. aeruginosa infection in the lung, urinary tract, skin, and/or blood stream, and in particular the patient with P. aeruginosa infection after lung transplant or who is suffering from pneumonia, cystic fibrosis, bronchiectasis, bladder infection (cystitis), kidney infection (pyelonephritis), skin infection (cellulitis, burn wounds, diabetic foot, post-surgical wounds), and/or sepsis, and blood stream infection.

Moreover, considering the survival capability of phages in omni-environment, the composition can be used for disinfecting the surface of medical device or instrument, furniture or walls in a hospital set, by applying to the surface the composition, or a fish tank or aquafarm in agriculture and fishery set, by applying to the water. For such use, the composition may be formulated into a liquid, aerosol, or powder form.

### BRIEF DESCRIPTION OF THE DRAWINGS

FIG. 1 illustrates whole-genome-based phylogenic tree (with average nucleotide identity grouping) and taxonomic position of UF_RH1 among closely related phages of genus Septimatrevirus. Three series of color boxes behind the tree indicate viral taxonomy position (family, genus, and species), and the last one shows the GC content (%) for each virus.

FIG. 2A shows electron microscopy and phylogenetic analysis of UF_RH5 (Ackermann 2012). Negative staining of UF_RH5. High-voltage (HV) is 120 kV with magnification power of 250,000-fold.

FIG. 2B shows the schematic image and virus size,

FIG. 2C is a phylogenetic tree illustrating the taxonomic relationship of UF_RH5 with closely related phages belonging to the genus Septimatrevirus. The tree was generated using the genome-BLAST distance phylogeny method (GBDP). The scale bar indicates the number of substitutions per site. FIG. 2D provides further phylogenetic information of UF_RH5,

FIG. 3A is a phylogenetic tree illustrating the taxonomic relationship of UF_RH6 with closely related phages belonging to the genus Samunavirus (SM1, vB_PaeS_FBPa45, BHU-1, Pa BHU-15, Pa BHU-17, and PaMx31). Two other phages (Stenotrophomonas phage vB_SmaS_DLP_2 and Pseudomonas phage vB_Pae_Kakheti25) are Pseudomonas phages belonging to the genus Septimatrevirus. The tree was generated using the genome BLAST distance phylogeny (GBDP) method. The scale bar indicates the number of substitutions per site.

FIG. 3B is a phylogenetic tree for UF-RH7. FIG. 3C is a phylogenetic tree of UF_RH9.

FIG. 4 is a graph showing Endotoxin Tolerance Limit=5.0 EU/kg (non-intrathecal drug).

FIG. 5A is a summary table of the results of phage addition to the bacteria.

FIG. 5B is a graph presenting the result of phage effect against the growth of MDR P. aeruginosa. Cocktail 1: A blend of three specific phages, each at a designated percentage. Cocktail 2: A blend of five specific phages, each at a designated percentage.

FIG. 6 are graphs presenting the results of phage effect against the growth of MDR P. aeruginosa at different pH (A) and different temperature (B).

FIG. 7 is a graph presenting the result of phage effect against the growth of quinolone-resistant P. aeruginosa.

FIG. 8A is a graph presenting the impact of phage concentration on bacterial counts. Bacteria: 10³ CFU; Phages: 103-1011 PFU.

FIG. 8B is a graph presenting the impact of phage concentration on larvae survival rates. Bacteria: 10³ CFU; Phages: 103-1011 PFU; Larvae: n=8 each group.

FIG. 9A highlights the interactions between bacteria, phages, and PBMCs over time. Bacteria (with PBMC) is now shown in red, demonstrating the growth pattern of bacteria in the presence of PBMCs. Bacteria+Phage (with PBMC) is depicted in black, indicating the suppressed growth of bacteria due to the presence of phages in the PBMC mixture. Bacteria Only is represented in blue, illustrating the exponential growth of bacteria without the influence of PBMCs or phages. Phage (PBMC+Phage) is in yellow, with the concentration remaining constant throughout the observed time period. Bacteria: 10³ CFU; Phages: 105 PFU; PBMC: 10⁴ cells.

FIG. 9B shows the trends in cell viability across different conditions. PBMC+Bacteria is depicted in red, highlighting a decrease in cell viability over time. PBMC+Phage is shown in black, indicating stable cell viability at 100% throughout the experiment. PBMC+Bacteria+Phage is represented in blue, with a notable drop in viability to 50% at the last time point. PBMC+Mitogen is in yellow, showing a gradual decrease in cell viability. PBMC Only is plotted in green (added for distinction), remaining consistently at 100% viability. Bacteria: 10³ CFU; Phages: 105 PFU; PBMC: 10⁴ cells.

FIG. 10 is a diagram presenting the potential dressing product on wounds.

FIG. 11 is a diagram presenting the potential nebulizing product for lung infection.

### DETAILED DESCRIPTION

### A. Definitions

Embodiments of materials and methods are described herein; any methods and materials similar or equivalent to those described herein can be used in the practice of or testing of the invention. Unless defined otherwise, all technical and scientific terms herein have the same meaning as commonly understood by one of ordinary skill in the art to which the invention pertains. In describing and claiming the present invention, the following terminology will be used.

It is also to be understood that the terminology used herein is for the purpose of describing particular embodiments only, and it is not intended to be limiting.

As used herein and in the appended claims, the singular forms “a,” “an,” and “the” include plural references unless specifically stated otherwise.

Other than in the operating examples, or where otherwise indicated, all numbers expressing quantities of ingredients or reaction conditions used herein should be understood as modified in all instances by the term “about.” The term “about” generally refers to a range of numbers that one of skill in the art would consider equivalent to the recited value (i.e., having the same function or result). In a specific embodiment, the term “about” includes a stated numerical value as well as a value that is +/−15% of the stated numerical value. For example, about 5.75 M includes 5.75 molar as well as 6.61 M and 4.89 M, and all 1/10 values in between. In many instances, the term “about” may include numbers that are rounded to the nearest significant figure.

As used herein, the term “bacteriophage” refers to bacterial viruses with a DNA or RNA genome, which is usually protected by a membrane or protein shell (capsid). Bacteriophages infect bacteria either to replicate to large numbers and cause the cell to lyse (lytic infection) or, in some cases, to integrate into the bacterial genome without killing the host bacteria (lysogenic infection). In the lytic infection, viral DNA and proteins are rapidly synthesized and packaged into virus particles, leading to the lysis (destruction) of the host bacteria and then sudden release of progeny virus particles (virions). In contrast, in the lysogenic infection, the phage DNA is inserted into the host bacterial chromosome (prophage state), replicated together with bacterial chromosome, and transmitted to daughter cells. Lysogeny can sometimes significantly affect the host (lysogenic conversion). Under certain conditions causing DNA damages, the prophage is excised and initiates a lytic cycle, leading to the formation of progeny viruses and lysis of the host. (Fortier L C, Sekulovic O. Importance of prophages to evolution and virulence of bacterial pathogens. Virulence. 2013 Jul. 1; 4 (5): 354-65.)

As used herein, the term “lytic activity” refers to phage activity leading to cell lysis of a target bacteria as determined by a plaque assay.

As used herein, the term “plaque assay” refers to phage titration, which is a process for isolation of phage clones and quantitation of the number of viable phage particles in a sample, which is presented as plaque-forming units (pfu) per unit volume, indicating active phage particle numbers in a unit volume of a phage suspension. For plaque assay, 10-fold serial dilutions of a phage sample are carried out through 10-6, mixing each 10-fold serial dilution of the phage sample with aliquots of phage-sensitive bacteria suspended in bacterial culture medium; incubating the mixture of phage and bacteria for 10 minutes to allow for adsorption (attachment) of the phage to the bacteria; preparing tubes containing top agar (0.3-0.75% agar in bacterial culture medium) warmed at the 50° C. water bath; adding certain volume of phage and bacteria mixture (50-500 μL, usually 100-200 μL) into a top agar tube, vortex, pouring onto the appropriately labeled bottom agar plate (1-1.5% agar in bacterial culture medium), one plate for each phage dilution; allowing the top agar to solidify (about 5-10 minutes); and inverting the plates and incubating the plates at 30° C. or 37° C. Phage-infected and lysed bacteria release phages that infect and lyse the surrounding bacteria in the top layer, and multiple rounds of infection and lysis continue until the area of the infected bacteria on the plate is cleared, which appears as individual holes, or plaques, in an otherwise confluent bacterial lawn. Since each plaque is caused by a single viable phage when there are much less phages than bacteria, counting the number of plaques can be assumed as the number of plaque-forming units in the original suspension.

In the specification, the term “phage cocktail” means a phage mixture comprising two or more phages of the invention, or variants or progeny thereof, each of which have been isolated from the environment or human from which they were originally found or have been produced by means of a technical process such as genetic engineering or serial passage techniques. Each of the phages recited herein are not found together in nature, thus, a combination of two or more of the disclosed phages does not pertain to a naturally occurring substance. Also, the phage cocktail will typically include different percentages of the two or more phages in the phage cocktail or phage cocktail composition and may be adjusted such that a certain phage has a higher or lower percentage in the composition relative to another phage.

As used herein, the term “plaque isolation” refers to recovering one single plaque with e.g., an Eppendorf tip and subsequently suspending it in a small volume (e.g., 500 μL or 1.5 mL in a tube) of phage buffer. This can be applied to a small volume (few mL) bacterial culture or spread on the surface of a host agar plate for bacterial lysis.

As used herein, the term “phage propagation” refers to amplifying phages by starting from a single plaque, from phage suspension, or floating agar in a plate that has confluent lysis. Bacterial lysis can be done in a liquid bacterial host culture or on a plate by using the double agar layer technique with top and bottom agar. When using a plate for bacterial lysis by phages, the bottom agar has usual agar concentration (1-1.5%) containing culture nutrients for the host; the top layer has the agar concentration (0.3% to 0.75%) containing the host bacteria mixed in this “soft agar” to form a homogenous thin layer of bacterial lawn. The phages can be dropped onto the top layer or added already into the soft agar. Then, the plate is incubated by shaking carefully on a plate shaker for few hours. The soft agar layer with confluent lysis is scraped off, centrifuged for removing agar and bacterial cell debris. The supernatant is filtered through a 0.45 μm filter, and then through a 0.2 μm filter to get a phage stock solution. When using liquid bacterial culture for bacterial lysis, the phages are added to the bacterial culture of a log phase OD₆₀₀ being cultured in a shaking incubator at 37° C., letting the bacteria and phages grow together until lysis occurs, i.e., the culture media becomes almost clear or transparent. This process needs to be observed because some phage-resistant bacterial cells may overgrow and make the culture turbid again. (dsmz.de/fileadmin/user_upload/Collection_allg/DSMZ_Cultivation_of_Phages.pdf)

As used herein, the term “antibiotics” refers to antimicrobial substance active against bacteria for the treatment of bacterial infections, which usually either kill or inhibit the growth of bacteria. General antibiotics can be classified into to: (1) bacterial cell wall or membrane synthesis inhibitors, e.g., penicillins and cephalosporins; (2) protein synthesis inhibitors (e.g., macrolides, tetracyclins); and (3) DNA synthesis inhibitors (e.g., fluoroquinolones gyrase inhibitors and sulfa antibiotics inhibiting bacterial folic acid synthesis). Standard or traditional antibiotic agents include, but are not limited to: (1) cell wall or membrane synthesis inhibitor antibiotics, e.g., penicillins or beta-lactam compounds: pencillins (e.g., penicillin G, penicillin V, isoxazolyl penicillins, oxacillin, cloxacillin, flucloxacillin, dicloxacillin, nafcillin, methicillin, ampicillin, amoxicillin, piperacillin, ticarcillin, carbenicillin, aminopenicillins, carboxypenicillins, ureidopenicillins, temocillin, azlocillin, mezlocillin, mecillinam); cephalosporins and cephamycin: (e.g., cefazolin, cephalexin, cephradine, cefadroxil, cefuroxime, cefaclor, cefamandole, cefonicid, cefprozil, ceforanid, cefoxitin, cefmetazole, cefotetan, cefoperazone, cefotaxime, ceftazidime, ceftriaxone, cefepime, ceftaroline fosamil, cephalothin, cephapirin, cefpodoxime, ceftibuten, cefdinir, ceftizoxime, ceftriaxone, cefepime, cefditoren, cefixime, ceftibuten, cefacetrile, cefaloglycin, cefalonium, cefaloridine, cefatrizine, cefazaflur, cefazedone, cefroxadine, ceftezole, cefuzonam, cefcapene, cefdaloxime, cefdinir, cefditoren, cefetamet pivoxil, cefmenoxime, cefteram, ceftiofur, cefoperazone, ceftazidime latamoxef, cefclidine, cefluprenam, cefoselis, cefozopran, cefpirome, cefquinome, ceftobiprole, flomoxef (or oxa-1-cephamycin), ceftobiprole); carbacephems (e.g., loracarbef); carbapenems (e.g., biapenem, doripenem, ertapenem, imipenem, imipenem-cilastatin, meropenem, tebipenem pivoxil, faropenem, panipenem/betamipron, razupenem (PTZ-601), thienpenem (thienamycin)); monobactams (e.g., aztreonam, tigemonam, nocardicin A, or tabtoxinine β-lactam); beta-lactamase inhibitors (e.g., clavulanic acid, sulbactam, tazobactam); glycopeptides (e.g., vancomycin, teicoplanin); lipoglycopeptide (e.g. oritavancin, dalbavancin and telavancin); daptomycin, fosfomycin, bacitracin, cycloseine, isoniazid; polypeptide antibiotics (e.g., polymyxin B, or polymyxin E (colistin)); (2) antibiotics targeting bacterial ribosome subunits, the 30S and the 50S subunits; dactinomycin (or actinomycin D), chloramphenicol; tetracyclines (e.g., tetracycline, chlortetracycline, doxycycline, oxytetracycline, demeclocycline, lymecycline, meclocycline, methacycline, minocycline, rolitetracycline, or tigecycline); macrolides (e.g., erythromycin, clarithromycin, azithromycin, fidaxomicin, telithromycin, carbomycin A, josamycin, kitasamycin, midecamycin/midecamycin acetate, oleandomycin, solithromycin, spiramycin, troleandomycin, tylosin, or roxithromycin, dirithromycin, roxithromycin); aminoglycosides: (e.g., streptomycin, rhodostreptomycin, kanamycin, neomycin, amikacin, gentamicin, netilmicin, tobramycin, paromycin, apramycin) apramycin, plazomicin, arbekacin, and streptomycin); lincosamides (e.g., lincomycin, pirlimycin and clindamycin); ansamycins: geldanamycin, naphthomycin, rifamycins (e.g., rifampicin (or rifampin), rifabutin, rifapentine, rifalazil, or rifaximin); quinupristin-dalfopristin, mupirocin, streptogramins (e.g., streptogramin A, streptogramin B), oxazolidinones (e.g., linezolid, tedizolid), spectinomycin; (3) DNA replication inhibitor antibiotics: fluoroquinolones and quinolones (e.g., nalidixic acid, norfloxacin, ciprofloxacin, levofloxacin, gatifloxacin, moxifloxacin, gemifloxacin, lomefloxacin, ofloxacin, pefloxacin, moxifloxacin, rosoxacin, enoxacin, fleroxacin, nadifloxacin, rufloxacin, balofloxacin, grepafloxacin, pazufloxacin, sparfloxacin, temafloxacin, tosufloxacin, clinafloxacin, garenoxacin, stifloxacin, trovafloxacin, prulifloxacin, cinoxacin, flumequine, oxolinic acid, piromidic acid, pipemidic acid); sulfonamide antibiotics (e.g., sulfacytine, sulfisoxazole, sulfamethizole, sulfadiazine, silver sulfadiazine, sulfamethoxazole, sulphapyridine, sulfadoxine, sulfathalidine, sulfacetamide sodium, mafenide, co-trimoxazole, sulfasalazine, sulfanilamides, sultiame, sulfadimethoxine; pyrimidines (e.g., trimethoprim, pyrimethamine, and more than 200 drugs; https://go.drugbank.com/categories/DBCAT000349); others (pyrazinamide, ethambutol, streptomycin, ansamitocin); and any combination thereof can be used.

As used herein, the term “multidrug resistance” refers to antibacterial resistance which happens when bacteria develop the ability to defeat the drugs designed to kill them. That means bacteria are not killed and continue to grow. CDC typically uses this term to refer to an isolate that is resistant to at least one antibiotic in three or more antibiotics classes. Multidrug resistance mechanisms fall into four main categories: (1) limiting uptake of a drug; (2) modifying a drug target; (3) inactivating a drug; (4) active drug efflux. There are two main ways that bacterial cells can acquire antibiotic resistance. One is through mutations that occur in the DNA of the cell during replication. The other way that bacteria acquire resistance is through horizontal gene transfer through plasmids or transposons coding for resistance to a specific agent. Examples of bacteria resistant to antibiotics are methicillin-resistant Staphylococcus aureus (MRSA), vancomycin-resistant Enterococcus (VRE), multi-drug-resistant Mycobacterium tuberculosis (MDR-TB) and carbapenem-resistant Enterobacteriaceae (CRE) gut bacteria. Generally, MDR is defined as non-susceptibility to ≥1 agent in >3 antimicrobial categories, extensively drug-resistant (XDR) as non-susceptibility to >1 agent in all but ≤2 categories, and pandrug-resistant (PDR) as non-susceptibility to all antimicrobial agents listed. (Magiorakos A P et al., Multidrug-resistant, extensively drug-resistant and pandrug-resistant bacteria: an international expert proposal for interim standard definitions for acquired resistance. Clin Microbiol Infect. 2012 March; 18 (3): 268-81.)

As used herein, the term “subject” or “patient” encompasses the whole phyla of animal kingdom including arthropoda, mollusca, chordata, for example, crab, lobster, shrimp, squid, oysters, clams, mussels, or other shellfish, eel, fishes, amphibians, reptiles, birds, and mammals that include domestic-, farm-, zoo- and wild animals, primates, and humans, who may be suffering from P. aeruginosa infection, particularly an infection caused by MDR P. aeruginosa strains in the lung, urogenital organ, skin or blood stream. For example, the subject has P. aeruginosa infection after lung transplant or is suffering from pneumonia, cystic fibrosis, bronchiectasis, bladder infection (cystitis), kidney infection (pyelonephritis), skin infection (cellulitis, burn wounds), and/or sepsis.

As used herein, the term “treatment” in the context of pharmacological or medical meaning refers to intervention of disease, disorder, condition or symptoms to obtain a desired physiologic effect. “Treatment” includes: inhibiting the disease, disorder, condition, or symptoms thereof, such as, arresting its development or progression, and also includes relieving, alleviating, or ameliorating the disease, disorder, condition, or reducing one or more symptoms thereof, such as, for example, reducing infection.

As used herein, the term “administering” refers to introducing an agent to a subject, and can be performed using any of the various methods for drug or composition delivery known to those skilled in the art. Routes of administering include, but are not limited to oral administration, parenteral administration (subcutaneous (SC/SQ: <1 mL), intravenous (IV: 1-20 mL), intradermal (ID: <0.2 mL), intramuscular (IM: <4 mL), intraperitoneal (IP), intraarterial, intracardiac, intraarticular, and intraspinal injection), rectal administration by way of suppositories or enema, local/topical administration directly into or onto a target tissue, nasal administration (nebulizer, nasal spray, inhalation), or administration by any route or method that delivers a therapeutically effective amount of the drug or composition to the cells or tissue to which it is targeted.

As used herein, the term “therapeutically effective amount” refers to an amount effective, at dosages and for periods of time necessary, to achieve the desired therapeutic results. A therapeutically effective amount is also one in which any toxic or detrimental effects of compounds of the invention are outweighed by the therapeutically beneficial effects.

As used herein, the term “composition” refers to a pharmaceutical composition, meaning a mixture of substances suitable for administering to an individual, which includes one or more pharmaceutically active ingredients. For example, a pharmaceutical composition may comprise a certain amount of phage particles in solution or lyophilized dry powder as well as suitable pharmaceutical excipients.

As used herein, the term “excipient”, in the context of pharmaceuticals, refers to any substances other than the active ingredient or agent, contained in pharmaceutical dosage forms. The excipients are considered as inert substances, i.e., they do not have any active role in therapeutics, but they can be used to support the process to produce an effective product. Examples of excipients are active pharmaceutical ingredient excipients, binder excipients, capsule shell excipients, carrier excipients, coating systems excipients, controlled release excipients, diluent excipients, disintegrant excipients, effervescent system excipients, emulsifier excipients, film former excipients, flavor excipients, high-functionality excipients, lipid excipients, lubricant excipients, modified release excipients, penetration enhancer excipients, permeation enhancer excipients, pH modifier excipients, plasticizer excipients, preservative excipients, sachet filling excipients, solubilizer excipients. solvent excipients, surfactant excipients, sustained release excipients, taste masking excipients, thickener excipients, viscosity modifier excipients, blending excipients, filler excipients, compaction excipients, direct compression excipients, dry granulation excipients, hot melt extrusion excipients, wet granulation excipients, rapid release agent excipients, film formation excipients, increased bioavailability excipients, dispersion excipients, solubility enhancement excipients, stabilizer excipients, capsule filling excipients, powder blends excipients, tablet compressibility excipients, etc. (https://www.americanpharmaceuticalreview.com/25335-Pharmaceutical-Raw-Materials-and-APIs/25283-Pharmaceutical-Excipients/)

As used herein, the term “dosage” refers to the administering of a specific amount, number, and frequency of doses over a specified period of time, and the term “dose” refers to a specified amount of medication taken at one time. A “dosage regimen” refers to the number of doses of a drug, medication, or an agent that a patient is supposed to take (or to be administered) over a specified period of time, and the individual doses that comprise the regimen are usually scheduled.

As used herein, the term “dosage form” refers to a pharmaceutical preparation in which a specific mixture of active ingredients of a drug and inactive components (excipients) are formulated in a particular shape or form to facilitated administration and accurate delivery of active ingredients, and/or to be presented in the market. Solid dosage forms include powders, granules, capsules, tablets, cachets, pills, lozenges, gummies, suppositories. Semi-solid dosage forms include ointment, creams, paste, gels, poultices. Liquid dosage forms include collodions, droughts, elixirs, emulsions, suspension, enemas, gargles, linetuses, lotion, liniments, mouth washes, nasal drop, paints, solutions, syrups. Gaseous dosage forms include aerosols, inhalations, and sprays. (https://thepharmapedia.com/pharmaceutical-dosage-form-pharmaceutics/pharmacy-notes/)

As used herein, the terms “administer” and “administration,” when used with respect to a drug or an agent (including antibodies and antibiotics), means providing the drug or agent to a subject using any of the various methods or delivery systems for pharmaceutical compositions known to those skilled in the art. For example, the administration of the drug can be oral, nasal, parental, topical, ophthalmic, or transdermal delivery of the drug in the form of solid, semi-solid, lyophilized powder, or liquid dosage forms.

As used herein, the terms “co-administration” refers to the administration of a first active agent before, concurrently, or after the administration of a second active agent such that the biological effects of the two (or more) agents overlap.

As used herein, the term “nucleic acid” refers to any polyribonucleotide or polydeoxyribonucleotide that may be unmodified RNA or DNA or modified RNA or DNA. Thus, for instance, nucleic acid as used herein refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or double-stranded, or a mixture of single- and double-stranded regions.

As used herein, “sequence identity” or “identity” in the context of two nucleic acid- or polypeptide sequences refers to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically, this involves scoring a conservative substitution as a partial rather than a full mismatch, thereby increasing the percentage sequence identity. Thus, for example, where an identical amino acid is given a score of one and a non-conservative substitution is given a score of zero, a conservative substitution is given a score between zero and one. The scoring of conservative substitutions is calculated, e.g., as implemented in the program PC/GENE (Intelligenetics, Mountain View, Calif.).

### B. Overview

Here is presented the genome sequences of five novel lytic bacteriophages, called UF_RH1, UF_RH5, UF_RH6, UF_RH7, and UF_RH9, which target clinically isolated Pseudomonas aeruginosa.

UF_RH1 has a genome size of 42,567 bp and classified as a member of the Siphoviridae family and the Septimatrevirus genus. UF_RH1 shares genetic similarities with Stenotrophomonas phage vB_SmaS-DLP_2. The genome sequence of UF_RH1 comprises a nucleic acid sequence of SEQ ID NO: 1 presented in this disclosure.

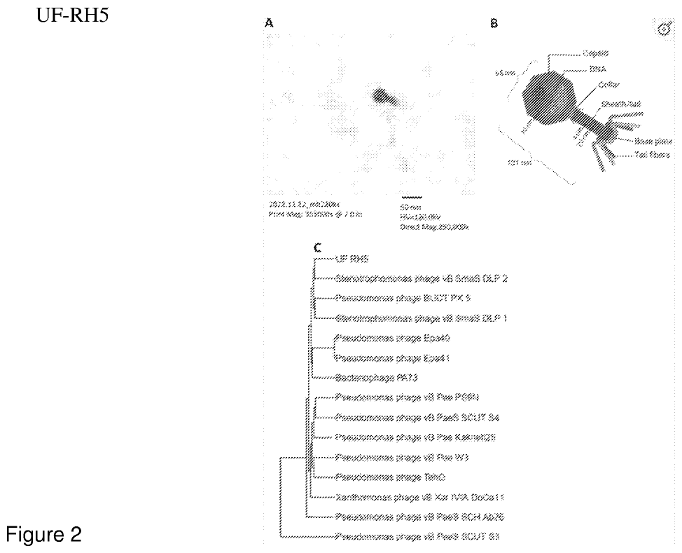

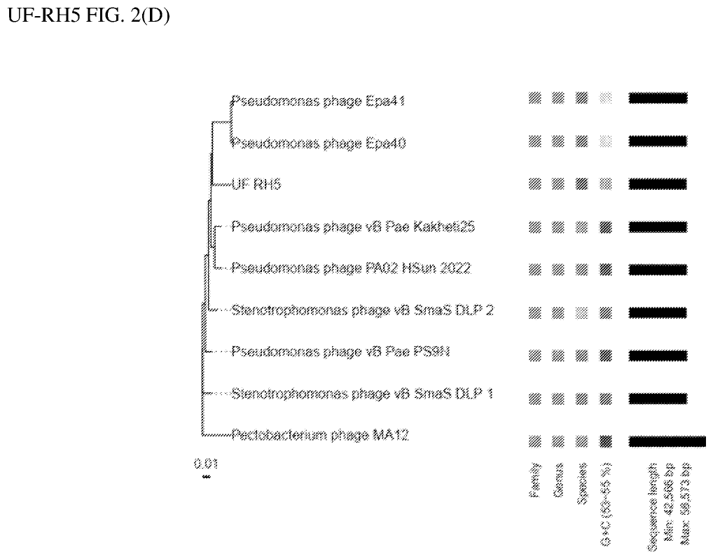

UF_RH5 belongs to the Siphovirus morphology family, Septimatrevirus genus, with a 42,566-bp genome with a GC content of 53.60%, encoding 58 proteins. Under electron microscopy, UF_RH5 exhibits a length of 121 nm and a capsid size of 45 nm (FIG. 2). The genome sequence of UF_RH5 comprises SEQ ID NO:2.

Another lytic phage named UF_RH6 was isolated from a respiratory secretion sample from a patient with pulmonary P. aeruginosa. UF_RH6 belongs to the family Caudoviricetes and the genus Samunavirus. Its genome is 94,715 bp in length and encodes 130 proteins. The genome sequence of UF_RH6 comprises SEQ ID NO:3.

The genomes of other two lytic phages, UF_RH7 and UF_RH9, were sequenced. UF_RH7 belongs to Casjensviridae family and has a genome length of 58,217 bp and encodes 82 proteins. UF_RH9 belongs to Caudoviricetes class and has a genome length of 42,609 bp and encodes 55 proteins. The genome sequence of UF_RH7 comprises SEQ ID NO:4, and that of UF_RH9 comprises SEQ ID NO:5.

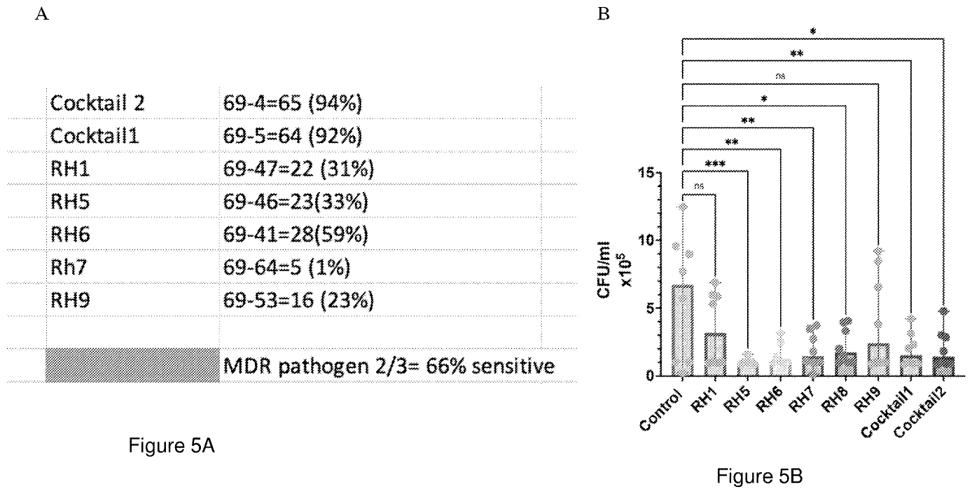

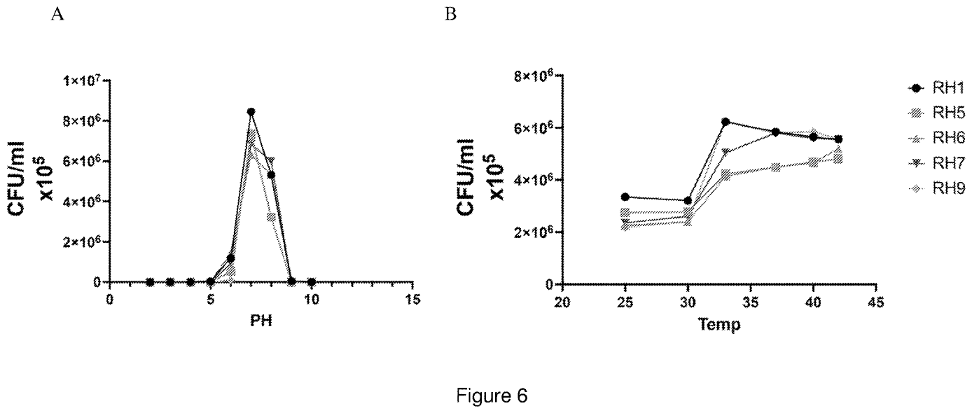

Next, the antibacterial effect of each phage, 3-phage containing cocktail, and 5-phage containing cocktail was tested on P. aeruginosa (FIG. 5). These phages and phage cocktails suppressed bacterial growth.

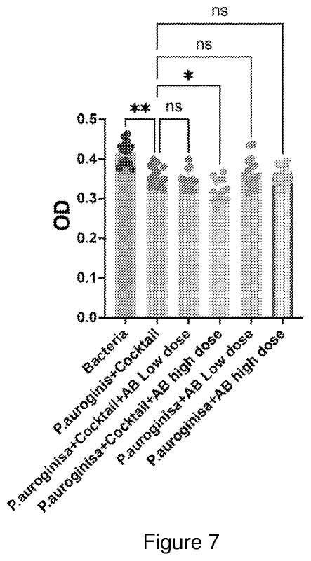

When the phage cocktail was tested on quinolone sensitive P. aeruginosa, its efficacy was better than an antibiotic (FIG. 7). When the phage cocktail was used together with, the combination suppressed more bacterial growth than the phage cocktail alone or AB alone.

### C. Interaction Between Phages, Pseudomonas Bacteria, and Larvae

When various concentrations of phage cocktail were tested using an in vivo model of bacteria-infected wax worm larvae, as the total amount of phages injected into the larvae decreased, bacterial counts increased, and larvae survival rate declined. At a high phage concentration (1011 PFU), significant suppression of bacterial proliferation was observed, with larvae survival rate at 100%.

In other embodiments, the dynamic interaction between phages, Pseudomonas bacteria, and wax worm larvae (in vivo) over a 24-hour period following bacterial and phage exposure was investigated. Specifically, the effects of varying phage concentrations on larvae survival were explored in the presence of a constant bacterial count. Data were organized according to initial phage concentration (Plaque Forming Units, PFU) and subsequent bacterial count (Colony Forming Units, CFU), along with the survival rate of the larvae.

The findings of this study contribute to the growing body of evidence on the role of bacteriophages in controlling bacterial populations and enhancing host survival, aligning with the outcomes of similar research in the field. The results here, showing a significant reduction in Pseudomonas CFU at higher phage concentrations with concurrent increases in larvae survival rates, suggesting a critical role for phages in microbial control within ecological and clinical settings.

Moreover, the positive correlation between phage concentration and larvae survival rates observed in our study mirrors the work of Dabrowska et al. (2005), who found that phage therapy could significantly improve survival in bacterially infected hosts. The relatively high R2 score of 0.822 in this analysis underscores a strong and consistent relationship between phage concentration and host survival, further supported by the quantitative insights provided by Hagens and Loessner (2010), who highlighted the potential of phages as biocontrol agents.

However, the studies presented herein extend beyond the scope of these previous works by offering a quantitative analysis of how specific phage concentrations correlate with both bacterial suppression and host survival rates. This dual focus provides a nuanced understanding of phage-bacteria-host dynamics, emphasizing the balance between bacterial control and maintaining a non-toxic environment for the host, a concept also explored by Skurnik et al. (2007) in their discussion on the therapeutic potential of bacteriophages.

Contrasting these findings with those of Smith et al. (2003), who reported on the variable efficacy of phages across different bacterial strains and host species, suggests that the interaction dynamics we observed may not be universally applicable. This variability highlights the need for tailored phage therapy strategies, as suggested by Kutter et al. (2010), who advocated for a personalized approach to phage treatment based on specific host-pathogen-phage interactions.

In conclusion, the studies disclosed herein add valuable data to the discourse on phage therapy, reinforcing the notion that phages can be a powerful tool against bacterial pathogens while also supporting host survival. Future investigations should aim to dissect the mechanisms underlying phage efficacy and host response, potentially paving the way for novel therapeutic applications that leverage the specificity and potency of phages against bacterial infections.

### D. Phages vs. Pathogens in the Immune Arena: Deciphering Bacterial Dynamics and Host Responses for Next-Generation Therapeutics

The interaction between bacteriophages, their bacterial targets, and the host human immune system is a critical aspect of the therapeutic use of phages. Peripheral blood mononuclear cells (PBMCs), key components of the immune response, play a pivotal role in the body's defense against infections and in mediating inflammatory processes. Understanding the dynamics between PBMCs, bacteriophages, and bacteria is essential for assessing the viability of phage therapy, particularly concerning its safety, efficacy, and the immune system's potential influence on therapeutic outcomes.

A study was conducted to investigate the growth patterns of bacteria and bacteriophages over time in an experimental setup involving PBMCs. By examining the interactions within this tripartite system, the research seeks to elucidate the effects of phages on bacterial populations in the presence of immune cells and to assess the implications for cell viability. Such insights arc crucial for advancing the application of phage therapy in clinical settings, where the immune system's role in treatment outcomes must be thoroughly understood.

By situating this investigation within the broader context of antimicrobial resistance and phage therapy research, this study contributes to the growing body of knowledge needed to harness the full potential of bacteriophages as a viable alternative to traditional antibiotics. As the global health community continues to grapple with the challenge of antibiotic resistance, the exploration of phage therapy, informed by comprehensive research into its mechanisms of action and interactions with the host immune system, represents a forward-looking approach to developing new therapeutic strategies.

The findings from this study offer valuable insights into the interactions between bacteria, phages, and peripheral blood mononuclear cells (PBMCs), with significant implications for understanding bacterial growth dynamics and phage therapy applications. Here are discussed the implications of these results, focusing on bacterial growth, phage stability, and cell viability in various experimental conditions.

In one aspect, the methodology of co-culturing PBMCs, bacteria, and phages provides a foundation for understanding the complex dynamics at play in bacteriophage therapy, specifically regarding the interplay between bacteriophages, bacteria, and the host immune system. Through careful experimental design and rigorous data analysis, this study aims to contribute to the optimization of phage therapy as a viable alternative to traditional antibiotics.

Overall, the interactions of phages with host immune cells, the stability of phage populations, and the implications on cell viability remain critical considerations for the clinical translation of phage therapy (Wright et al., 2009). Future research should continue to address these aspects, focusing on optimizing phage therapy to ensure safety, efficacy, and minimized resistance development.

### E. Embodiment Examples

The findings in this disclosure underscore the potential of phage therapy as a viable alternative to antibiotics, especially crucial in the face of rising antibiotic resistance.

The following are non-limiting examples of phage applications.
(1) Infection control for targeted elimination of pathogenic bacteria resistant to antibiotics; prevention of bacterial outbreaks in healthcare and community settings; and treatment of biofilm-associated infections, such as those in cystic fibrosis.
(2) Agricultural applications such as biocontrol agents for plant pathogens to enhance crop protection and use in livestock to prevent and treat bacterial diseases for reducing antibiotic use.
(3) Food safety control for reduction of foodborne pathogens to prevent spoilage and contamination; and application in food packaging to extend shelflife and maintain product quality.
(3) Diagnostic utilization of phages in biosensors for the rapid detection of bacterial pathogens.
(4) Dental Applications such as: phage therapy for the treatment of periodontal diseases caused by bacterial infections; and use in oral hygiene products to control harmful oral bacteria.
(5) Veterinary medicine for treatment of infectious diseases in companion and farm animals; and use as a preventive measure in animal husbandry to promote health and reduce antibiotic resistance.
(6) Environmental decontamination such as: application in bio-remediation processes to degrade environmental contaminants; and use in water treatment facilities to reduce bacterial loads and improve water quality

Disclosed herein are five novel phages able to infect strains of P. aeruginosa are introduced, which can be utilized for the formulation of a pharmaceutical composition, which comprise at least one phage selected from UF_RH_1 comprising SEQ ID NO: 1, UF_RH_5 comprising SEQ ID NO: 2, UF_RH_6 comprising SEQ ID NO: 3, UF_RH_7 comprising SEQ ID NO: 4, UF_RH_9 comprising SEQ ID NO: 5, but a variant or variants thereof having a nucleic acid sequence of at least 97% sequence identity therewith, or a fragment thereof may be used for the composition. A preferred embodiment is a composition comprising at least three phages selected from them.

In certain embodiments, the composition is formulated into a solution for parenteral administration.

In certain embodiments, the composition is formulated into a solution for nebulizer.

In certain embodiments, the composition is formulated into a solution or aerosol for nasal spray.

In certain embodiments, the composition is formulated into aerosol or dry powder for inhalation.

In certain embodiments, the composition is formulated into a lotion, cream, gel, an emulsion, ointment, or dry powder for topical administration.

In addition, such compositions may further comprise pharmaceutically acceptable excipients for nasal, parenteral, or topical administration. As used herein, the term “excipients” refers to substances that are added to therapeutic products to improve stability, bioavailability, and manufacturability. Examples of excipients are listed below.

Further, a spray container, which is pressurized or not, containing the composition can be contemplated, such as nasal spray or inhalation spray, and the spray container may comprise other antibacterial substances such as an antibiotic/antibiotics or antibodies or fragment thereof targeting the surface of the pathogens, which are in the dosage form of liposome, solution, or lyophilized dry powder.

In other embodiments, methods of treating a subject having P. aeruginosa infection arc provided, and the methods comprise administering an effective amount of the composition described above, through nasal, parenteral, and/or topical route.

Composition embodiments comprising at least one phage described in this disclosure can be administered alone or co-administered with antibiotics, especially antibiotics against gram-negative bacteria. Antibiotics can be administered through oral, parenteral, topical, and/or nasal route together with the composition or separately before, after, or at the same time with the administration of the composition. In addition, some antibodies against the surface antigens of the target bacteria may be contemplated for co-administration with the composition herein.

The subjects suitable for the treatment using the composition are patients with P. aeruginosa infection in the lung, urinary tract, skin, abdominal infections, and/or blood stream, and in particular patients who has P. aeruginosa and/or infection after lung transplant or are suffering from pneumonia, cystic fibrosis, bronchiectasis, bladder infection (cystitis), kidney infection (pyclonephritis), skin infection (cellulitis, burn wounds), and/or sepsis.

For the treatment of bacterial infection, an effective amount of the phage or phage cocktail is administered to a patient through nasal (nebulizer, nasal or inhalation spray), parenteral (intravenous, intramuscular, intraperitoneal, subcutaneous, etc.) or topical route. Pharmaceutical composition for each administration route is formulated into a proper dosage form and comprises necessary excipients. For example, the phage or phage cocktail can be dissolved or suspended in saline or other buffers to be manufactured as a solution for nebulizer mist, nasal spray, or intravenous injection, and each of solutions for different route administration may have similar or different excipients.

For example, parenteral administration, the composition may comprise lyoprotectants (e.g., human albumin, lactose monohydrate, maltose/trehalose/sucrose, mannitol, dextran, inulin, fructose), micro-encapsulating agents (e.g., aliphatic polyester such as polyglycolide, polylactide, and their copolymers, phospholipids/lecithin, phosphatidic acids, phosphoglycerol, phosphoserine, phosphorethanolamine, phosphocholine, PEGylated phospholipids, hydroxypropylcyclodextrin, Betadex sulfobutyl ether sodium), solubilizers and emulsifiers (e.g., N-methyl 2-pyrrolidone, PEG, polysorbates, polyoxyl 35 castor oil, polyoxyl-15-hydroxystearate, polyvinyl pyrrolidone, propylene glycol, sodium cholesteryl sulfate, sorbitan esters, poloxamer, 2-pyrrolidone, diacylglycerols, monglycerol), tonicity agents (e.g., dextrose, glycerin, mannitol, NaCl, KCl, sorbitol/sorbitol solution), solvents and cosolvents (water miscible such as propylene glycol, PEG low molecular weight, glycerin, ethanol, 2-pyrrolidone, and N-methyl-2-pyrrolidone and water immiscible such as ethyl oleate, benzyl benzoate, vegetable oil, soybean oil, sesame oil, peanut oil, castor oil, almond oil, and cottonseed oil), viscosity-building agents (e.g., sodium carboxymethylcellulose (Na CMC), methylcellulose, gelatin, polyvinyl pyrrolidone), antioxidants (e.g., ascorbic acid, acetylcysteine, sodium ascorbate, sodium metabisulfite, sodium bisulfite, and tocopherol), chelating agents (e.g., EDTA), preservatives (e.g., methyl paraben, ethyl paraben, propyl paraben, butyl paraben, benzoic acid, sodium benzoate, EDTA, cetrimide, benzyl alcohol, benzalkonium chloride, thimerosal, and phenylmercuric salts), buffering agents (e.g., acetate, citrate, tartrate, phosphate, triethanolamine (TRIS) buffer).

For nasal administration, the composition may comprise a suspending agent and thickener (e.g., colloidal carboxymethyl cellulose (CMC) and microcrystalline cellulose (MCC), MC, HPMC, pectin, PEGs), preservatives (e.g., methyl paraben, ethyl paraben, propyl paraben, butyl paraben, benzoic acid, sodium benzoate, EDTA, cetrimide, benzyl alcohol, benzalkonium chloride, thimerosal, and phenylmercuric salts), penetration enhancer (e.g., polysorbates, poloxamers, PEGs, propylene glycol, EDTA), tonicity agents (e.g., dextrose, glycerin, mannitol, NaCl, KCl, sorbitol/sorbitol solution), buffering agents (e.g., acetate, citrate, tartrate, phosphate, triethanolamine (TRIS) buffer). A composition for nebulizer solution may comprise buffering agents (e.g., phosphate buffer, citrate buffer), EDTA chelating agent, ethanol cosolvent, pH modifier (e.g., HCl, NaOH, sulfuric acid, tartaric acid, citric acid,), preservatives (e.g., methyl paraben, propyl paraben, benzalkonium chloride), surfactant (e.g., polysorbate 20, polysorbate 80, sorbitan laurate.), and tonicity agent (e.g., NaCl). A pressurized metered-dose inhaler composition may comprise propellants (e.g., hydrofluoroalkanes), surfactants (e.g., oleic acid, sorbitan trioleate, lecithin), pH modifier (e.g., citric acid), lubricant (e.g., PEG 1000, PEG 600), cosolvent (e.g., ethanol, glycerol), suspending agents (e.g., Povidone K25, K30). A dry powder-optionally lyophilized-inhaler composition may comprise carriers (e.g., lactose monohydrate), surfactants (e.g., dipalmitoyl phosphatidylcholine, distearoyl phosphatidylcholine), lubricant (e.g., magnesium stearate).

For topical administration, the composition may comprise gelling agents (e.g., carbomer, carrageenan, chitosan, gelatin, gellan gum, pectin, ploxamer, poly(ethylene) oxide, polycarbophil, pullulan, HEC, HPMC, MC, alginates, Na CMC, xanthan gum, acacia, agar, guar gum, tragacanth, modified starch, povidone), humectants (e.g., corn syrup, glycerin, lactic acid, PEGs, propylene, glycol, sodium lactate, sorbitol, trehalose, xylitol), cream and ointment bases (e.g., cetostearyl alcohol, cetyl palmitate, fatty alcohols, hard fat, lanolin, lanolin alcohol, hydrogenated castor oil, mineral oil, petrolatum, glyceryl behenate, hard paraffin, soft paraffin, stearic acid, beeswax (white or yellow), carnauba wax, emulsifying wax, microcrystalline wax), solubilizers and emulsifiers (e.g., cocoylcaprylocaprate, decyl oleate, diethylene glycol monoethyl ether, dimethyl isosorbide, glyceryl monooleate, isopropyl myristate, medium chain triglycerides (MCT), octyldodecanol, oleyl alcohol, oleyl oleate, polyoxyethylene alkyl ethers, polyoxyethylene stearates, propylene glycol monocaprylate, propylene glycol monolaurate, sodium cetostearyl sulfate, lecithin, cyclodextrins, docusate sodium, glyceryl monostearate, hydrogenated vegetable/cottonseed/palm kernel oil, MCT, N-methyl-2-pyrrolidone, poloxamer, PEG, polysorbate, PEG castor oil derivatives, propylene glycol, polyoxyglycerides, sodium lauryl sulfate, sucrose esters), suspending agents and thickeners (e.g., magnesium aluminum silicate, MCC and Na CMC, propylene glycol alginate, acacia, carbomer, carrageenan, colloidal silicon dioxide, gellan gum, HEC, HPC, HPMC, maltitol, MC, pectin, PEGs, polyvinyl alcohol, povidone, Na CMC, sorbitol, sucrose, xanthan gum, tragacanth, gelatin, guar gum, kaolin, phospholipids), preservatives (e.g., methyl paraben, ethyl paraben, propyl paraben, butyl paraben, benzoic acid, sodium benzoate, EDTA, cetrimide, benzyl alcohol, benzalkonium chloride, thimerosal, and phenylmercuric salts), and stabilizers including pH modifiers (e.g., lactic acid, citric acid, tartaric acid, ascorbic acid, and their sodium salts), antioxidants referred to above, and chelating agent EDTA.

Excipients for other dosage forms can be found in Remington: The Science and Practice of Pharmacy, 23rd Edition or higher version.

Composition embodiments can be administered once a day, twice a day, thrice a day for one day, couple of days, three days, four days, five days, six days, a week, two weeks, three weeks, a month or a longer period. Daily dose (pfu) is to be determined empirically, depending on the severity of P. aeruginosa infection. For example, patients can be administered with 10⁵-10¹⁵ PFU, optionally 10⁹-10¹¹ PFU of a single phage or combination of at least two phages intravenously twice daily; or with the same dose by inhalational nebulization. Patients can also receive antibiotic treatment with the phage treatment against their own target isolate. Initial duration of phage treatment can be 6 months, but it can be shorter or longer courses of treatment directed by clinical and microbiologic responses. Anti-Pseudomonas regimens can be adjusted by the treating clinicians as needed based on tolerability to phage-containing pharmaceutical formulation and lab tests during phage treatment.

Although the main subject of this invention is human intervention, any animals including both vertebrates and invertebrates, and even some mushrooms, algae and plants, which can be infected by P. aeruginosa, can benefit from treatment with the phages listed above or phage cocktail comprising any of them. For example, the composition comprising the phages or phage cocktail against MRD strains of P. aeruginosa can be scattered as dry powder or solution to the fish tank or aquaculture for oysters, clams, mussels, or other shellfish, or algae farm.

Considering the aspect of bactericidal effect of phages without affecting human immunity as demonstrated in Example 7, the composition can also be developed into hygiene control products. For example, the composition can be utilized as a disinfectant for cleaning the surface of medical device and instruments, and/or hospital furniture or walls, by applying a liquid, aerosol, or powder composition to the surface directly or to wiping cloth.

In addition, a method for quantifying antibiotic effect of a phage or a phage cocktail is disclosed using wax worm larvae. Based on Example 6, the method comprises steps of:
i. injecting a certain amount (cfu) of a bacterial strain, which is to be infected with the phage or phage cocktail, into wax worm larvae;
ii. waiting about 20-60 minutes;
iii. injecting a phage or phage cocktail at various pfu contained in a certain volume of injection solution, into the larvae of step i;
iv. waiting about up to 24 hours;
V. counting surviving larvae and comparing the number of surviving larvae with control larvae, which is not injected with the phage or phage cocktail.

### EXAMPLES

### Example 1. Complete Genome Sequencing of the Novel Pseudomonas aeruginosa Phage UF RH1

### 1.1. Materials and Methods

A P. aeruginosa phage named UF_RH1 was isolated by introducing 10 μL of filtered wastewater collected from a sewage treatment plant (Alexander Orr Water Treatment Plant located at Miami, FL) to 400 μL of P. aeruginosa (strain DJ06) in logarithmic phase and then performing single-plaque isolation using double-layer agar (Jiang et al., 2020). The purity of the phage was confirmed after five rounds of single-plaque isolation. Phage DNA was extracted using a commercial kit (QIAamp DNA Mini kit; Qiagen, USA). The Nextera XT library preparation kit (Illumina, San Diego, CA) was used to create a phage DNA library, which was sequenced on the Illumina NovaSeq 6000 platform with 150-bp paired-end reads. The raw reads were cleaned up with the Cutadapt program v2.8 (Martin, 2011) to remove low-quality bases and sequencing adaptors and then searched against the genome of P. aeruginosa from the NCBI genome database using the read mapper of the STAR package to remove potential host DNA contamination (Dobin et al, 2013). The unmapped paired-end reads were further assembled with the software MetaWRAP v1.2.0 (Uritskiy et al, 2018), and the assembled consensus sequences with a length of more than 5,000 bp were evaluated by QUAST v5.0.2 (Gurevich et al., 2013) and analyzed with the metagenomic software Centrifuge v1.04b (Kim et al, 2016). The quality of the viral genome completeness and identification of closed genomes were assessed using the CheckV v1.01 package (Nayfach et al., 2021). The viral genome candidates were further analyzed with NCBI BLASTn to determine the taxonomic relationships of the phage (Donkor et al., 2014), and Victor was used for phylogenetic analysis (Meier-Kolthoff & Göker 2017). Open reading frames (ORFs) were identified by GeneMarkS (Besemer et al., 2001), and the genome was annotated based on PHASTER (Arndt et al., 2016) and BLASTp results (Donkor et al., 2014). PhageTerm was used to determine the phage termini (Garneau et al., 2017). The IRNA sequences were determined by ERNAscan-SE v2.00 (Lowe & Chan 2016), while virulence factors and antibiotic resistance factors were detected using ResFinder v4.00 (Bortolaia et al., 2020) and the Antibiotic Resistance Genes Database (Liu & Pop 2009), respectively, with default parameters for all software tools.

### 1.2. Results

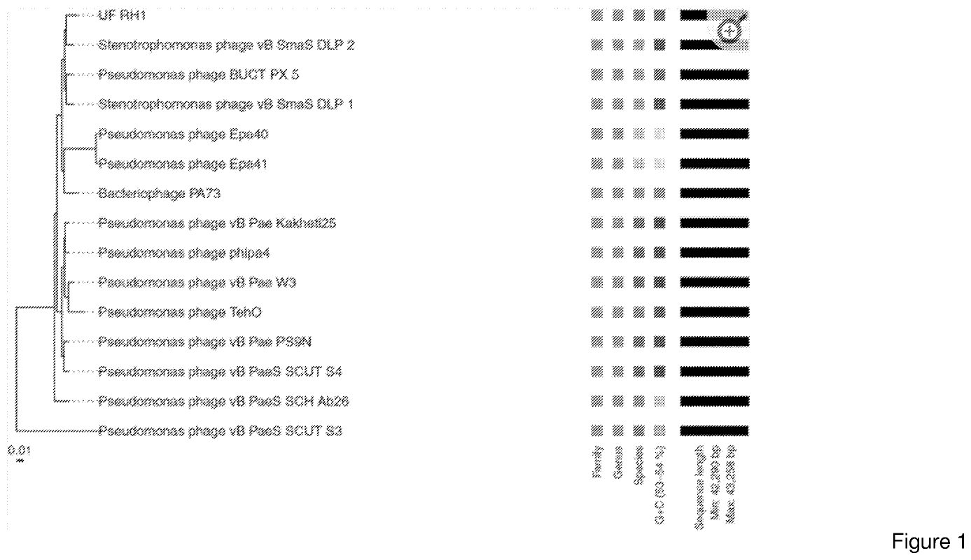

The genome of Pseudomonas phage UF_RH1 is a liner double-stranded DNA (dsDNA) genome with a length of 42,567 bp, a GC content of 53.56%, 931,200 total reads, and 23,360× average read coverage. PhageTerm predicted a circularly permuted genome for UF_RH1. It includes 57 open reading frames (ORFs), and it shares sequence similarity with members of the genus Septimatrevirus (FIG. 1). Stenotrophomonas phage vB_SmaS-DLP_2 (Peters et al., 2015) is a closer phage to UF_RH1 (Table 1). The annotated proteins of UF_RH1, such as dead box helicase, RecB exonuclease, central tail hub, and holin, exhibit 99%, 98%, 98%, and 86.36% similarity, respectively, to the corresponding annotated proteins of Stenotrophomonas phage vB_SmaS-DLP_2. DNA polymerase and endolysin also show the highest similarity of 99.8% and 92%, respectively, to the proteins encoded by Pseudomonas phage TehO. The tail length tape-measure protein also displays the highest similarity of 99.58% to the tail protein of Pseudomonas phage vB_PaeS_C1. UF_RH1 does not contain tRNA, virulence, and antibiotic-resistant genes.

TABLE 1
Genome sequence coverage and nucleotide identity of UF_RH1 with their closest relatives ^{a}

| | Sequence coverage (nucleotide identity) of: | | | | | | | |
|---|---|---|---|---|---|---|---|---|
| | | | | | | | vB | |
| Phage (GenBank | | vB Pae- | vB SmaS- | | | | PaeS_SCUT- | |
| accession no.) | UF_RH1 | Kakheti25 | DLP_2 | Epa41 | Epa40 | phipa4 | S4 | PA73 |
| UF_RH1 | 100 | 95 | 97 | 92 | 91 | 95 | 94 | 94 |
| (OQ259603.1) | (100) | (96.2) | (97.8) | (94.8) | (94.8) | (94.1) | (97.2) | (94) |
| vB Pae-Kakheti25 | 95 | 100 | 95 | 91 | 90 | 97 | 98 | 93 |
| (IQ307387.1) | (96.2) | (100) | (97.08) | (96.8) | (96.8) | (97.9) | (96.8) | (97.6) |
| vB SmaS-DLP_2 | 97 | 95 | 100 | 89 | 89 | 95 | 95 | 93 |
| (KR537871.1) | (97.8) | (97.08) | (100) | (97.36) | (98.37) | (97) | (97.3) | (98.33) |
| Epa41 | 92 | 91 | 89 | 100 | 99 | 89 | 89 | 92 |
| (MI118305.1) | (94.8) | (96.8) | (97.36) | (100) | (100) | (97.5) | (98.34) | (97.67) |
| Epa40 | 91 | 90 | 89 | 99 | 100 | 89 | 89 | 92 |
| (MT118304.1) | (94.8) | (96.8) | (98.37) | (100) | (100) | (97.5) | (98.34) | (97.75) |
| phipa4 | 95 | 97 | 95 | 89 | 89 | 100 | 98 | 94 |
| (OK539825.1) | (94.1) | (97.9) | (97) | (97.5) | (97.5) | (100) | (96.88) | (98) |
| vB PaeS)_SCUT-S4 | 94 | 98 | 95 | 89 | 89 | 98 | 100 | 91 |
| (MK165658.1) | (97.2) | (96.8) | (97.3) | (98.34) | (98.34) | (96.88) | (100) | (97.64) |
| PA73 | 94 | 93 | 93 | 92 | 92 | 94 | 91 | 100 |
| (DQ163913.1) | (94) | (97.6) | (98.33) | (97.67) | (97.75) | (98) | (97.64) | (100) |

^{a} All values are %. All phages are classified as Pseudomonas phage except vB_SmaS-DLP_2, which is classified as Stenotrophomonas phage.

### Data Availability:

The complete phage genome sequence was deposited in GenBank under the accession number OQ259603. The raw data are available in the NCBI Sequence Read Archive (SRA) under BioProject accession number PRJNA936202, SRA accession number SRS16838829, and BioSample accession number SAMN33343989.

### Example 2. Complete Genome Sequencing of a Novel Pseudomonas aeruginosa Phage, UF RH5

### 2.1. Materials and Methods

A P. aeruginosa phage named UF_RH5 was isolated by adding filtered wastewater (10 uL) collected from a sewage treatment plant (Alexander Orr Water Treatment Plant located at Miami, FL) to strain DJ06 of P. aeruginosa (400 μL) in logarithmic phase. Using double-layer agar, a single plaque was isolated (Jiang et al., 2020), and the purity of the phage was verified via single plaque isolation. Phage DNA was extracted from phage lysate (5 mL) using a QIAamp MinElute virus kit (Qiagen, USA). The Illumina Nextera XT library preparation kit was used for DNA library preparation, and sequencing was carried out using a Illumina NovaSeq 6000 instrument (paired-end 150-bp cycle). Bcl2fastq v2.20 was utilized to demultiplex reads, and Cutadapt program v2.8 was used to remove sequencing adaptors and low-quality bases (Martin 2011). Using the read mapper of the STAR package, P. aeruginosa DNA was removed from the data (Dobin et al., 2013). The unmapped paired-end reads were then assembled using Meta WRAP v1.2.00 (Uritskiy et al, 2018), and the assembled consensus sequences with lengths of >5,000 bp were evaluated by QUAST v5.0.2 (Gurevich et al., 2013). Centrifuge v1.04b was utilized to analyze the assembled consensus sequences (Kim et al., 2016). CheckV v1.01 was applied to evaluate the viral genome completeness and to identify closed genomes (Nayfach et al., 2021). The taxonomic identity of the virus was characterized by NCBI BLASTn using the nucleotide collection database (Donkor et al., 2014). Victor was used for phylogenetic analysis (Meier-Kolthoff & Göker 2017). GeneMarkS was used to identify open reading frames (ORFs) (Besemer et al., 2001). The genome was annotated based on PHASTER (Arndt et al., 2016) and BLASTp results (Donkor et al., 2014). We used a comprehensive database of nonidentical protein sequences using the default threshold (expect threshold=0.05 and matrix=blosum62) as the reference database for the BLASTp search. PhageTerm was used to determine the phage termini (Garneau et al., 2017). (RNA sequences were determined by tRNAscan-SE (Lowe & Chan 2016). To detect virulence factors and antibiotic resistance factors, ResFinder v4.0 (Bortolaia et al., 2020) and the Antibiotic Resistance Genes Database (Liu & Pop 2009) were used, respectively. Default parameters were used for each software.

### 2.2. Results

The genome of Pseudomonas phage UF_RH5 is a linear double-stranded DNA (dsDNA) with a length of 42,566 bp and a GC content of 53.60%, which was sequenced with an average read coverage of 21,320× and a total of 912,112 reads. PhageTerm predicted a circularly permuted genome for UF_RH5. The genome comprises 58 ORFs and belongs to the Siphovirus morphology family and the genus Septimatrevirus, as evidenced by sequence similarities with other members (Table 2 and FIG. 2). At genome sequence level, Pseudomonas phage vB_PacS_SCUT-S4 is close to UF_RH5. The genome of UF_RHS did not contain (RNA, virulence, or antibiotic-resistant genes. These results confirm the classification of UF_RH5 as a member of the Siphovirus morphology family and the genus Septimatrevirus.

TABLE 2
Genome sequence coverage and nucleotide identity of UF_RH5 with their closest relatives ^{a}

| | Percentage of sequence coverage (similarity) of: | | | | | | | |
|---|---|---|---|---|---|---|---|---|
| Phage | | vB Pae- | | | | | vB PaeS_SCUT- | |
| (GenBank | | [symbol] | vB SmaS-DLP_2 | Epa41 | Epa40 | phipa4 | S4 | PA73 |
| accession no.) [symbol] | UF_RH5 | ( [symbol] ) | ( [symbol] ) | ( [symbol] ) | ( [symbol] ) | ( [symbol] ) | ( [symbol] ) | ( [symbol] ) |
| UF_RH5 | 100 | 95 | 97 | 92 | 91 | 95 | 94 | 94 |
| ( [symbol] ) | (100) | (96.2) | (97.8) | (94.8) | (94.8) | (94.1) | (97.2) | (94) |
| vB Pae- | 95 | 100 | 95 | 91 | 90 | 97 | 98 | 93 |
| [symbol] 25 | (96.8) | (100) | (97.1) | (96.8) | (96.8) | (97.9) | (96.8) | (97.6) |
| ( [symbol] ) | | | | | | | | |
| vB SmaS-DLP_2 | 97 | 95 | 100 | 89 | 89 | 95 | 95 | 93 |
| ( [symbol] ) | (97.8) | (97.08) | (100) | (97.36) | (98.37) | (97) | (97.3) | (98.3) |
| vB_Pae_P59N | 95 (97) | 91 | 89 | 100 | 99 | 89 | 89 | 92 |
| ( [symbol] ) | | (96.8) | (97.36) | (100) | (100) | (97.5) | (98.34) | (97.8) |
| vB_Xar_ [symbol] | 94 | 90 | 69 | 99 | 100 | 83 | 89 | 92 |
| DoCa11 | (96.7) | (96.8) | (98.37) | (100) | (100) | (97.5) | (98.34) | (97.7) |
| ( [symbol] ) | | | | | | | | |
| phipa4 | 95 | 97 | 95 | 89 | 89 | 100 | 98 | 94 |
| ( [symbol] ) | (96.9) | (97.9) | (97) | (97.5) | (97.5) | (100) | (96.88) | (98) |
| vB PaeS_SCUT- | 94 | 98 | 95 | 89 | 89 | 98 | 100 | 91 |
| S4 | (97.3) | (96.8) | (97.3) | (98.3) | (98.3) | (96.3) | (100) | (97.6) |
| ( [symbol] ) | | | | | | | | |
| [symbol] | 96 | 98 | 98 | 92 | 92 | 94 | 91 | 100 |
| ( [symbol] ) | (97.5) | (97.6) | (98.33) | (97.67) | (97.75) | (98) | (97.64) | (100) |

^{a} All phages are classified as Pseudomonas phage except vB_SmaS-DLP_2 and vB_Xar_IVIA-DoCa11 which is classified as Stenotrophomonas and Xanthomonas phages, respectively. [symbol]
indicates data missing or illegible when filed

Data availability.

The complete phage genome sequence was deposited in GenBank under the accession number OQ319036. The raw data are available in the NCBI Sequence Read Archive (SRA) under BioProject accession number PRJNA938088, SRA accession number SRR23613576, and BioSample accession number SAMN33424506. Raw sequence data include unmapped nonphage reads.

### Example 3. Complete Genome Sequence of Pseudomonas aeruginosa Phage UF RH6, Isolated from Human Lung

### 3.1. Materials and Methods

Respiratory secretions were obtained from a 65-year-old male patient who was diagnosed with Pseudomonas aeruginosa pneumonia and was admitted to the University of Florida hospital (University of Florida Health, Jacksonville, FL, USA). The collection of human leftover samples for phage isolation was granted ethical approval by the University of Florida institutional review boards (approval number IRB202102636). A respiratory secretion sample (1 mL) was mixed with SM buffer (9 mL; Thermo Fisher Scientific, USA) and filtered through a 0.2-μm syringe filter. Then, 10 μL of filtered sample was added to 400 μL of P. aeruginosa (strain DJ06) cultured in brain heart infusion broth and was incubated at 37° C. for 20 min. The phage was isolated using double-layer agar by incubating the plates at 37° C. for 24 h (Jiang et al., 2020). The phage was purified via single plaque isolation. DNA was extracted from phage lysate (5 mL) using the QIAamp MinElute Virus kit (Qiagen, USA). The Illumina Nextera XT library preparation kit was used for DNA library preparation, and sequencing was carried out using an Illumina NovaSeq 6000 system (paired-end 150-cycle mode). Bcl2fastq v2.20 (Illumina) was utilized to demultiplex reads, and Cutadapt v2.8 was used to remove sequencing adaptors and low-quality bases (Martin, 2011). Using the read mapper of the STAR package, P. aeruginosa DNA was removed from the data (Dobin et al., 2013). The unmapped paired-end reads were then assembled using Meta WRAP v1.2.00 (Uritskiy et al., 2018), and the resulting consensus sequences with lengths of >5,000 bp were evaluated by QUAST v5.0.2 (Gurevich et al, 2013). Centrifuge v1.04b was utilized to analyze the assembled consensus sequences (Kim et al., 2016). CheckV v1.01 was applied to evaluate the viral genome completeness and to identify closed genomes (Nayfach et al., 2021). The taxonomic identity of the virus was characterized by NCBI BLASTn (Donkor et al., 2014). PhageTerm was used to determine the phage termini (Garneau et al., 2017). VICTOR was used for phylogenetic analysis (Meier-Kolthoff & Göker 2017). GeneMarkS was used to identify open reading frames (ORFs) (Besemer et al., 2001). The genome was annotated based on PHASTER (Arndt et al., 2016) and BLASTp (Donkor et al., 2014) results, and IRNA sequences were determined by tRNAscan-SE (Lowe & Chan 2016). ResFinder v4.0 (Bortolaia et al., 2020) was used to detect virulence factors, and the Antibiotic Resistance Genes Database (ARDB) (Liu & Pop. 2009) was used to detect antibiotic resistance factors. Default parameters were used for all software.

### 3.2. Results

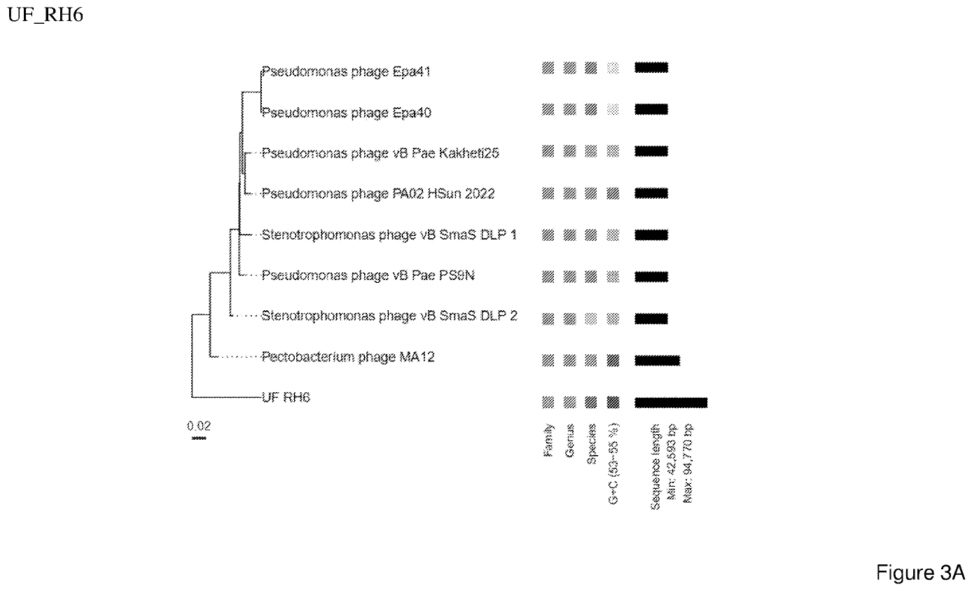

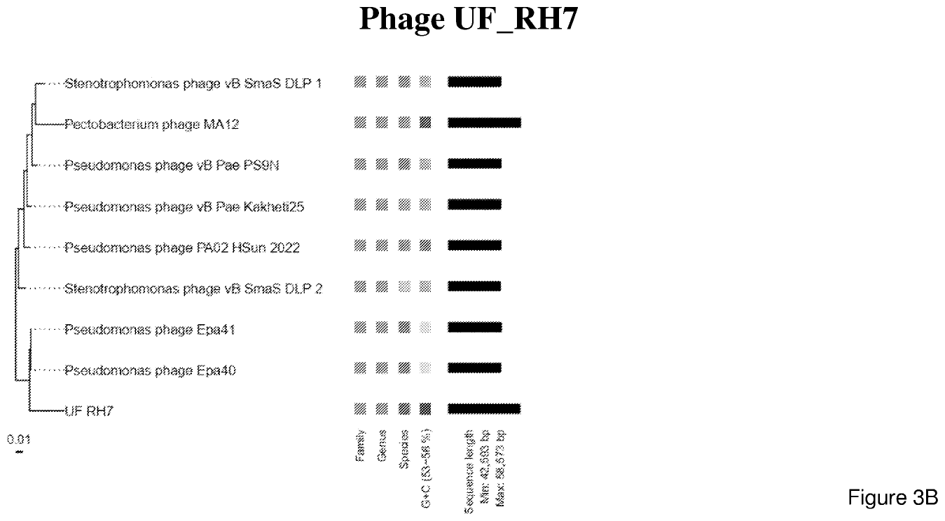

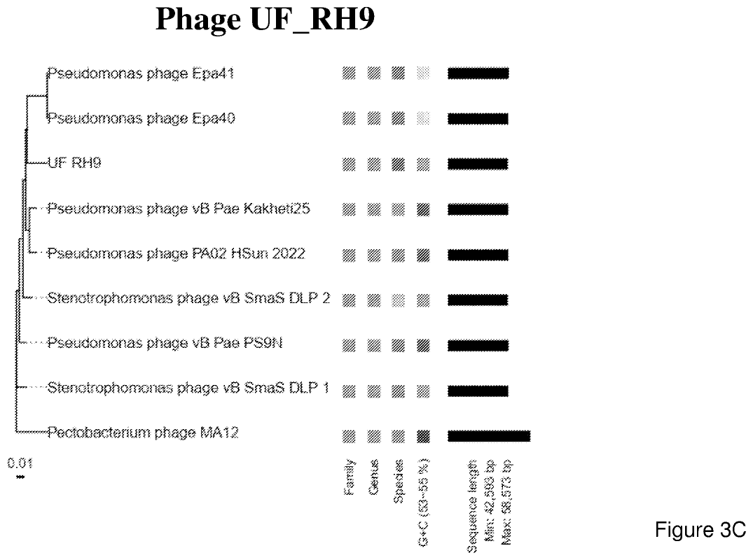

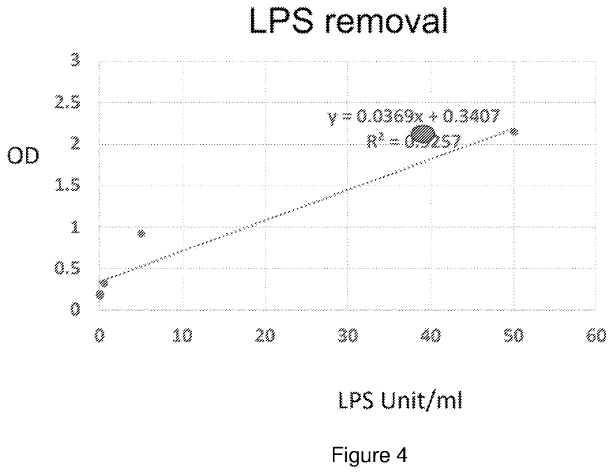

For the sequenced sample, 24,108,049 raw reads (150-bp read length) were obtained. After removal of host DNA, only one contig with a length of >5,000 bp (94,715 bp) was assembled from the remaining reads (15.8% [3,809,071 reads]), with coverage of 10,768×. The genomic structure of Pseudomonas phage UF_RH6 (GenBank accession number OQ383211.1) is composed of linear double-stranded DNA, spanning a length of 94,715 bp and exhibiting a GC content of 55.30%, PhageTerm predicted a circularly permuted genome for UF_RH6. CheckV results showed the completeness of the sequence. The genome comprises 130 ORFs and belongs to the family Caudoviricetes and the genus Samunavirus, as evidenced by similarities to other members (Table 3 and FIG. 3). UF_RH6 shows the greatest nucleotide identity (99.08%) to Pseudomonas phage SM1 (GenBank accession number NC_041877.1). The genome of UF_RH6 contains a IRNA sequence; however, no virulence or antibiotic resistance genes were detected.

TABLE 3
Genome sequence coverage and nucleotide identity of UF_RH5 with respect to its closest relatives ^{a}

| | Sequence coverage (%)/identity (%) with respect to phage: | | | | | |
|---|---|---|---|---|---|---|
| | | | vB_PaeS_FBPa4S | Pa BHU-15 | Pa BHU-17 | BHU-1 |
| | UF_RH6 (GenBank | SM1 (GenBank | (GenBank | (GenBank | (GenBank | (GenBank |
| | accession no. | accession no. | accession no. | accession no. | accession no. | accession no. |
| Phages | [symbol] ) | [symbol] ) | [symbol] ) | [symbol] ) | [symbol] ) | [symbol] ) |
| [symbol] (GenBank | 100/100 | 97/99.08 | 97/98.8 | 88/96.8 | 87/96.6 | [symbol] |
| accession no. | | | | | | |
| [symbol] ) | | | | | | |
| SM1 (GenBank | 97/99.08 | 100/100 | 99/98.58 | 80/98.5 | 89/98.39 | 90/98.46 |
| accession no. | | | | | | |
| [symbol] ) | | | | | | |
| [symbol] (GenBank | 97/98.8 | 99/98.58 | 100/100 | 90/98.1 | 89/98.39 | 90/98.99 |
| accession no. | | | | | | |
| [symbol] ) | | | | | | |
| Pa BHU-15 (GenBank | 88/90.8 | 90/98.0 | 90/98.1 | 100/100 | 93/100 | 93/100 |
| accession no. | | | | | | |
| [symbol] ) | | | | | | |
| Pa BHU-17 (GenBank | 87/96.6 | 89/98.39 | 89/98.39 | 93/100 | 100/100 | 93/100 |
| accession no. | | | | | | |
| [symbol] ) | | | | | | |
| BHU-1 (GenBank | 87/97.1 | 90/98.46 | 90/98.99 | 93/100 | 93/100 | 100/100 |
| accession no. | | | | | | |
| [symbol] ) | | | | | | |

^{a} All phages are classified as Pseudomonas phages. [symbol]
indicates data missing or illegible when filed

### Data Availability.

The complete phage genome sequence was deposited in GenBank under accession number OQ383211.1. The raw data are available in the NCBI Sequence Read Archive (SRA) under BioProject accession number PRJNA941099, SRA accession number SRR23702725, and BioSample accession number SAMN33589860.

### Example 4. Genome Sequencing of Pseudomonas aeruginosa Phages; UF RH7 and UF RH9

### 4.1. Materials and Methods

The collected raw sewage sample from a treatment plant (Alexander Orr Water Treatment Plant located at Miami, Florida, USA) was filtered through 0.2 micron filter and was added to fresh broth culture of P. aeruginosa (strain DJ06) (10 μL sewage was mixed with 400 μL bacteria). The mixture was incubated at 37° C. (20 min) and was cultured using double-layer agar by incubating the plates at 37° C. (24 h) (Jiang et al., 2020). Two different phages were isolated. The phages were purified via single plaque isolation and then purified after five rounds of single plaque isolation. DNA was extracted from 5 mL lysate of each phage (QIAamp MinElute Virus Kit, Qiagen, USA). DNA library for phages were prepared using Illumina's Nextera XT library preparation kit and were sequenced using Illumina NovaSeq 6000 (paired-end 150 cycle), Bcl2fastq v2.20 (Illumina) was used to de-multiplex reads. Cutadapt program v2.8 was utilized to remove sequencing adaptors and low-quality bases (Martin 2011). P. aeruginosa DNA was removed from the data using the read mapper of the STAR package v.2.5 (Dobin et al., 2013). MetaWRAP v1.2.00 (Uritskiy et al., 2018) was used to assemble the unmapped paired-end reads using NCBI Viral databases. QUAST v5.0.2 (Gurevich et al., 2013) was used to measure the quality analysis of the data, Centrifuge v1.04b (Kim et al., 2016) was used to analyze the assembled consensus sequences (>15,000 bp). Each phage had only one assembled contig over 15,000 bp. Consend v.29 was used to determine to phage termini (Gordon & Green, 2013). Check V v1.0.1 was used to determine the completeness and accuracy of phage genome (Nayfach et al., 2021). Taxonomic identity of the sequences was determined by NCBI BLASTn [Nucleotide collection (nr/nt)] (Donkor et al., 2014). Open reading frames (ORFs) were defined for each virus by GeneMarkS v2 (Besemer et al., 2001), and the genome of each phage was annotated by PHASTER and BLASTp (non-redundant protein sequence) (Donkor et al., 2014; Arndt et al., 2016). The presence of ERNA sequences, virulence factors, antibiotic resistance genes were evaluated by (RNAscan-SE v2.0 (Lowe & Chan 2016), ResFinder v4.0 (Bortolaia et al., 2020), and Antibiotic Resistance Genes Database (Liu & Pop 2009), respectively. Default parameters were used for software tools.

### 4.2. Results

After post trimming and filtration of bacterial host sequences, the genome was assembled for both phages. The Pseudomonas phage UF_RH7 genome is a 58,217 bp long with circular double-stranded DNA (dsDNA) genome with a GC content of 56.4%, 21,196,642 total reads, and 29,432×average read coverage. The Pseudomonas phage UF_RH9 genome is a 42,609 bp long with linear dsDNA genome with a GC content of 53.6%, 53,009,336 total reads, and 21,391×average read coverage.

CheckV results showed high-quality (>90% completeness) and high confidence (0%-5% error) of the genome of each phage. UF_RH7 and UF_RH9 include 82 and 55 ORFs, respectively. UF RH7 only shares high DNA similarity (~81%) and 70% sequence coverage with Pectobacterium phage MA11 (GenBank: NC_053014) and Pectobacterium phage MA12 (GenBank: NC_053015). UF_RH9 shares high DNA similarity to our previously reported Pseudomonas phages UF_RH1 (Hashemi et al, 2023a) and UF_RH5 (Hashemi et al., 2023b) (Table 4). In the genomes of both phages, we did not detect any tRNA sequences, virulence genes, or antibiotic resistance genes.

TABLE 4
Genome sequence coverage (top number in each cell) and nucleotide
identity (bottom number) of UF_RH9 with their closest relatives

| Phages | Percentage of sequence coverage and similarity | | | | | | |
|---|---|---|---|---|---|---|---|
| {GenBank | | | | vB Pae- | PA02_HSun- | | |
| accession | | UF_RH1 | UF-RH5 | Kakheti25 | 2022 | Epa41 | Epa40 |
| number} | UF_RH9 | (NC_072810.1) | (OQ319036.1) | (IQ307387.1) | (OM234791.1) | (MT118305.1) | (MT118304.1) |
| UF_RH9 | 100 | 99 | 99 | 95 | 95 | 94.8 | 94.8 |
| | 100 | 99 | 100 | 96.2 | 97.2 | 92 | 91 |
| UF_RH1 | 99 | 100 | 99.9 | 96.2 | 97.2 | 97 | 94.8 |
| (NC_072810.1) | 99 | 100 | 99 | 95 | 95 | 97.9 | 92 |
| UF_RH5 | 99 | 99.9 | 100 | 96.8 | 97.2 | 95 | 97.2 |
| (OQ319036.1] | 100 | 99 | 100 | 95 | 95 | 97 | 92 |
| vB Pae- | 95 | 96.2 | 96.8 | 100 | 98 | 96.8 | 96.8 |
| Kakheti25 | 96.2 | 95 | 95 | 100 | 98 | 91 | 90 |
| (IQ307387.1) | | | | | | | |
| PA02_HSun-2022 | 95 | 97.2 | 97.2 | 98 | 100 | 97.2 | 97.2 |
| (OM234791.1) | 97.2 | 95 | 95 | 98 | 100 | 92 | 91 |
| Epa41 | 94.8 | 94.8 | 97.2 | 96.8 | 97.2 | 100 | 100 |
| (MT118305.1) | 92 | 92 | 92 | 91 | 92 | 100 | 99.9 |
| Epa40 | 94.8 | 94.8 | 97.2 | 96.8 | 97.2 | 100 | 100 |
| (MT118304.1) | 91 | 91 | 91 | 90 | 91 | 99.9 | 100 |

### Data Availability

The complete phage genome sequence was deposited in GenBank under the accession numbers OR589135 and OR604635 for UF_RH7 and UF_RH9, respectively. The raw data for UF_RH7 are available in the NCBI Sequence Read Archive (SRA) under BioProject accession number PRJNA1019333, SRA accession number SRR26125953, and BioSample accession number SAMN37480981. The raw data for UF_RH9 are available in the NCBI Sequence Read Archive (SRA) under BioProject accession number PRJNA1021600, SRA accession number SRR26223087, and BioSample accession number SAMN37563411.

### Example 5. Antibiotic Effect of Individual Phages and Phage Cocktail

The isolated phages (i.e., RH1, RH5, RH6, RH7 and RH9) and phage cocktails comprising 3 or all 5 of them were tested against P. aeruginosa. FIG. 5 shows the results of phage test against P. aeruginosa. The treatment of bacteria with individual phages, RH1, RH5, RH6, RH7 and RH9, Cocktail 1, which is a blend of three specific phages, and Cocktail 2, which is a blend of five specific phages (RH1, RH5, RH6, RH7 and RH9) suppressed bacterial growth.

In this experiment, monophage treatment, which involves the application of only a single phage when sufficiently wide host-range phages are available, showed some successes. However, phage cocktails (multiple phage types possessing a diversity of host-ranges) may be a better way to increase the success of phage therapy by reducing the risk of the development of phage resistant bacteria. The different potential susceptibility of the bacterial strains to the phages can be addressed by using a phage cocktail to treat the wide range of bacterial strains/species that can cause clinical infections.

Further, in FIG. 7, the antibacterial effect of the phage cocktail on a P. aeruginosa strain resistant to quinolone antibiotics, was compared with Cipro, and proved that the phage cocktail was as effective as antibiotic. Combination use of the phage cocktail and AB further reduced bacteria growth. This result suggests that a phage cocktail can be a new approach to replace or to be used with antibiotics against drug-resistant bacteria.

### Example 6. In Vivo Test of the Phage Cocktail's Antibacterial Effect

### 6.1. Materials and Methods

The study utilized a detailed and controlled experimental approach to investigate the impact of bacteriophage cocktails on the survival of larvae infected with Pseudomonas aeruginosa.

Initially, larvae were carefully weighed, and only those with a body weight of approximately 25 mg were selected for inclusion in the study. These selected larvae were then randomly assigned into groups to ensure a balanced distribution for the experiment.

The inoculation process involved the administration of P. aeruginosa directly into the larvae. This was achieved using an insulin syringe equipped with a 26-gauge needle, through which a precise 10-microliter volume containing the bacteria was injected into each larva. The bacterial concentration was standardized to 3000 Colony Forming Units (CFU) to maintain consistent infection levels across all experimental groups.

Following the bacterial injection, a 30-minute interval was observed to allow for the initial interaction between the larvae and the bacterial pathogen. Subsequently, a bacteriophage cocktail was introduced to the system, employing the same injection technique to ensure accuracy and consistency. Each larva received a 10-microliter dose of the phage cocktail, with phage concentrations varying across the different experimental groups. These concentrations were quantified in Plaque Forming Units (PFU), allowing for a precise measurement of the phage dose administered.

Statistical analysis, including correlation coefficients and regression models, was applied to understand the relationships between phage concentration, bacterial counts, and larvae survival rates.

### 6.2. Results

The primary aim was to assess the effect of different phage concentrations on the ability of P. aeruginosa to proliferate within the larval hosts and to determine how these concentrations influence larval survival rates. Survival was monitored and recorded over a 24-hour period following phage administration, providing critical data on the interaction between the bacteriophages, the bacterial pathogen, and the larvae.

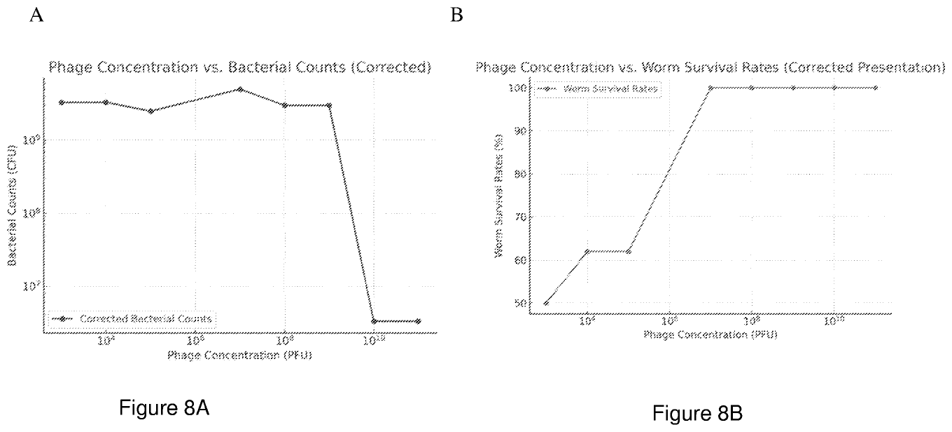

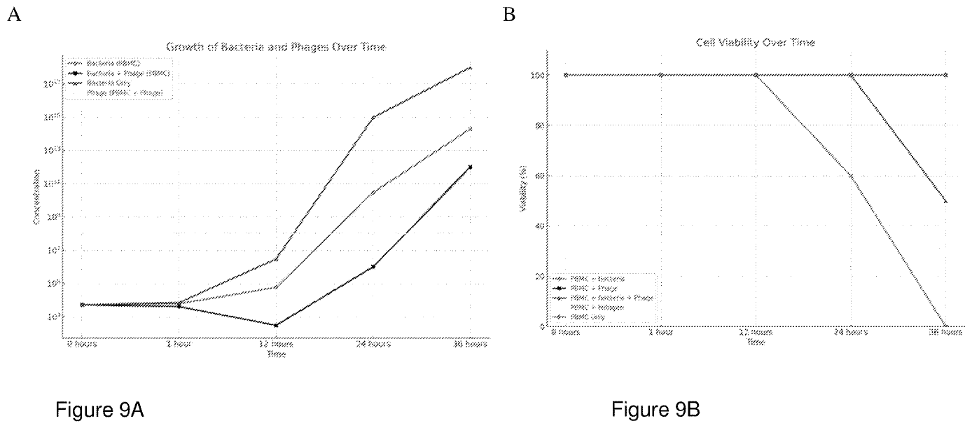

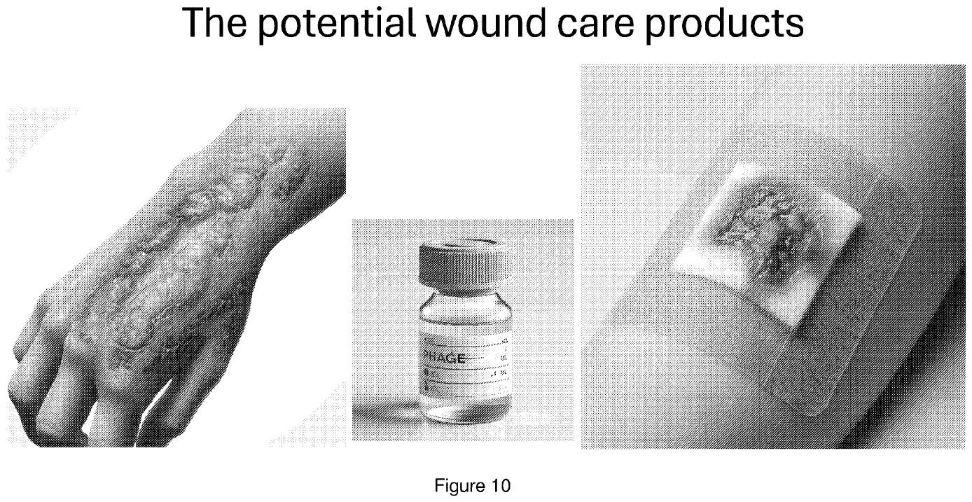

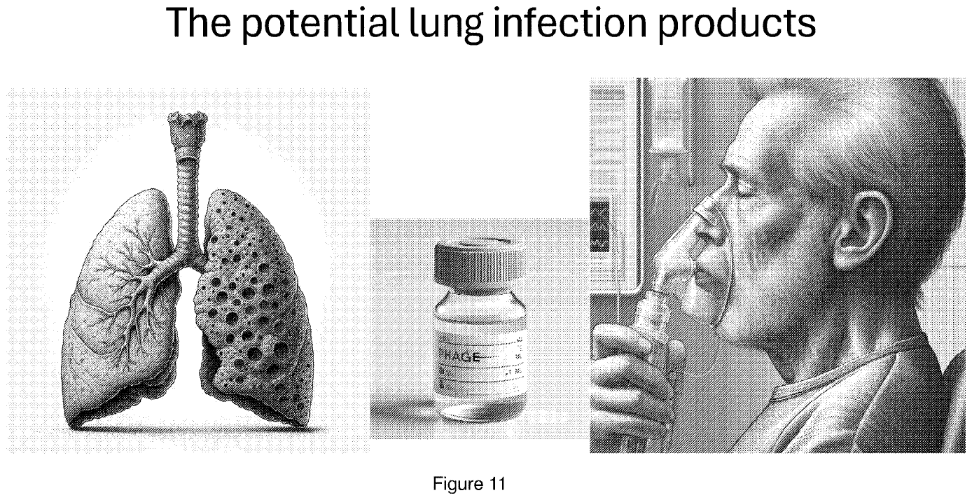

At a high phage concentration (1011 PFU), significant suppression of bacterial proliferation was observed, with larvae survival at 100%, and a decrease in phage concentration led to increased bacterial counts, indicating lower phage effectiveness (FIG. 8). At a phage concentration below 105 PFU, larvae survival dropped to 62%, further declining to 50% at 103 PFU.

FIG. 8A shows phage concentration vs. bacterial counts. In the graph, the slope is-0.342, the intercept is 25.793, and the R² score is 0.509. This model indicated a negative correlation between phage concentration and bacterial counts, with approximately 50.9% of the variability in bacterial counts explained by phage concentration.

FIG. 8B shows phage concentration vs. larvae survival. In the graph, the slope is 2.993, the intercept is 35.147, and the R² score is 0.822. A positive correlation was found between phage concentration and larvae survival, with about 82.2% of variability in survival rates explained by phage concentration.

TABLE 5
Summary of the wax worm larvae test results.

| | 24 hours after exposure | | |
|---|---|---|---|
| | | Phage counts | Pseudomonas |
| Time 0 | %, N = 8 | (PFU) | Counts (CFU) |
| Worm alive | 100% | — | — |
| Phage: 10¹¹ PFU | — | 2 × 10¹⁰ | — |
| Bacteria: 3000 CFU | — | — | 3.3 × 10⁶ |
| Worm alive | 100% | | — |
| Phage: 10¹⁰ PFU | — | 3 × 10⁹ | — |
| Bacteria: 3000 CFU | | — | 3.3 × 10⁶ |
| Worm alive | 100% | — | — |
| Phage: 10⁹ PFU | — | 3 × 10⁹ | — |
| Bacteria: 3000 CFU | — | — | 5.50 × 10⁷ |
| Worm alive | 100% | — | — |
| Phage: 10⁸ PFU | — | 3 × 10⁹ | — |
| Bacteria: 3000 CFU | — | — | 1.80 × 10⁸ |
| Worm alive | 100% | — | — |
| Phage: 10⁷ PFU | — | 5 × 10⁹ | — |
| Bacteria: 3000 CFU | — | — | 2.64 × 10⁸ |
| Worm alive | 62% | — | — |
| Phage: 10⁵ PFU | — | 2.5 × 10⁹ | — |
| Bacteria: 3000 CFU | — | — | 9.48 × 10⁸ |
| Worm alive | 62% | — | — |
| Phage: 10⁴ PFU | — | 3.3 × 10⁹ | — |
| Bacteria: 3000 CFU | — | — | 1.8 × 10⁹ |
| Worm alive | 50% | — | — |
| Phage: 10³ PFU | — | 3.3 × 10⁹ | — |
| Bacteria: 3000 CFU | — | — | 5.85 × 10⁹ |

### Example 7. The Effect of Phages on Pathogens in the Immune Arena: Deciphering Bacterial Dynamics and Host Responses for Next-Generation Therapeutics

### 7.1. Materials and Methods

### 7.1.1. Study Design and Experimental Groups

This study employed an in vitro experimental design to investigate the interactions between bacteriophages, bacteria, and peripheral blood mononuclear cells (PBMCs). Five main experimental groups were established: PBMC+Bacteria, PBMC+Phage, PBMC+Bacteria+Phage, PBMC+Mitogen (as a positive control for immune activation), and Bacteria only, alongside a control group comprising PBMCs only to assess baseline cell viability.

### 7.1.2. Bacterial and Phage Cultures

Bacterial cultures were initiated using a specific strain with an initial concentration of 5000 colony-forming units (CFU) for relevant groups. Phage cultures were prepared with a starting concentration of 100,000,000 plaque-forming units (PFU) for groups involving phage treatment. The phages selected were known to specifically target the bacterial strain used, ensuring the relevance of observed interactions.

### 7.1.3. PBMC Isolation and Culture

PBMCs were isolated from human blood samples using density gradient centrifugation. The cells were then cultured in appropriate media and conditions to maintain viability and functionality, ensuring that the immune response could be accurately assessed.

### 7.1.4. Co-Culture Conditions

For groups involving co-cultures of bacteria, phages, and PBMCs, the respective cultures were combined under sterile conditions, with careful monitoring to maintain the integrity and viability of each component. The experimental setup allowed for the simultaneous assessment of bacterial growth, phage activity, and cell viability over time.

### 7.1.5. Data Collection and Analysis

Bacterial growth was quantified at 0, 1, 12, 24, and 36 hours post-inoculation using standard plate count methods. Phage concentration was determined using plaque assays. Cell viability was assessed using trypan blue exclusion, with percentages of live cells recorded at each time point. Statistical analyses were performed to compare growth rates, phage stability, and viability across different conditions, with a particular focus on the impact of phage presence in the PBMC+Bacteria+Phage group.

### 7.2 Results

The findings from this study offer valuable insights into the interactions between bacteria, phages, and peripheral blood mononuclear cells (PBMCs), with significant implications for understanding bacterial growth dynamics and phage therapy applications. Here, we discuss the implications of these results, focusing on bacterial growth, phage stability, and cell viability in various experimental conditions.

### 7.2.2. Bacterial Growth Dynamics

The initial bacterial concentration set at 5000 colony-forming units (CFU) provided a controlled baseline for observing the impact of phages and PBMCs on bacterial proliferation. Notably, the presence of PBMCs alone led to an increase in bacterial numbers, albeit at a slower rate compared with bacteria cultured without any intervention. This suggests that while PBMCs may have some limiting effect on bacterial growth, they do not actively reduce bacterial numbers without the presence of phages.

At the initial time (Time 0), the concentration of bacteria was 5000 CFU in groups where bacteria were present (PBMC+ bacteria, PBMC+ bacteria+phage, and Bacteria only).

After 1 hour, the bacteria grew to 6000 units in the PBMC+ bacteria group, decreased to 4000 units in the PBMC+ bacteria+phage group, and increased to 7000 units in the bacteria only group.

At 12 hours, significant growth was observed in the bacteria only group (3,000,000 units), while the PBMC+ bacteria group grew to 58,000 units, and the PBMC+ bacteria+phage group significantly reduced to 300 units.

By 24 hours, there was an explosive growth of bacteria in the bacteria only group, reaching 1E+15 units. The PBMC+ bacteria group reached 30,000,000,000 units, and the PBMC+ bacteria +phage group had 1,000,000 units.

At 36 hours, the bacteria in the bacteria only group reached 1E+18 units, the PBMC+ bacteria group had 2E+14 units, and the PBMC+ bacteria+Phage group had 1E+12 units.

The addition of phages to cultures containing both bacteria and PBMCs resulted in a marked reduction in bacterial growth. This effect was most pronounced at the 12-hour mark, where bacterial units in the PBMC+ bacteria+phage group drastically decreased to 300 units, contrasting sharply with the explosive growth observed in the Bacteria only group. These findings underscore the efficacy of phages in controlling bacterial populations, even in the presence of host immune cells like PBMCs. However, the eventual increase in bacterial numbers in the PBMC+ bacteria+phage group by 36 hours to 1E+12 units, although significantly lower than the Bacteria only group, indicates a complex interplay between bacterial resistance mechanisms, phage lytic activity, and the immune response.

### 7.2.3. Phage Growth Stability

Phages were initially at 100,000,000 PFU in the PBMC+phage and PBMC+ bacteria+phage groups. This concentration did not change over the observed time period, remaining constant at 100,000,000 PFU throughout the experiment in both the PBMC+phage and PBMC+ bacteria+phage groups. This constancy suggests that, in the provided experimental conditions, phages were able to maintain their population size without significant replication or decline. This could be attributed to a balance between phage lysis of bacterial cells and the rate of phage inactivation or removal by PBMCs. The steady phage numbers also indicate their potential for sustained bacterial control without rapid depletion, an important consideration for phage therapy applications.

### 7.2.4. Cell Viability Insights

Cell viability decreased over time in cultures with mitogens (positive control) and those mixed with bacteria and phages, indicating the detrimental impact of bacterial growth even in the phage presence on cell health. For instance, cell viability in the PBMC+mitogen group decreased from 100% at the start to 30% at 36 hours. Similarly, in the PBMC+ bacteria+phage group, cell viability was 100% alive until 24 hours and dropped to 50% at 36 hours, highlighting the complex interactions at play. But viability of PBMC styed 100% over the time.

While phages can reduce bacterial load, their presence along with bacteria significantly affects PBMC viability, possibly due to the immune response to bacterial components and the physiological stress from phage-mediated lysis. Interestingly, PBMC viability remained at 100% in conditions without bacteria, suggesting that phages alone do not adversely affect PBMC health.

### 7.2.5. Efficacy of Bacteriophages in Bacterial Control

This study demonstrated a significant reduction in bacterial growth upon the introduction of bacteriophages, particularly in the PBMC+ bacteria+phage group. This finding is consistent with literature demonstrating that bacteriophages can effectively reduce bacterial populations both in vitro and in vivo, highlighting their potential as alternatives to antibiotics (Smith and Huggins, 1983; Chanishvili et al., 2012).

### 7.6. Phage Stability

The constant concentration of phages in these experiments contrasts with other studies where phage populations have been shown to either increase due to bacterial lysis or decrease due to inactivation by host immune responses (Parracho et al., 2012). These results suggest a balance between phage replication and inactivation, a dynamic that varies widely across different phage-host systems (Weinbauer et al., 2004). We assessed phage stability on wound dressings (hydrogel) at room temperature and found that the phages remained stable on the dressing pad for at least six weeks. The phages demonstrated stability for at least seven months when stored in tubes on the lab bench.

### 7.2.7. Impact on Cell Viability

Here was reported decreased cell viability in cultures containing both bacteria and phages, underscoring the complex interactions among bacterial growth, phage activity, and host cell responses. Similar observations have been made in other studies, where the dynamics of phage-bacteria interactions indirectly affected cell health, particularly through the release of endotoxins from lysed bacteria (Dabrowska et al., 2005).

### 7.2.8. Interaction with Immune Cells

These findings highlight that PBMC viability remained unaffected in the absence of a bacterial challenge, suggesting phages alone do not adversely impact immune cell health. This aligns with research indicating that while phages can elicit an immune response, they are generally well-tolerated by the host immune system (Górski et al., 2012).

### 7.2.9. Bacterial Resistance

Although not directly addressed in this study, the observed increase in bacterial numbers over time hints at emerging resistance or adaptation. The literature extensively documents the rapid evolution of bacterial resistance to phages, driven by the selective pressure of phage predation (Labrie et al., 2010). Strategies such as phage cocktails and engineered phages are being explored to counteract this resistance (Lu and Koeris, 2011).

TABLE 6
Summary of the findings in PBMC, bacteria, and phage study media

| | On cell culture media | | | | | On bacterial culture |
|---|---|---|---|---|---|---|
| | PBMC + | PBMC + | PBMC + Bacteria + | PBMC + | PBMC | media |
| | Bacteria | Phage | Phage | Mitogen | only | Bacteria only |
| | Time 0 | | | | | |
| Bacteria | 5000 | — | 5000 | — | — | 5000 |
| Phage | — | 100000000 | 100000000 | — | — | — |
| cell | 100% alive | 100% alive | 100% alive | 100% alive | 100% | — |
| viability | | | | | alive | |
| | 1 hours | | | | | |
| Bacteria | 6000 | — | 4000 | — | — | 7000 |
| Phage | — | 100000000 | 100000000 | — | — | — |
| cell | 100% alive | 100% alive | 100% alive | 100% alive | 100% | — |
| viability | | | | | alive | |
| | 12 hours | | | | | |
| Bacteria | 58000 | — | 300 | — | — | 3000000 |
| Phage | — | 100000000 | 100000000 | — | — | — |
| cell | 100% alive | 100% alive | 100% alive | 80% alive | 100% | — |
| viability | | | | | alive | |
| | 24 hours | | | | | |
| Bacteria | 30000000000 | — | 1000000 | — | — | 1E+15 |
| Phage | — | 100000000 | 100000000 | — | — | |
| cell | 60% alive | 100% alive | 100% alive | 50% alive | 100% | |
| viability | | | | | alive | |
| | 36 hours | | | | | |
| Bacteria | 2E+14 | — | 1E+12 | — | — | 1E+18 |
| Phage | — | 100000000 | 100000000 | — | — | |
| cell | dead | 100% alive | 50% alive | 30% alive | 100% | |
| viability | | | | | alive | |

### REFERENCES

1. Abedon, S. T., Kuhl, S. J., Blasdel, B. G., & Kutter, E. M. (2011). Phage treatment of human infections. Bacteriophage, 1 (2), 66-85.
2. Ackermann H-W. 2012. Bacteriophage electron microscopy. Adv Virus Res 82:1-32. doi: 10.1016/B978-O-12-394621-8.00017-0. [PubMed] [CrossRef] [Google Scholar]
3. Alexander J, Navas D, Flowers M, Charles A, Carr A. 2020. Isolation of lytic bacteriophages with broad host range activity against Pseudomonas aeruginosa strains isolated from respiratory samples from cystic fibrosis patients intended for therapeutic application. Open Forum Infect Dis 7: S801-S801. doi: 10.1093/ofid/ofaa439.1795 [CrossRef] [Google Scholar]
4. Arndt D, Grant J R, Marcu A, Sajed T, Pon A, Liang Y, Wishart D S. 2016. PHASTER: a better faster version of the PHAST phage search tool. Nucleic Acids Res 44: W16-W21. doi: 10.1093/nar/gkw387. [PMC free article] [PubMed] [CrossRef] [Google Scholar]
5. Au, T. Y. & Assavarittirong, C. Combating antimicrobial resistance: an evidence-based overview of bacteriophage therapy. Postgrad Med J, doi: 10.1136/postgradmedj-2022-141546 (2022).
6. Besemer J, Lomsadze A, Borodovsky M. 2001. GeneMarkS: a self-training method for prediction of gene starts in microbial genomes. Implications for finding sequence motifs in regulatory regions. Nucleic Acids Res 29:2607-2618. doi: 10.1093/nar/29.12.2607. [PMC free article] [PubMed] [CrossRef] [Google Scholar] Chegini Z, Khoshbayan A, Taati Moghadam M, Farahani I, Jazireian P, Shariati A. 2020. Bacteriophage therapy against Pseudomonas aeruginosa biofilms: a review. Ann Clin Microbiol Antimicrob 19:45. doi: 10.1186/s12941-020-00389-5. [PMC free article][PubMed][CrossRef][Google Scholar]
7. Bortolaia V, Kaas R S, Ruppe E, Roberts M C, Schwarz S, Cattoir V, Philippon A, Allesoe R L, Rebelo A R, Florensa A F, Fagelhauer L, Chakraborty T, Neumann B, Werner G, Bender J K, Stingl K, Nguyen M, Coppens J, Xavier B B, Malhotra-Kumar S, Westh H, Pinholt M, Anjum M F, Duggett N A, Kempf I, Nykäsenoja S, Olkkola S, Wieczorek K, Amaro A, Clemente L, Mossong J, Losch S, Ragimbeau C, Lund O, Aarestrup F M. 2020. ResFinder 4.0 for predictions of phenotypes from genotypes. J Antimicrob Chemother 75:3491-3500. doi: 10.1093/jac/dkaa345. [PMC free article] [PubMed] [CrossRef] [Google Scholar]
8. Cesta, N. et al. Bacteriophage therapy: an overview and the position of Italian Society of Infectious and Tropical Diseases. Infez Med 28, 322-331 (2020).
9. Chan, B. K., Abedon, S. T. & Loc-Carrillo, C. Phage cocktails and the future of phage therapy. Future Microbiol 8, 769-783, doi: 10.2217/fmb.13.47 (2013).
10. Chanishvili, N. (2012). A literature review of the practical application of bacteriophage research. *Bacteriophage*, 2 (2), 2012.
11. Chegini Z, Khoshbayan A, Taati Moghadam M, Farahani I, Jazireian P, Shariati A. 2020. Bacteriophage therapy against Pseudomonas aeruginosa biofilms: a review. Ann Clin Microbiol Antimicrob 19:45. doi: 10.1186/s12941-020-00389-5. [PMC free article] [PubMed] [CrossRef] [Google Scholar]
12. Chen, P. et al. Bacteriophage therapy for empyema caused by carbapenem-resistant Pseudomonas aeruginosa. Biosci Trends 16, 158-162, doi: 10.5582/bst.2022.01147 (2022).
13. Clokie M R J, Millard A D, Letarov A V, Heaphy S. 2011. Phages in nature. Bacteriophage 1:31-45. doi: 10.4161/bact.1.1.14942. [PMC free article] [PubMed] [CrossRef][Google Scholar]
14. Dabrowska, K., Switała-Jeleń, K., & Weber-Dąbrowska, B. (2005). The potential role of bacteriophages in treating infections. American Journal of Pharmacogenomics, 5 (5), 271-281 15. Dabrowska, K., et al. (2005). Interaction of bacteriophages with their host cells. *Cellular and Molecular Life Sciences*, 62 (21), 2005.
16. David Hou, C. F. et al. Cryo-EM Structure of a Kinetically Trapped Dodecameric Portal Protein from the Pseudomonas-phage PaP3. J Mol Biol 434, 167537, doi: 10.1016/j.jmb.2022.167537 (2022).
17. De Soyza, A. et al. Developing an international Pseudomonas aeruginosa reference panel. Microbiologyopen 2, 1010-1023, doi: 10.1002/mbo3.141 (2013).
18. Dobin A, Davis C A, Schlesinger F, Drenkow J, Zaleski C, Jha S, Batut P, Chaisson M, Gingeras TR. 2013. STAR: ultrafast universal RNA-seq aligner. Bioinformatics 29:15-21. doi: 10.1093/bioinformatics/bts635. [PMC free article] [PubMed] [CrossRef] [Google Scholar]
19. Donkor E S, Dayie N T, Adiku T K. 2014. Bioinformatics with basic local alignment search tool (BLAST) and fast alignment (FASTA). J Bioinform Seq Anal 6:1-6. [Google Scholar]
20. Folmer, R. H., Nilges, M., Konings, R. N. & Hilbers, C. W. Solution structure of the single-stranded DNA binding protein of the filamentous Pseudomonas phage Pf3: similarity to other proteins binding to single-stranded nucleic acids. EMBO J 14, 4132-4142, doi: 10.1002/j.1460-2075.1995.tb00087.x (1995).
21. Friedberg, E. C. Dark repair in bacteriophage systems: overview. Basic Life Sci 5A, 125-133, doi: 10.1007/978-1-4684-2895-7_16 (1975).
22. Garneau J R, Depardieu F, Fortier L-C, Bikard D, Monot M. 2017. PhageTerm: a tool for fast and accurate determination of phage termini and packaging mechanism using next-generation sequencing data. Sci Rep 7:8292. doi: 10.1038/s41598-017-07910-5. [PMC free article] [PubMed] [CrossRef] [Google Scholar]
23. Gavric, D. & Knezevic, P. Filamentous Pseudomonas Phage Pf4 in the Context of Therapy-Inducibility, Infectivity, Lysogenic Conversion, and Potential Application. Viruses 14, doi: 10.3390/v14061261 (2022)
24. Gordon D, Green P. 2013. Consed: a graphical editor for next-generation sequencing. Bioinformatics 29:2936-2937. doi: 10.1093/bioinformatics/btt515 [PMC free article] [PubMed] [CrossRef] [Google Scholar] 25. Górski, A., et al. (2012). Bacteriophages and immunomodulation. *Future Microbiology*, 7 (7), 2012.
26. Gurevich A, Saveliev V, Vyahhi N, Tesler G. 2013. QUAST: quality assessment tool for genome assemblies. Bioinformatics 29:1072-1075. doi: 10.1093/bioinformatics/btt086. [PMC free article] [PubMed] [CrossRef] [Google Scholar]
27. Hagens, S., & Loessner, M. J. (2010). Application of bacteriophages for detection and control of foodborne pathogens. Applied Microbiology and Biotechnology, 86 (3), 975-986.
28. Hanlon, G. W., Denyer, S. P., Olliff, C. J., & Ibrahim, L. J. (2001). Reduction in exopolysaccharide viscosity in Serratia marcescens by bacteriophage infection. Journal of Bacteriology, 183 (21), 6271-6275.
29. Hashemi Shahraki, A. & Mirsaeidi, M. Phage Therapy for Mycobacterium Abscessus and Strategies to Improve Outcomes. Microorganisms 9, doi: 10.3390/microorganisms9030596 (2021).
30. Hashemi Shahraki A, Vahed M, Dinparastisaleh R, Mirsaeidi M. 2023. Complete genome sequencing of a novel Pseudomonas aeruginosa phage, UF_RH5. Microbiol Resour Announc 12: e0039623. doi: 10.1128/mra.00396-23 [PMC free article] [PubMed] [CrossRef][Google Scholar]
31. Hashemi Shahraki A, Vahed M, Mirsaeidi M. 2023. Complete genome sequencing of the novel Pseudomonas aeruginosa phage UF_RH1. Microbiol Resour Announc 12: e0013923. doi: 10.1128/mra.00139-23 [PMC free article] [PubMed] [CrossRef] [Google Scholar]
32. Jault, P. et al. Efficacy and tolerability of a cocktail of bacteriophages to treat burn wounds infected by Pseudomonas aeruginosa (PhagoBurn): a randomised, controlled, double-blind phase 1/2 trial. Lancet Infect Dis 19, 35-45, doi: 10.1016/S1473-3099 (18) 30482-1 (2019).
33. Jernigan, J. A. et al. Multidrug-Resistant Bacterial Infections in U.S. Hospitalized Patients, 2012-2017. N Engl J Med 382, 1309-1319, doi: 10.1056/NEJMoa1914433 (2020).
34. Jiang Y-H, Liu J-Q, Zhao C-Y, Yu S, Sun Y-B, Shi H-Y, Huang H-L. 2020. Isolation and genome sequencing of a novel Pseudomonas aeruginosa Phage PA-YS35. Curr Microbiol 77:123-128. doi: 10.1007/s00284-019-01792-8. [PubMed] [CrossRef] [Google Scholar]
35. Kim D, Song L, Breitwieser F P, Salzberg S L. 2016. Centrifuge: rapid and sensitive classification of metagenomic sequences. Genome Res 26:1721-1729. doi: 10.1101/gr.210641.116. [PMC free article] [PubMed] [CrossRef] [Google Scholar]
36. Koskella, B. & Meaden, S. Understanding bacteriophage specificity in natural microbial communities. Viruses 5, 806-823, doi: 10.3390/v5030806 (2013).
37. Kutter, E., De Vos, D., Gvasalia, G., Alavidze, Z., Gogokhia, L., Kuhl, S., & Abedon, S. T. (2010). Phage therapy in clinical practice: Treatment of human infections. Current Pharmaceutical Biotechnology, 11 (1), 69-86.
38. Labrie, S. J., et al. (2010). Bacteriophage resistance mechanisms. *Nature Reviews Microbiology*, 8, 2010.
39. Lyon, R. et al. Membrane lipid renovation in Pseudomonas aeruginosa-implications for phage therapy? Environ Microbiol 24, 4533-4546, doi: 10.1111/1462-2920.16136 (2022).
40. Lister, P. D., Wolter, D. J. & Hanson, N. D. Antibacterial-resistant Pseudomonas aeruginosa: clinical impact and complex regulation of chromosomally encoded resistance mechanisms. Clin Microbiol Rev 22, 582-610, doi: 10.1128/CMR.00040-09 (2009).
41. Liu B, Pop M. 2009. ARDB-antibiotic resistance genes database. Nucleic Acids Res 37: D443-D447. doi: 10.1093/nar/gkn656. [PMC free article] [PubMed] [CrossRef][Google Scholar]
42. Lowe T M, Chan P P. 2016. tRNAscan-SE on-line: integrating search and context for analysis of transfer RNA genes. Nucleic Acids Res 44: W54-W57. doi: 10.1093/nar/gkw413. [PMC free article] [PubMed] [CrossRef] [Google Scholar]
43. Lu, T. K., and Koeris, M. (2011). The next generation of bacteriophage therapy. *Current Opinion in Microbiology*, 14 (5), 2011.
44. Lyczak, J. B., Cannon, C. L. & Pier, G. B. Establishment of Pseudomonas aeruginosa infection: lessons from a versatile opportunist. Microbes Infect 2, 1051-1060, doi: 10.1016/s1286-4579 (00) 01259-4 (2000).
45. Maddocks, S. et al. Bacteriophage Therapy of Ventilator-associated Pneumonia and Empyema Caused by Pseudomonas aeruginosa. Am J Respir Crit Care Med 200, 1179-1181, doi: 10.1164/rccm.201904-0839L E (2019).
46. Martin M. 2011. Cutadapt removes adapter sequences from high-throughput sequencing reads. EMBnet j 17:10-12. doi: 10.14806/ej.17.1.200. [CrossRef] [Google Scholar]
47. Meier-Kolthoff J P, Göker M. 2017. VICTOR: genome-based phylogeny and classification of prokaryotic viruses. Bioinformatics 33:3396-3404. doi: 10.1093/bioinformatics/btx440. [PMC free article] [PubMed] [CrossRef] [Google Scholar]
48. Nayfach S, Camargo A P, Schulz F, Eloe-Fadrosh E, Roux S, Kyrpides N C. 2021. CheckV assesses the quality and completeness of metagenome-assembled viral genomes. Nat Biotechnol 39:578-585. doi: 10.1038/s41587-020-00774-7. [PMC free article] [PubMed] [CrossRef] [Google Scholar]
49. Pappas, G., Saplaoura, K. & Falagas, M. E. Current treatment of pseudomonal infections in the elderly. Drugs Aging 26, 363-379, doi: 10.2165/00002512-200926050-00001 (2009).
50. Parracho, H. M. R. T., et al. (2012). The role of regulated clinical trials in the development of bacteriophage therapeutics. *Journal of Molecular and Genetic Medicine*, 6, 2012.
51. Peters D L, Lynch K H, Stothard P, Dennis J J. 2015. The isolation and characterization of two Stenotrophomonas maltophilia bacteriophages capable of cross-taxonomic order infectivity. BMC Genom 16:664. doi: 10.1186/s12864-015-1848-y. [PMC free article] [PubMed] [CrossRef] [Google Scholar]
52. Reyes-Cortes, R., Martinez-Penafiel, E., Martinez-Perez, F., de la Garza, M. & Kameyama, L. A novel strategy to isolate cell-envelope mutants resistant to phage infection: bacteriophage mEp213 requires lipopolysaccharides in addition to FhuA to enter Escherichia coli K-12. Microbiology (Reading) 158, 3063-3071, doi: 10.1099/mic.0.060970-0 (2012).
53. Shariati A, Azimi T, Ardebili A, Chirani A S, Bahramian A, Pormohammad A, Sadredinamin M, Erfanimanesh S, Bostanghadiri N, Shams S, Hashemi A. 2018. Insertional inactivation of oprD in carbapenem-resistant Pseudomonas aeruginosa strains isolated from burn patients in Tehran, Iran. New Microbes New Infect 21:75-80. doi: 10.1016/j.nmni.2017.10.013. [PMC free article] [PubMed] [CrossRef] [Google Scholar]
54. Skurnik, M., Pajunen, M., & Kiljunen, S. (2007). Biotechnological challenges of phage therapy. Biotechnology Letters, 29 (7), 995-1003.
55. Smith, H. W., and Huggins, M. B. (1983). Successful treatment of experimental Escherichia coli infections in mice using phage: its general superiority over antibiotics. *Journal of General Microbiology*, 129, 1983.
56. Smith, H. W., Huggins, M. B., & Shaw, K. M. (2003). The control of experimental Escherichia coli diarrhoea in calves by means of bacteriophages. Journal of General Microbiology, 129 (8), 2659-2665.
57. Tacconelli, E. et al. Discovery, research, and development of new antibiotics: the WHO priority list of antibiotic-resistant bacteria and tuberculosis. Lancet Infect Dis 18, 318-327, doi: 10.1016/S1473-3099 (17) 30753-3 (2018).
58. Tamma, P. D. et al. Safety and microbiological activity of phage therapy in persons with cystic fibrosis colonized with Pseudomonas aeruginosa: study protocol for a phase 1b/2, multicenter, randomized, double-blind, placebo-controlled trial. Trials 23, 1057, doi: 10.1186/s13063-022-07047-5 (2022).
59. Torres-Barcelo, C. & Hochberg, M. E. Evolutionary Rationale for Phages as Complements of Antibiotics. Trends Microbiol 24, 249-256, doi: 10.1016/j.tim.2015.12.011 (2016).
60. Uritskiy G V, DiRuggiero J, Taylor J. 2018. MetaWRAP—a flexible pipeline for genome-resolved metagenomic data analysis. Microbiome 6:158. doi: 10.1186/s40168-018-0541-1, [PMC free article] [PubMed] [CrossRef] [Google Scholar]
61. Van Nieuwenhuyse, B. et al. Bacteriophage-antibiotic combination therapy against extensively drug-resistant Pseudomonas aeruginosa infection to allow liver transplantation in a toddler. Nat Commun 13, 5725, doi: 10.1038/s41467-022-33294-w (2022).
62. Weinbauer, M. G. (2004). Ecology of prokaryotic viruses. *FEMS Microbiology Reviews*, 28, 2004.
63. Wittebole, X., De Roock, S. & Opal, S. M. A historical overview of bacteriophage therapy as an alternative to antibiotics for the treatment of bacterial pathogens. Virulence 5, 226-235, doi: 10.4161/viru.25991 (2014).
64. Wright, A., et al. (2009). Phage therapy: an alternative to antibiotics in the age of multi-drug resistance. *World Journal of

What is claimed is:

1. A composition comprising at least one phage, or at least two phages, selected from a group comprising UF_RH1, UF_RH5, UF_RH6, UF_RH7, and UF_RH9.

2. The composition of claim 1, wherein UF_RH1 comprises a nucleic acid sequence of SEQ ID NO: 1, a variant thereof having at least 97% sequence identity therewith, or a fragment thereof.

3. The composition of claim 1, wherein UF_RH5 comprises a nucleic acid sequence of SEQ ID NO: 2, a variant thereof having at least 97% sequence identity therewith, or a fragment thereof.

4. The composition of claim 1, wherein UF_RH6 comprises a nucleic acid sequence of SEQ ID NO: 3, a variant thereof having at least 97% sequence identity therewith, or a fragment thereof.

5. The composition of claim 1, wherein UF_RH7 comprises a nucleic acid sequence of SEQ ID NO: 4, a variant thereof having at least 97% sequence identity therewith, or a fragment thereof.

6. The composition of claim 1, wherein UF_RH9 comprises a nucleic acid sequence of SEQ ID NO: 5, a variant thereof having at least 97% sequence identity therewith, or a fragment thereof.

7. The composition of claim 1, wherein the at least two phages comprise a sequence selected from SEQ ID NOs: 1, 2, 3, 4, and 5, or wherein, optionally, the composition comprises 3-5 phages comprising a sequence selected from SEQ ID NOs: 1, 2, 3, 4, and 5.

8. The composition of claim 7, wherein the composition comprises other phages that can infect Pseudomonas aeruginosa.

9. The composition of claim 1, 7 or 8, wherein the composition optionally comprises pharmaceutically acceptable excipients for parenteral, nasal, or topical administration.

10. The composition of claim 1, 7, 8 or 9, wherein the composition is formulated into a solution for parenteral administration.

11. The composition of claim 1, 7, 8 or 9, wherein the composition is formulated into a solution for nebulizer.

12. The composition of claim 1, 7, 8 or 9, wherein the composition is formulated into a solution or aerosol for nasal spray.

13. The composition of claim 1, 7, 8 or 9, wherein the composition is formulated into aerosol or dry powder for inhalation.

14. The composition of claim 1, 7, 8 or 9, wherein the composition is formulated into a lotion, cream, gel, an emulsion, ointment, or dry powder for topical administration.

15. A spray container, which is pressurized or not, containing the composition of claim 1, 7, 8 or 9.

16. The spray container of claim 15, wherein the spray container optionally comprises an antibiotic or antibiotics.

17. A method of treating a subject having an antibiotic-susceptible- and/or antibiotic resistant Pseudomonas aureginosa infection, the method comprising administering an effective amount of the composition of claim 1, 7, 8 or 9 through parenteral, nasal, or topical route.

18. The method of claim 17, wherein the composition is optionally co-administered with an antibiotic or antibiotics, and wherein the antibiotic or antibiotics are administered through oral, parenteral, topical or nasal route, together with the composition or separately before, after, or at the same time with the administration of the composition.

19. The method of claim 17, wherein the subject has P. aeruginosa infection in the lung, urinary tract, skin, and/or blood stream.

20. The method of claim 19, wherein the subject has P. aeruginosa infection after lung transplant or is suffering from pneumonia, cystic fibrosis, bronchiectasis, bladder infection (cystitis), kidney infection (pyelonephritis), skin infection (cellulitis, burn wounds), and/or sepsis.

21. A method of disinfecting a surface, comprising applying to the surface the composition of claim 1, wherein the composition is formulated into a liquid, aerosol, or powder form.

22. A method of disinfecting a fish tank or aquafarm, comprising applying to the water the composition of claim 1, wherein the composition is formulated into a liquid, aerosol, or powder form.

23. A method for quantitative analysis of antibiotic effect of a phage or a phage cocktail, comprising steps of:
i. injecting a certain amount (cfu) of a bacterial strain, which is to be infected with the phage or phage cocktail, into wax worm larvae;
ii. waiting about 20-60 minutes;
iii. injecting a phage or phage cocktail at various pfu contained in a certain volume of injection solution, into the larvae of step i;
iv. waiting about 2-24 hours;
V. counting surviving larvae and comparing the number of surviving larvae with control larvae, which is not injected with the phage or phage cocktail.

24. A wound dressing comprising a absorbent pad or gauze comprising an amount of the composition according to any of claims 1-8 and 14, and optionally an adhesive for holding the absorbent pad or gauze in place on skin of a patient.


SEQUENCE LISTING (5 sequences)

SEQ ID NO: 1 - DNA, 42609 bp, unidentified
  source 1..42609: mol_type = genomic DNA
gcgaccgcgaagttcgacggcacctatggaaccgggcgcacgtaaaggcgttgaagctggccggaattatcgccgtcggttgcaacccatacgacccgaccattaccgccgacgttgcgtcttgggcgattaaccttgtcgtcgccgacgttcgcaacttgcttgcccggttcgatgctggcgaaattggcatagacaacgacgaaacgaaacagcttgcgaaggttattgcaaccgttaaagatttcgtcgtttcaccttggccggacgttgcgaagtacgcaggcgaaggaatgtccaaccttcattcgaaccgaattgttccgtacagctacgttcaacgcaggcttgccgctgttgccgtctttcgtaaagaccgcatcggcgcaagcggggcaatcaagcgcgccttgaagaccctttgcgaacgcggcgatttgcaggaagtaagccgggccacactggcgaaggattacggaacaagcgccgttgcatacatggtcgcgcatccgggcgttttcggcctgtaacgcgcgcccgccagtgtgggcagaacgggccgccttcgggcggctttttcgtgcctgtaaaaaaagttcgtcgtacctgttgacgtagcttcgaactgtacgtagaatacaaaccatcgaagcaaacaacgaaggagtaaacgaaatgacctacgccgaagccaagaaagccgctaacaagaaaaacgccggtattgtttacactggcgacgcgggtaacggttgcacgttcgaagtttattactgcgaatggcgcaagcgtcaaatttggacgaccgtaacgccgaacggaacccgcattgtataaaccatgccgacggcccttggcacgcgctaggggccgcgcagcctgcccacaatgaccgaaggacgaccgaccatgattaagccgcaacgttggtacgaaataaccgaagaagaaggccgcaaggttttgaacgtgtacgacgcgccttacaaagaacaggcgacaagttcaagccatacaaagcccgaagatttgcagtattaccgaacaaacttcaaacttgaaaagctttggaaggagtaacaaccatgtacgcccattacttcgccagcaagaaaacaaagttcgttgtaatcagcgacggccctacgcccaacggcaaacgaattgcggtaagcggcaaggtcgaagcgcgcaagattgcgaaggaacacgacgcgcagccttggaacttctaaacttcaaacttcgaccgcttcggcggtctttttgtttgtacggcgtataacacgacggcccgcgtataatggcccgtcataacgcttaaacccttgatacataaggcttttaacggatttatagcaagtataaggtttaacgccgttccacctgtaccggggcgactctttattaaaagtcactattgattaatagtaatatttttactattatattgtattatattattatatttcctttattttcaaggacttagcagctatacgacccgttcaatccgtataatgctttatcaattgattaattgttgacacttgataaaaagtaagctaggcttgcactattgattaaggggcttgctatgccgaaaccaacgacgaagaagggccgaccaaagggctttattccgaagcctgcgaactgcggccagtgtttgcgcgaagtagcgccgaacatgaaaatttgtccgtattgcttgaagtcaaccgggcttatatcgactgcgaaggagttaaagaaatgaccgaagaaacgaagaaggttcaaattttaccgtcgtcgccgaaggcgcttgctttgtgcgaagtttcgacggacaatcaaccggaacgcacgccggacgaagacaacgcgaaatatccgtttcgttcgttgcaagtcggcgaatcgttcgcgataccgtttgcagatacgaaccccggaacgcttgcgtcgcttcgttcgtcggccagttcatacggcaagcggcttaaccgcaagtatcgcgttttcgttcacgacgaatataagtgcgtcgaagtggcgcggctgaaatgaacagcgacgcgcggcccaatgtgggcaaaatggttcgcgtgtatgggtcgtcgtatcttggccttcgcgcgcagtatccgaacgcctttgccatccaaccaacccggacgggctatcaagccgtcgtaatgattgaagaaggggcaaacaatgtacgaaatcgaaaccggccataaggtgccgaagcaacgtcgcaaggctggaagcgtgccgaaggccgttcaacaggcgttcaacgactacgccgaagcgtataaggccgtttacggcgttcgcccgctgtcgttcacgtatgaccgcgaaagcggctttattcgtgtcgaaagtagcggcggcgtaagcgttaaaaggctgcgcgaaatgacgaaacaacttcgttaccgcaaaggctgaaaaaagttgttcgcaggtattgacgaagcgtcgaaggcgtcgtactatatacccataccaacgaacacaaggaaccgccgaaatgaaaaacgcacaagtcgcagcaatggaagccaaaatgtcgaaggttgcaggcgcagccgtagaacttaccattcgcgccgaacgcgcctttacgttcagcttcgaaagcgtaaacgaagaagccgccgcaaagctggcaaagttcttcgaaggccaagcgaaagtcgaagttgaacacgacgaagaatgcggttccttcgtttacgtcgattgcaagtaatcaacagcgcgtcgggcatttcgtcccggtcgttcagcaagtcggcggcgttctgcaagcgttcgacgtgttcaggctggaacgcgccggggttggcttcgtgtacttcctgcgcgacttccagaacatcgcccaaggcttcggccagcttttcgaccaagccaagaattttggcgcggtcttctttatccgggccggaagcttcgacggcctgtttaatgtcgcatacgatgcacattgttataactccttcaaaaatgccgggttgcgcccgacgcgctgttggccctgctgttggccgcccttgggcgcggcgtcaaggtcgaaacaatccgcgtcgaactgcgcgaagacgaaacgccagaacaggccgtagggcgtgtaatggccgaacgcgaagccggggccgcgacgaaacactaaccaaagcgccccggctgttgtatagtcggggcttctttctaacatggggaacgaaagaacatggcggacgaagtagaccaaacagccgaccgaatagaaaacgaagtaagctttacggtcggcgaactttgccgaaaggccgcagcgatacccgcaggccgtccgggcgaatgtgatttttgcggcgaacacttcgcgcgcgttgtcgaagtgaccgacccgcgaacgggcgaagtcgtcgaaagctgcggtcgctgtcgtgaccgaaggggcatcgcatgacgcccgcaacccttgccaagtctggcaccgaacacgcgcaccaaacggcgctttttgcttatgctgccgtcgcatacttgcacggcttcgacgtagccgacgaatggtgcaagaccggcaaactaccgaagcgcgacccggacgcaccgcccaaggtgcccgcccttgaatggttccacgccataccgaacggcggcagtcgcggcgacgacgacaagtcccgcaagatacgcggcgcgcagttgaaggccgaaggcgtgcggcagggcgtagccgatacgttcttgccttggccgtctggcgattggcacggcctttatatcgaaatgaagaagccgaccgaacgtccgaagtcggaaaccgcaaaaggcggcgtatcggatgaacaacgaagattcggcgaatacgcaaagcgcgtcggctatggttgggtcgtctgctatgattgggaacaagcggcgtcgtaccttcgttcctatatcgaatggggttcgtaatggggctttcgtgccttgcctgcgaacttacccgcgcccggttgaaagcggcggcgctggcccttgtgggctggccccttgaccgcatcgccgggcatttgtccgtttgctatggcgaacggtattacgtcgaaggcggcaagctgtatcgcgcgtcgaagttgccgccgtatcaaccgtacttgataaggggcgaagaatgaaaaagaccgacgaagaattgatagacgaagcccgcagcattgcgcgttcgaaacgctgtttcgtcgtaacgaagccgggttattggctgctgtaccgcgaatgcacgccgcgtaacgtatgcgtcggcaaacggtcgtcggcttcggcgttgctacaacttgcgaaaaaggcttgacgcagcgtcgaagctgtcgtacacttcttcgtaagttgaacgaaccaaggagtaacgaccatgcccgcatataacgtaacaaccgaattcgaaatgaccgacggtcgcgttctgtcgtgcgaatacgtcttgtcggttacgcccggtcgctattccggcccttgggaagattgttacccggacgaatccgacgcaggcgaaccgacgtattttatcgacggcgaagaagtcgattacaaagaatttccaaaaggcttggacgtaatcgccgacaagctttacgaagctggcgacggcgaatacaattacaatcaatccgaaatcgaacgcgattacgaaccggattgcgattacgattattaacgaaggagtaacgaaaatgtcttggcttatcggttggggcgtactttccgtcggcgtcgttgtcggcttcttgcttgcggccttgctgcgggccaatgggcgcgacgtgccggttccgtcggctaccgttaccgccctgtcgccgcaggaacgcgcccgcatgggctacgcactggcaagggctggcgacttgctggacagcggcgaccttgaatacgtgtcgggcggctttatggtgcaagacgtagggcacggcgtcgtaattatcaaaggcgatttgtcgttgcgggttgccgaagatgaataaggacgaagcgcgcgaaatgttcgaagccgtcaaggccaaccgggcgaagattgacggttgcgcccgccatcgcttcgaaatcggcgacccgccgtacacgttcgggcaaaagttcacttgttcgaactgcggcggaacaatggacgccgtacaggcgttccgatactgtcaaggcttcaaggccgcaggcggcgacccgaacgaagttataccgggttacgaataacgcttgacaagcggcgaaggttcgacgaaacttaacacgtacccgacaaaggagttacgaacatgaatttcgacccgctgtttcttaccatcggactttcggcgcttgccttggcttgctacttgaagttcggccaaataaaagcgaagcgcgaacaagaccgccgcattcgctaagccatcgaacgcgcgcaggttgtgaaggcacgcggcgaaattcgcggcaagaagcttgccaacgaacgcgaatggggcttggccgaagattacaaccccggcaagttctggccgcagtaatggcgataatctacgcaccgcgcgaagccctttccaaatgggacgcgcgatttatcgaactggccgaacacgtcgccggatggtcgaaagggccgcgtaagcgcatcggcgcggctatcgtgcgcccgaatcggtcgattgcatcgcttgggtacaacggcccgccgcgcggcttcgacgacgccgtttttctgaacatgacgcgcgacgaacaacacgcggtcgtaattcatgccgaagacaacgcaattcgacaggctgcgccgggcgaagacttgtccgcgtgtacgctgtacgtttcgccgctgtttccgtgcgccatttgtgccgaccgcatcgttcgggccggtatccgtcgcgttgtcgcttattgtgggcatatatcgccagactggcgcgcgtctgccgacgaagccgaacgcatctttaaccaagcgggcgtcgaatgcctgtttacaatggattaacgaacatgaaaccgctagaccattcgcaaatcgaaggcttgttgttctatcaagacccaacgacgcaaaactatatcggcgcaccgaaggccgacccgttgaaccctgccggaagcttcgtcgcattcgggccgcgtgttacggttgacccggcgttgtataacttgcttgccgcagcgccgacaatgtaccaagccctttcgatgcaatacgcttaccttcaagcgcaaatcgaagttatcgaacgcttgccaaaggcgggcgaacttgataagctgttgtcgtcgttaatcgaagcgcagaacgcttgcctaatggcccaacgtatcgcgcaagtcggttacgaaaatgtcgcaaattcacttgacaaaggttcgaagcggtcgtaagcttgaaccatcgaaacgaacaaaggtgcccacaatgaaccgtaagcttcttgttatcgctggaatcgtcgccttcgtcgcgttgcttggcatggtcgggcaaatggacttcgaagacgaagcccgcgcgcacctgttgtattgtaacaacgtagcgaacggcgtttggccggattacgacaaaatttacgattctgaatgcacgcccgaacgcatcgcagaaatctaagaaattttgcgttgaccgcttgacacgaagaacggtcggcgtaaaatcaacccgtcgcaacaacgcgacataaccttaacgttaaggagttacacaaaatggctaccaagaaatcgaccgcgaagaagaacaccgccgccccggttatcggccttgccgaaattgttgcagccgggccgaatggtctgtatacttcgcctgccgtacatggcccgcttgtcgaagccggtatggtggaaatcaacccggcaatgaccaacgaagcgggcgaaatcgcaacccgcgcaacccaaaaaggaatcgaagaaatgaaccagaacaccgaaaccggcaccgccgcagcttccgccccggtcgctacttccttcaccatcgaagacaacgtaccgatgccgaccggcagcggtcgcggtcgcggcggcaacgtctacccgttcgacgcgctggaagtcggccaaagcttcttcgttccgaacagcgaagaaaagccgaacgccgcgaagtcgcttgcttcgaccgtttccagcgcaaccgcccgtttcgctgttccgtccgaagacggcgcgaccaagaccaacaagaagggcgaaaccgtgcccgtcatggtcgaaactcgcaagttcgttgttcgcgctgtcgaaggcggcgcgcgcgtctggcgcaccaagtaaccggcgcgggcttcggcccgctacggccaacagttcgacgaaagcccggccaagcgccgggctttttgttgtctgtacgtttctttcgctttgctttttctagccatttgtgttaatccgtgctaatctgcgccctatgttgtgatttatcaaataccgggcggggcgtcgaataatgaacgaacaacaattttttaacgtagctgtcgcaattatcggcgctttgggcggatggttaatgcgggtaatgtggcagtcgttgaaagaacttcaaaaccaagatgcaaggttggccgaaaaggtcggaaatatcgaagttcttgttgccggacaatacgtcaagcgcgacgatatggacagaacaaccgccgcactttttgcgaagcttgaccgcatcgaagacaaactagataaaaaggccgacaaatgaagaagcccgaacttatcgaagattggcgcaattggtggcgcttttggtcggttcgactggccgcagtcggcaccgccgttactggcgttcttatcgcgtttcccgacgcagccttggccgcatgggccatacttcccgaagaattcaagtcggcaattccgccgcagtatatgccgcttatcggcgtcggcgtattcgcgctttccatcgttgcgcgccttatccgtcaagaaaagttgaaggggccgaccgatgaataacgaacttccttgggtcgccgaagcccgaaaatatatcggccttcgcgaaaacacttcgaagaccgaacataaccccgtaatcgttaaaatgcttgaacgcatgggcgcgttttcgaacgaatcccgcgcatggtggcgcgaagacgaaacgccgtggtgcggcctgttcgtcggcttttgcttgggcgttaccgggcgcttcgtcgtgcgcgaatggtacagggcgcgggcttgggaatcgccgcagcttacgaagcttgaccggcccgcatacggggcgcttgtgaccttcacgcgcgacggcggcggccacgtcggttttattgtgggcaaggatgcgcgcggaaatcttatggttcttggcggtaatcagtcgaacgccgtaagtattgcaccgttcgcagtatcccgcgtaaccggctatttctggccgtcgttctggcgaaacaagaccgcagttaaaagcgttccgtttgaagaacgttattcgctgccgctgttgaagtcgaacggcgaactttcgacgaatgaagcgtaaaggggcatcgtatgttttccgtaatttgggccaagtttggcaaatggcttgccgcaatcggcgcgggtcttgtcatgctggcaagtatcttttatgccggtcgcaagtccggcgcaaccaaggccgaagccaacgcagcaaacgaacgggcaaacgaccgcgaagcaatcgcagttcgccaagttaacgaagcccgcgaagcttccgaacaacaggtaaaggcggtacaaaatgcgaaagaagttgcttccgataattctacttttgacgacgacgaagttactaggcggctgcgcgacgaatggtcgcgcgactgaaccgcaaagcatcgccgacgcagtacaaacgaagcccgttgtaatcgacacggcttgtaaatgggttgcgcccatttggatttcgaaggccgacggcttgaccgctggcacggcccggcaaattctgaaccataacgaagccgtcgaacgcaattgcggcccgcagtcgccgcctaaatctgcggacaagccttaacagaacgcggggcttgcggtatagtgaagctaacgacgtataaactaggccgacactatgtccgacgaactgaaagaaagcgaagaaaaggccgcatacgcggcccttcttttgaaagaacgcgacccgttcaaagccgcgcttgccctattcccgaacaatacgaatcgggcgctatgggtcgcgaaccattggccgaacgacgccgaagttaaagccgaacaagcccgccttaccgaagaagcgggcgatatggcatttttgccgggcaaagccgaccttgcgcgcgacatttggcagcgtatgcaaggtacaacccttccgaacggcgtaaccattccgccaacgcccgaagaatacgcaaagcttgcgaagctttacgcagacgttcgcgggttcatcgaaaaaccgcaaacaaacgtaaacgtaacgacgaacgtaaatcgtgtcgtcgaaatgcccgtatttgcaaacgaaagcgaatgggaagcggaagcggcacggcagcaacgcgagttgttagaaaatgcacgcactcgccattaaagaagacgggacgaaacaagttccgtatgaagtcgtatttaagccgctgccgggttcgcagacaatcgcgctttgttcgatggcggcgcatacgctgtacgaaggcgcgcgcggccccggcaaaacgcttacgcagcttatgcgcttttaccgtaacgtcggcaagggttacggtaaattctggcgcggcgttatcttcgatttggaattcgaccatttgggcggacttgtcgccgaatcgaaaaaatggttcggcgacaatggcaaattaaaagacggggcgaagttttacgaatcaacttcggcttataaatgggtttggccgactggcgaagaattgttgtttcgccatgttaagaagcttgcggattacgaaggcttccacggtcacgaatacccgtttatcggatggaacgaattaacgaagcatccaagcggcgacctttacgacaaatttatgtcggtcaaccgctgtacgttcgacccgattaaagacacgccgaaagacccgaaaaccgggcgttatttgacgccgaacggtcaaccgctgccgcctgtaaagtgcgaagtattcagcacgacgaacccaagcggccccggtcacaattgggtaaagcgtcgttttattacgattgccccgcgcggcaccgtagttcgtcgcgaaattcagatttacaacccggcgaccgaaaaagaagaaacgcacgtaattagccaaatcgctatcttcggttcgtataaggaaaacccataccttccggcttcgtatatcgccgaactggaaagtatcaaagaaccgaacttgcggaaggcttggctttacggcgattgggacgttaccgcaggcggcgcaatcgacgacctttggcaatcgcatatacacgttgtaccgcgcttcgtcattccgccaagctggcgaatcgacagaacatacgacgacgggtcttcgcatccgtttagtgtgggctggtgggcggaagcggacggcaccgaagcgaccatagttctttcggatggcaccgaatttacgttttgcccgcaaccgggttcgcttgttcagttgttcgaatggtacggatgcgcgaaagacgaaaagggcgaatatataccgaataaaggtttgaagctttcggcgtcgaatatcgcacaaggcataatcgaccgcgaaatttcgttaatggcaaacggttggatttcgtcgcaaccttggcccggccccgccgacaatcgaattcgccaagtaatcgacagcgaactagatacgaccgaaaagctaatgtcgaagaagggcgttcgttggctggaatcggacaaatcgccgggttcgcgcgtaatcggtttgcagcttttacgcgaccgattagaagcgtctgttaattgtgaagggccgggtatatactttatgcaaaattgcattgcaagtattgaattgctgcccacattaccccgcgacgaaaagaagattgacgacgtagacacgaaagccgaagaccatgtttacgatatggtgcgttatcgcgtattgaagggcgcgaacaaagcggcgacgaaatttaaacttgtaatgccaacttaaaggaaccttgaaaatgtctagcccgaacgtttcgtttattcgccccgaacttgcaaagcttttgccgctttattatttgattcgcgacgcaatcgcaggcgaaccgaccgtaaaggctgcgcggacgacttaccttccgatgccgaacgccgaagaccaatcgaaagaaaataaggcgcggtacgaagcttatttgaaacgcgccgttttctataacgttgcgcgtcgaactcttttcggtttggtcgggcaagtgttcatgcgcgaccccgtaattaaggtgccgtcgttgcttaacccgcttgttgcgaacgcaaccggaacgggtatcaaccttacgcagcttgcgaaaaaggccgtatcgctaaaccttgcgtattcgcgggccgggctgttggttgattacccgacgaccgaagccgaaggcggggcgtcgattgccgaccttgaagccgggcgcattcgtccgacgttgtacgtatattcgccgactgaaattatcaattggcgaacaaccgaccggggcgcggaagaagtcttgtcgttggtcgtactgttcgaaacatggtgcgccgccgatgatggtttcgaaatgaaaatgtcgggccaattccgcgttttgcgtttggacgaagaaggctattacgtccatgaaatttggcgcgaaccgcagccaacgaaagccgatggttcgaagattccgaagggcaattatcaacaacacgtcgtttataagccgacagacgaaaacggcaatcgtcttaccgaaattccgtttatgttcatgggttcggaaaacaacgacccgaacccggataacccgaacttttacgacctttgttcgctgaatatggcgcattatcgcaattcggcggattacgaagaaagttgttatatcgtcggacagccgacgccggttcttaccggacttaccgaagaatgggtaaacaacgtacttaaaggaaccgttgcgttcggttcgcgcggcggcattccgcttccagccggggccgacgcgaagttgcttcaagcgaacgaaaataccatgcttaaagaagcaatggacgccaaagaacgccaaatggtcgcacttggcgcaaagcttgtcgaacaaaaggaagtgcaacgcacggcaaccgaagccgaattggaagccgcttcggaaggttcgacgctttccagtgcaaccaagaacgtttcggcggcctttgaatgggcgttgaaatgggcggcccgttgggtcggccaagccgacagcggcgttaagttcgaactgaatacagatttcgacattgcccgcatgacgccggaagaacgccgttcgcttgttgaagaatggcagaaaggcgcaattaccttcgaagaaatgcgaaccggcttgcgtaaagctggcgtcgcaaccgaagacgacgccaaggcgaaagaaaaaatcgccaaggataccgccgaagcaatggcccttgctgccccggccaacgtgccgggcgatggaagcggcggcgataatgtgggcaacagcgaataaggggcgcaatcatggcactatcggacaatacgcgactgtacgatattgcgacgcgccttgctgtttacgtcgaagacgtgaaagtttggcagtcccgacaattcgggttcgtacttcgcgaagtaaacttggaattaacgcgccttcttggtcgtgttcgttataagacccttgacggtctttcgaaggcgcagttaaacaagcttgtttcggaattacgggaatcgcaatcgaagatttacagcgcatacacgcaaacgctacttgaacagttaaaggaattcatgcgggccgacttggaagtaaaccggcgcgcatgggttacgggttatatcgaacttgacggcgaagaaagcgacggcattatttcggacgaagaagcgatacagtttcttttagaagtacccaacgccgattcgaaccctttgttcggtttggccgctgttacaggaagcgacgaacgtatttggtcgcaagttacgaatacgccaattccggcaaatggtttgtacttgctgccgtttatcaagacgtttgcgaattcggcacaagctggcgtcgaaagcatgattcgcaaagcttgggcgaaccgttggacggttgaagaaaccttaacggcacttgtcggcgatggaaccgcagcgcagggcacgccgtcgcagttgcaccgggtcaacgcgcaagccgcttcggtaatccatacggcaaccgcgcacgttgcggcggtcgttgcggctggcgttatgtctgcggttttcggtcgttacgtttggtattcggtaatcgacggaagaacaacggacatttgcataagtcgaaatcgtcgaatttatcgcttcggcgaagggccgttgccgcccgcgcatatccgttgccggtcgcacgttgccccggcaaataccgctagtgacttggccgaagaaacgttttatacttgggttgcccgtcaacctgcccaagtgcaagacgacatattgggcgacgaaggcggcgaagctttgcgcgatggtcgcttaaaggcgaaagatattccgaagtatgatgcggacaggccgctaacttacgaagaattccgacgcaagattaaagaaattctttcccgctgattcggtgaatcggcataaccgcaaggagtccttgaaatggcactgaaaaagaaacttaccaaagaagaacacgcgaagctttcggacgcgctgaaagccgaatatatcgaagacggcgacggcttccgccttgacgttgacggcgacgaagatacgggcgctttgaagcgcgccaaagaccgcgaagcgcagttgcgcaaagacgccgaaaaggaagcgaaggaacttcgcgaacggctggaatccatcgaaggcgacgacgcgcgcaagaaaggcgacattgcgacgcttgaaaagtcttggcaatcgaagcttgaaaaacagcgcgaagaatacgaagccaagctttccaagcttaccgcgcatacgacgaaaacgcttgtcgataacgtcgcgtcgcagcttgcccacaagatttcgaacgccccggcgcttatcatgccgcatatcaagtcgcggcttatggcagacttcgaaggcgacacgccggttacgcgcgttctggacaaggacggcaagccgtcggcccttactatcgacgaactggcaaacgaattcgttgcaaacaaggatttttctgctattatcacggctagtaaggcgtccggcggtgccggtaagccttcgcagaacggcggcggtgccccgaaatttaacggtcaatccgacaaacccgccgacctttcgaagatgaatcccgcagaacttgcggccttcattaaagaagcgaaggcaaccgaaactaaggacgcttaatcatggcactttccgaccttgctgtttactccgaatacgcttattcggctttctccgaaaccctgcgccaacaggttgatttgttcaacgctgcgacgggcggggctatcatgctgcaatccgcagcgcaccaaggcgacttttccgacgttgcctttttcgccaaggtaactggcggccttgttcgccgtcgtaacgcctacggttccggcaccgtcgccgaaaaggtcttgaaacaccttgtcgatacgtcggttaaggtcgcagccggtacgccgcctgttcgccttgaccccggccagttccgttggattcagcagaacccggaagtcgcaggcgcggcaatggggcagcaactcgccgtcgatacgatggcggatatgctgaacgttggtcttggttcggtctattcggcactttcgcaggttgcggcggttgtttacgacgcgaccggcaatactgcgccggacgacggcccgacttggaacaaccttaacaacggccaagccaagttcggcgaccaatcttcgcagattgccgcatggattatgcacagtacgcccatgcacaagctgtacagcaacaacctgtcgaactccgaacgcctgtttacttacggcaccgtgaacgttgttcgcgacccgttcggcaagcttcttgttatgaccgattcgccgaacctgtttaccgcaggtacgccgaacgtttatcacattcttggccttgtgccgggcgcggttatgattggtcaaaacaacgacttcgacgcgaacgaagaaaccaagaacggcgacgaaaacattattcggacttaccaagccgaatggtcgtacaacgtcggcgttaagggcttcgcttgggacaaggcgaacggcggcaagtcgccgaccgacgcggcgttgttcacttcgaccaattgggacaagtacgcaacttccgaaaaggatttggcgggcgttgtcgtcgaagttcactaacggacggcagacggacggggcttcggccccgttcttctagtccttcacaaattcaaaggagtttcgaagatgaaaccggcaaaaattctgttcttcgttgacggcaacgcaccgaccccggaagacttcgcagccgccgccgacctgaacgcgcaagttatgttcaggaacgcccgcgctgttccgtccgaaccgcattcgctggaaatctgcgacggcgtagcgggcaaggttccgaagctgtacgccgacgcttacccggaagcgggcgaagcaatcaagaagaaggccgccgaactgaaagcccttacttcgaaggtcggcgacgccccggcaccgaaggccagcggcaaggccgccgacaagcccgcagccgctacgcagggccagaccccggcgcagggccagaaagccccgcagggcggcagcgccccggcttggaaccccaacccggcgcagtaaccaacggacggcctgtcgcttattgtgggcaggccgtttcaacaacgaactaaggagtcttcgcaatggcaacgaaaaaggttgtttatttcacggcaggcattaacgcaacggctggcgaacttgccgacattgcgaagcttaacgcagccgccgaaccgcaatacgaagttttggtagccaacggcgcagcaaatgccgaatacggcgaaagcgaccgaattattccggctgattttgtcgcgggtacgattcccgacgtttaccgcgttgacggcgacccgctggaagacccggtttatcccgaaatcgaccccgacgcaatcccgaatcaggcgcttacggcaacgcaagcaatcgttaacgacgcgcaagttctggacgttgacggcggcggtactgttacgcttaccatcgaaggcggcgttattaccggcgttgtttacgccgctggcgaataaggcggttgacgcatggcaattacaatcgttgttgaagacggaagcggcgtaacaaacgcgaacagttacgtaagcgtcgcagacgcgcgcatttacgcttcgaatcgcggcgtcgaacttccgtcggacgacgacgaattggccgccatgctaattcgttcgacggattacctagaagcgcaggcgtgccgctatcaaggccgccgcacttcttcggcgcaggctttggaatggccgcgaactggcgtctttttgaactgcgacgaagtaccgtcgaacgttattccgaaatcgctaatcggcgcgcaagttcaacttgcgattgccgttaacgacggtttcgaccttcaaccgaacatttcgccgcaagactacgttattcgcgaaaaggtcggcccgattgataccgaatacgcagacccgaccgccgttggcatcatgcccacatttacagcggttgacgcgcttcttgcgccgctgtttggcgaatgcgcggcgaacaagtttgcgcttcgaacgataagggtttaacgaatggcacaattcgaccgcgcaattaaaacggcgcaacgccttatcgcaaagaacggcgaaaaggtgaaatggcgcgttatcgaagacgcgacgccaacagacccgaacaagccttgggaacccggcccggcattgccggacgacaaagacgttaccatttgctttttgccggttgaccggcagacgcaagaaactttcaacttcattaaaggcaccgaagttccgaaaggttcagtaatggggcttatgggcaatgttccgtttgaaccgaatttgaaagacgttgtaattcgtaacggggtcgaacttcgcttggcttatatcgacgtactttcgcctaacgggcaaaaggtactttacacgatggtatttcaagcatgattgaattcgaccaagttaacgacgaagtaaacgcgctttttcttgcaacttggaacgcaggaagcgcggcaattgcgggttatgttcccgaaattcgttggcagggcgtgcaatatcgcgacttgccggacggttcgaagttttgggttcggctgtcgaagcaaaccgtttttgaagaacaagcgaccctttcaacctgcgaaggagtaccggggcaaagaaaatacacggcgtcgggtcttgtcttcgtgcaaatcttttgcccgaaatcgaatacgcaagcgttcgaacttgggcagaaattggcgaaactttcccgtaatgcttttcgcgggaaatcgacgccgggtaaggtttggtttcgcaatacgcgaattaacgaacttccgcccgaagaactttacgaacggtttaacgtcgttaccgaatttgaatacgacgaaataggttaaggagttcttaacatggtatgcgaaattgccaaaatcgacagcaacattaccggacttgccttcgccgaagaagaatgcttgaagcaacttccgacgacgcccgtttggtacggactggaacccaacagctattcggacttcggcggcgaactttcgaccgttgcccgcgcgcctatcgacccgtcgcgccagaacaagaaaggcacgattaccgaccttgacgcatcgggcggcttcaacgccgactttacgaagaccaaccttacgcgaattctgcaaggcttcttcttcgcagacgcgcgcgaacttccttcgacgcagccgcttaacggcgcatcggttgcgcttaccggcgttaccgcagttgacagcacttacgccgcagcttccgggcttggcgtgtttggtgccgatatgctggtatatgcgaccggctttgccaacgcggcgaacaacggccttaaaaccgtcgtttcggctaccgctgcgggcgtcgttgttgctgaaaccctgattgacgaaaccccgcccgcaggcgcgaagctggaatgcgtcggtcgccaacttgccgccgcagacgcgaacattgccgtaactggcaacgtcgtttcgcttatcgttaccgctggcgactttacgaccatgcctgaactgttccccggtcgttgggtcttcgtcggcggcgatgcgacggcaaaccgcttcgctaataatgtgggctatgcccgcattaagtcggttgcagcgaaggcgcttgttttcgacgacgtgacttggcaggcgaccaacgaaaccggaaccggcaagtcgattcgtcttttcgtcggaactgttatcaagaacgaaaagaccccggcacttatcaagcgtcgttcgtatcaaattgaacgcaccttgggcgaaggtttgaacggtacgcagtgcgaatatctggaaggcgcagtaccgaacgaatttacgttgaacattccgcaggccgacaagctgaacgccgacctttcgtttgttgcttgcgataacacctatcgcagcggcgacgttggcgacgaacagaaaagcggaacccgcgtgcctgcgccgggcgaagatgcgtttaacacttcgtcggacatttaccgaatcaagatggccgttcacgacgacgcttcgtcgaatcctgccgccctgttcggttatgtttccgaagcgaacgtttctattaacaacaacgttacgccaaacaaggcggtcggcgttttgggcgcgttcgacacttcggcgggtaacttcgaagtcggcggttcgattaccgcttactttacgaccgtcgcagcggttaaggccgttcgcgcgaacgccgacgttggtttgtcggttatcgctgcggccaagaacgccggtttcgtgttcgatattccgttgcttggtttgggcggcggtcgtctgaacgtcgaaaaagacgcgccgattaccgttccgctggaacccgcaggcgcagaaaacgcgaacggctatacgatgctgtacgaagtgttttcttatctgccgaatctggcaatgccggactaattgcggtaaactcaaagggccgggtaataccggccctttcttcattcaatcggagtaaatcaaatgtctggactgtttaagcaattcaaaacgaattcgaccaaggaagtcgaaggcgtcgaaatcgaatttcccgaagcgcagaacgacgacggcaccgttcccacgttcatcatttcccgcatgggcaaatcgaacaaggcgtattcaaaggcgcttgacgcggcgacccgtccttatcgccgtcaagtcgaacttggcacgctgaaaaacgaagtcgccgaatcgctgtttatgggcgtgttcgtcgataccgtgttgcgcggctggaagaacgttcagggcgaagacagcaaggaaattgcgtattcgaaggacgccgcaatttcgcttcttaccgaactgccggacgtttacgaacgtttgcaggaagaagccaagttggcttcgaacttccgcgataacgctttggaagccgaagcaaaaaactaacggaagttttggcgcacctgttggaacttggcccgcacgaacaggcgatagcaaagcaagcgatgcgcgcggggcagccgttacccgaacgcatagcgaacgcgccagaacttgaagtaggcttgcagttgtatttgcaagccttcttcgacttagatagcgaacggtcgcacggcaacggtttaacgccgataccttggacaagtatggcggcttacgcaagggctttcgagtttgacgaagaacaaaccgaagacttgttttactttatgcgaaagcttgattcggaacacttgaagaaactagcggacaaacagaaagcggcggcatcgaatggcaaaaagcctgctagacctagccgataggctggaaaagaaagcgaaggcaatagacgaagcggcgtcacaaaacgccgtcgataccgctttagctattgtgggcgacttggcgtacaaaacgcccgtagatacttcgcaagctttgtcgaactggattgtaacgctagaaagtccgtcgggtcaacaaatcaagccgcattttccgggttcgcagggttcaacgcaacgcgcttcggcggctgaaacgttgaattctgcgaagcttgttttacgaaacaagaagcccagccaagcgatatttattacgaacaaccttccgtatatccgcagactgaacgacggttattcggcacaagccccggcggggttcgtcgaacgcgccgttttgattggtcgcaaaatgcgtaagaagttcaagattaaggattagaagaaatggccgacgaaaacatagaaattaaggttcaagacaaagtttcgccgtccatttcgacgaagcttcgcacaattgccagcgaagcccgcaacgccgacgccgcagttaagaaccttcaaacgcagctttcggcaatcaacgtcggcggcttgtcgcagcttatcaacgcatcggccagcgcaacgcgccagcttcaacaaggcgcgcttgccgcgcaacggctggcaaccgaacagcagcgtaccgcaacagccgcagcgcaggccgccgcagcccaacagcgcgtcgcgacggctgcaacccaaggggcaaccgcgcaagccaaccttgccagcgccacgcagcgcacgcagacggcccaacagcagacggcgacagccgcgcagcgcctagcgacagaacagcagcggacagccgttcagacggccaacgcggcggcagcgaacgaccgggccgcccttgccgccctgcggctgcaacaggcgcaagaccgggccgccaattcgacccgcaacgcgacttcggcccttggcggatacattcgaaccgctgccggaattcttggcgtaaccatttcggcgaacgcaattcttgcaagcgccgacgcatacgttacgcttcaaaacaagttgcagaacgtaacgaagtcgcaagaacaagttaacacgcttacgaacgaacttttcgaactgtcgaaccgcacgcgcgcgggtattgaagaaacggcgacagcgtttacacgcttcgaccgtgccttggcgtttatgggtaagtcgcaagaagattcgttgcgaatgaccgaaaccattaacaaggctttgattgtttccggtgcaacagcgcaagaagcgtcgtcggctttgttgcagctttcgcagggctttaacgccggtaagttgcaaggcgacgaattccgcgccgtttcggaaaatatgccaatcgttttggacgcagtagcgaaggcgcttaacgttccaatcaaccgcgttaaggaactttcgaccgaaggtaaaattacttccgaagttctgtttgaagctttcaagcttatacaagaccaagtagacgcaacttttaacaagacgacgcccacaatcggccaaagcttgacggttcttcgcaacaacgcaattcagttcttcggcgaattgaacaaagcgaccggctttactgccgggctttcgcgcgcgattctttggcttggcgaaaacttgaagacggttgccgttattgtcgcagggcttggcgtcgcattgcttgtcgccttcggtgccccgttggttggtgcgctggccggtgcgacggctgcggtaaaagcctttacgctggcgcttgcttcgaacccgattggccttatcgtcgtcgcgctttcgactgcaattgcgtatctaacgcttttccgcgacgaaattaacttgggaatcgacgacgttacgacgttgggcgatttcttccgcgcgacctttgaaggcatcggccaagcaatcgacgacgttaaaaccatcgcgtcgcaattgtgggcagaacttagcgacttcgcaagcgcagcccttggcgaaattacttcgaacgttgacgattcgacttcttcttggtcggacagttacgccgcattctttcaaaccaaccgtacaggttgggccggtgcgctggaaaacgccgcaaaagttcttgacgcaatcgccgggcttttgaccggggccgcaacgttcgcaggccgtgcaatggccgaagttgttatttcggttcaaaacggaatcgcaaacgcttacaacgttgtcgcgggttggattgaaaacgtaacgaataaagccattgaagccgcgaacaagcttcgcgcgatggtcggtaagtcggcttacgaacttgtccaatttgaacgcatgggcagcgcaggccaaaccgaattcgaatcttggggcaagctttgggcgcaatcgctggaagacggatttaacagccaaggcggcgcaatgcaaaacctgttgaacggccttttcgaccgttcgcagcaaatcggcgcgcaacgccgggccgcaggaagcgcgcagcttcgcgggtctggcgcgtcgcagcttgccggggctaccgacgcgaacgcggcgaaggctgcggaacgtcgcgcgctggcaatggaaaaaatcaatacgcagcttgataacgaattggcgcgtatgttccagcttcaaccgcagcgcgaagcgcaagcgaagttcgaccaaatcgaagaaagcttgattcagaaaaagattaagcttacgtcggaagaagccgaagcaatcaaagcgaagattaaggccgttcaagacgcgaccgaagttcaacgccagttcgacgcaatttacgccgaagctgttaacccgttgaaggaatacaacgcttcgcaggaagcggcgaacaagcttcttcaaatgggcgcaattacgcaggaacagcacgcccgcgcagtaacgaaggcgtcggaagcttacgcaaattcgcaagacccgttgcggcagtataaccgcgaccttgaacagcaattgcagcttttgcaaatgctgccgaagcaacgcgaaatcgaacagcagattatgcaagttcagaacgatttgcttgcaaagggcatcgttctaaacgaaaccgaattggcgcagcttcgcgaaaagctgttgcttattcagcaagtaaacgccgtttcgcagcaagaagcgtcgttgttggacgccagcgtaaacaagcgccaacagtttatcgaccaacttaaagcaattcagaacttgcgcaacaacagcggaagcggtttcaacgcaggcgaccaagccgaagcaactaacagcatgttgcaaggcatgggtattgatacaacgaactttcaaacgcagcttaacgcgcaattggcgcaatatcaaacttacgtcgaacagcttaaaatgctgaacgaacagcgtttgattagcgaccaagaatacgccgccgcaagtatgcaacttgaattgcagcggcaaaacctgtacttgaattcggcaagtagtttcttcggaaacttggccgcgcttcaacagtcgggaaataagaaaatggcggcggtcgggaaggccgcagcaatcgcgcaagcgatgattaatacgtatcaatcggcaacgtcggcttatgccgcgatggcgtcgattccttacgtcggcccggcgttgggcgcggcggcggctgcggctgcaattgccgcaggcttggcgaacgttcagcaaatccggtcgcagaataccgggtttaagtcgggcggttttaccgggtcaatgggcgtaaacgaagtcgcgggcgtcgtacacggccaagaattcgttatgaacgcatcggcaaccaaccgcattggcgttgcagaccttcaagccctgcaaacgggcgcggccagtgttcagcgcaacgatgaacaggcaatggcggctagtgtgggcaggggcggcaattccgaacccgcgcccgcgccggttgtcaatgtaccttttagcgccgttgtcgtacaatcgaaagaagcggccttggccgggcttaaatctgcggaaggccgggctttcattctggaaactatcgaacaaaacggcggcaccgtcgcaaaaatcgtcggagttaaataaatgggttacgcaattggaacagttacaaaaggcggcggcgacgattgccattatcaagttttggcaattatcaaaacgctggccgaagcgaacggatggacgacgcttcgttacgtatcaaccggactttaccgcgaattgattttgaaaggcgttggcttgtccggtcttgaagaaatctttgtagggttcaagtgttaccaaagcgtttcgggcgattattacaacataaattgcggcgtatttattggttacgttgccggaaacagtttcgaaacgcagccgggcgcgcaatttagcgcagtgcaagcccacaataacgcggttacgtattttataaccgcaaacgcccaacgaatcgtcggatgctttaaggtcggtacgccggtttacgaacatttttacgtcggcaagttctttccgtatgcacggccaggcgaatacccttcgccgcttgtttgcggcgctatgctgaacggggccgaagcaacgcgattcgacagcacggcgcaggttttcccgtatccgggttattactattcgacggcgcaaaatcggttgtatcaacgcgaccaagcgggcaactggattcgtccgtattgttacccttttacgcaaggttcttcgttgtcaaacgcacttgcggggccgcagggttcgaacacgcttgttcctgccgacgtgtattatcaacttgaaccaattattttggcgcagcaaacaagcgacgtaaacccttcgaacgtatggggcgaattcgacggcgtttatttctgttccggctttaacaacggcgtcgaaaacgtagttcaggaaggcggaagttctgtcgttgaccaaaccgggcttaccgtacttcaagccgtagacgctattaaggcggtcggcgggcgggcgtttgtaatgttgcaaaacgtctatcgtacttcttggcgcgattttatcgctttggaaatgaaataatggcatacattacaggaaccgccgcaagccttgcggatttgaaaacggcaattgaaaacgtttgcacggcgaacggttgggcgctggccgacggcatattgtcgaaaaacggggcgtttttcaagcttaccgcgaacgccggaacatacccgtcgcttgttctgaacggcggcaccagcaagaccggcagcacgctaaacggccagccgacgcagccgcaggcgaacgccggggttatgattggaagcccaaacggaaatacaatcgtttttccgattaattacgaaatttatttgtttaccgacccggacgaagtttattgcgtaattaactataattcggacttttaccaacaactttcgttcggtaagtcggatattccgggcattggcggggccgccgcttggtttaccggaagcatgggaacaggcgtcagcttgacaagtacaagcacaagccgcgtgtatatgtcgtcgtcgcaaacaagttgcggaacaacgccgtataacggcatcgcctgcggcctttggttcgaaagcggccaaggcggttcgtacttttcttctttcgttcattcgaacttagacggcatttcgaattggcgttgcacaggtaacgtaaccgtcggaactggcggatacggtatttcttacgcggcttctttgcttggttcattgccgaatctttccaaccaagcaacgattttgcttccggtcaaggcgattatttacagaaacgacgcaggaagaacaatcgcagccaacccgaaaaacgttcgctatctgcgaatcgacaacgttgttccgggcgaaattgtaacgtttggggccgaccaatggaaagtatacccgttccatcgcaaggattcgacccaacgaaacggcgtcatttggtcaaccggcgcgcagcattcgggaacttggggctacgccattaaatacactggaagctaaaagatggccggaagacttggaacatttttgtcggcgcaaagcttcgtatacggaaacgaaaacccgaatatttccgcagacgttggcaattacgaacctgcggattatgcgccgcttcttacatacgacccgcgcgatacagcattgcgcggcggatattcggcgtacaaaatagcgccttcgaatcgctggcctattgtgggcaaagaacaaagcaacttcgggcgcgattattattatcgcattcacgtttcgccgttggtcttggacttgcaaacggtcgcgtcgtcgcaaacgcgcgaattcaacgtttggaacgcatggcccgaccgtgcggcgaatcttgacgacattttggtaagtaatccggtcggtattgaaattacaggccaagcgacgccgtatgcaatgccgccgcttcaagaacttacttacgaaataaccgtaggcgtttcggggccgccgaatattaacgttgaagttcaatttgacttttccaacgttcccgacccgctgccgattctgattacaggaacgcgcgcggttaagttcaacattgtgcccgaagttccattgaacgaaacttgggaatggttaagcgacaacattgtagcggttgacggaacagaacaacgcatagcacttcgcggcgaaatgccgcgtattgaagaacggttgaaagtaattttcgattctgtcgaagccgtgcgccgtttttacggcgacgttatgtcggcagttggtcggctttggattcccgaatttcagtacgcgacccgaattactgccgcaagcaataccggcgcgcttcaaatttatttcgaccgaacgaagaccgacgttcgcgatactgaatacgtcttgattcaaacgccgcttgttgctgcacttgtcgaaatcgacgtattgacagccgacggggctacgcttggttcgccgctgttgttcgacattccggcagggtcgttaatcattccgggttcgcccgcgcttattgacgacaatacagcacttgcgcggtattccgtcgataccgtcggcgaagtagaaattaacgcgaagatgattcgccagcgttcgcaacttacccgacccggcgcaaccgtcgcacttgaacagttcggcggcgtgccagttttgacgaagcgtccgcttgcaaacgaccttgtacgcgacgaagtttcaaccggccaaatttcaatcgacaatcaaaccggacttgccgacattattagccgttgggactatagccgcatcggcgggccgcgaagcttcaaagtaaaccgtatttcgaatcccgaagaattggattattggaaagcgattcttgcgtatgcacgcggacaggcgcgcaagttttggatgcccacatatcgaaccgatttggctttggccgtgcaaccgtccgacgcgacttcaagctataccgtcgaagggtcggagtatgccgaaaagatttggccgattatcacgcatcgttatatcgaaatcgaaacagcgtccggcattcatcgaaccgaaatattgggcgcaactttgcttgaaactggcaatacaattcttcttcttggtacgccgcttccgaccggggccggatggatggaagttaagcgaatttcgtacttgttgcctgtacgccttggcgacgataaagtagaatggaagcattacggccttgaatcaatcttgaatctttcaattcgaacggcggaaccctaatgtctgattacgacgacaaagaagaaagcgtaggcgatagcgcgccatttgaactttacgaattcgtcggttcatatcgcaattattacatgacttccgacgttctggcgcacgtcttcaacagtgcgacatataaccccgttccgggtttgaagcgcagtacgttaaaagttggaacgcacgaagacgacaacgtagatatgacggttacgcttccgattacggaacaaatcgttaaagactatgcgtttcaaactacgccgccgtctttggttttgacgatttaccgtcgccagcgcgacgccgcaacttgggtcgcatactggaaagggccgattgcttcaatttcggttgacgacgaatttgtaacgttcagaacgccaagcaagttcggttcgatgctgtcgggcaatataccgaacgtttacgtacagccgccttgcaataacgttttgttcgacgaacgttgcaaggtaagccgcgtaagcaattcgctagatacgcaggtttcggcggtcgaaggtcgtaacatttcgattccgtcgcttggcggttttcccgacggttggtttatcggcggcgaaatcgcaatacctgcgcgcaacgaacgtcgaatgattgtcgcgcaagccggggcaattctaacggtaaattacgaattcgcgcgtatttctgtcggcacttcggttcaggttacggcaggttgcgaccattcttacaacggggccaacggttgccccaagttcaacaatcaaccgaacttcggcggttgcccgtttgttccgggcgaatcgaataacgtatttcaaagcggggttcattaacaaatgtggattgcaattgtcttcgtcgtcgccttcttgcttatggcggcgcttatgccgaaaccgaacgtcgaaaacgcacgcgcggcgaaacttggcgacttccaatttccgcgttctaagtacggcgacccaatgccgctagtgtggggaacagtgcgccagaaatcgccgataacggcttggtttggcgacttccgcccggtgccaattcgtaagaaaatgaagaccgggcttttcagttcgaagaaagttacggtcggttataaaaactatcttggcattgattgcattttgtgccttggccccggcgttcgacttcgcaagttttgggccggtacttaccttgtttgggaaggcaacttaacttcgtccggcgatatttacattaacaagccgaacttgtttggcggcgaagatgaacgcggcggcctgcaaggtacaatttcgttttacgacggtcgttttgacccgccgcaagattcttacttggtttcgaagattggcccgaatgttccggcttacaacggctttgcgcgtgcgttgttcaaagccttttatattggaacaggtacgacgccggaaccgttcagcttcgaaattcagcgcattacgtccggcttgcacgcgacttattcaattatgccaaacgggttggacgttaacccgatggaaattgtttacgacgcaatgacgcaaaaatggggccgcttcggcaaccttgaaagcgaaattgatatgccttcgtttacggcctgcgcgcaaacgctgtataacgaaggtttgggtatgtcgcttattgttcaatctgcgattaccggaaaagacttgttggaagaagtaatgcgcgtcgccgacggcgtgttgtatcaagacccggcaacggcgaagattgttgcgaagcttgttcgccaagattacacgatttccgaacttcttgttcttgacgaatcttccgttaaggaacttcgcaatttccagaaaacaacatgggaaaacacttttaaccaatgccgggttacgttcaagaaccgcgctagtagttacgacgacagcgtagcaattacccaagacttcgcaaatatcaactttcaaaaccgcgttaagtctacggaagtttcgacgcccggcgttaccgacgcaacggtagcaagcaagattgccgcgcgccaactttcaattttgaacgttccgctttacaagtgcgacttgaccgttaaccgcaaggcgcagaacttgcggcccggaagcgtgttcgttcttaattgggcaccgttcggcatttcaaacatggttatgcgtgttacgaaaattgatttcggcgaacttacgtctaaccaaatcaagctttcttgcgttcaagaccgcttttcgtcgtcttcgcttacgttcgcaccgcccgaaggttcaggatggacgccaattagcacggaaccgacgccggttacaacgcggcttctttttacgccgcctgcgttccttaccggatacgacgacagcgaaaccccggcttcgttcgattctgccgggcgtctgtaccttgccgcagttgcgccgggttctacgtccatttcttacgacgcaatgacggccccggacaatttcagttcgtcgcctattctgtcgcttgaaagcgccccgtacaacggcggcggcgtgttgctgaacgcctacgcttcgacagtcgccagcgaaacccgcaacgatacaagcggaagccttgtcgtaactggcgtgtcgcaagcaagcattgaaggtttgcagcaatacacgacgctagaccaagcgcgcgacggttcggccttcctgctgattaataacgaactgttcgtttatgtgggcttcgttgacaatggcggcggtcaagttacgttcccgaacgtgtatcgcggcgttctggatacagtgccgggcaatcatgctgcgaacgaccgcgtttggttcattagcggaaccgacggacttttgcccgaacttatttcggtcggaacgacgcgatacgttaagcttttggatacgacttcgggcggcaagcttgatatttcgttggccccgtcgttcagcgggtcgcttaccaaccgggccgggttgccgctgccgccgcagtacctaacgcttgcaggaagccgcacgccaagcccacaagtaggcgcaacgtcgattgctgttgcatggcgcaaccgcagccgggccgataccctgttgcgcgtgtatgacgacacggcgaacacgcgggaagctggcaccgaaacccgcgttcgctggcgcgtaggcggcggcgcttacacgaccgttacgacttcgggaagttcaacaacccttgacgtaacgggcaagtacggtacgctagaagttattgtagattcgtttattacggcgaattcgaaatattcgacgtacagcgattcgcttacaatgaccctaacggcaccgccgccgccgtaatcaaaagaacgacagcgcggcggttttggccttcttcaaccgcccgcagtcgtacaacatttcgaccgcttcgttaatgtaccaatcgtaatttatatcgtcgggaaattccgtcggcaagtccattacagggcgtgcgccgtcggttttcccgactttgtttccgctggatacgtaagcaatatacccggcttcgttcttcgggtaataccaacgaacaaccttgccaagataacgaccgttcttttcgccgccgccgcgcacgttctttactgcaacgaagcgccggaagtcgcggcattccttaatcgtcttttcgaccggcgttccgtcaacaaggaagcgaacaacagcgtcggaacaaatcagggtttcggggttcttcgaaagaatcgaattcaaggccgacccgcgttcgcaataagcgcccttcgtttttgcgcccaattgttcgtcaagaaagcgcgcttcggggtcgccgccgtcttccttgattgcgacataactattaacgtcgcggctgaatacggctttataccgcgtttcttcggtcttgtatcctgtccattcttcccaagccgcaataagcgcccttacttcgtcgtgccgcgatttgtgatacttcgaaataatgccgtcggtattgccggaaataacttcaatgccgatttgttccaaagcttcgataagcataagcaacacaagttgcccggtaatcgttacttgcaacataagttgcggcgcgtaaagcgtcgaatacttgttgccaagtttgccgaagcttccgttaatggtaatcttcaagctgtcggcaacggtcttccatttcttcgcggctgcgcggtcgcccgctttctttgccttagccgcttcggctttggcatgaattcgcgtatttacgattttttcgtaaacggtcaagaaagcttcgcccaagtgcggcgggcaaagtctttgattaaggattgttcgcgggtaaaacgattctacgtcgttgtctgccaaaatgatttcgtcggtcgcatagtgcgccgttttcttttcggtagaatgcaaaccgcccatgcctagtttgtacgtcgaaccgccgatggttactttcagcttttccagttcgggcggcatgattggcgaacccaagccgtcaagatagaagcgggcgtttcgcacgacttccagcatttcgcgaagctgcgtcgtttgatagcaaacgaagtcgggcacgtcgtaaactaagaccgtatcggcggataatgtgggcttcttcggataatagccaaggactttttgaagttcggagttaataaccgcttcggctacttgtgcgtctgatttcgaccgaaggtctacgccgtattcttccgacatttcggcccgcaatttcaattcgggcgcaagttcgttaaacaaaagttcggtatttgaaaggtcgttgcagcaataaggccgcactatttcggcgtcttcctgcgtcaatacatggtcttcgggaaacggcaagtcttgcatacgttcgcaatgcaagcgccccgcgtacagcttcaacgaaccttgaagcggcgcaacttcgataaggtcgatatgattgtatcggccaatttgtacgccgtacttcttttcgaacgcgaacggcgttaccttttcggttccatagttcggcccgcttttaatgatgaaatcggacgcttctttaagcttcgcgcagcttgccccgcgtgcggctagttcaatcatcggcaagtcataattgcgcgaattgaagccgacgatacagaaccgccaaagcatccaaagaagcttcgtcgggttaaaatcatggtcggggctgcgttcaaacgcgacgaactttccgttatcaagcgacttaaacgctacgtaaaagaaatttacgtaagtttcaacgtcgaagacgaagacacttccggcaggcaatgccgttaattcttcgtccgacataaattcaacaggccgcaaagcctgtcgaatgcttgcggccagcttatcgacgacgcggcttttctttgcaacaataaagccttgttcgttaatcatgcgaaagccccttaaaacggtatgtcgtcttcgtaattgtccgacggattatgcgtcgcttcgctgccaaggtcaagacccataagaacgccgcgcaggttttcgccgaaaaatacgaccttgtttgcttccggtgcgaaatgcgctttcttaaacgcatgttcgacggaaagaagcaacttcgcagaaaagcccattcgttccggcaagccttcgattttgtacgtcgaagcttcttctttgtgcatcttcgacaaaaccgccccgtcgtcgaaaaatacgtttccgttcgggctgaacgactgaatcgcgcgaacgccgttgtaaaaatcgtcgggaaccggccaagggttcaagccttcaacttcgaaaacgacttggtaattcgggtaacgttcgccgtaaagttgcgttttgataaacgccccgttttcgaaatagaacgttgccgacgacgccgaaaagccgaagccggtaagggccgggccagccttggcgacagcgaccgccgcgcacttcggaagcatcatgccgggcggaaggtcgataccgtgccacgcttccagcaaggccgccccgttggtcgcgacggcgcttccggcttgcagcaagacggcggcataggtcgcattcggtgcgccgtctgtcgcaagccctgcgaccgcagcgaaggccgttttaatccggtcgtcgattgtggcgcattgcgggtcgggcgcggtaatcggcacgtcgtcgaagcccacacaaggcaccaacgcccggaacgcgccagacgatacggcaagggcgttcgcggtaagctgcgtaatcgacagttcttcgccgaccttcgacagcgcgtcgataaactgcaacgtatgcgggcacgctaccaagtcttcttcgataggatgcgcgacggtcaaaaccccgtcggaagccgccgcccaattgtgggcaatgtgcccgaactgaacgttcgtcggcccggcctttttctgcgcgatggcaacgaacttcaacgccgccaaaagcgacgccgcagggcttggccccttggcctttttctttccggctgcggcttgcctgcgcgcccttggctttttcttcggctgttcggcggctggcgcttcgtcttcgggcggaagcggaatattactgtcgattttcattttcgaccccttcttcgtcaagtttcgaaattgccgcttgaagatagttcgccaagtctagcgtttcttctagcgcgtgttgcaaccaatcgcgaagcgacaacgggttattatccgtcgttacgccgtactttgcaagtccaacgattgaacgttgcaacagtaattcgcggttcttttctacgtttttatcgggcgatatttcggccatttgttcaccattcagcggaaagaacttcgggatactttttgttcacccatacgcgaacacggcgcggcgtgcgaagttctgaaacacgctgcaaagcttgataagtcgtcggcggcggttcttcggcgtgccgctgccgccaccaatcgcgcgaacgtttcgcagcaagccccggatgttcaagcattataaattcgttgaacatttggaagccgcaaaagtaactaaccttaatcatcggcggtttcgttagatttccgtttgcgtcgcgcttttcgtgcaaattgtaaagtaccttttgaacgtcgaaatattcgactatcggcgcgtcgcttcgcagcggttccagcgttccggcagtcgcaaacaacttcgtttcaaaagtaaattcggtgccgcagttgatacactgtcgggccgcagcgtgattatatacgccgcatacgtcgcaaatacgaacgggcgcgtcgccgccgcctttgcccggtctgttcggaattcgcgggtcgttgattgggccaagccttcgaacgtttccggcgaagtccgcgtacaggcaattttgtttgccagtatccggcgacggtcgggttccgcgtcccaacttttgaacgtgtttgcccggcgatagcgtcgggttcaaatcgccgataaagtcaatcggcggatggtcgaaacccgttgtaagtttctgccccgataccaagccgcgaagttcgccagctttgaaagcgcgcaaacgttcggtattggtcttcgaaggcaacttggaatgaaccggcagaacttccaagccgtaagactgaattacgttcgcgacgtgttcggtattatcaatgccagttgcgaagataagccaagtcgaacggtcgtaagccatttcggtcatttcccgaacagcgttaaaaacaacttcgtcggtatctacggcggcttctagttgcttgctgttgaattcgccagccgttacgcctactttcgacaagtcgatttcggttcgcgtcggccttgcgataagcggcgacaagtagccttcggcgataagtcggttaaacgattcgacgccggttatgtcgtaacaaatgtcggtaaaaattccgttttcggtaataagccccattttcattcgatagggcgttgccgtaaatccgataactttaagatgcgggttaatcttgcgaagttcggcgataatgtattggtaatgcgaatcttccttgtcgcttaacaaatggcattcgtcgattaacagcaagtcgcgccaaccgaaatggcgaagatgcgccgggcgtccgtcgtcttcttcaagcccgcgtttaatcgcaggcgctacggactgaacgccgccgaatacgattggcataatcatatcgcgggaattcaagcccgccgaataaatgcccattggcgcggtcggccaaatcgacataagcttttcggcgttctgttcgattagttctttaacgtgcgtaagcatcattattcgttgattcggccagtaaccgaaaattcgacgaataaagtttgcaattacgaccgactttccggttcctgtcggcatggcgacgacggggtttccgaccccgccgcgctggaaataatcgaatattgaatattccgcttcgtcttgataccaacgcggaacgtaaatattagacattctttgcaaccgctgtaattggcaagtacgaatcgcaacccgtcgggataaaatcgcccggaattacgccattgtgaacttcgcaaaaccattcgccgttttcgacagggcgcgacgctttgcagcttcggcaattcttttcgggaattgcgcccttgtggcaaatgtctttcgccgcgcaatatccgcacttgtgaaacgtcggattatccgacaagcgcggcggcgcttcctgcgaaagaatgatttgttcggccttttccttcatttgctggcctagccgatggtctagctttacaacttcgcaatgcaagtcgtcgtcgttcttgttaatgttcaggtaaagaacataacgaaaacgatattcgttgccatacgtcgaagtttgggcgaaatgttgcggcttacttactgccattccttgcgacattaagtccgaaaagcccttgcctgttccgttggtcttgaattccagcaaaacgggttcgtcgataccgtagcgttcgggaagccgggcgataccgtcaagcgacccgccgaagtgccccataacgccggaaatgcggtattgtgggaattccaagccgtcggctttcgcgcgggctacatgctgcaaatacgcggggttttccggcgtaattcgttcggccagtccgtcgccttcttcgccttcggcaaggatgcaatacgaatcgctttccgcgtgatacgcgaagccgtcgcggttttcgtaccaacattgaacgccgataccttccagccattcaataaagcgcgcttcttcacgatgcccacgattaaacaagcgttgcttgcgcccgtcggtttgttcgcggaaacaccaacggaaaatataccaaagcttccgcttgcattcgtcgccaatcaaagacgcgccaaggtgccgacggtgcccgccgtcatacgtgcgaacgcaatattcgtctatatcttctttaatgcgctttgcaagggcggtcgcaacgccgggcgcgtcaaggtctacgctttgttttcctgcgtcggatttcgtcggctgcgcgtcgttcagcgttaattgtgcgttcttcgcgtctttgctgttttgcatcgttcttcaccttgcgccgcataagggcttcgtaattgttcgctagataatccgccgaaagtgtcgcaacgtctttaagttggtctttcgacagccaagatatatggcattccgacgggtcgattcctaattggttcgcaagccaattgtacgccttcgcgcgcgacattaagccggtttgccaaaggcggtcgaattcgttatgggctttcgttcgaagctggcgcgttgttctgtcggccatacgacccaacggaataaacgtgcccggatggcaaccaacggcggcgcgacaatcgttgcaataataaatgtgcggccagttgccataagttcgcccgtaaatcttgtcgttcgttgttagttcaatgttgaacgaacaacaagtatcgcattgttcgggcgtcggcaatgcgtcttttattttcgacataagttcgcatactccttaaaaagaaacgccggggccggttaagaccccggcgaatctacgccgcgacggcgttacgccttaacgctggctccaaggtgccgcgcctgccggtgcgccgccctgttgccagccgccgccctgcgcgggctgctgttgggcgggctgttgggcaggctggccgccccatgccggttggccgccctgcggggccgggttctgcgccggttggccgccccatgctgcgccctgcggctgttgggccggttgctgcgcgggctgctgctgtccgccccaagcggcaccgcctgcgggctgctgcggctggccctgcggggcaccgccccaagcgccgttcggctggccctgcggctgctgttgctgcggctgcgctgccggtgcgcctgcgccagccttgcccggttcgttgccgttcatatcgaacacctttttaacttcggtatattgcgggtcgtttttctgcggcccgacttcgataagaaacggcaggttgtgaagctgcgacgaatcggtaacttggaacacgccgataacgtggcagattgccgaaagctggcgatgcgcgatttcgacggtctgttggttggaatggtacaggttcaggcgataagcgccggtcgtgccttgctgcgggccgtcgataatccgcaagttcagttgcagataaccgccgtcgttcgccttgttggccttgacttcggaagattcgatgataaccggatgcttgccaatcggcaggcttccgacgccttgggtcgggtcgtactgttgggcgttgaacgcttggataagttgcatgatatttcaccttttcgaaaagtcttcgccattggttacggcggcgaagtaaaccgttttattgcatcgctttcgcgaacaatgcggtaaggtcgggctgttcaagttcgttaagattgcccaaacggtcgcgcgcgaaaacttcgggaatttccttcgttcgcagcgcccgaaccggcttcggcattccgggtacgcttgcttcgcccaagtgcataacgttatcgaacaaatgcggaactttaacgttaaggtctttgcccggaaagaacgggcgtttttgcataatcggttcgtaagtaacttcgccgttctgcaaaatcgtttgccgtccgttttcgacaagggcttgtttcgcaatcattacgatatgcttttgcggcatgtaatacaagtcgtttgcgattttcattacacgttcggacatattgccgtaagccttcataccgtgttttaccttgcccaattcgtcggccaagataatttcggcgatgttcgaaatactgtcaatacccaacgtatcgaaattcgcagcttcgcgcgatttcataaaccattcgaagaattcgacgataagcgcgggcgtgtacgcttcccatgcgggaacgttcgaaccgcgcatggacagcataccgggttcggtcacaagcaaaaccggacgcggcgcagtattgataagcggcgttttgccggaaccgggcgcaccgaatacgacggactttacgccgtagcggcgggccagttgcgacgccggttttaattgcgacatttgcataacttgcaccttcgggaatggccccggcgcttgtgggcaaccggggcggggttgttacttcgacttcggcgcgacaagttcaagcgcgggcgttgcttccttcgacgttacaacttcgtcgataagcttacggaacttgtcgggaaggttcttgtattcggtaacggaaagttcgggcttccattttaccaaacgttcggcgataaattcgccttcggggccggtcttttcgattttctgcaaagccttttcgacggcttcttggccgcctacaaagctgcggttaatcttgaaaacggctttcagtttgtagcctgcgccaagttcgacgttttcggttccttcgcgcaaagcttcggggtcgaaagcgaaagcgtttttcagaacttcggcgcgcaatgcggattcggcttctttcgctgcggcaagcgccttcgttgcttcctgccattgcagaatcaaagcgtcgcggttagcgttttcgttgaaagacattgttgttactccttcggttgggccgccgcacaattgcgccggtatgtacgaactatacgacggcccttcgttgctgtcaagcccctttttcaagaatttttcgttcttcgaacaagtcgcgattgttggcgtcgaattcggtaaaagcgttcgggaagcgacgacgaagcttcgcgatgttgaccgactgcgcttcgccgaagttcgaaccgatagcacgcagcaaaagggcatcgtaccaaaagccgtcgccgacttcttcaagcgcgttaacagcgtcgaaagcttcgccttcggctgcggcgcttagggcttccaacagttcgcccgcttcggtcgccttgccgatgattgcatgaatgatattcacggcggcggcgtcgtcttcggggcggtcggaaatccattcgggaagcttcgccagcgtcgccgcgttggcttccggggccggaacgccagtgtcgcgaccgtagaacatggctttcttgattgcgtcaagcttgttaagggcttcgacggcttcggcgatggtttcgcggaagtaggccagcggcacgcggtcgccgtaatacttatccgacgcggttacgtgcgcttcttcgatatagtcgaaggtctttgcaggttcgttcatacgttcaccttttggaagggccgggcaccatgcgccagcgttccagcactgtaaccgacagcgcctttcctgtcaacaacaattgacagcaagcaagaagccgggtaagctgcccacaattgctagaaggtggcgttatggacattgaagaacttacgaagcgggtaaagaaagcaagcgtcgggcggtcgctgcgggcactggccgcgcaatgcgacgtttcgcacgaacttataagaaagcttttgcttgccaattctgcgccgaacataacggcgacgacttataacaagattgacaaagggttggcaaagaatggactttaacaatattccgcacgaaatgcggatttacccgcaatgggtcgtttggcgatacgaagacacggattcaaagaaaccgacgaaggttccgtattcagcaaaaaccgggcaccttgccagcgtaaccgacccgaacacttgggcagggttcgacgaatgcgttaacgctttgtcgtccggttggtatgccggaatcggtttcttattgactgaaaacgacccgtattcgtttatcgaccttgacgacacaaaaggcgaccaaacggcgcttgaccgccaaataaagatttttaacgaattcaacagctacgccgaacggtcgccttccggttcggggttgcatatcatcgttaagggcgcgattccatcgggccgccgtcgttcgtttatcgaagtttattcttcgcttcgttatatgacaatgaccggcgacgtttaccgcaacgcaccgattaacgattgtaacgaactgttaaacattctttggggccaaatggggcaaggttcggttgccgtcgcgcattacgccggacttgccgaagccaaggaaaccgacgaacaggtttataaccgcgcagtcgccgcagccaatggcgacaagttcgccgaactgtacgcgggcaaatgggaaggcatgtacgcttcgcaatccgaagccgactttgccttggtcgatattatcgcgttttatacgcagaaccgggcgcagatttcgcgaatgtttcgcgcgtcgggcttggggcagcgtgacaaagcaaaacgcgacgattacgtttcgtacatgctgaacaagtgtttcgaccgcatgttgccgcccgttgacgttgacggattgcgcaacaagctagacgaagcaatagcaaagaaagaagccgccgacagggccgccgcgttgtcgcagaacagcgaagcgacgccgcatccgaaagccccggccccgaaccttaacgaagcttcgaaggtatacagcgtgccgcccggattagtcggagaaatcgcgcaatacatttacgcacaagcgccgcgcccggtgcccgaaatcgcgttggccggtgcccttggtttggtcgccggtattgtgggcagggcgtacaacatttccggcactggccttaatcaatacgttctgttgttggcaccgaccggaacaggtaaagaagccatcgcaagcggcattgataagctaatggcgcaggttatccgaaccgttcccgctgcgtcggactttatcggccccggcgaaattgcttcgtcgcaagcgattatcaagtatatgtcgcgcggcccaacgtcgttcgtttccttggtcggcgaattcggcatttaccttcaacaaatggcaagcgtaaacgcaccgccgcaccttaccgggcttcgtcgcttcctgttggatgcttacaacaaatcgggcgaaggcaaggtacttcggccttcgatttattcagacaaggacaagaacaccgccgccgttctttcgccgtcctttaccttgcttggcgaatcgacgcccgaaaagttttacgaaggtttgcacgaaggtttgatttccgaaggtctgttaccgcgttttacgatgattgaatatcacggcgaacgcccgcctttgaatccggggcacctgtcggcccaaccttcgttcgaacttatcgacaggctttcgacgttgtgcgcccatgcgctaatgttgaacagccagcataaggcgattcacgttcaaaccgacgcaaccgcccgcgaactgttccagcaattcgacgcgcattgcgacgcgaacattaacacaagcgaccgcgaagttcgacggcacctatggaaccgggcgcacgtaaaggcgttgaa

SEQ ID NO: 2 - DNA, 42566 bp, unidentified
  source 1..42566: mol_type = genomic DNA
ccgttgggtcggccaagccgacagcggcgttaagttcgaactgaatacagatttagacattgcccgcatgacgccggaagaacgccgttcgcttgtttaagaatggcagaaaggcgcaattaccttcgaagaaatgcgaaccggcttgcgtaatgctggcgtcgcaaccgaagacgacgccaaggcgaaagaaaaaatcgccaaggatcccgccgaagcactggcccttgctgccccggccaaggtgcggggcgatggacgcggcggcgataatgtgggaaacagcgaatacggggcgaaatcatggcaataccgcacaaaacgcgactgtacgatattgcgacgcgccttgctgtttacgtcgaagacgtgaaagtttggcagtcccgacaattcgggttcgtacttcgcgaagtaaacttggaattaacgcgccttcttggtcgtgttcgttataagacccttgacggtctttcgaaggcgcagttaaacaagcttgtttcggaattacgggaatcgcaatcgaagatttacagcgcatacacgcaaacgctacttgaacagttaaaggaattcatgcgggccgacttggaagtaaaccggcgcgcatgggttacgggttatatcgaacttgacggcgaagaaagcgacggcattatttcggacgaagaagcgatacagtttcttttagaagtacccaacgccgattcgaaccctttgttcggtttggccgctgttacaggaagcgacgaacgtatttggtcgcaagttacgaatacgccaattccggcaaatggtttgtacttgctgccgtttatcaagacgtttgcgaattcggcacaagctggcgtcgaaagcatgattcgcaaagcttgggcgaaccgttggacggttgaagaaaccttaacggcacttgtcggcgatggaaccgcagcgcagggcacgccgtcgcagttgcaccgggtcaacgcgcaagccgcttcggtaatccatacggcaaccgcgcacgttgcggcggtcgttgcggctggcgttatgtctgcggttttcggtcgttacgtttggtattcggtaatcgacggaagaacaacggacatttgcataagtcgaaatcgtcgaatttatcgcttcggcgaagggccgttgccgcccgcgcatatccgttgccggtcgcacgttgccccggcaaataccgctagtgacttggccgaagaaacgttttatacttgggttgcccgtcaacctgcccaagtgcaagacgacatattgggcgacgaaggcggcgaagctttgcgcgatggtcgcttaaaggcgaaagatattccgaagtatgatgcggacaggccgctaacttacgaagaattccgacgcaagattaaagaaattctttcccgctgattcggtgaatcggcataaccgcaaggagtccttgaaatggcactgaaaaagaaacttaccaaagaagaacacgcgaagctttcggacgcgctgaaagccgaatatatcgaagacggcgacggcttccgccttgacgttgacggcgacgaagatacgggcgctttgaagcgcgccaaagaccgcgaagcgcagttgcgcaaagacgccgaaaaggaagcgaaggaacttcgcgaacggctggaatccatcgaaggcgacgacgcgcgcaagaaaggcgacattgcgacgcttgaaaagtcttggcaatcgaagcttgaaaaacagcgcgaagaatacgaagccaagctttccaagcttaccgcgcatacgacgaaaacgcttgtcgataacgtcgcgtcgcagcttgcccacaagatttcgaacgccccggcgcttatcatgccgcatatcaagtcgcggcttatggcagacttcgaaggcgacacgccggttacgcgcgttctggacaaggacggcaagccgtcggcccttactatcgacgaactggcaaacgaattcgttgcaaacaaggatttttctgctattatcacggctagtaaggcgtccggcggtgccggtaagccttcgcagaacggcggcggtgccccgaaatttaacggtcaatccgacaaacccgccgacctttcgaagatgaatcccgcagaacttgcggccttcattaaagaagcgaaggcaaccgaaactaaggacgcttaatcatggcactttccgaccttgctgtttactccgaatacgcttattcggctttctccgaaaccctgcgccaacaggttgatttgttcaacgctgcgacgggcggggctatcatgctgcaatccgcagcgcaccaaggcgacttttccgacgttgcctttttcgccaaggtaactggcggccttgttcgccgtcgtaacgcctacggttccggcaccgtcgccgaaaaggtcttgaaacaccttgtcgatacgtcggttaaggtcgcagccggtacgccgcctgttcgccttgaccccggccagttccgttggattcagcagaacccggaagtcgcaggcgcggcaatggggcagcaactcgccgtcgatacgatggcggatatgctgaacgttggtcttggttcggtctattcggcactttcgcaggttgcggcggttgtttacgacgcgaccggcaatactgcgccggacgacggcccgacttggaacaaccttaacaacggccaagccaagttcggcgaccaatcttcgcagattgccgcatggattatgcacagtacgcccatgcacaagctgtacagcaacaacctgtcgaactccgaacgcctgtttacttacggcaccgtgaacgttgttcgcgacccgttcggcaagcttcttgttatgaccgattcgccgaacctgtttaccgcaggtacgccgaacgtttatcacattcttggccttgtgccgggcgcggttatgattggtcaaaacaacgacttcgacgcgaacgaagaaaccaagaacggcgacgaaaacattattcggacttaccaagccgaatggtcgtacaacgtcggcgttaagggcttcgcttgggacaaggcgaacggcggcaagtcgccgaccgacgcggcgttgttcacttcgaccaattgggacaagtacgcaacttccgaaaaggatttggcgggcgttgtcgtcgaagttcactaacggacggcagacggacggggcttcggccccgttcttctagtccttcacaaattcaaaggagtttcgaagatgaaaccggcaaaaattctgttcttcgttgacggcaacgcaccgaccccggaagacttcgcagccgccgccgacctgaacgcgcaagttatgttcaggaacgcccgcgctgttccgtccgaaccgcattcgctggaaatctgcgacggcgtagcgggcaaggttccgaagctgtacgccgacgcttacccggaagcgggcgaagcaatcaagaagaaggccgccgaactgaaagcccttacttcgaaggtcggcgacgccccggcaccgaaggccagcggcaaggccgccgacaagcccgcagccgctacgcagggccagaccccggcgcagggccagaaagccccgcagggcggcagcgccccggcttggaaccccaacccggcgcagtaaccaacggacggcctgtcgcttattgtgggcaggccgtttcaacaacgaactaaggagtcttcgcaatggcaacgaaaaaggttgtttatttcacggcaggcattaacgcaacggctggcgaacttgccgacattgcgaagcttaacgcagccgccgaaccgcaatacgaagttttggtagccaacggcgcagcaaatgccgaatacggcgaaagcgaccgaattattccggctgattttgtcgcgggtacgattcccgacgtttaccgcgttgacggcgacccgctggaagacccggtttatcccgaaatcgaccccgacgcaatcccgaatcaggcgcttacggcaacgcaagcaatcgttaacgacgcgcaagttctggacgttgacggcggcggtactgttacgcttaccatcgaaggcggcgttattaccggcgttgtttacgccgctggcgaataaggcggttgacgcatggcaattacaatcgttgttgaagacggaagcggcgtaacaaacgcgaacagttacgtaagcgtcgcagacgcgcgcatttacgcttcgaatcgcggcgtcgaacttccgtcggacgacgacgaattggccgccatgctaattcgttcgacggattacctagaagcgcaggcgtgccgctatcaaggccgccgcacttcttcggcgcaggctttggaatggccgcgaactggcgtctttttgaactgcgacgaagtaccgtcgaacgttattccgaaatcgctaatcggcgcgcaagttcaacttgcgattgccgttaacgacggtttcgaccttcaaccgaacatttcgccgcaagactacgttattcgcgaaaaggtcggcccgattgataccgaatacgcagacccgaccgccgttggcatcatgcccacatttacagcggttgacgcgcttcttgcgccgctgtttggcgaatgcgcggcgaacaagtttgcgcttcgaacgataagggtttaacgaatggcacaattcgaccgcgcaattaaaacggcgcaacgccttatcgcaaagaacggcgaaaaggtgaaatggcgcgttatcgaagacgcgacgccaacagacccgaacaagccttgggaacccggcccggcattgccggacgacaaagacgttaccatttgctttttgccggttgaccggcagacgcaagaaactttcaacttcattaaaggcaccgaagttccgaaaggttcagtaatggggcttatgggcaatgttccgtttgaaccgaatttgaaagacgttgtaattcgtaacggggtcgaacttcgcttggcttatatcgacgtactttcgcctaacgggcaaaaggtactttacacgatggtatttcaagcatgattgaattcgaccaagttaacgacgaagtaaacgcgctttttcttgcaacttggaacgcaggaagcgcggcaattgcgggttatgttcccgaaattcgttggcagggcgtgcaatatcgcgacttgccggacggttcgaagttttgggttcggctgtcgaagcaaaccgtttttgaagaacaagcgaccctttcaacctgcgaaggagtaccggggcaaagaaaatacacggcgtcgggtcttgtcttcgtgcaaatcttttgcccgaaatcgaatacgcaagcgttcgaacttgggcagaaattggcgaaactttcccgtaatgcttttcgcgggaaatcgacgccgggtaaggtttggtttcgcaatacgcgaattaacgaacttccgcccgaagaactttacgaacggtttaacgtcgttaccgaatttgaatacgacgaaataggttaaggagttcttaacatggtatgcgaaattgccaaaatcgacagcaacattaccggacttgccttcgccgaagaagaatgcttgaagcaacttccgacgacgcccgtttggtacggactggaacccaacagctattcggacttcggcggcgaactttcgaccgttgcccgcgcgcctatcgacccgtcgcgccagaacaagaaaggcacgattaccgaccttgacgcatcgggcggcttcaacgccgactttacgaagaccaaccttacgcgaattctgcaaggcttcttcttcgcagacgcgcgcgaacttccttcgacgcagccgcttaacggcgcatcggttgcgcttaccggcgttaccgcagttgacagcacttacgccgcagcttccgggcttggcgtgtttggtgccgatatgctggtatatgcgaccggctttgccaacgcggcgaacaacggccttaaaaccgtcgtttcggctaccgctgcgggcgtcgttgttgctgaaaccctgattgacgaaaccccgcccgcaggcgcgaagctggaatgcgtcggtcgccaacttgccgccgcagacgcgaacattgccgtaactggcaacgtcgtttcgcttatcgttaccgctggcgactttacgaccatgcctgaactgttccccggtcgttgggtcttcgtcggcggcgatgcgacggcaaaccgcttcgctaataatgtgggctatgcccgcattaagtcggttgcagcgaaggcgcttgttttcgacgacgtgacttggcaggcgaccaacgaaaccggaaccggcaagtcgattcgtcttttcgtcggaactgttatcaagaacgaaaagaccccggcacttatcaagcgtcgttcgtatcaaattgaacgcaccttgggcgaaggtttgaacggtacgcagtgcgaatatctggaaggcgcagtaccgaacgaatttacgttgaacattccgcaggccgacaagctgaacgccgacctttcgtttgttgcttgcgataacacctatcgcagcggcgacgttggcgacgaacagaaaagcggaacccgcgtgcctgcgccgggcgaagatgcgtttaacacttcgtcggacatttaccgaatcaagatggccgttcacgacgacgcttcgtcgaatcctgccgccctgttcggttatgtttccgaagcgaacgtttctattaacaacaacgttacgccaaacaaggcggtcggcgttttgggcgcgttcgacacttcggcgggtaacttcgaagtcggcggttcgattaccgcttactttacgaccgtcgcagcggttaaggccgttcgcgcgaacgccgacgttggtttgtcggttatcgctgcggccaagaacgccggtttcgtgttcgatattccgttgcttggtttgggcggcggtcgtctgaacgtcgaaaaagacgcgccgattaccgttccgctggaacccgcaggcgcagaaaacgcgaacggctatacgatgctgtacgaagtgttttcttatctgccgaatctggcaatgccggactaattgcggtaaactcaaagggccgggtaataccggccctttcttcattcaatcggagtaaatcaaatgtctggactgtttaagcaattcaaaacgaattcgaccaaggaagtcgaaggcgtcgaaatcgaatttcccgaagcgcagaacgacgacggcaccgttcccacgttcatcatttcccgcatgggcaaatcgaacaaggcgtattcaaaggcgcttgacgcggcgacccgtccttatcgccgtcaagtcgaacttggcacgctgaaaaacgaagtcgccgaatcgctgtttatgggcgtgttcgtcgataccgtgttgcgcggctggaagaacgttcagggcgaagacggcaaggaaattgcgtattcgaaggacgccgcaatttcgcttcttaccgaactgccggacgtttacgaacgtttgcaggaagaagccaagttggcttcgaacttccgcgataacgctttggaagccgaagcaaaaaactaacggaagttttggcgcacctgttggaacttggcccgcacgaacaggcgatagcaaagcaagcgatgcgcgcggggcagccgttacccgaacgcatagcgaacgcgccagaacttgaagtaggcttgcagttgtatttgcaagccttcttcgacttagatagcgaacggtcgcacggcaacggtttaacgccgataccttggacaagtatggcggcttacgcaagggctttcgagtttgacgaagaacaaaccgaagacttgttttactttatgcgaaagcttgattcggaacacttgaagaaactagcggacaaacagaaagcggcggcatcgaatggcaaaaagcctgctagacctagccgataggctggaaaagaaagcgaaggcaatagacgaagcggcgtcacaaaacgccgtcgataccgctttagctattgtgggcgacttggcgtacaaaacgcccgtagatacttcgcaagctttgtcgaactggattgtaacgctagaaagtccgtcgggtcaacaaatcaagccgcattttccgggttcgcagggttcaacgcaacgcgcttcggcggctgaaacgttgaattctgcgaagcttgttttacgaaacaagaagcccagccaagcgatatttattacgaacaaccttccgtatatccgcagactgaacgacggttattcggcacaagccccggcggggttcgtcgaacgcgccgttttgattggtcgcaaaatgcgtaagaagttcaagattaaggattagaagaaatggccgacgaaaacatagaaattaaggttcaagacaaagtttcgccgtccatttcgacgaagcttcgcacaattgccagcgaagcccgcaacgccgacgccgcagttaagaaccttcaaacgcagctttcggcaatcaacgtcggcggcttgtcgcagcttatcaacgcatcggccagcgcaacgcgccagcttcaacaaggcgcgcttgccgcgcaacggctggcaaccgaacagcagcgtaccgcaacagccgcagcgcaggccgccgcagcccaacagcgcgtcgcgacggctgcaacccaaggggcaaccgcgcaagccaaccttgccagcgccacgcagcgcacgcagacggcccaacagcagacggcgacagccgcgcagcgcctagcgacagaacagcagcggacagccgttcagacggccaacgcggcggcagcgaacgaccgggccgcccttgccgccctgcggctgcaacaggcgcaagaccgggccgccaattcgacccgcaacgcgacttcggcccttggcggatacattcgaaccgctgccggaattcttggcgtaaccatttcggcgaacgcaattcttgcaagcgccgacgcatacgttacgcttcaaaacaagttgcagaacgtaacgaagtcgcaagaacaagttaacacgcttacgaacgaacttttcgaactgtcgaaccgcacgcgcgcgggtattgaagaaacggcgacagcgtttacacgcttcgaccgtgccttggcgtttatgggtaagtcgcaagaagattcgttgcgaatgaccgaaaccattaacaaggctttgattgtttccggtgcaacagcgcaagaagcgtcgtcggctttgttgcagctttcgcagggctttaacgccggtaagttgcaaggcgacgaattccgcgccgtttcggaaaatatgccaatcgttttggacgcagtagcgaaggcgcttaacgttccaatcaaccgcgttaaggaactttcgaccgaaggtaaaattacttccgaagttctgtttgaagctttcaagcttatacaagaccaagtagacgcaacttttaacaagacgacgcccacaatcggccaaagcttgacggttcttcgcaacaacgcaattcagttcttcggcgaattgaacaaagcgaccggctttactgccgggctttcgcgcgcgattctttggcttggcgaaaacttgaagacggttgccgttattgtcgcagggcttggcgtcgcattgcttgtcgccttcggtgccccgttggttggtgcgctggccggtgcgacggctgcggtaaaagcctttacgctggcgcttgcttcgaacccgattggccttatcgtcgtcgcgctttcgactgcaattgcgtatctaacgcttttccgcgacgaaattaacttgggaatcgacgacgttacgacgttgggcgatttcttccgcgcgacctttgaaggcatcggccaagcaatcgacgacgttaaaaccatcgcgtcgcaattgtgggcagaacttagcgacttcgcaagcgcagcccttggcgaaattacttcgaacgttgacgattcgacttcttcttggtcggacagttacgccgcattctttcaaaccaaccgtacaggttgggccggtgcgctggaaaacgccgcaaaagttcttgacgcaatcgccgggcttttgaccggggccgcaacgttcgcaggccgtgcaatggccgaagttgttatttcggttcaaaacggaatcgcaaacgcttacaacgttgtcgcgggttggattgaaaacgtaacgaataaagccattgaagccgcgaacaagcttcgcgcgatggtcggtaagtcggcttacgaacttgtccaatttgaacgcatgggcagcgcaggccaaaccgaattcgaatcttggggcaagctttgggcgcaatcgctggaagacggatttaacagccaaggcggcgcaatgcaaaacctgttgaacggccttttcgaccgttcgcagcaaatcggcgcgcaacgccgggccgcaggaagcgcgcagcttcgcgggtctggcgcgtcgcagcttgccggggctaccgacgcgaacgcggcgaaggctgcggaacgtcgcgcgctggcaatggaaaaaatcaatacgcagcttgataacgaattggcgcgtatgttccagcttcaaccgcagcgcgaagcgcaagcgaagttcgaccaaatcgaagaaagcttgattcagaaaaagattaagcttacgtcggaagaagccgaagcaatcaaagcgaagattaaggccgttcaagacgcgaccgaagttcaacgccagttcgacgcaatttacgccgaagctgttaacccgttgaaggaatacaacgcttcgcaggaagcggcgaacaagcttcttcaaatgggcgcaattacgcaggaacagcacgcccgcgcagtaacgaaggcgtcggaagcttacgcaaattcgcaagacccgttgcggcagtataaccgcgaccttgaacagcaattgcagcttttgcaaatgctgccgaagcaacgcgaaatcgaacagcagattatgcaagttcagaacgatttgcttgcaaagggcatcgttctaaacgaaaccgaattggcgcagcttcgcgaaaagctgttgcttattcagcaagtaaacgccgtttcgcagcaagaagcgtcgttgttggacgccagcgtaaacaagcgccaacagtttatcgaccaacttaaagcaattcagaacttgcgcaacaacagcggaagcggtttcaacgcaggcgaccaagccgaagcaactaacagcatgttgcaaggcatgggtattgatacaacgaactttcaaacgcagcttaacgcgcaattggcgcaatatcaaacttacgtcgaacagcttaaaatgctgaacgaacagcgtttgattagcgaccaagaatacgccgccgcaagtatgcaacttgaattgcagcggcaaaacctgtacttgaattcggcaagtagtttcttcggaaacttggccgcgcttcaacagtcgggaaataagaaaatggcggcggtcgggaaggccgcagcaatcgcgcaagcgatgattaatacgtatcaatcggcaacgtcggcttatgccgcgatggcgtcgattccttacgtcggcccggcgttgggcgcggcggcggctgcggctgcaattgccgcaggcttggcgaacgttcagcaaatccggtcgcagaataccgggtttaagtcgggcggttttaccgggtcaatgggcgtaaacgaagtcgcgggcgtcgtacacggccaagaattcgttatgaacgcatcggcaaccaaccgcattggcgttgcagaccttcaagccctgcaaacgggcgcggccagtgttcagcgcaacgatgaacaggcaatggcggctagtgtgggcaggggcggcaattccgaacccgcgcccgcgccggttgtcaatgtaccttttagcgccgttgtcgtacaatcgaaagaagcggccttggccgggcttaaatctgcggaaggccgggctttcattctggaaactatcgaacaaaacggcggcaccgtcgcaaaaatcgtcggagttaaataaatgggttacgcaattggaacagttacaaaaggcggcggcgacgattgccattatcaagttttggcaattatcaaaacgctggccgaagcgaacggatggacgacgcttcgttacgtatcaaccggactttaccgcgaattgattttgaaaggcgttggcttgtccggtcttgaagaaatctttgtagggttcaagtgttaccaaagcgtttcgggcgattattacaacataaattgcggcgtatttattggttacgttgccggaaacagtttcgaaacgcagccgggcgcgcaatttagcgcagtgcaagcccacaataacgcggttacgtattttataaccgcaaacgcccaacgaatcgtcggatgctttaaggtcggtacgccggtttacgaacatttttacgtcggcaagttctttccgtatgcacggccaggcgaatacccttcgccgcttgtttgcggcgctatgctgaacggggccgaagcaacgcgattcgacagcacggcgcaggttttcccgtatccgggttattactattcgacggcgcaaaatcggttgtatcaacgcgaccaagcgggcaactggattcgtccgtattgttacccttttacgcaaggttcttcgttgtcaaacgcacttgcggggccgcagggttcgaacacgcttgttcctgccgacgtgtattatcaacttgaaccaattattttggcgcagcaaacaagcgacgtaaacccttcgaacgtatggggcgaattcgacggcgtttatttctgttccggctttaacaacggcgtcgaaaacgtagttcaggaaggcggaagttctgtcgttgaccaaaccgggcttaccgtacttcaagccgtagacgctattaaggcggtcggcgggcgggcgtttgtaatgttgcaaaacgtctatcgtacttcttggcgcgattttatcgctttggaaatgaaataatggcatacattacaggaaccgccgcaagccttgcggatttgaaaacggcaattgaaaacgtttgcacggcgaacggttgggcgctggccgacggcatattgtcgaaaaacggggcgtttttcaagcttaccgcgaacgccggaacatacccgtcgcttgttctgaacggcggcaccagcaagaccggcagcacgctaaacggccagccgacgcagccgcaggcgaacgccggggttatgattggaagcccaaacggaaatacaatcgtttttccgattaattacgaaatttatttgtttaccgacccggacgaagtttattgcgtaattaactataattcggacttttaccaacaactttcgttcggtaagtcggatattccgggcattggcggggccgccgcttggtttaccggaagcatgggaacaggcgtcagcttgacaagtacaagcacaagccgcgtgtatatgtcgtcgtcgcaaacaagttgcggaacaacgccgtataacggcatcgcctgcggcctttggttcgaaagcggccaaggcggttcgtacttttcttctttcgttcattcgaacttagacggcatttcgaattggcgttgcacaggtaacgtaaccgtcggaactggcggatacggtatttcttacgcggcttctttgcttggttcattgccgaatctttccaaccaagcaacgattttgcttccggtcaaggcgattatttacagaaacgacgcaggaagaacaatcgcagccaacccgaaaaacgttcgctatctgcgaatcgacaacgttgttccgggcgaaattgtaacgtttggggccgaccaatggaaagtatacccgttccatcgcaaggattcgacccaacgaaacggcgtcatttggtcaaccggcgcgcagcattcgggaacttggggctacgccattaaatacactggaagctaaaagatggccggaagacttggaacatttttgtcggcgcaaagcttcgtatacggaaacgaaaacccgaatatttccgcagacgttggcaattacgaacctgcggattatgcgccgcttcttacatacgacccgcgcgatacagcattgcgcggcggatattcggcgtacaaaatagcgccttcgaatcgctggcctattgtgggcaaagaacaaagcaacttcgggcgcgattattattatcgcattcacgtttcgccgttggtcttggacttgcaaacggtcgcgtcgtcgcaaacgcgcgaattcaacgtttggaacgcatggcccgaccgtgcggcgaatcttgacgacattttggtaagtaatccggtcggtattgaaattacaggccaagcgacgccgtatgcaatgccgccgcttcaagaacttacttacgaaataaccgtaggcgtttcggggccgccgaatattaacgttgaagttcaatttgacttttccaacgttcccgacccgctgccgattctgattacaggaacgcgcgcggttaagttcaacattgtgcccgaagttccattgaacgaaacttgggaatggttaagcgacaacattgtagcggttgacggaacagaacaacgcatagcacttcgcggcgaaatgccgcgtattgaagaacggttgaaagtaattttcgattctgtcgaagccgtgcgccgtttttacggcgacgttatgtcggcagttggtcggctttggattcccgaatttcagtacgcgacccgaattactgccgcaagcaataccggcgcgcttcaaatttatttcgaccgaacgaagaccgacgttcgcgatactgaatacgtcttgattcaaacgccgcttgttgctgcacttgtcgaaatcgacgtattgacagccgacggggctacgcttggttcgccgctgttgttcgacattccggcagggtcgttaatcattccgggttcgcccgcgcttattgacgacaatacagcacttgcgcggtattccgtcgataccgtcggcgaagtagaaattaacgcgaagatgattcgccagcgttcgcaacttacccgacccggcgcaaccgtcgcacttgaacagttcggcggcgtgccagttttgacgaagcgtccgcttgcaaacgaccttgtacgcgacgaagtttcaaccggccaaatttcaatcgacaatcaaaccggacttgccgacattattagccgttgggactatagccgcatcggcgggccgcgaagcttcaaagtaaaccgtatttcgaatcccgaagaattggattattggaaagcgattcttgcgtatgcacgcggacaggcgcgcaagttttggatgcccacatatcgaaccgatttggctttggccgtgcaaccgtccgacgcgacttcaagctataccgtcgaagggtcggagtatgccgaaaagatttggccgattatcacgcatcgttatatcgaaatcgaaacagcgtccggcattcatcgaaccgaaatattgggcgcaactttgcttgaaactggcaatacaattcttcttcttggtacgccgcttccgaccggggccggatggatggaagttaagcgaatttcgtacttgttgcctgtacgccttggcgacgataaagtagaatggaagcattacggccttgaatcaatcttgaatctttcaattcgaacggcggaaccctaatgtctgattacgacgacaaagaagaaagcgtaggcgatagcgcgccatttgaactttacgaattcgtcggttcatatcgcaattattacatgacttccgacgttctggcgcacgtcttcaacagtgcgacatataaccccgttccgggtttgaagcgcagtacgttaaaagttggaacgcacgaagacgacaacgtagatatgacggttacgcttccgattacggaacaaatcgttaaagactatgcgtttcaaactacgccgccgtctttggttttgacgatttaccgtcgccagcgcgacgccgcaacttgggtcgcatactggaaagggccgattgcttcaatttcggttgacgacgaatttgtaacgttcagaacgccaagcaagttcggttcgatgctgtcgggcaatataccgaacgtttacgtacagccgccttgcaataacgttttgttcgacgaacgttgcaaggtaagccgcgtaagcaattcgctagatacgcaggtttcggcggtcgaaggtcgtaacatttcgattccgtcgcttggcggttttcccgacggttggtttatcggcggcgaaatcgcaatacctgcgcgcaacgaacgtcgaatgattgtcgcgcaagccggggcaattctaacggtaaattacgaattcgcgcgtatttctgtcggcacttcggttcaggttacggcaggttgcgaccattcttacaacggggccaacggttgccccaagttcaacaatcaaccgaacttcggcggttgcccgtttgttccgggcgaatcgaataacgtatttcaaagcggggttcattaacaaatgtggattgcaattgtcttcgtcgtcgccttcttgcttatggcggcgcttatgccgaaaccgaacgtcgaaaacgcacgcgcggcgaaacttggcgacttccaatttccgcgttctaagtacggcgacccaatgccgctagtgtggggaacagtgcgccagaaatcgccgataacggcttggtttggcgacttccgcccggtgccaattcgtaagaaaatgaagaccgggcttttcagttcgaagaaagttacggtcggttataaaaactatcttggcattgattgcattttgtgccttggccccggcgttcgacttcgcaagttttgggccggtacttaccttgtttgggaaggcaacttaacttcgtccggcgatatttacattaacaagccgaacttgtttggcggcgaagatgaacgcggcggcctgcaaggtacaatttcgttttacgacggtcgttttgacccgccgcaagattcttacttggtttcgaagattggcccgaatgttccggcttacaacggctttgcgcgtgcgttgttcaaagccttttatattggaacaggtacgacgccggaaccgttcagcttcgaaattcagcgcattacgtccggcttgcacgcgacttattcaattatgccaaacgggttggacgttaacccgatggaaattgtttacgacgcaatgacgcaaaaatggggccgcttcggcaaccttgaaagcgaaattgatatgccttcgtttacggcctgcgcgcaaacgctgtataacgaaggtttgggtatgtcgcttattgttcaatctgcgattaccggaaaagacttgttggaagaagtaatgcgcgtcgccgacggcgtgttgtatcaagacccggcaacggcgaagattgttgcgaagcttgttcgccaagattacacgatttccgaacttcttgttcttgacgaatcttccgttaaggaacttcgcaatttccagaaaacaacatgggaaaacacttttaaccaatgccgggttacgttcaagaaccgcgctagtagttacgacgacagcgtagcaattacccaagacttcgcaaatatcaactttcaaaaccgcgttaagtctacggaagtttcgacgcccggcgttaccgacgcaacggtagcaagcaagattgccgcgcgccaactttcaattttgaacgttccgctttacaagtgcgacttgaccgttaaccgcaaggcgcagaacttgcggcccggaagcgtgttcgttcttaattgggcaccgttcggcatttcaaacatggttatgcgtgttacgaaaattgatttcggcgaacttacgtctaaccaaatcaagctttcttgcgttcaagaccgcttttcgtcgtcttcgcttacgttcgcaccgcccgaaggttcaggatggacgccaattagcacggaaccgacgccggttacaacgcggcttctttttacgccgcctgcgttccttaccggatacgacgacagcgaaaccccggcttcgttcgattctgccgggcgtctgtaccttgccgcagttgcgccgggttctacgtccatttcttacgacgcaatgacggccccggacaatttcagttcgtcgcctattctgtcgcttgaaagcgccccgtacaacggcggcggcgtgttgctgaacgcctacgcttcgacagtcgccagcgaaacccgcaacgatacaagcggaagccttgtcgtaactggcgtgtcgcaagcaagcattgaaggtttgcagcaatacacgacgctagaccaagcgcgcgacggttcggccttcctgctgattaataacgaactgttcgtttatgtgggcttcgttgacaatggcggcggtcaagttacgttcccgaacgtgtatcgcggcgttctggatacagtgccgggcaatcatgctgcgaacgaccgcgtttggttcattagcggaaccgacggacttttgcccgaacttatttcggtcggaacgacgcgatacgttaagcttttggatacgacttcgggcggcaagcttgatatttcgttggccccgtcgttcagcgggtcgcttaccaaccgggccgggttgccgctgccgccgcagtacctaacgcttgcaggaagccgcacgccaagcccacaagtaggcgcaacgtcgattgctgttgcatggcgcaaccgcagccgggccgataccctgttgcgcgtgtatgacgacacggcgaacacgcgggaagctggcaccgaaacccgcgttcgctggcgcgtaggcggcggcgcttacacgaccgttacgacttcgggaagttcaacaacccttgacgtaacgggcaagtacggtacgctagaagttattgtagattcgtttattacggcgaattcgaaatattcgacgtacagcgattcgcttacaatgaccctaacggcaccgccgccgccgtaatcaaaagaacgacagcgcggcggttttggccttcttcaaccgcccgcagtcgtacaacatttcgaccgcttcgttaatgtaccaatcgtaatttatatcgtcgggaaattccgtcggcaagtccattacagggcgtgcgccgtcggttttcccgactttgtttccgctggatacgtaagcaatatacccggcttcgttcttcgggtaataccaacgaacaaccttgccaagataacgaccgttcttttcgccgccgccgcgcacgttctttactgcaacgaagcgccggaagtcgcggcattccttaatcgtcttttcgaccggcgttccgtcaacaaggaagcgaacaacagcgtcggaacaaatcagggtttcggggttcttcgaaagaatcgaattcaaggccgacccgcgttcgcaataagcgcccttcgtttttgcgcccaattgttcgtcaagaaagcgcgcttcggggtcgccgccgtcttccttgattgcgacataactattaacgtcgcggctgaatacggctttataccgcgtttcttcggtcttgtatcctgtccattcttcccaagccgcaataagcgcccttacttcgtcgtgccgcgatttgtgatacttcgaaataatgccgtcggtattgccggaaataacttcaatgccgatttgttccaaagcttcgataagcataagcaacacaagttgcccggtaatcgttacttgcaacataagttgcggcgcgtaaagcgtcgaatacttgttgccaagtttgccgaagcttccgttaatggtaatcttcaagctgtcggcaacggtcttccatttcttcgcggctgcgcggtcgcccgctttctttgccttagccgcttcggctttggcatgaattcgcgtatttacgattttttcgtaaacggtcaagaaagcttcgcccaagtgcggcgggcaaagtctttgattaaggattgttcgcgggtaaaacgattctacgtcgttgtctgccaaaatgatttcgtcggtcgcatagtgcgccgttttcttttcggtagaatgcaaaccgcccatgcctagtttgtacgtcgaaccgccgatggttactttcagcttttccagttcgggcggcatgattggcgaacccaagccgtcaagatagaagcgggcgtttcgcacgacttccagcatttcgcgaagctgcgtcgtttgatagcaaacgaagtcgggcacgtcgtaaactaagaccgtatcggcggataatgtgggcttcttcggataatagccaaggactttttgaagttcggagttaataaccgcttcggctacttgtgcgtctgatttcgaccgaaggtctacgccgtattcttccgacatttcggcccgcaatttcaattcgggcgcaagttcgttaaacaaaagttcggtatttgaaaggtcgttgcagcaataaggccgcactatttcggcgtcttcctgcgtcaatacatggtcttcgggaaacggcaagtcttgcatacgttcgcaatgcaagcgccccgcgtacagcttcaacgaaccttgaagcggcgcaacttcgataaggtcgatatgattgtatcggccaatttgtacgccgtacttcttttcgaacgcgaacggcgttaccttttcggttccatagttcggcccgcttttaatgatgaaatcggacgcttctttaagcttcgcgcagcttgccccgcgtgcggctagttcaatcatcggcaagtcataattgcgcgaattgaagccgacgatacagaaccgccaaagcatccaaagaagcttcgtcgggttaaaatcatggtcggggctgcgttcaaacgcgacgaactttccgttatcaagcgacttaaacgctacgtaaaagaaatttacgtaagtttcaacgtcgaagacgaagacacttccggcaggcaatgccgttaattcttcgtccgacataaattcaacaggccgcaaagcctgtcgaatgcttgcggccagcttatcgacgacgcggcttttctttgcaacaataaagccttgttcgttaatcatgcgaaagccccttaaaacggtatgtcgtcttcgtaattgtccgacggattatgcgtcgcttcgctgccaaggtcaagacccataagaacgccgcgcaggttttcgccgaaaaatacgaccttgtttgcttccggtgcgaaatgcgctttcttaaacgcatgttcgacggaaagaagcaacttcgcagaaaagcccattcgttccggcaagccttcgattttgtacgtcgaagcttcttctttgtgcatcttcgacaaaaccgccccgtcgtcgaaaaatacgtttccgttcgggctgaacgactgaatcgcgcgaacgccgttgtaaaaatcgtcgggaaccggccaagggttcaagccttcaacttcgaaaacgacttggtaattcgggtaacgttcgccgtaaagttgcgttttgataaacgccccgttttcgaaatagaacgttgccgacgacgccgaaaagccgaagccggtaagggccgggccagccttggcgacagcgaccgccgcgcacttcggaagcatcatgccgggcggaaggtcgataccgtgccacgcttccagcaaggccgccccgttggtcgcgacggcgcttccggcttgcagcaagacggcggcataggtcgcattcggtgcgccgtctgtcgcaagccctgcgaccgcagcgaaggccgttttaatccggtcgtcgattgtggcgcattgcgggtcgggcgcggtaatcggcacgtcgtcgaagcccacacaaggcaccaacgcccggaacgcgccagacgatacggcaagggcgttcgcggtaagctgcgtaatcgacagttcttcgccgaccttcgacagcgcgtcgataaactgcaacgtatgcgggcacgctaccaagtcttcttcgataggatgcgcgacggtcaaaaccccgtcggaagccgccgcccaattgtgggcaatgtgcccgaactgaacgttcgtcggcccggcctttttctgcgcgatggcaacgaacttcaacgccgccaaaagcgacgccgcagggcttggccccttggcctttttctttccggctgcggcttgcctgcgcgcccttggctttttcttcggctgttcggcggctggcgcttcgtcttcgggcggaagcggaatattactgtcgattttcattttcgaccccttcttcgtcaagtttcgaaattgccgcttgaagatagttcgccaagtctagcgtttcttctagcgcgtgttgcaaccaatcgcgaagcgacaacgggttattatccgtcgttacgccgtactttgcaagtccaacgattgaacgttgcaacagtaattcgcggttcttttctacgtttttatcgggcgatatttcggccatttgttcaccattcagcggaaagaacttcgggatactttttgttcacccatacgcgaacacggcgcggcgtgcgaagttctgaaacacgctgcaaagcttgataagtcgtcggcggcggttcttcggcgtgccgctgccgccaccaatcgcgcgaacgtttcgcagcaagccccggatgttcaagcattataaattcgttgaacatttggaagccgcaaaagtaactaaccttaatcatcggcggtttcgttagatttccgtttgcgtcgcgcttttcgtgcaaattgtaaagtaccttttgaacgtcgaaatattcgactatcggcgcgtcgcttcgcagcggttccagcgttccggcagtcgcaaacaacttcgtttcaaaagtaaattcggtgccgcagttgatacactgtcgggccgcagcgtgattatatacgccgcatacgtcgcaaatacgaacgggcgcgtcgccgccgcctttgcccggtctgttcggaattcgcgggtcgttgattgggccaagccttcgaacgtttccggcgaagtccgcgtacaggcaattttgtttgccagtatccggcgacggtcgggttccgcgtcccaacttttgaacgtgtttgcccggcgatagcgtcgggttcaaatcgccgataaagtcaatcggcggatggtcgaaacccgttgtaagtttctgccccgataccaagccgcgaagttcgccagctttgaaagcgcgcaaacgttcggtattggtcttcgaaggcaacttggaatgaaccggcagaacttccaagccgtaagactgaattacgttcgcgacgtgttcggtattatcaatgccagttgcgaagataagccaagtcgaacggtcgtaagccatttcggtcatttcccgaacagcgttaaaaacaacttcgtcggtatctacggcggcttctagttgcttgctgttgaattcgccagccgttacgcctactttcgacaagtcgatttcggttcgcgtcggccttgcgataagcggcgacaagtagccttcggcgataagtcggttaaacgattcgacgccggttatgtcgtaacaaatgtcggtaaaaattccgttttcggtaataagccccattttcattcgatagggcgttgccgtaaatccgataactttaagatgcgggttaatcttgcgaagttcggcgataatgtattggtaatgcgaatcttccttgtcgcttaacaaatggcattcgtcgattaacagcaagtcgcgccaaccgaaatggcgaagatgcgccgggcgtccgtcgtcttcttcaagcccgcgtttaatcgcaggcgctacggactgaacgccgccgaatacgattggcataatcatatcgcgggaattcaagcccgccgaataaatgcccattggcgcggtcggccaaatcgacataagcttttcggcgttctgttcgattagttctttaacgtgcgtaagcatcattattcgttgattcggccagtaaccgaaaattcgacgaataaagtttgcaattacgaccgactttccggttcctgtcggcatggcgacgacggggtttccgaccccgccgcgctggaaataatcgaatattgaatattccgcttcgtcttgataccaacgcggaacgtaaatattagacattctttgcaaccgctgtaattggcaagtacgaatcgcaacccgtcgggataaaatcgcccggaattacgccattgtgaacttcgcaaaaccattcgccgttttcgacagggcgcgacgctttgcagcttcggcaattcttttcgggaattgcgcccttgtggcaaatgtctttcgccgcgcaatatccgcacttgtgaaacgtcggattatccgacaagcgcggcggcgcttcctgcgaaagaatgatttgttcggccttttccttcatttgctggcctagccgatggtctagctttacaacttcgcaatgcaagtcgtcgtcgttcttgttaatgttcaggtaaagaacataacgaaaacgatattcgttgccatacgtcgaagtttgggcgaaatgttgcggcttacttactgccattccttgcgacattaagtccgaaaagcccttgcctgttccgttggtcttgaattccagcaaaacgggttcgtcgataccgtagcgttcgggaagccgggcgataccgtcaagcgacccgccgaagtgccccataacgccggaaatgcggtattgtgggaattccaagccgtcggctttcgcgcgggctacatgctgcaaatacgcggggttttccggcgtaattcgttcggccagtccgtcgccttcttcgccttcggcaaggatgcaatacgaatcgctttccgcgtgatacgcgaagccgtcgcggttttcgtaccaacattgaacgccgataccttccagccattcaataaagcgcgcttcttcacgatgcccacgattaaacaagcgttgcttgcgcccgtcggtttgttcgcggaaacaccaacggaaaatataccaaagcttccgcttgcattcgtcgccaatcaaagacgcgccaaggtgccgacggtgcccgccgtcatacgtgcgaacgcaatattcgtctatatcttctttaatgcgctttgcaagggcggtcgcaacgccgggcgcgtcaaggtctacgctttgttttcctgcgtcggatttcgtcggctgcgcgtcgttcagcgttaattgtgcgttcttcgcgtctttgctgttttgcatcgttcttcaccttgcgccgcataagggcttcgtaattgttcgctagataatccgccgaaagtgtcgcaacgtctttaagttggtctttcgacagccaagatatatggcattccgacgggtcgattcctaattggttcgcaagccaattgtacgccttcgcgcgcgacattaagccggtttgccaaaggcggtcgaattcgttatgggctttcgttcgaagctggcgcgttgttctgtcggccatacgacccaacggaataaacgtgcccggatggcaaccaacggcggcgcgacaatcgttgcaataataaatgtgcggccagttgccataagttcgcccgtaaatcttgtcgttcgttgttagttcaatgttgaacgaacaacaagtatcgcattgttcgggcgtcggcaatgcgtcttttattttcgacataagttcgcatactccttaaaaagaaacgccggggccggttaagaccccggcgaatctacgccgcgacggcgttacgccttaacgctggctccaaggtgccgcgcctgccggtgcgccgccctgttgccagccgccgccctgcgcgggctgctgttgggcgggctgttgggcaggctggccgccccatgccggttggccgccctgcggggccgggttctgcgccggttggccgccccatgctgcgccctgcggctgttgggccggttgctgcgcgggctgctgctgtccgccccaagcggcaccgcctgcgggctgctgcggctggccctgcggggcaccgccccaagcgccgttcggctggccctgcggctgctgttgctgcggctgcgctgccggtgcgcctgcgccagccttgcccggttcgttgccgttcatatcgaacacctttttaacttcggtatattgcgggtcgtttttctgcggcccgacttcgataagaaacggcaggttgtgaagctgcgacgaatcggtaacttggaacacgccgataacgtggcagattgccgaaagctggcgatgcgcgatttcgacggtctgttggttggaatggtacaggttcaggcgataagcgccggtcgtgccttgctgcgggccgtcgataatccgcaagttcagttgcagataaccgccgtcgttcgccttgttggccttgacttcggaagattcgatgataaccggatgcttgccaatcggcaggcttccgacgccttgggtcgggtcgtactgttgggcgttgaacgcttggataagttgcatgatatttcaccttttcgaaaagtcttcgccattggttacggcggcgaagtaaaccgttttattgcatcgctttcgcgaacaatgcggtaaggtcgggctgttcaagttcgttaagattgcccaaacggtcgcgcgcgaaaacttcgggaatttccttcgttcgcagcgcccgaaccggcttcggcattccgggtacgcttgcttcgcccaagtgcataacgttatcgaacaaatgcggaactttaacgttaaggtctttgcccggaaagaacgggcgtttttgcataatcggttcgtaagtaacttcgccgttctgcaaaatcgtttgccgtccgttttcgacaagggcttgtttcgcaatcattacgatatgcttttgcggcatgtaatacaagtcgtttgcgattttcattacacgttcggacatattgccgtaagccttcataccgtgttttaccttgcccaattcgtcggccaagataatttcggcgatgttcgaaatactgtcaatacccaacgtatcgaaattcgcagcttcgcgcgatttcataaaccattcgaagaattcgacgataagcgcgggcgtgtacgcttcccatgcgggaacgttcgaaccgcgcatggacagcataccgggttcggtcacaagcaaaaccggacgcggcgcagtattgataagcggcgttttgccggaaccgggcgcaccgaatacgacggactttacgccgtagcggcgggccagttgcgacgccggttttaattgcgacatttgcataacttgcaccttcgggaatggccccggcgcttgtgggcaaccggggcggggttgttacttcgacttcggcgcgacaagttcaagcgcgggcgttgcttccttcgacgttacaacttcgtcgataagcttacggaacttgtcgggaaggttcttgtattcggtaacggaaagttcgggcttccattttaccaaacgttcggcgataaattcgccttcggggccggtcttttcgattttctgcaaagccttttcgacggcttcttggccgcctacaaagctgcggttaatcttgaaaacggctttcagtttgtagcctgcgccaagttcgacgttttcggttccttcgcgcaaagcttcggggtcgaaagcgaaagcgtttttcagaacttcggcgcgcaatgcggattcggcttctttcgctgcggcaagcgccttcgttgcttcctgccattgcagaatcaaagcgtcgcggttagcgttttcgttgaaagacattgttgttactccttcggttgggccgccgcacaattgcgccggtatgtacgaactatacgacggcccttcgttgctgtcaagcccctttttcaagaatttttcgttcttcgaacaagtcgcgattgttggcgtcgaattcggtaaaagcgttcgggaagcgacgacgaagcttcgcgatgttgaccgactgcgcttcgccgaagttcgaaccgatagcacgcagcaaaagggcatcgtaccaaaagccgtcgccgacttcttcaagcgcgttaacagcgtcgaaagcttcgccttcggctgcggcgcttagggcttccaacagttcgcccgcttcggtcgccttgccgatgattgcatgaatgatattcacggcggcggcgtcgtcttcggggcggtcggaaatccattcgggaagcttcgccagcgtcgccgcgttggcttccggggccggaacgccagtgtcgcgaccgtagaacatggctttcttgattgcgtcaagcttgttaagggcttcgacggcttcggcgatggtttcgcggaagtaggccagcggcacgcggtcgccgtaatacttatccgacgcggttacgtgcgcttcttcgatatagtcgaaggtctttgcaggttcgttcatacgttcaccttttggaagggccgggcaccatgcgccagcgttccagcactgtaaccgacagcgcctttcctgtcaacaacaattgacagcaagcaagaagccgggtaagctgcccacaattgctagaaggtggcgttatggacattgaagaacttacgaagcgggtaaagaaagcaagcgtcgggcggtcgctgcgggcactggccgcgcaatgcgacgtttcgcacgaacttataagaaagcttttgcttgccaattctgcgccgaacataacggcgacgacttataacaagattgacaaagggttggcaaagaatggactttaacaatattccgcacgaaatgcggatttacccgcaatgggtcgtttggcgatacgaagacacggattcaaagaaaccgacgaaggttccgtattcagcaaaaaccgggcaccttgccagcgtaaccgacccgaacacttgggcagggttcgacgaatgcgttaacgctatgtcgtccggttggtatgccggaatcggtttcgtattgactgaaaacgacccgtattcgtttatcgaccttgacgacacaaaaggcgaccaaacggcgcttgaccgccaaataaagatttttaacgaattcaacagctacgccgaacggtcgccttccggttcggggttgcatatcatcgttaagggcgcgattccatcgggccgccgtcgttcgtttatcgaagtttattcttcgcttcgttatatgacaatgaccggcgacgtttaccgcaacgcaccgattaacgattgtaacgaactgttaaacattctttggggccaaatggggcaaggttcggttgccgtcgcgcattacgccggacttgccgaagccaaggaaaccgacgaacaggtttataaccgcgcagtcgccgcagccaatggcgacaagttcgccgaactgtacgcgggcaaatgggaaggcatgtacgcttcgcaatccgaagccgactttgccttggtcgatattatcgcgttttatacgcagaaccgggcgcagatttcgcgaatgtttcgcgcgtcgggcttggggcagcgtgacaaagcaaaacgcgacgattacgtttcgtacatgctgaacaagtgtttcgaccgcatgttgccgcccgttgacgttgacggattgcgcaacaagctagacgaagcaatagcaaagaaagaagccgccgacagggccgccgcgttgtcgcagaacagcgaagcgacgccgcatccgaaagccccggccccgaaccttaacgaagcttcgaaggtatacagcgtgccgcccggattagtcggagaaatcgcgcaatacatttacgcacaagcgccgcgcccggtgcccgaaatcgcgttggccggtgcccttggtttggtcgccggtattgtgggcagggcgtacaacatttccggcactggccttaatcaatacgttctgttgttggcaccgaccggaacaggtaaagaagccatcgcaagcggcattgataagctaatggcgcaggttatccgaaccgttcccgctgcgtcggactttatcggccccggcgaaattgcttcgtcgcaagcgattatcaagtatatgtcgcgcggcccaacgtcgttcgtttccttggtcggcgaattcggcatttaccttcaacaaatggcaagcgtaaacgcaccgccgcaccttaccgggcttcgtcgcttcctgttggatgcttacaacaaatcgggcgaaggcaaggtacttcggccttcgatttattcagacaaggacaagaacaccgccgccgttctttcgccgtcctttaccttgcttggcgaatcgacgcccgaaaagttttacgaaggtttgcacgaaggtttgatttccgaaggtctgttaccgcgttttacgatgattgaatatcacggcgaacgcccgcctttgaatccggggcacctgtcggcccaaccttcgttcgaacttatcgacaggctttcgacgttgtgcgcccatgcgctaatgttgaacagccagcataaggcgattcacgttcaaaccgacgcaaccgcccgcgaactgttccagcaattcgacgcgcattgcgacgcgaacattaacacaagcgaccgcgaagttcgacggcacctatggaaccgggcgcacgtaaaggcgttgaagctggccggaattatcgccgtcggttgcaacccatacgacccgaccattaccgccgacgttgcgtcttgggcgattaaccttgtcgtcgccgacgttcgcaacttgcttgcccggttcgatgctggcgaaattggcatagacaacgacgaaacgaaacagcttgcgaaggttattgcaaccgttaaagatttcgtcgtttcaccttggccggacgttgcgaagtacgcaggcgaaggaatgtccaaccttcattcgaaccgaattgttccgtacagctacgttcaacgcaggcttgccgctgttgccgtctttcgtaaagaccgcatcggcgcaagcggggcaatcaagcgcgccttgaagaccctttgcgaacgcggcgatttgcaggaagtaagccgggccacactggcgaaggattacggaacaagcgccgttgcatacatggtcgcgcatccgggcgttttcggcctgtaacgcgcgcccgccagtgtgggcagaacgggccgccttcgggcggctttttcgtgcctgtaaaaaaagttcgtcgtacctgttgacgtagcttcgaactgtacgtagaatacaaaccatcgaagcaaacaacgaaggagtaaacgaaatgacctacgccgaagccaagaaagccgctaacaagaaaaacgccggtattgtttacactggcgacgcgggtaacggttgcacgttcgaagtttattactgcgaatggcgcaagcgtcaaatttggacgaccgtaacgccgaacggaacccgcattgtataaaccatgccgacggcccttggcacgcgctaggggccgcgcagcctgcccacaatgaccgaaggacgaccgaccatgattaagccgcaacgttggtacgaaataaccgaagaagaaggccgcaaggttttgaacgtgtacgacgcgccttacaaagaacaggcgacaagttcaagccatacaaagcccgaagatttgcagtattaccgaacaaacttcaaacttgaaaagctttggaaggagtaacaaccatgtacgcccattacttcgccagcaagaaaacaaagttcgttgtaatcagcgacggccctacgcccaacggcaaacgaattgcggtaagcggcaaggtcgaagcgcgcaagattgcgaaggaacacgacgcgcagccttggaacttctaaacttcaaacttcgaccgcttcggcggtctttttgtttgtacggcgtataacacgacggcccgcgtataatggcccgtcataacgcttaaacccttgatacataaggcttttaacggatttatagcaagtataaggtttaacgccgttccacctgtaccggggcgactctttattaaaagtcactattgattaatagtaatatttttactattatattgtattatattattatatttcctttattttcaaggacttagcagctatacgacccgttcaatccgtataatgctttatcaattgattaattgttgacacttgataaaaagtaagctaggcttgcactattgattaaggggcttgctatgccgaaaccaacgacgaagaagggccgaccaaagggctttattccgaagcctgcgaactgcggccagtgtttgcgcgaagtagcgccgaacatgaaaatttgtccgtattgcttgaagtcaaccgggcttatatcgactgcgaaggagttaaagaaatgaccgaagaaacgaagaaggttcaaattttaccgtcgtcgccgaaggcgcttgctttgtgcgaagtttcgacggacaatcaaccggaacgcacgccggacgaagacaacgcgaaatatccgtttcgttcgttgcaagtcggcgaatcgttcgcgataccgtttgcagatacgaaccccggaacgcttgcgtcgcttcgttcgtcggccagttcatacggcaagcggcttaaccgcaagtatcgcgttttcgttcacgacgaatataagtgcgtcgaagtggcgcggctgaaatgaacagcgacgcgcggcccaatgtgggcaaaatggttcgcgtgtatgggtcgtcgtatcttggccttcgcgcgcagtatccgaacgcctttgccatccaaccaacccggacgggctatcaagccgtcgtaatgattgaagaaggggcaaacaatgtacgaaatcgaaaccggccataaggtgccgaagcaacgtcgcaaggctggaagcgtgccgaaggccgttcaacaggcgttcaacgactacgccgaagcgtataaggccgtttacggcgttcgcccgctgtcgttcacgtatgaccgcgaaagcggctttattcgtgtcgaaagtagcggcggcgtaagcgttaaaaggctgcgcgaaatgacgaaacaacttcgttaccgcaaaggctgaaaaaagttgttcgcaggtattgacgaagcgtcgaaggcgtcgtactatatacccataccaacgaacacaaggaaccgccgaaatgaaaaacgcacaagtcgcagcaatggaagccaaaatgtcgaaggttgcaggcgcagccgtagaacttaccattcgcgccgaacgcgcctttacgttcagcttcgaaagcgtaaacgaagaagccgccgcaaagctggcaaagttcttcgaaggccaagcgaaagtcgaagttgaacacgacgaagaatgcggttccttcgtttacgtcgattgcaagtaatcaacagcgcgtcgggcatttcgtcccggtcgttcagcaagtcggcggcgttctgcaagcgttcgacgtgttcaggctggaacgcgccggggttggcttcgtgtacttcctgcgcgacttccagaacatcgcccaaggcttcggccagcttttcgaccaagccaagaattttggcgcggtcttctttatccgggccggaagcttcgacggcctgtttaatgtcgcatacgatgcacattgttataactccttcaaaaatgccgggttgcgcccgacgcgctgttggccctgctgttggccgcccttgggcgcggcgtcaaggtcgaaacaatccgcgtcgaactgcgcgaagacgaaacgccagaacaggccgtagggcgtgtaatggccgaacgcgaagccggggccgcgacgaaacactaaccaaagcgccccggctgttgtatagtcggggcttctttctaacatggggaacgaaagaacatggcggacgaagtagaccaaacagccgaccgaatagaaaacgaagtaagctttacggtcggcgaactttgccgaaaggccgcagcgatacccgcaggccgtccgggcgaatgtgatttttgcggcgaacacttcgcgcgcgttgtcgaagtgaccgacccgcgaacgggcgaagtcgtcgaaagctgcggtcgctgtcgtgaccgaaggggcatcgcatgacgcccgcaacccttgccaagtctggcaccgaacacgcgcaccaaacggcgctttttgcttatgctgccgtcgcatacttgcacggcttcgacgtagccgacgaatggtgcaagaccggcaaactaccgaagcgcgacccggacgcaccgcccaaggtgcccgcccttgaatggttccacgccataccgaacggcggcagtcgcggcgacgacgacaagtcccgcaagatacgcggcgcgcagttgaaggccgaaggcgtgcggcagggcgtagccgatacgttcttgccttggccgtctggcgattggcacggcctttatatcgaaatgaagaagccgaccgaacgtccgaagtcggaaaccgcaaaaggcggcgtatcggatgaacaacgaagattcggcgaatacgcaaagcgcgtcggctatggttgggtcgtctgctatgattgggaacaagcggcgtcgtaccttcgttcctatatcgaatggggttcgtaatggggctttcgtgccttgcctgcgaacttacccgcgcccggttgaaagcggcggcgctggcccttgtgggctggccccttgaccgcatcgccgggcatttgtccgtttgctatggcgaacggtattacgtcgaaggcggcaagctgtatcgcgcgtcgaagttgccgccgtatcaaccgtacttgataaggggcgaagaatgaaaaagaccgacgaagaattgatagacgaagcccgcagcattgcgcgttcgaaacgctgtttcgtcgtaacgaagccgggttattggctgctgtaccgcgaatgcacgccgcgtaacgtatgcgtcggcaaacggtcgtcggcttcggcgttgctacaacttgcgaaaaaggcttgacgcagcgtcgaagctgtcgtacacttcttcgtaagttgaacgaaccaaggagtaacgaccatgcccgcatataacgtaacaaccgaattcgaaatgaccgacggtcgcgttctgtcgtgcgaatacgtcttgtcggttacgcccggtcgctattccggcccttgggaagattgttacccggacgaatccgacgcaggcgaaccgacgtattttatcgacggcgaagaagtcgattacaaagaatttccaaaaggcttggacgtaatcgccgacaagctttacgaagctggcgacggcgaatacaattacaatcaatccgaaatcgaacgcgattacgaaccggattacgaaccggattgcgattacgattattaacgaaggagtaacgaaaatgtcttggcttatcggttggggcgtactttccgtcggcgtcgttgtcggcttcttgcttgcggccttgctgcgggccaatgggcgcgacgtgccggttccgtcggctaccgttaccgccctgtcgccgcaggaacgcgcccgcatgggctacgcactggcaagggctggcgacttgctggacagcggcgaccttgaatacgtgtcgggcggctttatggtgcaagacgtagggcacggcgtcgtaattatcaaaggcgatttgtcgttgcgggttgccgaagatgaataaggacgaagcgcgcgaaatgttcgaagccgtcaaggccaaccgggcgaagattgacggttgcgcccgccatcgcttcgaaatcggcgacccgccgtacacgttcgggcaaaagttcacttgttcgaactgcggcggaacaatggacgccgtacaggcgttccgatactgtcaaggcttcaaggccgcaggcggcgacccgaacgaagttataccgggttacgaataacgcttgacaagcggcgaaggttcgacgaaacttaacacgtacccgacaaaggagttacgaacatgaatttcgacccgctgtttcttaccatcggactttcggcgcttgccttggcttgctacttgaagttcggccaaataaaagcgaagcgcgaacaagaccgccgcattcgctaagccatcgaacgcgcgcaggttgtgaaggcacgcggcgaaattcgcggcaagaagcttgccaacgaacgcgaatggggcttggccgaagattacaaccccggcaagttctggccgcagtaatggcgataatctacgcaccgcgcgaagccctttccaaatgggacgcgcgatttatcgaactggccgaacacgtcgccggatggtcgaaagggccgcgtaagcgcatcggcgcggctatcgtgcgcccgaatcggtcgattgcatcgcttgggtacaacggcccgccgcgcggcttcgacgacgccgtttttctgaacatgacgcgcgacgaacaacacgcggtcgtaattcatgccgaagacaacgcaattcgacaggctgcgccgggcgaagacttgtccgcgtgtacgctgtacgtttcgccgctgtttccgtgcgccatttgtgccgaccgcatcgttcgggccggtatccgtcgcgttgtcgcttattgtgggcatatatcgccagactggcgcgcgtctgccgacgaagccgaacgcatctttaaccaagcgggcgtcgaatgcctgtttacaatggattaacgaacatgaaaccgctagaccattcgcaaatcgaaggcttgttgttctatcaagacccaacgacgcaaaactatatcggcgcaccgaaggccgacccgttgaaccctgccggaagcttcgtcgcattcgggccgcgtgttacggttgacccggcgttgtataacttgcttgccgcagcgccgacaatgtaccaagccctttcgatgcaatacgcttaccttcaagcgcaaatcgaagttatcgaacgcttgccaaaggcgggcgaacttgataagctgttgtcgtcgttaatcgaagcgcagaacgcttgcctaatggcccaacgtatcgcgcaagtcggttacgaaaatgtcgcaaattcacttgacaaaggttcgaagcggtcgtaagcttgaaccatcgaaacgaacaaaggtgcccacaatgaaccgtaagcttcttgttatcgctggaatcgtcgccttcgtcgcgttgcttggcatggtcgggcaaatggacttcgaagacgaagcccgcgcgcacctgttgtattgtaacaacgtagcgaacggcgtttggccggattacgacaaaatttacgattctgaatgcacgcccgaacgcatcgcagaaatctaagaaattttgcgttgaccgcttgacacgaagaacggtcggcgtaaaatcaacccgtcgcaacaacgcgacataaccttaacgttaaggagttacacaaaatggctaccaagaactcgaccgcgaagaagaacaccgccgccccggttatcggccttgccgaaattgttgcagccgggccgaatggtctgtatacttcgcctgccgtacatggcccgcttgtcgaagccggtatggtggaaatcaacccggcaatgaccaacgaagcgggcgaaatcgcaacccgcgcaacccaaaaaggaatcgaagaaatgaaccagaacaccgaaaccggcaccgccgcagcttccgccccggtcgctacttccttcaccatcgaagacaacgtaccgatgccgaccggcagcggtcgcggtcgcggcggcaacgtctacccgttcgacgcgctggaagtcggccaaagcttcttcgttccgaacagcgaagaaaagccgaacgccgcgaagtcgcttgcttcgaccgtttccagcgcaaccgcccgtttcgctgttccgtccgaagacggcgcgaccaagaccaacaagaagggcgaaaccgtgcccgtcatggtcgaaactcgcaagttcgttgttcgcgctgtcgaaggcggcgcgcgcgtctggcgcaccaagtaaccggcgcgggcttcggcccgctacggccaacagttcgacgaaagcccggccaagcgccgggctttttgttgtctgtacgtttctttcgctttgctttttctagccatttgtgttaatccgtgctaatctgcgccctatgttgtgatttatcaaataccgggcggggcgtcgaataatgaacgaacaacaattttttaacgtagctgtcgcaattatcggcgctttgggcggatggttaatgcgggtaatgtggcagtcgttgaaagaacttcaaaaccaagatgcaaggttggccgaaaaggtcggaaatatcgaagttcttgttgccggacaatacgtcaagcgcgacgatatggacagaacaaccgccgcactttttgcgaagcttgaccgcatcgaagacaaactagataaaaaggccgacaaatgaagaagcccgaacttatcgaagattggcgcaattggtggcgcttttggtcggttcgactggccgcagtcggcaccgccgttactggcgttcttatcgcgtttcccgacgcagccttggccgcatgggccatacttcccgaagaattcaagtcggcaattccgccgcagtatatgccgcttatcggcgtcggcgtattcgcgctttccatcgttgcgcgccttatccgtcaagaaaagttgaaggggccgaccgatgaataacgaacttccttgggtcgccgaagcccgaaaatatatcggccttcgcgaaaacacttcgaagaccgaacataaccccgtaatcgttaaaatgcttgaacgcatgggcgcgttttcgaacgaatcccgcgcatggtggcgcgaagacgaaacgccgtggtgcggcctgttcgtcggcttttgcttgggcgttaccgggcgcttcgtcgtgcgcgaatggtacagggcgcgggcttgggaatcgccgcagcttacgaagcttgaccggcccgcatacggggcgcttgtgaccttcacgcgcgacggcggcggccacgtcggttttattgtgggcaaggatgcgcgcggaaatcttatggttcttggcggtaatcagtcgaacgccgtaagtattgcaccgttcgcagtatcccgcgtaaccggctatttctggccgtcgttctggcgaaacaagaccgcagttaaaagcgttccgtttgaagaacgttattcgctgccgctgttgaagtcgaacggcgaactttcgacgaatgaagcgtaaaggggcatcgtatgttttccgtaatttgggccaagtttggcaaatggcttgccgcaatcggcgcgggtcttgtcatgctggcaagtatcttttatgccggtcgcaagtccggcgcaaccaaggccgaagccaacgcagcaaacgaacgggcaaacgaccgcgaagcaatcgcagttcgccaagttaacgaagcccgcgaagcttccgaacaacaggtaaaggcggtacaaaatgcgaaagaagttgcttccgataattctacttttgacgacgacgaagttactaggcggctgcgcgacgaatggtcgcgcgactgaaccgcaaagcatcgccgacgcagtacaaacgaagcccgttgtaatcgacacggcttgtaaatgggttgcgcccatttggatttcgaaggccgacggcttgaccgctggcacggcccggcaaattctgaaccataacgaagccgtcgaacgcaattgcggcccgcagtcgccgcctaaatctgcggacaagccttaacagaacgcggggcttgcggtatagtgaagctaacgacgtataaactaggccgacactatgtccgacgaactgaaagaaagcgaagaaaaggccgcatacgcggcccttcttttgaaagaacgcgacccgttcaaagccgcgcttgccctattcccgaacaatacgaatcgggcgctatgggtcgcgaaccattggccgaacgacgccgaagttaaagccgaacaagcccgccttaccgaagaagcgggcgatatggcatttttgccgggcaaagccgaccttgcgcgcgacatttggcagcgtatgcaaggtacaacccttccgaacggcgtaaccattccgccaacgcccgaagaatacgcaaagcttgcgaagctttacgcagacgttcgcgggttcatcgaaaaaccgcaaacaaacgtaaacgtaacgacgaacgtaaatcgtgtcgtcgaaatgcccgtatttgcaaacgaaagcgaatgggaagcggaagcggcacggcagcaacgcgagttgttagaaaatgcacgcactcgccattaaagaagacgggacgaaacaagttccgtatgaagtcgtatttaagccgctgccgggttcgcagacaatcgcgctttgttcgatggcggcgcatacgctgtacgaaggcgcgcgcggccccggcaaaacgcttacgcagcttatgcgcttttaccgtaacgtcggcaagggttacggtaaattctggcgcggcgttatcttcgatttggaattcgaccatttgggcggacttgtcgccgaatcgaaaaaatggttcggcgacaatggcaaattaaaagacggggcgaagttttacgaatcaacttcggcttataaatgggtttggccgactggcgaagaattgttgtttcgccatgttaagaagcttgcggattacgaaggcttccacggtcacgaatacccgtttatcggatggaacgaattaacgaagcatccaagcggcgacctttacgacaaatttatgtcggtcaaccgctgtacgttcgacccgattaaagacacgccgaaagacccgaaaaccgggcgttatttgacgccgaacggtcaaccgctgccgcctgtaaagtgcgaagtattcagcacgacgaacccaagcggccccggtcacaattgggtaaagcgtcgttttattacgattgccccgcgcggcaccgtagttcgtcgcgaaattcagatttacaacccggcgaccgaaaaagaagaaacgcacgtaattagccaaatcgctatcttcggttcgtataaggaaaacccataccttccggcttcgtatatcgccgaactggaaagtatcaaagaaccgaacttgcggaaggcttggctttacggcgattgggacgttaccgcaggcggcgcaatcgacgacctttggcaatcgcatatacacgttgtaccgcgcttcgtcattccgccaagctggcgaatcgacagaacatacgacgacgggtcttcgcatccgtttagtgtgggctggtgggcggaagcggacggcaccgaagcgaccatagttctttcggatggcaccgaatttacgttttgcccgcaaccgggttcgcttgttcagttgttcgaatggtacggatgcgcgaaagacgaaaagggcgaatatataccgaataaaggtttgaagctttcggcgtcgaatatcgcacaaggcataatcgaccgcgaaatttcgttaatggcaaacggttggatttcgtcgcaaccttggcccggccccgccgacaatcgaattcgccaagtaatcgacagcgaactagatacgaccgaaaagctaatgtcgaagaagggcgttcgttggctggaatcggacaaatcgccgggttcgcgcgtaatcggtttgcagcttttacgcgaccgattagaagcgtctgttaattgtgaagggccgggtatatactttatgcaaaattgcattgcaagtattgaattgctgcccacattaccccgcgacgaaaagaagattgacgacgtagacacgaaagccgaagaccatgtttacgatatggtgcgttatcgcgtattgaagggcgcgaacaaagcggcgacgaaatttaaacttgtaatgccaacttaaaggaaccttgaaaatgtctagcccgaacgtttcgtttattcgccccgaacttgcaaagcttttgccgctttattatttgattcgcgacgcaatcgcaggcgaaccgaccgtaaaggctgcgcggacgacttaccttccgatgccgaacgccgaagaccaatcgaaagaaaataaggcgcggtacgaagcttatttgaaacgcgccgttttctataacgttgcgcgtcgaactcttttcggtttggtcgggcaagtgttcatgcgcgaccccgtaattaaggtgccgtcgttgcttaacccgcttgttgcgaacgcaaccggaacgggtatcaaccttacgcagcttgcgaaaaaggccgtatcgctaaaccttgcgtattcgcgggccgggctgttggttgattacccgacgaccgaagccgaaggcggggcgtcgattgccgaccttgaagccgggcgcattcgtccgacgttgtacgtatattcgccgactgaaattatcaattggcgaacaaccgaccggggcgcggaagaagtcttgtcgttggtcgtactgttcgaaacatggtgcgccgccgatgatggtttcgaaatgaaaatgtcgggccaattccgcgttttgcgtttggacgaagaaggctattacgtccatgaaatttggcgcgaaccgcagccaacgaaagccgatggttcgaagattccgaagggcaattatcaacaacacgtcgtttataagccgacagacgaaaacggcaatcgtcttaccgaaattccgtttatgttcatgggttcggaaaacaacgacccgaacccggataacccgaacttttacgacctttgttcgctgaatatggcgcattatcgcaattcggcggattacgaagaaagttgttatatcgtcggacagccgacgccggttcttaccggacttaccgaagaatgggtaaacaacgtacttaaaggaaccgttgcgttcggttcgcgcggcggcattccgcttccagccggggccgacgcgaagttgcttcaagcgaacgaaaataccatgcttaaagaagcaatggacgccaaagaacgccaaatggtcgcacttggcgcaaagcttgtcgaacaaaaggaagtgcaacgcacggcaaccgaagccgaattggaagccgcttcggaaggttcgacgctttccagtgcaaccaagaacgtttcggcggcctttgaatgggcgttgaaatgggcggc

SEQ ID NO: 3 - DNA, 94715 bp, unidentified
  source 1..94715: mol_type = genomic DNA
caagcgcttgcacctcaaaagagcggcgaggggagagtcgtagagagcggcgctgttcgtcgccaagggggctgttggtgatgctggtcagccactccagacgctctaccacgctcgacgaccaatccgccgagaaagaccagatggtgatacggcttccgctgatctggatgacgacggtggtgccatcggagaacttgatagtgatcttcccctcaattacaggcgggccgttggcgtcaatgcctattttccaaatacgctgctccagggctttcagtaccaaaggtgggttcggttgcccgctgatcgttaccccactatcgccatcgtactcaatgctcaaaagcgtgacaggcacgaaatacgcattccagattttcaggtccaactgttgcgaaccgaccacgttcccgagttcgaagttgatgctattgaggttgatcttgttgtagatcagccgcaggagtagagggtagtccgcacctacccgttccttgtggatgaggtctatcggggcgttctgctggtaggagctaggtgccgccaaattgaaccgccaagcaagcccagtctcttggttgactttcatctttcgcaagttcgtactgcggcgacccccggcggtttgccagggggacttgaaggtaattccagaaaagttagccacacatcacctccggatgcgataggcgttgccgcgttgcaaggagctggttagggcactaatgcctacgcggaaagccgggaatacgatccattcctccgaaccgtaaatctggacctgggctggtgagcagaacgacatatcgcaaaaagcgaagtctgccacctcccccaacgggcgtaagcgtgactggttaccgatcatgtaaacccggttcgggatcagaaccgtctcgcccgtcatctgattggcactggcgtcgagatagctgtcgtccggatggtaggtatccgacccgtggcccagggccgaccacccccggtcgccagcgcccgcgttgttggtatacacaccaagtagctcccgaccggcgatctctccatcccgaatatcgacttggcaacgcgagttattcacgctgcggaacgagtcccatacatattgcgtcgcagcatcagagaggccgttaccttcgctggaggaggggttatacgacagtttatgatgctgcgtatagaaacctccgccatagctagagccgcgcttgtccaaggtaccaataccgaagtgggcgtagaccttggactgcacctctactaccagatgcacgtactgctcagttacgaacagccagtaagtggtgtagggcccggcctgcttgaggtcgaaccagcccccagcggcagggtagttggatgcagaccagcccggctgcctatgtgcaggtagcgcagcattgaagccagtattgttgtagacatagaacccctggttctgcgcaatcgcttgcccgaaggggttgccgttctcggcgtccacggtaggtccctgcccgtagcgaaacgaccagtacccgccgcttgccgaatgcaaagcccagccgttaccgccgccagagatagtgtccttggtccagccattagctgccgcgaaattgaccatagtatcgatcaactcggaaatcgtacttactggtccagtctggtaagccatcagttagccctcagtgcaagtaggttgggggaatcagtacggaacgcagactggaacaccacatgagtggcgccattggcgacaatcgtgtcttctgagccgttgttgagaacaggcacccagaacaacccgtcagcctcgccgaaagggatactcccctgcgagtcgcgtaccagcaacgctacgggcagtagagggaaactaccatccagggcatcacgtacgagcaatagccggtttcgcgcggcttggtccggggcatagcgtccggtaggcattacgcaacgaagccccgcgtacggcccgcccataccgccatagaaatcccaccattgtccggacggtgtacgtagggctgccctacccatcgtggggctggagtggttcgaagaagagtccgaccagcgaacaccctcgtacgcagtgtgagagccccaaaccgccagagggtaagggaactcgtccggcgggcaagggggcaggatgaacccagcccctacagtggtgaagaccgacgataccttagcggtcacccaccaacggcgaccattagctacgaaccagtacggcatggcttggttccagaagcatccgccttgagtaaacgggcaggggttaatcagaccctgcgtaagatcacggaaacccggggtaagggcggacaggttaagccctgtaccgcccataatccgccagttgtagtagtcggtagccgtgttcgccgccgtggagatagcggtataaatctgatccaggccagacagccccttggacctgaacgctacgtgccggcggttgaggcccgatacttcctggccgttatacggtagggtttgatcgtataacacctcgtacgcctgcccggctgcaaccaagtccgggtcggtagtgaggaacgcgaccagcttatccagaaggtcgttaaagttggccgcttggccttgactgaatgccattgctctctccttagttaatgccgagggcttgacggaacttctggggctgtttgccgatatggaccagaagtaacttctgaccttgcgacccttgcagcgcctccagcacagtttgttcgctaagcaccggttgcacgttgaaaatcggctgcatggtatcaccactggtgccagctccgcttccgccttttccaagattgtcacgatggcgcgggtcttgtttggtcaagacttcctcaccacgctctcctatgatcggaacctcattggcctgtagcccaggttgcgagccgataatacccccagtgtggtggcgaaccgcattggcaaatacagaaacgggagcataggaacgacgcatggtagtacgtccgataatccctccagtgtgcgccataccgaaggctccgccagcagcccccaggccgctcgacgccgaagaggaggccgaagaggcgtagatagcggagatgatctggccgatcaaacttcctagcccaccaagcaaactcccgaaccccccgctaaccgcctgggccacgccctgcacccctgatactaccgagctaagacccgagtccaacaaaccgcctggagtaaaagcagactctagcgtaccgattactccttgctggtccttgtccgtcttcgacgccggggcttcctcgtcccctgcacccctacccatgatccggtctagccaactgctaggcgtggcattggggtcagctacagcgccatccagcccggggataccatcggtaagacgcacatagagtggattcaatgcagagccgtctggtcctgcttcggacgtggcgccgaagaggcttgcgaagaacccgccgattccaccctctccgctggagcctaggacattcatgatcatatcggatagaccttcggaagctttctccgatatagacatggtgaccgaccgcaggaagtcgcttagcaggttctcggtatctgcggaagcgatggagttaaagaggtcttcaaaagctccacgaataccttcgttgatctctttagctacctcgttgccctggagctggagtttctccagtttcaattgtgcgtcgtcgatagtttgctgaagctctttgtagcgcaggctattgcggtcgagccctttcccggatgcctcggctgctgcgatctctgcgcgactacgctctacgatagatttttgataggcgatctccttcttacgaagctcatccaacgcagccaccccctcaaggcgggtaagagccccggcacccaccgccagatcgatacgatctcgcagattgtcgaatcctttaccagtaaggtcggcttcgcgttgcacttgccccagagcggattctagggcttcaaagcgacccatcatttcggtgaatccgatcagcggaaccaactccgggaagtcggcagcgttcgccttcatcttgtcgatgaagtcgcggtatttgacctgagccgcttcaagggcaacttggaacccttccccatctgtagccgcgccgaagaactccacgagactgttgcgctgctcgacgatagagtccgtgaactcccgcatggcattgctaagctcccggtccgtagtggccgccagcgcatcgcgttgtttcagcaagacatctatctgcccttgcgcttgcgcgacctgagcctgcatgcgtacctgttcggcttggttcttgccagtgctggaatccgccgcaagattcttcatctcgtcctgaatagcagccagctcctggcggcgcagtttcagttcgatatcgagattgctgcgggtgatagccatgcgctgtgcatagtattccttaatcgagaccaaggaccggctatacgaaaactcaagcgcttcctctcgctccttgttggcttgtttctccaagtttgcaagcgcttgcagttcggctttcgccagctctacccgggctcgactaagacggcgcgcatcgctatcggaggtcggcgtaaaggaaccagtacctgaagttggcttggtaggaacagcagcggcaccggtcagcttggcaccctcggcacttacctgccgcagcagattaagcgtgtcttggatataagctaccggttgttgcagtacgacttgcaagtattgcagtgaaccaagaaccgcatctccgaggttgtttttacgagcatcagcaataaagccatccagccgggtgcgcaggtcttgcagttggccgttcgccgcagcgtaaccggactgggcttcgtcgtatgccgtttgcaggtttttcaagttctcctgctgagccgaagtagactgatccccagtggcctgtacgatctgctcattcaggtcggacatcttcttggcgaaatcatcgcgtgctttagacgcagcgatgaacggatcagccaaggtttcgaaggctttggccgagtcctgcgtaaacagctcggagagtacctttttaaccttctcggcattgtcggctgcgttttggccgaactggttcaacccccgctcgctttcgtcggtgaacataccaagacgtttctgcgcatcctccaaggactgagtgagagccgctacgcggtcttgcgcaatctttaccgcttgctgattggcaatcgcgcccatgccagtatcgtcgaagagaacaggcatgtcggctattgccagagattgttccaagtctttcttggccttctccagctcggtttttaccgcagcaatgtccgcttcggctttctgtcgccattccggcaaaccctgaatatcggtagttgcttgctcttgggaagccaggacgccgcgctgcaactcgaacagttgcttcatggcctcactacgaacgtcgttgtccgaatcggtcaagttcgagcgaatacgagtaactacgtcatcccgcacccggccacttgcatccgcataggcttttagtccgtcgatggcgtcgagccgagctttgtttgccgcattgtaggccgcaatgaggtctcgctgattttgggcctcacgccgggacgcttccgaggtaagacccatggcgtcggttagcttctggaagccagcgccgatgttctccagaaccccgaaggcatccaaggcgaggtagatgatcgacaaccagaagaagacgcgaccggcaatagcgatcaacgcttggaacccggcacttacaatacgcagtgccgaccccaacatcgagaagccggcagcggcccgggtggtagcagcttgcgcagcggtaaggctggcattcgcgcgattggcagcttggtcggcttggttgagagccgaagccgcaagacgtgcttggttggtcagaaccatcatccctgggcccgccgctactcggtcgaactggctcagagactgcatgaaggccagacgtgcagccgctacctcttgcacgccagcttgttgaacagcctggatgcggcgagcgtagtaggcgttgatgccggccagcgagcgacgctggtaggcttcctcttgggcaatggcctctgcctgggcgagattggcttggcgcaccccggcccggctaccctgagcccgtgcctccgctcgcgccgcgtctccttgagtgacgatagcggtaagacgtgcttgatgctgctgttcagcccgttgaagtcgttcgttgagctgttgctgtacttgcgcggcgcgctgagctaccgctgcctgggccgtagcaataccgccacgggcggcggttacctgcccgctcgccgcgttgaccgcatcgttacgctgacgcaaagccatttgcgcctgtaggttacgggtttcggctgcggtctgcgcttcacgggccctagaggctgcaatctccgcttgggtctccgcctcaaccgcctgaatgtacgcgcgccgctgggccatgagttcccggatacgggtaatctgggtctggaacgcggtattggctacttggctggcgttgcgttgctgctcgctagatcgcgcaaagtccaacccgaggcgagtcatggccgcgccgaggactgggacgctggacagcgcgctaaggaaccactgacccaagcggatagctattagggttttagcgatctgtacaaagttctcccagttaacattcaggtccgctatgaacttgatgcccgtagcgatgctatcgaacatatcgagaaacgcgttacctaccgactccgcgaagtggataaccgccgggtctgccaaggctttgttgagttgggcgatatacgctgcaagacgcggaccgaacacctctccagctttgatacccagtgcttggaagctgtttccggctttggccagctcggcgtccagagtcctcaagacggtgcgctgctcttggatggcgctagtgccggaacggtacccctggactgcgttgttcaggttgttatccagaacggagttattggcatcctgaacgaacttggtcagaagcactccgatacgaccgcccccggacagctccttgattaccttctgctgctgatcagcagagagttttcgcagcccgtcgagatacattttcagggcaccgatgccgtctttctggagctgttgcatccagtcgttgacgcttaccccgagaagcttggagaacttgtctgcgcgaccgtaaaactcggcgaagattttggagagcgaagtaccggcaacttcaggcgatacgccgaaatcgatagcggacgcggcaagacctaccgactgttcaagtttaagcgacccggcagcatcaccaatacgtttgaccacatcgagtagctgctcgccactcgccgtggaattgttggacgcttggttgaacgccgagactacagactcgatatcgcgcaggctgatcttgaagatcgaagcgatcttaccgatattggtaccagcatcttcagcagtgaggtcgagtacggcagccatccgggataccgactcggagaattggcggataccctccacaccctctcggcctaagccttgctggcccgccgcagccgcgatcttggctaagtcttcggcagacacgttgatacggcgcgacagatcaaccaacgagtcgccaagctgtttgatctgcgtatccgtgaagtttgtggttttttgcaccccggccatctcgcgctcgaaaccacgaatagcctggatggggaaggatgcaatcttcagggcagagagggccgaagaaacggcccccacaaatcggataagccggctttccaactgggagagttggggagttaggttatcctgcccgtttatgcgtaccgtcagttcgctttcgtttgccatcttttggctctccagtttcggcatcgcctaggagagccatgtggcgcgcaagctctttgctacctccaaccatcgcgccaatactggcagtcaggtccgtgacggttgcttttcgcctggacgcctcaagcctccccgcagcgcttagaaaaataactacctgctgtagggtgtaatcggggatgtccttgaacgagtggccgtagctgaccagcttagccactgtagtctccagccagtagcccggcgggcctacttccttgctcgcggtcttcccgtctcctccggcgcctttcttcctacgccgcttttttacttcttcactttcgctttgttcatccggggaccaaccgccgcaacgaaggcttgaaagagcaggggtccattctgggtaaaaaaactgaagttcctcgcgaaaatgccgtaacacactaagcacacgtcggtgaactccaactcctctacttcttccttggtgaggttagtaaacagatcaacatattgcggcaaaagatcggcggcgccggtaaaaagctgacggaccagatttccgacgatgccggcctcacgtaccaaaccttcagtatccagcttgtaaggatcgatgccctgttctagctgacgggtctgggcctcggagacgtattccagaagcttcactacttccggctccccgaggccacttaccatgccctggatcagatgcgtgagagggcccagatgcttcagtttgcacgcccggaacgttacatccttggtatacggctccgcgtcgttgccgttctcgtcttccttgacgatggttacctgaatggatgcgctgccctggtcgaacaactcctgcacagccgagagagtagaaagaagaacttcgcgattgcgcgattgcgcctgggtcatctcagtcattattccgatccttacttgcgtttgatggttggtttttgctcttggttgccgtcttcctgggcgtcgccctgccgttccttcacggtaaccttctgttcttccggttgatcgggagtggccatctcgacttcacctttgagcaacttacggcgggcgaaatcgcgactaacccaaaggtgggtataggtacggcccggaccgcagattacccgattgtactccatgtccacaaggagctttacgaatacatcgtctttgcgaagggcggtgttcggtgcaagcgtttgcattttcttctcctcaaaagaaaaggggccagtaggccccttcgataaaaccgctaccgattaggcggcggtgatcagagtctggcgatagtatttcgcatcaccttcgccagccagcgggtcttgcaggattttaccagcgatctcgaaggatgccacttcttcgttgatcagcgggaggttttgcagcggcgagaactgggcgcgcggaatctcgaccagcaccagttcttcgttcagggtattgataccctcgaaacgcaggtaacgttccggcgccgcgacctgggtcagagcttcgagacggcttacttcgccgaactcgtactcgacagtgccgtcgcccgcagtagcggtgtcgttgaaacgaatgaccccgaattccggatcgaccgaataggcagtagtcggtaccggcgccggagtgccgccagtggtggtgatgatgacagccgagacgttctggttagccagggcgacgtacgcacccggggcgatcttgatcggctcggcagctacggtaccggcggccttggtggcggtggaaccccagaagccgagagccaggtttttcagcgacagcgactcgcaggtgatggtgaaggtagcgttcttctcgctcacgatggtgaagtcgatagcccgttcgccagtcatcgattccttgtggtcggtctcggtggtggcgatggtgatctccagggcggtacagttgcccacctcgtagaggttgaggggtttgccggtaaccggatcgcgctcaccgatgatgaggcggccctggccagagtagtatgccgacttcggcacgggacggttggtagccatgaatggtctccttgtttacttcaacgacttcagtggtaggttgagcttccagacctgcgaatagtacagcatgtcactagacgagtctgcaatctccggcctttcttgtacgaagttccagacccgtacggcttcgggccgttttccggtctgtacgtcgctctggtaaatctctttgcgcagcagggtaaggatacgggctacgaagcctcgttggctacaccaagtatctaccttgccgccaatgatgatgctgaacaggaaatccgcaaacccagccccgttggtgtgggctgggttagtccgctcaggcatatcaggaccgggcttacagccttggtaggctacgccaaaaactacgttgcccatcgccatatcactttccgtacgttgctccagctcatccaggctgaagactccgactaccgaagcctggatgttggcccgctgcatcgcgcggatgatcttggttttcagatcattttcgaggaggtctaaaacctcgtcgtagtccgtaatgattgtgtctgccattagtccagtcccaggttattcgccttccggcgcaggaaagaatccaccgacttaacgtccaagcgtcctatgccgaggaagcggcgagccggtatcctgccgtcacgacttcccctattttggactgcggcgtactccgctacaccggggtcggtaatgcctatccgtactccggccccggtattgatgaaaagcgcccctgagataccgcccgtgatcttctgtatggagtcgcgaaggaaacctttacgaaccaacttttgggcgtccccgtagccttccgcctgcttacgtcgtagggtagcaggggcaaggtctttccaggggttctcgtcgggatcaaccatacggcgaaaccgatccatggtccgccgataaagcaaatccccgatctcggaagtgagggcggggagcgcaagatttcttcggattgtagcgatacgttgttggagtttcttggctccgcctacgtaccgttcgttcattaccatgctacagggctcccgttctccgtaggcgccgccgcaggtgctacggccacgcaatcgcgaataggccaaaccacgttaccgcgcgggcggtcgtagcgctggtagatgcgaatagccagctcccggcgcgctgcggctgttagttccccaagcgctacttctttcgggatggaggagttactgcttgctagcagccagaggcggagcgcggcctgcgcggcggcccgcatccagtcgggagtgcccttcagcactttggtaacttcgtcagcctcgaacccggcaatatactggatgcgataccgtccttttggcagggtaagctcaacgagacccatgcgctggtctacttcggtagtgcaagcgtttgcactgccatcggaattgaaaaccttaacgctgtccacttcattgatgaaggcgttagacaaacgcaggagaacccgattgttggactcgcacgacccgatctcgaaggtatccgcccaggtgtagcgatagaggtgctggacaccgaggcaatcctcgatacgggtgcggatgttgcgatggagagagtcaaataccgacggggaaggttcggtgcaacactggccagcctgggcagttactcccttgatgagggcttcgatttccccgttagaaagcagatacatgactctctcctcgcagttacttacttggccggcttactggcgcgctggcgggtcttggtggtcgtacgcttggcagagtcggcgctgtcttcaccgttgctttcgccgtcatcgccgccctggtcatcgtcgccctggtcatcgctgccatcgtcgtcctgatcgcccagaccgggctctacgcgttcagcgagctgcactttggctttctctcgttcggcgaattcactggtcacgtccgtgaaccatttcttcttcgagccgtccatgaactcggtgacgaactgggtgccttcgccgatcagatactcgatgtgagcgtcattggtgagacgaatgacgccgccctggtacaacggctcaccgacaacggtggtgagggtcagggttttcgggccgctggtaaggcgcaggtatttgacttgactcataactattactccgtttaaccgatcctaggagggataagaaggggcccgaaggcccctggccgatggattacggggtaccgccggggaagtcggcggcgttgtccacgataacgtgagtggccgcgatgggcgacaggccaccgaggttcttcaggcgaacaaccgcgtcgcggtcgtcgatctgggtgccgacccgggcggttaccacgatgatgatctcgcggctgcggatgtccttcgcggtttccagggtgatgtcgcggcgaatgccgaggatcaggttctggggtacggtcaccagaccgctctcgccgttttccttgatgccgatcatcggcgccggctcgatgggcaggccgtaggcgcgcacgtcgatggcgttctggagggcggagtcgccgtagccggtctgacgacgggcgacagcctgacggtagttgatctggttgtagaccgacacgaacgccttgagctgcgggagcagacgcgcgtacttctgcggcagggtcttctgggccagggcgaacacgtccggagtgataccggcggaggcgttgtccagaacgtgaacgttggccagcttcatccagccgtcgaacagcgcgaggtaggggtcggtagcgcccagggtggtgtcaccccagagtgccagctcttcgaggtccacggccacacgctcggccagcgagcgcatcaggtgctcctcgaaagaggcgccctcgatgttgtcttccagaacttcgtaggggatgcgcacttccgcgatcacctctttcgaggtcagggtgatcttgccgaagttcggcgccgcgcggtcggccttacgaacgtaacggtcgttggagccgtcgtcgttggccgaaccgtcctggcgcgcggcgcgcagtacacgactgtcgaagcccatgcggttgatgttgacctcgggggcgcccatgcggattacacgggcttgccggatcacggtcggttgttcgaggatgaagtcgatgaagcggttcgactgctgcggcagcagaacaccgcccgccgaggtgatctgcccgatggacaggtcggcgcgctttttcatgagttcgtacgctttcttggacatctctgtctctcctgttgctttactgatggcttgccttacagcacgcgccgggttgaattactcgtcgccgctctcgtcggcttcgtcttccagttcgctgatgtcccgcaggccgtggatgtcgcccagcatgccggagaagacactgccgtcgttacgggttacatcttcttccttgcgcggcttggtgatcggctcttcgccgctgcgaacgacggtggtgcccttcagctcttcgatctgttgcagcagaggctggacggcggcctttaccacttcctcggtttcgctgcggcgaatttgcgcttcctgctccaggcgctctttttcggcagcagcctgacgctcacgctcacgttcggcttcgcgctcggaacgcagcacctcggcgacggtggaggccaaatcctccttggttacatacgcggtgggggtggacttcatattcatctctttggactccttgacggtatcgctacgttgagcagaaattgtttcgggggttaccgggatcagcagcgagcgcggcaagctgtcgaggaaaccgttaacgtattcagcgaactgattcacgctgcgggtaaccaaggtctttttcgcctctaccgtcaattccgtgttccagaacaggatattgttaataacgctgtcgaggttgtacatcgcatcccgaacagcctcaccaacagtacggtcggccatgtaggagttgaaatccaactgcccccagccgtagccggagtaagccaactcattcacgaccagtacgaaacccgccggaatatcctgggagtcacctcgcgaaataactgcgacaaccccctcagacaactccacttggcgagtttctaccccatcctccaactccgcccgctgcaacacaaatttgccggacgaattgttgaggtctttttcatcgaagtcaacaccattttcagcgagccatgcttcgacttcttctcgcgacgcatctttgtcgtcgaaacgaatggcattgaccacgattttgtttttacccggagttccgctaaccgcttcggagcgtttaacgaatgcggttactccatcttccgtgatcttatgggcaaccagctcgtcgtccgtgcaagcgattgcactattgacggctttccacggctctccttcgttacgggtgatctgaagttcccccaggccgaagctttgcagactctcctttacctgctcctcggtcaaatctgccgggaactccagacgcacgagattggcggtgtcgctacggcgggcccgagccgggcggacgcgcttggaggtcgccggctcgtcaccatcggaacgaagtacagagaaaccacgctggttagccggtttcttacacatggtaatgaacgccggtttgctgatcgtgacgaggtttccttccacttcagtggaccgacgaatacgttggttcttactcattttagtctcccaggacttggaatcgatggttatgcccggctgcgtgcccggttaccgtatgggtggttatagggtggaaatgcccatccgtttctccggttgagccggagattaccttattcagtggccctagtaccactgaaaaagaatgggtatgtccgtcgaaagggtcaggctcagtagttccatgcaccaatacctgctccatctcgtccgaatacaacagagttatcggctctaggccgacaagagcctcgaagctgaatccattcaagtcacccgtaaggatgcgattccagatgtccgagtccagaacttttaccccgagcacccacgaacctgggatgaaatcaggatcgccttccctggcgatgaacgactccacaatgtagtacccggtattacggatatcctccccgtcgtgctccaaatcgagaccgtagttggcctccatgtaggcatcgcggaaccctacgatggcctctctggtatacacgtcgccgtagacgtttggggtatccggcaccaacaactcggcccacgctacttgctcccactcgtagccatccctggatatccggcgtaaagcaatttcattactcataggctttcaccctccctaccatgctccggggcccggaactcgatatcgccatcttgctccaatgacttggtagcgtcatctttgagactctggccggcatgagttgtcgggagcttgccagtagtggactctccgccgctgtttttgatggacctcgccataggttggtccatccagggttcatagccggcctcccctttctccggatattgcggcagctcggtttgcaagaaggtatttgccgcatcgatagcgctacgcggagttacgccacccatcacgttcagtgcggtcaacgccttgatgaccatttccggactgctgatcggcggaacgcgagataccaacttgcaggtcttcagggccagacctcgataaccgttgacaaccaacttattcagaagctcatccatgtcacttcgttcgggcccgaatacctgggcttcagccagcgcaattgccacttgcgcgttagcgtagttggtctcattacccataccgacaagcaccgcacctaggcgccaggaagaccgtactttagccatgttggctgcgtcatagtccttgaacaacgcgtccgactggcgggcatcggtaagtttctcgaccttcatctggatagtagagcctttgttatccagactgtctccttcacctaccgcttccacgatcatgatgcggttctggcgctcctggccggtgttctgcgtgaacatgttggtcaggttcttgaacgacgaggcagtaagccggccacccgaaaccgtcagcatcatcgggggcaccgtgttttcctgaaaatagcgcatgtttacttcttcagcctcgcggctgccgatgatcgagggaagctggttaatccagcgcggcactccgtaaggctcgttggacggcagtttcaggtgcagaatttcagtggcatcggctccgggggtatagccgtcttcgccctcgaagagaccagtattgcggttcatgcggcgcgggtcgccaaactctttgaaccacacggtccttgcgttcactcgttgaacgaatttgcgaaaacggcggaattcggtaacggtcgtaactcgacgaccacgggctatcgtgtagcgaacctcaacctcctgcgggtgcttgaccccaaggcgcgtatgcagcgcaggcgcatggcggaaaagactgagattgttggataggtcgcgaatagcctctacgaatccgtagcctactgattcccggtcacgcacagccgcatccaagaccgcttccaggttctggtcgctgtttgcgtactcgataaaagactgaagctcgtcagcctcgccagcattgatcttggcgccacgacgaatcggccctacttcccaccccggcttgactacgtttgtcacgtaggcgtcgatgcagggccgcaaggcgttagaggtttcgatgatctcgaaaagacgacccgggtcgtacggcggaagcagaataatcccgccggcagaatcaatgccggtccagtcctctagaaaatccacttgtcgcgaggtatcagcgtctgcacgtttgacctgaattacggtcgaacgcgcagtgatcgcggaggcccggcgacggcgcggaactcgttgggttgtttcactcatttcgtagcccccggtagacgtagccttcgctcttgagcatttcattcaggtcgatgttctgcccgtcagaagtttcaaggttgaggatcactacccaagcatgtgcagttttcttgactacagtggccgtaaccttcgagtggttgcaaaggtttgcaactcggtccctcagttttccagcttccgtatccgaaccctgccgtatcgcattgggtgcatccacatctttaagccgtacccgcacgcgcttatgcaagccatcaactcccaggtctaccaacaaaaccagatcgtctccggacaacacttcttgcaccaacgcaggaaatacggacgcagtttcattgcccatatcgtttctccctttccaaactggcgcgagcttacaacacaaactgaatcccgggcaataaaaagccccgcacttggcggggctctgtgcaaacgcttgcactatctcaggatatctctggccttggggtcggcttcgcgttcgacggctggcttaccgggctcaacataccttggctttcgagctgtagaaggatacgttcggaggcctgccgtggtttggcccccgtcagcatactccggagccggtgctgtagcggtacatcgaagctgtccagcatacccacaaggctacgcgtgcctttgtactcactcgacaaaaggtccgagaaatccggctccgtgaagcgtacgcacttgagaaacttgcccttcggcacctgccgcccgtttactacctgagcttccgaagttgtgacgtaggcaaagggaaatacgccgccagcctctacggatacgccttggtttttgtatttctccagagtggcctccatctcatcggcgttacggcagaacttggtgagattggactcgaacacccgcatccatacttctcggatatcgacgccagccaaattggccaagccgtggagaacggtcagaatatcgccgaggccatcgagaacttcgacgaaatcctgctcgtttatccctttctccaactcctctacttcggatttcaaaatgtcagtataccgctgtaggtcggcccagttcgggaagaacttattgccttcgcggttgccaaaaagctggtgcatgcgcgcatccagctcgcacatgtccaaacccttgggaaacatttgagaaaacgcggcttcggcgttgctattttttgcaagggtcatgtgactattcccatttttccagcctggaagaataactcttccggcgtccggttgttgttgagtttggcgtatatcagatgatcagggaaccgcgagtcgctgctatggccgttactcgtagcgccctcccggaggacctctaccaacttcatagagcctcccagctcgaatgccaaggaatccaggcactcaaactcatcgtagacgggctggccctgcccgtcgagaaagacacgcacatctgacacgacgatccttgagtgtccgttgtggtaggccgccatgatgcgctgctccatcttctcggtccagtacgattggcggcaagacaggcgccggtactcagtcccccacaactgcaaaatggttcgagaggtgttgtaccggtagagacaatcaccatccatggctttttcgaatacatcgaaaaattccttgtcagtgcagcggtcgattaccaagcgcgcttgaggctgtgtcttccactcgttgctaagtagctgaaccaccgcgaccccgaaggcttccgctacctcctcgtacagcggctcggcgtaggccatcctgatccagctatgccttgccaccattagattggccagagtgtctttcccgctgcgaggaagacccgtaagtcctactatctgcatgctgtcagccctctttcttttccctggccttaacggtccgggcgtcgtacgggttctcgcccatccggaaggggtacaacgagcagtcgtaggacgtgcaatccttgatcgactgtaccaggccgcccatacaatccacacaatgggagcgtatcgcagttttgtaactggtggccttagacttgtagttctggatgatcgcatcgtaatcaacacggtccgaccaagaatgaactacctcgcgctccggtggtgtttcggtgcgtacggggatcaactctaccggttttacgggctcctctaccccccgcgccggtcttacgcggactctaggcctcgctagagtggttttcttctcttgagcttcctgccctaccggagccgatcttttcggtcggatgcgactggacgttgccatttgctcttacctttttgactgtttcaatccacgacggctgtctatccgtcacccataacatcgttcgatgtgcgtccctatgatagcaggcccatacctcccacgactcccctacctttacgacctccacaaaagctggatcgaaaacggctaccccgcgatgcagttttttacctacaaaagtcacgaaactctcactgaactttatgtagcaatatccggaagtctccccatcctgcaccaagcaggtgttgctgattaccgagtggcgttttctgggggccctagacagaccgactaatccaaaatgtttccgttccttgttaatcatggacgttctattcccgtacaaacccgaatcttcgagtccccctaagcgattcgcgtaaagtgggagccctgctgggaggctggtacgctcgacggcggatagccagaactacggaacctacggccagcgcaactctctcctcctcgggcgtgtcggtcgcctctagcggcatgtactgcctgatagggtgcgaattaggcaaaccggaagtcgcataagacatagccgcagagggcgtaagcgccacgaatactacatgccgaccggcgccctgcccgtggatagcggcgtacctaatctctggctcctcgcgactaagagtttcgatgtattcgtagaaagcgtttgccgccgcccgaatgactaccaaaccaggggactttttattgtcacgccccagcctccacccttcggctctcgctgcgtcttccagggtggataccgcagattccacgaaagtaattttaccccggagtcgatccaaggcggcatttatgaaacttccgcgaattgtgctctgcgctctctcttgatgcgcagtctggtcgtagaaaagggcgtcaaagaactctgatgaaagctcatctatagttaaaatcgccgatgcggagccacgtatattgagcaccatttcgtctgcgccgatagccaaatcactgctaactacgacgtattctttgcccggaactggctcttgcatgtgcaagcacggcggaggtgcttgccttaaatcccttggagtttttcgcattttctcgctccactataactattagacgatcttcgtctacttctatgttcataatttccgagatgctaggatgctgtaggcaagaggtctgggcgtcttctgggctgaggtactccggcggtaccaactcgaagacaatcgagctgccggcccaatcgcgcccttctatccatttattgctacttctggggttggtgtaggcgctcacaatggacagacgcggaacgcccaaaacttttatgagtaagtcgtacgcgtatcccgcgaggtagaaagcttccctggttagcgggttacccatctgcggttcattgcttatgccgaccctgacaaacagctcatcaaagagccaatcgctgagcatcgtataagcgcttactcgaaaatgttgatactggcgtttgccgccgccgtggtacgtagggaagcccgccccctgccacccatgctcaaagccgtcttgcgtgatgtcgtgcagtctcagctctcgggcgggatcggaagccaacttccgaaaatgcggtttagccccgggcacgccaccgtattcgttctcgtaccacatcttacgcggcaggccactgagcacctcctgctgcgataccgcgacgcagaagaacggcgattcgctataggtgatgagcctagaaagtggcaagtccgccatatgcagtagagcgaattcgagcggtgcgccggttctgtcttgcgccccaggtgcaagcgtttgcacccattcgtgcacgcagatatctgcggctgccccgaggttccacatgtggtggctaggcttcctggggtcttgatacctgactatccgctcagaaagctcaagtgagatatagccataggagatcgagatgggaccggctgtccctaagacgggctctaacgcaaactcgcaaagtgctttcgcgttctccagacgaacatctaccagctcatcgccgctctcgaactcattgcgataccctttcgagtaaacggagtggcatcccataaaatccgaaagcatgaaatgcggcgatagacgggtttcagtaggattgagtatccgcatttcaatgcttccgacgcgatgaaatatccactaccttgccgatgcacttgtccgtggccggattaccggcatcgggtgcatgctccgcaatccgggcatatagcgtagacgctacatactcccgttgtgcgctgtctctgacaatctccagggtacccaagcgggcgccgtggagcacgtcaaagaaggcacggcggtaggtctcgaaataaacctccgtctgcgtagttactccttcatcgataaagctatcatcagggtgatgcagctccgccgcagcctggataagcgccgagtgatgcagattcatcccgagtttttggaaatcatctgcatcgataaaaagctgcgccgtattcaaaaaatcccctagagggatggcttgcgcgagaatccggtcgaactccgcgctaagcatttcttggttgtactggtccattgactaactccggggtctaatgctgcgcattatatcgaaaaacgaggattaaagtcacctagagcgccgtcttactcgggtcgtggatatccggcggcacactggctttggtgccattgtaggtgctagaggggtaggtaatctaccggcgtagagggtgtgcagatcgacgcgggccctaccggatagacaattacgcaaatacgactcccaaccgttgtagaccacgttcgggtagtcgcacgccggcaataggcaagtaccagcggacagcgcctcccgatctactagatgcttgtgttggtggtctatttgatcccacgccgcgtgcaagcgtttgcacattaaatcatacgctgcatccgtcataggtgattcgtcctgccagtagtaacaatacgaggctagcaggtagtgggagacaagtaagttaggcggaagtatttttacctcctccacccctagcattacgtgcccctccgaaccctgcgtatcgcaggagatttgctctcgtttttggagatggtgggtttcgccggtcgtactcttgccggcttcgccacgtcccctgcgcccgctgccgttggcggcggtagaccttttgccccacgctcgtacgcgtcctttagaccagtcgcgatagctgttactaggaaatccgtctctttatgccacgtattgagaacctgctgagcgtaggctacccattccttctgctccttcagtaaggactccagatgcttatgagcccggctgatattggctttctccatatcagtaaactccgaataatttggctattttcaactcggcgtccctgtccgggaacgggcactctctctgaatcttcgccagcgccccgccgagtcttttgtactccgcacggccccactcaacgtattccctcaacaactcttcgtctacctcgttctgccggtctttcacggtctcggctaagccacccgcgtggttggcgaaggacggcgatttgccttcgtgccgcatatcgagccacaccatgaaatagtgcagctctctggtccggtacatctgcttgattatgtctacgaactccgagtagaccggggttgcactgccgttgagaatgctcggcgccattcccgagagcatgactaccaagccctccatacggctccatatgccattccagattttgttaccctctgcaatgtggtaattgcgaagctccaaaagggtgtccaacagcggaccggacggcaaatcacccgtcagagccaaagcgcgctcagacagcttgtacccgtgagccatgcaggaagtgagctggtccgtcatgcccaaaagcgtccctaccgcgattgccgccccttccgtatacccggcttccatggccagctcttgccgcattgttgccgtttggggccacgacctttggccagatcatccatgcgcgctcgaattttaccttcgtcgaagctggagccaatcgccctcaacagtatctcaactctttgttcttgcatttctgaactccatggatacgttccctaaattttaccatctatcgcaccctagcctacctacagtaatcggcggcgagcgcgcctatgccggtcgtagatgcgcatagcctcagccgtattcaggccgttgtcgtctatgatctctatcagtttgcggccctgtcgaagacctagcagcagcatagaaagaatctgagcttcagaatagctagacgcggagggaggctccggtagagaagggtcggcggcccgatagcgggcgtaggcggtttggtagactccctggtccagtcccagttttcgacaggctaccgcgtgacagtctcctatggcgtaccgttctgctagagcctgtatagcctgggtttcgctttctgggcgagggccgcgtagtcgaaattgatactctcggcagattcgcaggacaacatcacgagggagccccaggtgcttcactgcctgcgtcaggctgtacccttgctctacgcagcgtttaacggcgctcgcatgggtacgttcaccaaggctcagaggaacatcaaacgggtgggtttctggcaatccactagcccgaggtgacttggatcggcgcgtggttttcggctttctactactgggctcgacgggcgggcactgttcaaccgagccaccattggcgagaaaggcagctacagcgtcgcctagggtattgcggtctatctgatccatcttctactcctgataactacctgtgaacactgtgcgacggccagatcgcacagtcaaggttcaggactcgtagatgttggcaagcccttggcgggctatcagtggcctgagactgctcaacgggtagttggcacttgccttcccggcacgattgaaggtttccacttcagcaaaaacttgcccggcgctcaggtagaaatcttcgataataccgaacccgccgggatgctccaccaacataccgcgtaccggcgtccataccttttccgggggaggtccgccatcgagcggcagacccgcgaggctggccgtcagcctgtcgaggatttcggagttcatgctgcggtgattttgcgaggccagtttaccgatctcggagcgcatcccaggcggcatacggaccacgaacttctcagcattacgcgtgtcgaatttgctcattttcttcctctcaatttctgcctcaaagtagatacaacttcaactaccggtacaggtgacggttttcacaaggacgcgttggccttgcacaccgcagccgaaaccagtaccgaggggctggggcggatcggctgagcgtgcagactggtgtaccagcgcttacctgcccaagccacgtaacccagaacgttggcctccaaatcgagcagcagaccgatgtcgtggtcggctgcaaccagcgccggcatgccggggttgtcctttgagagggcttgccgcccctcgatccactgatcgaaagcctgcggaagccgcaggtttacgaaacgaatatcgggcccgtaaaactcgtgcaactcggagtcgttagcgaaaactggcatttggttactcctcacttggttggtctgactcgtcgcggcagtctcttaactgccggcaattctggtttgggttgttccgctttacgacgtgctacggcagcaaatccgtatacatattcgcctatctcagatcggcaggcaggaagccaatcgaactcgctagggtcccaaagtaggtcacccgcttggatgcgctcatttatatccggatcgacaatttgaaattcttccggtgctgaagtaacggtcgtcatgctactcctccttgtctacggcagactaccccaaccttctacggttggctagctacacaggtatgcgattagcgccgtccatacctgtcagacagcctagttcagttattgttttggtaatggatttctgcccatagcgcgattcgtgcccgacgacaaacttgtagcggtcgtacaccaagcagtctgtgcatacgaagcctggggcgacagcgaacccaacttcgtaggccgtatcgtcggaccggatcatgcatacggtcagcccgttctcttggtacacctcgaaagtcacttcattcggtgcaagcgtttgcatttcaattctccttacattgatccgggcctctccgcttgcggcggggaacccgagctagaaccctgatcggctctatcgcagaggcgggtacggtctgcggggtctcttcattcggtatcgagatggttgatgtggccctggcctggctcgttaaccgccctcgccaagcggctactactctgtctatttctgcctccgcgtgtacggccagctctacgcgattgagtttttcgtagacaaaactttgagttccggcgatgaatctctctacctccagcctcgcactcgggacgcgccatggccgcgtatggctctcctccacttcagctatgcaggcgttcgcgaggcgtgtgatgcactccatggttgggtcactggaatactgactcaactggaaatccgatagggtttcaccttctcgggagactgggatggcgtcctctctctcgtacgtacaacagtagtcgagcataccgcgcattgcccagtcgtctttcctgtacccctcgttcgacattgcaacgtcacgcactgcggacattcgggccggcaaagacccgctaggttctgcctcgacgatctcgctagcccgagatagccctagccggtagcgatgcatagcccgaagtatcaagcacaacctacgtacgggttggtaaaatgcgtagaccagcaggctagcgcctaagagggtgacaaagagtacatcgctcatggctattcggactcttcttcgtcccgctgcgccaatcctcgggttccggtgtactggacttcgttgagaaactcggtgatgtctgcgcgtttctcgccggaatggagcgtaagcgttgcgttatggaggaggtcgcaagccttcttcaattgttgctccaatactaccgcatccgaccgccccaggccgttatgcaaaaccgagaaagccatatgccggttcgcatgtacggagaactgaagacttgatcgcacctcgatccctgacggtttatgacaaaccgctacccctgttttagtagccatagcacgggcccagctaccttccccgttgggtattgggtaggtgtagatcgatagctcggcactgtctatacggccatcccaagctacaggtttccgataaatcggggccagctcacccgcgtgcagtatcttgtacccgcgagcttcgaggcgcgcaccaagccagggatgcattacttgctcaacctgcacgtagctgtagttcagctctttcgccatggcctccagatgttcgagaactcgcgagaacacgcctcggcaatgaaactttgggtttaccgagatatttgacagcccgattgcgtactcgaaactatcggtctgccggtcgtgcgcgtacctaccggcccgcaaataaacctgcaccaaacttccgctggagaaccactcggaagccggagtatccaacacccgccttttggctacgaacaggctgatttcctggcgcattttggccgtgatatctactttcggtctttcattctcagacatttctatattctccatggagtcctataaaccaaagcaactaaacagcgttgggcccgaaagacctagtacccagcggggagtaataggctatatcatggccgtacgggcctctatgcaccatgccccgctctacaagctcatcgagccactggaatacccgagcgtccacgctcagtaatgccgaatagtctcgattgaaaatagccgcatggtgccgccgagcatattccgggataacgactacggcgatacccgtctcctcgacggcggcgaggaactggtccagggtttcccgcttgagcttctctgcttcagtttctttcgcgggtcgtactgcttttggcattgctagggctcctatacgggaattgacttaccagacctgcgtaatacaggtccaagtctgcacagcgttcaacaaggctatcgagtagccgcttttcgcgccgcagccgggcattcgacatgcgaaccccggcggcaaagaacaaaaaatttatgcgcgagagcgggcgtgcaagcgtttgcatttccagctcacggtccatattcaggcctcataaagcgcgaagaaggagatcggcgcctgcggtatgtagccgatagcgcaagtggccgggacatgtttgatattaaggtccatgcgattgctccagttatgccgattggcgattttcttcttggaacacgagactgggcaaactgcggaccaagaactcgtgctccagtacacagtaccggccttttacgacatggactatcttgtagccccggatgcgcacctgggtacggaacgctgagaactgcgggtcagattcaatcatgtcgggcaggtcaagcaacactctgaacgtttcgaagtgaatcgatgctgcgggaaggttgtccttcttgatttctgctaccaaacggtctgctgctttgcagacatgctcgaagtatgggggacgctgggccatggattactccctgtagtttgaagtggccatcatagtgtagccaatagccataaccccgtacctagacacagaatagcaagcagacgaacggccataacccagagggtagctatgatcgccactttgagaagatgccgagtgagggcgatacctctccgattacgtcccgcagaccaaatgcacgaaaaacgcacgaagtcttcgcgcgcgagtgagtaaaacaggcacccaatcgccataaaaaggcaaagcatggcctgcgccagaatcatactgcccccaggaccaagagcaggagatagctacggatggcgatatacaacaagccaagcaccgccgcgatggtaagtacaacgtagcttactcggtaccagaaatttacagtcttctgccgctggagcatcagctcaggatcacggcggccaccatggcaatcgcagttctcccgtatatagcgagcgcacgtaaccgaggcgctaaccagtagcaaacaccccaccatgagcagcagattgaaaaggatcaagtagcctgggtggctcactaccccgaaagcgatatcaagcatgacgacctcctatagctctggcccgagaagcagggttccgatgattactaccgccgagaacaggatcggggtaacacagaacgcgtaccatcgaaccagccggtgagccctggcgtatgaatcggccatggtgtacttgagcacccggcgattgacgaaggtggtgagcagaagcaggacgagtacgtaagcgacggacagccacaaggccatcccctggttgtggagttccatgcgggcctcacgattgcatgcgtagtgtgatggagagcagcgcggcgaggaatacgacggctaccaagataagagaggcggcgaggtagtgcagtgggcctcggtcgcctagatcggcaatcagtacacagagagcgatcaagaggagcattattgcccctgtgatgactatgctaactacagacatgtgcatgtctccggcgcgtagcgccctggttaatgggtgtcctctagggacaacccgatgttaactcaagacgctcgcgctatcgaccctcagtggtcaactacgccgggagtagggtgagttggggtatttgtagtgggctcgcctccatccaggcaccgccttgccgaaagaaggcacatgcctatggctacaagatatgccacgctctactacccgataaccagagcgcactcgccgatacagtagtcgctccatgtagaaggtttctcgtccgccacgcacctgggttcggcccctcatcaggcagttaccgccggaccagaaggctcgctgaggcatccagggtctcagccatttcatagcgcatgcgccttcgagaagaacgccacccactcagggtcagaagggtctcccgagaaaaaccgccgaaccaactccagagcacccaacagcttggcgtcgatgagtttctcggcgtcgatgtgtcgggtattgacatacccacgagccttcaaccggcactccgaatccgtacgccccgccgtggcggtcacgctaccgaaggagcagctaaactcatagacggcgccgtgcttaacgaaccgaacaccctgagttaccacatcgtagttcatctcgaaccccggcaaacccagaggcttgtcgtcccatgtagccagtgtcaaacgcatagtcacctcacagtttcacttctttgaatcgagccaagccggtttcttctttccagttggagtagaagaaccgatgtagcgcctttagggcctctccgattcgggcctcggcggccctgcgactagcagccgttcttaaacgagctaccgtgctcattatctcgtcttgtggcgctgcggcgaccactactagaccgtggttttcgtgcaagaaacgaatgcaggcccaagccccgtccagccggcgaaacactaaggttccgcgcttttcgtcgtagtccagatcgaagcactccagatggaccagggtaccatcgcaggtacgcacctccgttctcatagcggcacctcgcaattggagacgttggatttcggtagcttgccggctgtgacagacataccgagcatggcaggccatataacgaccgctacaaccaatgaaaactgcattggctcattggggcaagtcgggctcggtcgccggctacaggccccaataacaggcctcccaagaacaaatacactaccgccgctttgaagaaagacagaccaccgctcatagctacgcaccccatacggcgatcacgattactagcatggcagagtagagtatcaaccactcagatactaccaagatggagcgcagcctttcccggttacgcaggctcttactcaggcccaaacgcgcatcgcgttcagcctgggtctccccggtgaagcagtcacggtgcagtcgctggcgcgtgagttgctcgcaacaacggctaacgtacttttcgtttatgcataaggccagccatacccctacggcgagcactaactgagtagccagaatcttgaagatgagcatgctgcctcctacataaaatagaggtggacagtaccgtacacggctagcgccacggcggacccgcccactacaacgaagaccatggatatcaccaatatgagggccgccgttaccgaagtgagagcgttgtattgcgccatgtcggcagctacgtacccggccatcacccctactactacgagcacgcaacccagggctcccagcattgtcatttcgtccatactattcaacctcgcgtttttaacgtattgaaaattgcagcgcaggcgctacgactattctggcatccactccccagtctgtaagctgcaccgctagagggtcaggcggaagctgctcgtggcctacgtagtagtagctcttacctgctcggcccttgttcggatcgaatataaccaaactcccttcgcgcatatcggcaattacctgatggagaactcctcgcaaattcagcgacgctacgtataggaggtagagattgcctgggtgcagcgtatggccttccgcgtactcggcgcagatgcccagcttatccatgatctcgaatacggacatacttccgtcgcggtaccccgcatggtagctgtcaatgatagtggcagccggctgccctgatagcattgctaggcaggtgcatgcgcaggcatcgggtagaggctgggtctggtggactatgggtagagacatggttccttaacctatttcggggctcttatgcgcccgcagctcatgagaatgttaaaccgtgcgttttttggattctatcctgaaactgatgcgccatcatcctacagtgactcaggatagtgtcctgcctatatcgcgatggaagcttgctggaaagctggcttggacactaattgaatttgaaaatttcccataccctgaagcgatttttcgcattagttcagaaaccctgatcatgaccctgatcatgaaaagagcatgaccctggacatgaccctgctgcgatgagagctgcccgaatcatgagagagtcatgatgctgtctgccaacttcgcgtggtggtctggtggtctgcccgttgacgaaaaaagtgaatagggggtattcaaaatcccatgggtataaaaactgtgctgcgccacgaccgttcttggatagagaatttctatggtggattattcctatatggcatagggggtgcataaagaagggccccgattggagcccccgtgcaagcgcttgcgctagctatggaattcgctcgcatcattcggcttgcgttgaatggcgccgatggtgaacgacttgccctcggtataccgaacgacaatcgtatgctccagccatcccccttggatcattatctcgccgtcaatgatccttggctgagtacgttcccccagatagatatctacggaatgcacgccttcgccttgagcgtgtcgcaatgcgcgattgtaggtggccgcgaaatgttcaatggcccccttgtcggagaggtccaacggctcatagatatgagttacttgttcggctcccccgttcgggtggctggagtgtaccggatggtgaataacgataggtgcggacatcgtattgactcctttagtcgataaggaacatagcgcggggctgcgggttagctagcgcgtaggcctcagcctttgcccgaatgctgcccaggcgatcacgctctttcaggccttcgaagtaagcgcgttgctctgctgtgagatcgctataggccagttcctcaactatgacggtctcattagcctggacgcggaagatgcgagctacggcgctcactacgaggggtttacgtgccatgggatatctcctatagggttaggtagtggggcccgctatgggccccgttcgaggttacgcggccaggatcaagtcttgcgggatatgggcgatctcgttcgccttgccggtccggaagatggccaggaacgcatcgcgttcttcatcgcgtacgttaccctgacgggccccgaaggcgatacggcctaccttgctcaggctgatcatcttgtcggactcctcgaaattgcgagtcttgttaaggtggtaaccgactgcggtatcgcccattacgcgcgccagctcccggcgggaaactttgacgccttcgccgtacaggccgaaatactggagaacggcctcggtatgggctgcgagacgggccccggctaccggacggaactgcggatggataaagaaagctgcggtagtggctgctttcgccttggccgcgcggggcttggccgcgcctttgctggctgcggtttttgctttggccggagcttttgccttgggcttggctgccgctttcttgacgggcttggctgcgggcttggcggacgcggctttcttagccttggccggagcttcggtaacttgggcggcggtttcggcgatgttcttttcaatgagagcttgcatggtaggtcccctctgggtgacttgcctaggcggtattgcctggcttggaagtcataatatgtacctcacagagccaggtcaagcatattttcaaaataatttcataacacaaggcaagcgcttgcacctgctttccctaccagaaggaggaaacgggtgcgcgcaggcgtgcacggaccgtgccaaaagttgtagcacgtctgtaccccgcgaccagcggtgaagcacgtcaatggcgtgctacaggggtttccacttcaccagcgatcactgggccggaagcttcaccagtcaccagtaattaaatacacatgcccacacaggatcaccagttagtaatagggatgtgcccacacagaatcattcaccagtcaccagtgcagtaggaaaaagaaaagggcccgcagtgggccccgttctcagaggtagatttcttcaccgttctccagctcaacagtaggcgggtccatgtagtagtcaggggccagttcctcatccagccagtgcggctccggggtatcaccaatccctgcggtgcatttgttgcacaggcagttcccgtcgtgaaggtcgtccatcacagcccctccaggttgattactttctctactcgggccagatcggcatcccacacaccgttgtccatagaggagtccacgaggacttcggcgggcgaaccttggaagatgaagtacatggttccacggagttcttgcccgtttacgtccttgcgccagtagagccaagcttcctcggtctccataagcagcgatgcgatggagcctttcgctacggtctccagtacgtggcgttcttcgccgtcgctgtatgagtgtgcgcgccagcctgcctcttgcatccccaggatgatgcggcgaacggcggagccgtcggttttggcggcggtggtataggcggtcagggtcatgatgcgtcctctatctgagtgactgagatgcaaacgtttgcatggggttcattatgactcccctatataggaacgcaaccaacaccgaacctgggtgaatccgtttaccaggggcggggcgcttcaccagcagcgatcaccagacatcacaactcaccagcagcggtcgtcgtcagtcaccaggggattccaccagttacctatataggagatcaccagcagtcatcagttatagcgcgtacgtgcccacacagggtccacaagcgggcccctatatagaccaccagtcagtagcagtaattaatagcacatgtccacacaggatcaccagggagtaataacagagatggggccacaccgggccccgatccactaagtacgtttgaaccgaatccagttggcgaggccgttaccttggatattgtatttccgcgtagaggtagagatgccggaaggccacgcccagagcagctcatcgtccggcccatactcgcaccatccgccatcatccttcacgtcattatgagcttcactgatgagccgctcgcctaggtggatcaagccgagaatatggacaactatgtcgccctggcgcagctccagagtattgacgcgttcgaaaaagcgctcacgggtgtcgagattttcgtatagctccgcgatggtggacaagggcagatcgcccggcttgaaaaccatcctcttaaaaccatactgacggcaggcagcccggtgcgcctctgccatgctttcgcgcactatcaggtctaccagctttccggcgccgccgttctctgcgcgttcgtgtacatagatggcgtacataatgattctcctatataggtgcaagcgcttgcattagaggctgtcgaggtaagcgatatccaattcggtcggcggttccaagctatcgaggaacgaaagcccagcttcgacactggtagcaacgcagatcggggccaggccttggatcattccctgggaaaggatgaagctacctacatcgatcattccattggaagcttcgaacccttctacatacaggtcgccgtcgatctcctgcggcagggtgcaaaagacaagattgggataggtgctcggcatgatgtgccccctcctgggtgactgtttatttcgtaggcccccattatggtctccctatataggagtgcaatacctaccagaaacccaggtcgcaattaccacacgccctatataggagagcatcagtaggagcggctccacccagcggcccgggctcaccagcagccaccagcagcagaggccaccagtcaccagcagcgatcagcgggcagcaccagttatcaccagccatcaccagggagccataccatcagcgagtaccctatataggacagcagagagaacagtagtacatatcacatgcccacacagaatccacgatagctaccatccagcagtcagtagctcacgttagctaggacacaagtcacgccagccagcagaaggacacagatagctacagcagaggccaccccttgcctgttcacaaaatttttgctgcaaacattccacaggtagaacacaagctatctacttcctctatactctctctatgtatctatatagtcctatatggattctctcctatatatgtatttctttttgttctggccccgaaagttaggtaggagggccccggatggccctggctggactgtataggagggataggggataagatggctcaaagggagaggtacatatagaaggtctatatagggatttcctgtatagcgttcgggccccgaatgttcggcccggaaaagaaaagccccggagtgacccggggctgctgtgcaagcgcttgcaattgcctatacagggttagcgtttcttacctcgcttgattctccacaacaatgcgacgtgcgcctcatcgaaattgcgcaaggcttcgaacggtgcgggactcatcatagctccccctctactatagccttcagttcccggaatttgctaaagaggcgttcagccccggccaagcttagataccggttactatggtccgctacattgcggttataccactctgccacagcttccagcttggccctgttttcggatgcgcgttcctcggttttactgcggtcataaacgaaccgctgaatattgagaaactcgttagccccgaccagggaactacccgaagcgtcatactgcaaaactattccacgagcgtgcagagtgtaaccagccccggagcttacctgctgctcttcgatactccagtaatcgaactcggacaaatccaaataaagagtaccgcctattacgaagtgaccgccacgggcgatattgggtagctctgcgcgggctggccgtacctcggcaatctgtttaacttcagaggccgcctcgtagcagtatcgatccgccgagcgcgggaggtagccggtatcagaagtggccagaacgccagcctcgcgggtgtagaacttcagggactcgatctggcgacctctgaaggaccagatgttcataaagctttttgtacgcatttccattgtttatccccctctgggtgattagtaggtgcatagtagcgccgacaagtagttctcgctacctacagcgatcaatcgactttactgtgatacggcttatccacgtctacgagaatctcatcagtgaggtaggggtatagcggggcgaaagctttaccgtgcccgcatttaccagaaagacgctcaccaaccctgcccatgccacgatcccagaaaccggcgccgtgcccgttgcgagtgagccagagatcatggcccaggccttcccacgggtcgaactcgctactaacccgcgatgggtacaaatcgacaaaggcggcgcaatcggtcatgttctgcgcatagaaggcagagcaggtacgctcggcgtgggcgaggaatgagggggcagcttcgaagccttccagcgaatcgaggaattcttcagtttcaggatgtatgccgtcggacgcccacaatgcacaagcgatataaccctttgccattttttcgatacggtcagagattgccctgctcttgatcgtagagcggttgaaacgatccatatccgctgcatactctttagtgtcacagagcagattgcgcttggcgtcaaaaccttctttgaaagccttggcttgccatgaaccactcatagcaatagccaaatttccaaaggtcacggggtaaccttccggctccaggcaaccgagagcatgcaaagcaccggcgttctcatacccgaatccgtagtagtaggctcgatttttgatcatgatgtttcgccctttctaagtagatgcaagcgcttgcattagtaagcggtggccggttcgcggattaggttacgctctacaaggtctccccaacagccaataaccaacctttgatcgaagaacatgaccgtagtaacgtacggatcacccgcgttgcaatagtgaacgtgctctccagagcgcttgtactggcccaggtactctacgccacaggtatccaacaggcggtcaatgatgacacggcgtaattcacgggccccgggctgggtgtaatattcctcgatcagagtatcggtctccgggaagtgtttgcgcacttcgtcaagatcgcctctccagatggcacgaatccgcttggccagggcgcggtccacatttttaaccaaatcgaacttttcgatacttgcggctacacgagtcacgataaccccctcatgggtagtagatggatatagcgaaattataacgccccggctaaggggcgcaagatacagcgatcagaccattccgtgcgtatagttgtacgtgctgattaccagagaatcgcgctctacatccgctttcttattcagcgtctcagactcaccgaagcagtggacatcttcgaggttcacgaaccctgcactatctacccacacgtcgaacctcggaaaatcttgcatcagtaaggccagattggcatgagccactgaggcgtgaactagcaaagtcgggaatatgacagggactagcttatagattcccgattccttgtgggtgagcctgaacattacatacttcatctcagtcattgaactggccccattacgtaaaaaagggagaaaaccaatactaatagagcggcgctaaccaaaagctcggttctactcggcatggcgtttcctcgaccggcggcgcaattgcaacgctttcgcacactcctcctcatagtcgatattagccaggataccggaaaacaaagcctccgtagcgcgtgcgcgttcgtggtcagcctgcgccgtaggcaagctatcccaatcgagcagagcggccttatcgatcccggttaccgcgctcaatacctgaatagctgcggcaatgcggatgatgtcggacggactaacccgcgccagcttacccacgatatctaacggcactacgggctccagcccgcaaacgcgaattactgcgcgtccgttctcagtacgtttcgacataaatttacctattgatatgtggcgtgcaaacgcttgcacgtccggaatttatcgcaaaaaatcgcaaactttctaaactttcggggtttaatagaattttactacttcgcccggcgcacgcgcgttcgagcagaagcctggcgcttgggtatacgcgcagccgcgtccgggtgtaactccagaaaccgggcaagaccttcttgcaaccattgttcaccgggcgtggtaagctgccagtaatccactgaacctgactcgaattgcacggcgaggccgttcttcttcagatactttattgcattcaagcctatttcacccctggcttctacaacggcacgaggaacataggccccgccgccattggtaaagcacagatacaggtccaccttgtcgcacggacgttcacgggtgaatacgcggactggtacggctttaatggagtttttgttgttcactgggcaccccctggttgcgcgccgtacgaaattctttccagcgctcatagttatcgacgaatacctgatacttcagtgcaaattcttcgctcaagcggcaagcgtctggccaaccgggattgcgctccatcgcccggatgaacttggaagccaacgtgcgatcagaggtgatgaaccgaaaagcgcacgagatttcgcgaaagtttccactgatcaggtatgcaccaacgtagggcttcctggccgcttcgaactctttccacgagggcgagagacgactaccgtgtctggaccatatgtaatcgtactctacaggctcggcttttttaaggcgcttcactgacgcgaactcggtgagcgtgtagaccacaatcatggacagaatgccatccaatacctgtcgtacatccggacgtgcggagtctcgatagctaacacaagacggcataaagctaagctctacggtgccgtctgcggaaagaataggcctatgcattgtttatcccctctgggtgactgacgaatacgaagtgattatagcaaaatatacgaggtttgggccgtacagtgattagtcggcatcggcgtaggcgggttcaccgtttccccacaagacgccatcctcccagatgtcgccattctcgtcgatgccagtgatggtggtcagcgcccaggtgtagtcggggtcgattcgaacctcgatactatcaccggagtagtgctccacatacttgatattgttgtcgatagccttctccagctcccagaagagcgcggccatgttcggcgcgttgttgccgtagaactcctgcaccttggcatagccgctgcatagccactggtacatcagcttgatacaatggcgtttgcttgggtccatgccgtgttcgttgcgacagtgccgtgccgcgtgggcgcagataaggcgcagaatggcgcgggtgccgtcttcgtcgtcaggctcttctacccgctcgaagtccagaggctcggccatttcgtaatcgctgtagtgcaggaagtcaccgaactcctggcccatacccttagcgtgttcctctacgctcatgcggctggtatgcgggttatcggcgaatgccgcccaatacccgccttcgaggcgaatccacacgatagcgccgaacgcctcggcttcgccggcgttgcctgcacgcggggtgaagtgctcaaggatgtcatgcgcggcgcccataccctgtacaggatcgaagtgcggcatggtggacataacccagccggaaaccccgcgttcgttttcggaagcgtcctgccaagtgaaattaacggtcttgcctgcaaagctcatggtaaccccctcatgggtagtatgcgataaacgaagtataacgcatccttgcgtgcttgggcgcctacagaggctcaatctccaaccatggcggaaactctgtttcagaatagcgccaaaggtcgaatatcgcctcctcatccacgcatattccaaatacccaacctacagaggctttgtccagatagagagtaactcggctctcgacggctacaccgagcgtgtccatcaagtggaaaattagagtatttacctcaatcggatggtaaacctccttaccgttgtagcttacctccagcaggttcgatacgttcgcgtaattcattttctgcgccgccggtaccagccgaaactgcggcgtgtcgaacagtaggttcattcggtgaatccgttaggtgaaggttaagacgccatagaaaacaaaacgcaaatatcatggccgccagctttgaaagtcgccatagtgctaagataccctcggtcatgcttcccccctaccatggccgccgccgtacgtgccaaccttgcgttctgtaccatcccagaacatccctaaattccgccatgtagtctccggtaacctcgtcgtggtattcctgcaattggcgctcgctagccgtagcgaatggcgggggagactgcggggacaatacaagattcccatcggaatcccgcacctcgacgatatagagtttagttgtcatgctggactctccggcattcaatacttacagacttatcataccttagcgggcgccgcccgtctgcgccggatagaaccatctcttttcttgcctcgcactccacgagaggaactaccttactggtagccgtagcgcatccattctctggcgtgcataccgtgactaccattaccgccagagggcccaggatagtgatcacagcggactctccatggcgttcagagctatctccgcatcgcgaatagcgatccccatacggcagtcgccctcgcaataggaatgacaacgctcatgctctgctattagctgctgcacgagtttgcgcgtttgttccttcagcgggaattctcgacgaaggctaagcgtggccatccacttggcctttactttcgtctgcgctactgagcggtaacctccatccagaggatgtttgtcccggttggttatggagatgaaccagtcatctctcgcgccccagaagactgcacgcccgttcgccttcagagcagaattgaaccgatgcgctacgcatccgtagatattactcaaaacggcgctatcttccacccgatcagtggtgagcgtatgggcgaggcttcccaccattcggaaagccccagagagcgactctacttcaaacatggcagtagccatgggtacagaagtcggtagagtctcgtccaaacgtccccactcaataataaatttgctcattactaaacccctttttcaggacacgtaggcgtgcaagcgtttgcacgccggccagttacggattagttagcttgcgcacggcaaccgaaagacgcgcttgtatatccttcagtatggcgcactcctcaaccggtagacgatccgaaagttcagcgagaaccgcctggctatacaggcactggacatacagattatggatttcgtcggtgctatgttgggccaaccactgataggctagcgccaacatatccctatcatagcctgatccctggaagctagtaccaacaaaaccccatatggaatcaacgtgctcagcatcctcccagggaacagaggagctgggccgagcctccaagatcacgccccagactccagaggtctttacttccctacggaatttttctttctgttttttaagctcatccgcgtcaatatcagtgactacttcagggttataagagtcgccacacaagtcctcgaaactacaactagagtcggcctccgtacgtaaccggaacaaaccgagccgatgaagaacacgaagataatccctatgttcggtatcgacggaagccagaatcttcagagtaatatcgccggactctagatcagaaattttaagcttttccatgattgttccctctgggtaacgaatattgaataacagtcagcgtcggactggcgagttagagtataatgatcaaacccggacaacaccgaaataagcgtagctctcgctcaggtccgcaatagagcgcatccaagactccatcatgttttctccagcaaacagttcttctactgcttgagccagacctaggtacgcactttcaccgacgggttcgccactctcgtcgtaaatttcttcgacgttaatactcgatagcagcccggttttaacgtcgcgcttgcgtggccgggccgagagcggaacggcgaacacatgcccgccaaaacctagaaaatagtccggcaggcaggtaccaaggtataccagcgagtatgaggtatctaccgttggttcattttcgagtcgccaaagaaaatctaaaacctgcccggcggattcacgcgatctttgcgcagacgctgacgataggcaatttccaggcttagcagtgagctggacattagttggccgaaactccgcagccgcgctaatcgcgatagctagcagcaactcggcggcagcttcagcggcgcctactttctcctgccgttgcatcaggcgtttgaaggtttcaccattatttttctcaagacgggtaagttcagcgagtgctttctcagaaaggctcgtcaacccttcccgtaccatcgatgcaatttcagttttcgttttcatgctagcccctctgggtactgtaggtatatggccccattatggagccgtctaattagttcgacaatagacagagatcaatcctctatgtacgtgtcgaggtgcgcatgaactatataaccttccgcgcgggcttgctggtgcgctgctacgatagcttgcgtttcgaatatagtaggcatgcgataaagctttcgctccggcggccaggaattcggtcggcggacatttaatactactgctatagtcggacctttatggggccggctctgcgcagcctttttggctttcttaggttccgggagccccaatgctttgcggatatcttgttctgtaagtttcacctaagcatcccctctgggtgactgaattagtgcaaacgcttgcattatacgcgatatgggattgtgtgctgctacagcgagcgaataccagaggcctactaccagaaagctaaatctgacagtcagtggggtagcacaagaagagttcaccagctcaacaccagtaaccagaaggagaccacccagcggccagcgggagctgccagtaatcgaacactgacgccggcgaataagcacaaaactgcactggggtgtagtaggggagcccgtatgcccacacagaatctggacagtagggggaggcgtatgcccacacaggatttagtaggggagcgtccatgcccacacaggatttcctttctacctctacggcatcggatagcgcgctacgatttcgtcaaccttagctacccattcggttaggtcagcaggttggcccgtcttggccgcttcgtactgagcctccatatacaaagggagtgactctacgccgtaggcttctctccgctgctgcctgacaagctgtcgcttctcctccaacagttcctcgggagttttcaaatgaccccaatcgatagtcatttcggcaactccaccgggccatcggcctcgatcagcaacggttcagggaagcgagcggcggcactggcatcgacggcaagcgggacccgcaggatcagctccaaccggccggcacgtcgcaggacgggaccagcgaaccagtccgacccgatagcctcggccggcaactcgccgccctcagggagcggtgtgaagtcgaattcctggccgtttacggtgagtacatcaccggttttgctcagcgttaggcgctcgtcacttcccggcagcggaacatacggcgacaacttgatgatcatcagaaccacctccccgtagcgattgcagaaatcgccgttgccgtgccagtcgcgcgcgaagaaatgtcgaatatacgcaacgtcacactcgttgtgctcgcgatcccaccaacggtaccccagcctgcgccggtaccccagcgaaacaaaccgacgctaacagtcgggcttcctgcgaacgtagcagggaaagaccagcctctagttccggtgaacagactaccgtagggagagtcaactgcctggtcagtaacggaggcgttgaaccaacaaatctgcgtaccgtctgccagcctaatgtattccccattcgcgttgctgccgcgctcaagaaccgatccgatcggcgagcctccagactgggctactgttccaagtaactgagcatcggacagcacgcggacccaaggctgccacgtactggccgccttacgacggaaatacagtatgtttgaagtacgcgggacgaatagctgaaccgcagtcgcaacgtcgtaagggtggtgatagagcatcgatccgatagggttcagcgaatcgatgccgggcggaaggttcgcccagggcgacgcgccgataccatagaacccgcactcgcccggtaccgtgttcgggtcagaaaccgctcggttgaacgtcaagctcttggggacgccgccgatgtagctcaaagcattcgcctgcgtagacgctccgagcattccgcgccctacgctcgtcagcggtgtctgcgcccaggtattggccccagtctgatatgggagttggtcaacgccacccgatagcgaactgaagttctgcaaacgggtatcgaatagattcgaccgcgcggcagcggcactcgtagcgccagtaccgccattaataatcggcacgaccttggaagtgtcgtaactggtggccggattagcctcgaactctagccacactatccctccggggtagaccttcaaggtgactatatcgtaggcgttaacagtggctgttgctccagtgttagacagaatcgtacggcccggagcggtgatcgaccatttaccacctaacgatccctggatcagcttcaaagagaagccttcagtgatcgaggcaggtaccgatagagttcgattactggcactattgaggtagacagacttacctgaccaatagtccgcaatggagactgttgtagtttcgtcccctatgatgactccagtctgtattaccccgccagcatgtaacgtaaccatacgcgggtctaagaactcgcgagtgaatcgctgcacgtaactgacatcggcgcgcatgtcgtcgaatacctccaacgccaacttgcctatagtaccgctagcttccgagttcttatcgataacccacttacgtcctatgaccgtgtttaccgtagtgctgttgcggacgcctactcgatgctcttcgagaaagccttgatcgagcgcgccgccgccaatcgcttgcaaataggtaaagcggccaacgtagtggccgcccccagttacgcttccgttagccccgttgaagatcatatctacgaagtcagcagctactacccctccattgctggcagaagagggcgcccaggtcgcctttagccatcgagcctgcttaacttggtcgaaagtagaaggtgccactgttaccgcagtagtcttctcctcgtacagatcggtcttcaaacgatagttaaggatgatgttttttacatcctgagctatcgcctgggctagtgctgatagacgttgtgcgaggctcatagttgttacgccttagctgcattatatacggctacgaggtcctgctctgggtcgccaacacccagattctgacaaccttgaagcttctcggcggtggtgaaagtctgcgctgcgtctacccgtacacgcttgcccaacgcctctgcgatagtggccgcgtagttaggatcgttgccgagggcgtcagccagctctttcagagtatccagtacctcgggcgcgccatttaccagatcacctactgccttggcgatacccgcagcgacagctttggatgtggtgtacttaacgtcatcggtacctgcaatcacgtcgtcgccagtggctttgctggtgatggccaatttaccagcgagttccgtagctgacttgtcggcagaccaagttacgtccgtgtcgccgatgccagcgttatcgtcgatcaccgcgccagactgcccgatcagactttttacttcgttgatcgcggctacgaggttgctcttagccgtcgtggtcagggcactgaggtcgcctatgctgttcgtgttgccatcgatatcagctttatacaggagtagagcggcgaccaaactagcaggagtgggagtgatgcccaggtctgccagattgccaatagacactagaatacctttgacatcggtaccgactgccgtagcgagctgattgagtttagtttcgagactcatggcattacccctttgcgagtatgtaatatgcgagtggatcggacgctatgtcgtccggagtgtacaacttactatcggagcccagcgccagattgttcccagggtctgtgcttatcgccgcctccaaattgtctatatttccgtccttgcccggcggcccaggagggcctacagatacctcagtcagcaggatcatcggctctagggccggaccaaattggaccaccgccccatctcccagtgtccaagctgtttcgtaccgcatcaccggtatgagcgtcatctcaaggggcataataaccccccggctttacagccaccttcccggtcggcgtggtgtacttgtagcctccgggcatggcatagtggatgtcgatatccaatgtgaccaacggccagttcgtggtatccaaagcctccgctcgatacagacctttggtcgcatccgtcatcgagataaccagctccgcaaccacttcactaccattgcggacttgggaacgaataacccagtccgtgatatccactggaattccatcctggctcacccggccctgtacaacaaagctatcgccacgggtgtggcacatcgttgggccattcataacgtatctcctcctgtaaatggtccatacagtagctcaaatcccaggcataaaaaagccccgagcgaacggggctttatgtcaaaacaaactacttcgataaaataccggaaagacggcgagtaagcaagtttttcgcaatagtcatacgcttctcgcagtccttatacagagccatcagttcgtcggaactcatctggctcagttcgtttttatccgggtcggattctcgctctatctcgtcccagacttcttgaagatagaacttaccgcagtaggtccgaatatcgccggattccggcacgcaccggaggtctacatcgtcgtctttttcgtcacgaatgtaaataaaggtaccatcgccatcttcatcgcaacgaagagcgtaggggtggccatggcgcaggtacggtccgcctgacgggacgaagaccacattttgcaaggcagccgcagcatccttcgcttcctcctcgctcaggttcgaccggaccagactcgcgcctacccctttctttttgcttacagtcatgtctatttcctcggttcgtaatagttgccgggccttgccccgcccggctggggtccaacgacggattctagatacggtctagactatgctgtattgcaaactacgtatagctacacagacctagtgcaagcgtttgcactaccgcctgattcttttccgagaagcaacggaatccacatacttgtatacgtggtagttcaaacgcacaccctctggtacgtgctgtacagagttctgagaacgcagatacgggtctacacgccagccatcctctttacaatgctccaacaggtatttttcccgctgctcagtagttacactaccgggcactaccacgctgcgcggttcccgaaccagaagctggccaatgtccggatcgatgttgacttcacattctgcctcttggcgggcaatgcccgtaagggccatagcgaacagagtcaagtccatgtctatatcgatcacttgaagcttagacgcgctatcgactacttgaatgctcatacggccattggagttacgtgcgattgtaaccttaccgactattttctttatcactcccatagctgtagctccgtagagtatggtagatagagcgggtgtttggggtctcccccagtggtcaagccaagatggcaaacaggcttaggctgctggcgcagcatgctgagaacgtcaataacacgctcacgcaacaccctgggaagctttttacgatcaccccagcaaggtaccaacagctccgcttcttgcaaaatatcaagcagatagacagactcggtttcgaacagagttccggccccgaacggatcgagatagactcgacgcaactcccgcacatccgccgcgcggtaagcaaacagattaccgacaaggatacggctatagccccattgccgggcgaagccgaccatcttccggatagtagggtcatctgatacagcatcggcagtagagggattgacccctacgaaggcgactgacctaggctcaaatgggcggtcaaggtttacactccgttctagccggtagcggtacaaaccacattcacttagtacggctgacatcattcacctcaccgagtagctcgctcaactcttcgatcagtccgagcgggatagagcccttgcaattggtaaccttgacgaactccaaagtacgtcgcagtagtccttcgctgaccgtcataccgttgatgcgcgccagttcgcgcaggcatgcgttatatccacgctgcatggccactccgtactccccgcattctcgtcgagcatcgccttcgttcaacggctccggcacaaccaccacccttgcgcgcaggtccgctagttcagcgcgaagttcctcgatctccatctccctgccgccgcactgctggcgggcagcatctccctgtgccgctgcgtcctcggccatggctagtttggcgcgcagtgccgcgacttcctccctgagcgcctgggcctcggcggcgagggcgtcgtagagtcccttgccaacaaattctccatccctgaacgcagttgtcagttccgccgcaactcggtcacaggtctttttgtgccccacatacagatcgcgcgtgccatttccggccctgacgcaatagggccagaacccttttcctgtctgctctactcggtatctttcactcatggccgaatctccaatgagcccttaagcagccagcgtgcgagccgttcgcgccagttcatgaatcgcgacttgccgcgagcgtttaccatcagaagaccgtcggctttctgatggacccggactgcatggctgtcgagcatgtgagaggcttccagcaagacatgcttggcctctgcttcgtgccggttcccgttgtagctcaggcaccgggcgactccctggcacttgcttgccagcctcgattcgtagtgttcctcactcatgacctacctccttgccgggcgcggtccaggcgctctatctcggccaggatcaaggcgccggcacgcacgtagttggcacgcgcgtcgcggggtttccaccacttcgccgagaacggccagatagccggcgcctcgtcattggctccgttgaggatgtatgccgctgcggcgcgcggaagttcggcggcgcaatagaggtcgtcgtgctccggcgtccatccctcggcggtgatctgccggcggcgctctgcctgaacgtcgagccatgcctgcggcacttccttgccgggcgcggcggcgagagagcgcagagcatcatggatggctcgcagatcatcgtcggtcggctccttgcgtaggtacagaacgaccgcgcgcggatgaccagtgtcgcgtccgatccctgacacttccggcgcgctgtgctgagcctgggcgggcgcgtcggcgagtatcgaaagcagttggcgctcagccgtaattcgggtgtcgtggtccttgtcgctcatcatgttgagaagcggctcaatcggaaccagcttccagccctccggtacgctgtgctgagccgagttgccgggcgcggcggcgagcagttttcgcgcaattgcttgagcatgctgttcgcgcacgatacagtaaccatgctcgtcgccttgctccgagtgctcagcattatcggcaaaaatctgcaagatcatgtcgtcatccggcacgctgtgctgagccttggcgaccgctagcgcctcgttgagccggaacccttcgtgcacccagtccgtcaacttcccttccagctccgcgaccctggccagggcgacttccagccgcttagcgttttcgtcgcgctcgtacatcactttcatgcacttttcgaggctgccaacagggtcacctttggactcactcaggcgctcgacttcggccagagcggcgtcgcgctcttgctctgctttcatgcgtagttcgtgcgcagacggcccatcagaaaacgctactgtcacgacaacagggcgtgccccttcctgaccatcaaggtggaggtcgaaatggaattcctgatagtgtttggcgccagacttaatcaactgctctattgcggcctcagcgatgttctgtgcaggtatatgcggctccagttccggctctgcctgctctgtctcgcagtcacaggggatcaagaccggctctccccatgggtgggcgccaccggaatctgcttcgccagtgccgcagcacttcggacagggcgcttcgatgtagtcgccgttgtgatcggtgtccgtcgtggaaacgactttcgagccgttgcaggtcgcgcacttcggggagggttgcgctagggaggagcgttgcagatcgcgcttcaggtcttcggcgaacatgagaagctgcggcccagtcaatggcactccgggcgcagtaatgccagccgcctgaatgcactcgccaagtgcgtttgccagcagcctgagttcatgtgctgcctgttcttgttcgtcgtgcgccagggcagcgcgggcttgtggccaattccaaagctcgcgggcaatgccgccgatctgttcagcccattcctcgttttcgtgaatgatcgagcacgaaaggttgtgcaattcgttaccgaggtctgacagtttgcgcgcatctccgcctgcctgctctaccgcaggatgtgccgggcacggatggacgagagaagcgtcggcagtgcatttcgagcagagcccagggccgccgcaccgcatctttactccatccgcacgcggaaaaacatgcccatgcccttcaatagcacgattgaacatttctgcccatctggcaacactagcagcgtgctcatctacggccataactgcaacggcgggttgtttcggcttgtgcctgactgcatccatcgacaggcttccatccggtctaacgagcaaataggctactatcgtaggcccacacaacccttccctttgggtaggttgagtattctcttgatcagccactacttatctccttctatgattgcatcaattgcagcgtcgagcttgttttcgaacaaatgagatactcggtctcgtacaactacaactccacgaccctcctgccgcaatgcgcggtatcgcgcagcatccttgcgcagcgcatcgttttctgacttgagcctgtcgatctcgtccagaagggcgaggacgatcttggggtcggatgctttttgaaaggctataactggcttgaacatccaatccgtagttatatcgaaatagctagcctgttcagcaagcctccgcaactctgcgtggtcggtcataggcatgtctcgtcgttggcactctcaatcagagtacgtacttccattccatcgctcagggaaacgccttgaagattcgggcccggatggaagtaacaatagccctaatcacgcacacagcgttattctcggtttggtcttcggagaagttggacagggcctcgtgtaccggcggctcgttggagatgcccaggcaggcctgccagtcggggaggtactcgcctttggctgcttcgtccggaatcgacagcgatgtggccgcattgaacggaatggagaaacgatggccatagccgatgtcgtctaccttgttcttgcccggtacaacatccttgacgccagcctcagcgagtttgcgaacgatacggcgctcccaatacgcaccgtcgtcgcggatgctggagttaccccgcagacccttctgcgccgacatggcgttgcgcacattgcgtggagtcgtggtttcgttctgatcgtcgaaggcatggtatgcgtagagaatcttcctcagtcttgggtcctgctcgaagcaggacggatcgaagacattcttgaattcgctaagctcccagggattaccgtcaggccctagactgccgttgcggtatgtgaagaacgcgactgagccacgagggcggcctacgtcgtctatcaccgtgatctcgtgggtaccgaataggtatcccttgtctagctgtttggcctcgtaacgtacaaatgaggcgtcgagaagccccatgaaagctttgacgctaagcgaagtagactctttattggggcagtgaaaggcaatcttctggccttcgaccgcgcacatggcgacgtagtgtagcgctcgggtggtgcggcccgtccgatagaagggctcaagtacatcagacattttccgatcctctgtctggtgtgggtagggatcactgagctagagccggcggggtggtcaacgttaccccctggggtgtacctcttgacgagagaggctagcgcggcgcccggcgagcattaccgtccacactgaagtgtgtcccctagtcatggcccgcaggctctagctcactgattcctgaaattggccaccttctgtttccagcgccggctatgccggtggtggccttgccagtctcgcccgaaggttagacagagtgacgggcactgcccgttgcgtagcagaaatatccacttgtgatcgaaggtcactatcgctaccttcgcgtaggcgcactgacaggcgtgcggtggagtgccgaccgtcagtgcgcctacgcgaaggccctcccctttctgttccaaggcaactacgggtactagccgtagcagggggaggaagcccggaggctacccgtcactccgcaggggtttaggctgcgaggcgaacgtcgaacgaagtatcgttggcgtttatcgatttgcgcgcgtcaaggtgcgtcgcctttccttgctgccttcgctaagtacgcgcagcgtcgaatcctgaacagccccatcagagacactctctagctggactttccgaatgatgttcctagcaaccacccggcagtatcgcagccaccttctcacacctaggaacgaacgtgctggccgagaatgtctttggtggagctggggggaatcgaacccccgtccgccacgtttcccctttgaagggattacagcaattccttactgcggcgcttcggagaagtccacgtagtaggcttggcccactttgaggttcttcgccacctcctccacgatggtcagatttacatgaccggaaggggtgaacttgccgaataccgcgttctcatcttcgggtttcccgctatcgggagaccatacagccccgaacgataccccacacatcctcggatcattagacccctggggttcagttacgttatggcagatcatcttgcagcgcatacggtacatgacacttctcctctcgatggtgcaggggttgtgtttccgtgttgcgcctgctgtttggtttctcctggcgatcctcccctacttcttcgcacccgacatgtgaaggtttcggagagggtcgccaggagaatgcaaacgtttgcacctaattggcagggggtcgctgccaccccctcggtccagtgtttcagccgtagccgcgattcgcggccatcgagcatcgggtcctggacgccccgccctttctcggtgaccaagccaaggtgaggtcattatgacggtcggacgctaccccgactagctacagtttagaacctctagacgacccccgcaacgacccgcgcgcatccgcagagttttgaggtagaggccctcaccctcctactaacctgctcggaggggattgacgcaaacgtgcctagtcgcgtgggcgctctgttgctacaactagagcgcgggcgcattgtgcctacttcccagggccgtgctacgcattccaaaattggagtgcctaacgtcagcgctaacgtgctttccgccctgccacctactccccatattagccaatgtcaccattggatgggagcctcacgtttcgtagcgtgttgcgtttatcaccccggtgctaccgggtctcgccatacgtgtgagccctaggcggttgattcagcccagctaggttgaacccaggacccacacgtatggggagaaaacgggctcgacggaagtagtgaaaataacgcagagcccgctctacgccctcaataatactcttttgaaaagtagtgtcaactacacatggctgtgtcgcggtttggtatagacgctccacttcatagcagcccaccctcccggctcctcttccgcctcacgtatcgcgtccctcatcaccgagatagccaacggagcaagggccgaatagctgatcggttttgctacttcgaactcaacctcgacaatacactgtagatcactatcgtgagaagcagaggatgccacttgcccctcaaatcgccataaacatttatgcgacgggcccgctaaatcaagatagaaaacaggctttacttctcccagcgtactaggcgttgcctggaaagtgctgaacagtttgtaccccataccgctccccgtaaaaagccgtcccgaaggacggctatagtggctggtgtctactagccggctaccttcgtggttatatcggctacgggtcgtgtttcctcccgacgcttgtgccagtcctcgacggcaccgagccatgcgcggacgaatccatgacgaaggcagtcatgtctgctgaacttcacaactgtgaggtttggcaagtcgcctgccagctcctcgaaaatcggcagccctttgatctcgaaccgcttcaggtcgcactgtgcttggtctccgcacatgatcaacctagaacgagggcctagacgggttatgaaagtatgcaactcctcgaaattgagattctgcacttcatcacccacgactaccgcattatcgaaattcaggctccgtagattgccagtcgtacggaactcgataaatccacgccgcttcatccactggtaagtggtggggtggccaaagatgtggccgaaggccgaaacatacggctcttcgaatttggcatctttctcttcttgggtaccgggtacgaagccctgctctcgctgctctacggcagaacgaacaaggatgattttctcaacctcattcctcactaaagaccctgctgccagatagcacgctaaggtagtcttgcccgccccggcagagccgagcatggcgagatggtcaccttggcagtaggcctcgatagccgcacgctggttatccgaacccggctccagcggtaccagatcatgcaatgtccaagacttctttacgggcccctccattctggcctcgaagtcatcccgcatcacatcgtcatcgcggcgatttttacggctaccgtttcgagctttgccctgttcacctttagccatgtgttactcctttgctcacggtttgatagtgctcaccgaagcatcaccgttgtagcgaccgcgattgcgatagctctgctcttccggcaccggttcatgctcgaagaacttgagctgcccgatcagcataccgggccttagcaccagcgcatgatgacgggtcatgttcttcagttcaagggtgagtaccgagctactgaacccggcatccacccaacacgcgttgaggtgctccaagccattcctggcgatggaggacttcaatgagaattcacaggataaccagtccgggaggttaaactgctccacagtatgggccagtacaaattccattggctccagcctgatccgtcccttatgttccctgaaacacacagattccctggccgccaaatctacattcagcaaaggaggttctgtgaaatagtcctcgtcgaaaggtgcgtcttctactagaaaggtgcaacccagacgaacgtctatgcttgcagcgtttatctggctccgatcctcaaggccactaagatagccccggtccactacttcgcaaagacgggtataggaaagaatggtcatagtatctccgtgcaaacgcttgcattaaaaacgtcgatcatcgtcaaaacgttcgccttcgaacctgccggccatatcaccgccatcgatctcgtgaacggcagcccaagtgcttcggcgttccagcatattacaatacgcgccgcatacagcatcggatacgtccttactacccatcatcggatgatctatcttgtccttatgcgtgtcgtactccaactccagtatttcattcagcaagatgggatcatcgagaagggctacacgcctgtcgtagagtgcatcacggaattgcttgtacggcgcgctggtacgatccaccgaaacctccttggagggcatgcggtccttacgccattgctggatggactcgcggctatcaaagccgtcgtacgaaacgcctttaatcgggtagccatgcactttaataagggatcgcacccaggaccgtacctctgcaatctgaatctcgttattcgcatctggctcgatggaaagtgccatctctaccgtagccagcggcagccgttctgactggtccccggcccgctcaaagttactcatcccgtcgaagcgaaccatcgctacaccgcagcggtctccagtacgtgacaggtcgatatggacgtaccgaggcttgctcgggttggtgcatacgtgcccatgtcgaacttttggcataccgtcaacgccaaggatgacgtgatctttctcaaggaacgactccaaccgcttctcgtggccgagttcaacgcactcgtagacccggtagcgcattttgatgaacgggctgatcgcgttcgaggatataccaagaacgtctcgcatggcgtcatagggcttacgctggaagtccggcaaatattcgataggcaccagctctacccaggacccctccggcaccgcctcgtccggccctagaacacgagtatcgtgctgtacatcatttccgatcagtagacggaacgtctctccgcagaagttttcttttggctgaacttcgtactgcttcttgttgaaaatgtagacgttgttatggttgaacttctcggtctccgccttcagcttatcagtgaaatcacctttgtatcgggtagacgaactggcgtagatgatgccgatctgagggtaccgtttagggaaagtaccacgccgacgacgtaccacggtatcgtagaccgtctgcgcctgatcgtatacgccagagcgtcccgtggatacttctgcctttttggacttctgcaccacgttcatgaagttgatctcgtcaatcaaacacgcgatcaaggcttcaccgagaaccgcgtcttcattacccgctaccggcacaacacgaatgttcttctcgatgaacagcatttcgctgtcgatattcttgtccggtactccatacttctggaaccatggcatacgctcgatatacttcttcatcggcgcgtaaaccactttcttagtaacatgaggtttggcgcccatgatcgccatgacaatcgaggtagcggagggcagcccgtaccagccttgcgggttcttcatacacaacagaatgtgaagctggtacatgaaccccacgatggcctgggtagacttaccagaacgcgtacaacccatcaggcaggccgtatcaaaggccccaccgagagcgccaggagcggtatcgaggcctcgccaccagttctgattttcctcgacgattgcctttcgaacctcgggccacatcaccaagtcagtagcgcccaagaactccgcgctgtcgaggaaggtttcaatgctcacaggcatatgctctagctgcccaaggaactcgaacagcgtagcctccccatcgaccatgcgctccatggccgtacggtagagcaggccaaaaaatgcattatcgatatccttcgacacgctttctatggtgcgcagaaaagcttgaacacggcgtgttttcttcatggggctcacgttcatagctccataacctccccttcttccgggtcatccaacgtcagatcgtcgaaaccgcttccgtcacgtcggcgggttgtcgggggcccaccgcgcggcggcggaggtggaggctcgtcagcctgcatcaggtcttccagagcttccatagtccgagccataagcatctgtacatcgctcatggcagagccgtccgtagttcggcggaagcgcgcggcctcaaatacgccggcagactgtagtgtacgagtcttgtcggcttggccagccagcgcagtccgcatcgcagccaaacgcatggccgtaggtacggcattctcacccgctgccatagaggcgatctgcattgcctgaccgctcacctcgtcgtagaattccgactgaatgccgatgtactcgttcacatcgaattgacgggcccgttcagctagcatctttttccaggccgccctatctttctctaccgtcgatacagacacgttcagttgttgggcgatccgatccaatggaatcttgcgcatcaacaaccggccaactagattcaggcggtattcccgtgaaacactggctggggcatttcgctgcacatcaaacatccgctgagccggagagacatccgtcccaggctcggcatcatctgcacgagcacgggatactacaccgcgatgtacggcgaggggatcaatggcgtcatctacgtcatcgtgctcctgctcatcgaaatctcgtggagttcgcaagttactactgccagagttggaggcgaagcgtgccccaccccgcacccggcgcaaattgcggcgcgcaactgggcgacgcccatcttcggtccgatctgtcattctcgttctccgcctttacgacacacgatattgccgaacaccatgttgtaggtagtatcagagatattctccataccgaaatccttcaagtcgatgaggagtgcattacgcgtgcgcggccacatacttccggctagtgccttggtcttggcttcaatctcctcacgagagtagccacgctccattctccactctatgtactgacgatggagcatttcccctacttgtccaagcacctcgtatttcgacccgataaatagcaacccacccggcctaaccatttctaccaccttggcgagcactctgcgctgcattgcaggagtgatgaattgcaatacgtacgacagattaactacgtcatacgtacgacgatcatggaagaaagtgggcgcggtcagatcaaccagctccacatccgcccagggaaaatccttgcgcagatgctcaaccattggcagtgaatagtcgcaggcgtaagcatggaactctttaccgtagcgtctacttaatgccccgaggaagtggccgcgactggcgcccaggtcagctacggagttacggccaacttcgaacaactctctgcataactccgcatgaaggtcatgagcccgcaggaaatccggtatggaccggcacgccatgtccggaaaaatagcagccacctcggcatcgaattcaaacttagctgggttggccggcatatgcaccggcctgtcatcttttcttgtcagcatgatcgccgctccttgaatgcaaacgcttgcactatacagcttgtctctacgccttagctacagtggctagggaatcgcccgccccaatcgctttgcgaaagccgcacgagcctctttagctaaacccatacgcgagccgtcaagaaaaggcagatcgccaaattcgaaatccaaactctggcccagagctttcagattcggtgctttgggagcagacaaacggaagaagaaaacgttgccaccctcccaaaattcgactttgttccagaagcggtcaaagtagctacgagcttcgtcctgcgtcatgaatttctgaattttaggcctagagttgatgtcacccaaccgtatgcccggctctccgtccatcacgaagtagttaccgttccttaccccgccatactcgtaggagaaatcggaaatatctcggcacgttccgtaaaccgaagtatccagccgacagagtgcatgcacaataaccaataccttcaatcgatcctccgggaaaggaatcgaattcatcactgccgacataaaaatagaatcgaatttgtgcctaggatcagcgatagcggacaggaattgcttcgccatggaccggctatagtcggggctaggctctgcttcgttaagtacgggatcgacgcgatagggctcgaaatccattgcctcgaatcccttggtccgcaggtagggggctacacggcaaagcccagctccgaaatcgagaatggagttaccatgtatctcacggaaacgtagccaaaactccgtgctgtagctatccctgtcaagtagagatcgttcgccattggcccagaaacgaaaactcttcggcaccaaacctcggttatttgaaactcgccggaaggcaccaccacggagaaggtccttgaagtcctcgtcgatgtggaagtccatgctcaggtagttgagaacaatcttagcgacctcggcgatgcgatccgggatacggactacaggccactgatccaaacccttctcaatagcggcgaataggcggtggaccccatttactacctccccagactcactgactacgagtgggattttgacccccattcgaacgaacgccccggcggtgacaatggcctttttatcgtactggtctacgcaacccgccgcgaatactttgatcggttcgtttttgcaagcgtttgcatagaagttactgaagtcgcagtcgggcaaggactccagcgcttccagtgtttgtttcatgttgatccgggccactccggtgctgcctgtgtcgaaagcactgaagtcgttggtgacgcggttgaagagtatgttgatacccatcatctcctgctccgtgagcgatacgtacacggtcggcatggtggggattcccgcctccaggcataccctttgtcgctggtgcccagacaacaaagttccgtcctgttctcgcgcgaatatcggcatcaggaaccctaatttgacgatagacaaccgaagcaggcccagacgcgcagggtccgcctttcgcgggttcttgatgtcgggcgagtggttctccgtaggtacattgacgacagtaatcgaggtaagtttatcggctgcactcaccctggtgcggcgctgacgaagtgccatttggtgctccttaatccagtagcccaagccggttagcgatatcctcttccatcgccgcactgtcggcgttgtgggcacgccgcagaccgtcgaggaaattgcgaactgaggatgccggacggaaaaagacgaactctccgatcacaatacgcgcagttgccggggaacgccgcacaggaaggcgggcgctaccttcttcatcctcctccggcgtagctagaccagcagtgagtaggcaatcgtcagaaacaagctggcccaggcaatcgatctctgccgcgtcccaaccgaaatcagtgaaatcaaacatctccttgatcgcatacatttcctgcgccaagagtgcgtcatcccatgtagcaatctcagctactttgttgtctacgaggcggaaggcctgtacttgctccggagtaaggtgggctgcgcttacgaacggaagctcgatatagcctagttgcttggccgcttccgttcgcgtatggcccgcgaccagcacgttgttctcgtcgaggacaatcgggttcagaaagccaaattgctgaatagactttttgaccgcttcgatggcctgctcattgttacgggcgttgaactcgtaggggacaatctcctcgatagggatatagtcgattttgatccgtacgggctcagccgcacgtcggggcttcgcgttggttttggacgaagacgccgagcgccggctacgttgtagagccatgttttctctcctcaaagaaaaaggggctccccgctatggagagccccttcggggttacaggatgcgccgggcgttatttacggccacggctggccggcttggtcgccggtttggtcgctggcttggctgccggtttggcggcgggcttcgaagcgggcttggcgccggcgcgcttggccttggcgtccgctaccgctttttctgccacctctttgccgctctctttggcatcgacagccgcgccgccggcggtgttactggcgcgccactcgacgagaacttggaacagagcttcttcttcggtggccgcgccgaaggtggagacggcttccgagatgatgtcctgaaccagctcgcccttctcctgctcgaaggacaggttcaccttaacccgcaccttcttctcgcccggggtgccgccggtgccgccttcgcggtaggtctccttgaggaccgtggacaggtcgttggtggcggtctcgcctgcgagttcgatcagttcgccggcatcgacgccttcctcgttgaggtagccacacagcttggccgccttggaccagccgagagtcgatacgacttccgacgggttggcgatgccggcctggttgaactgaacgtagatcgcgatcagttgggtggctttacgcgcgccgaggttgaagtaggcctcgacgaacttgccccaaccgccctgctctgcgtaggcagggtccatgtcacgccagggcacttcgcgcttgatgtggtacagcatgccgccgatctggaactcggtggtggcgatctcgttttccatggtctggacggcagcgatcagatcgtcgttgtcgtttaccagatcgagaacggacgggtcttcctgctccagatcgggaacgtcggccagtaccgggtcgcccgcgtcttccggctcttcttcgacctgcttggccactgctttgcccttggccggcgctttttcggcagccttgcccttggccggcgccttgcccttgtcggcagctttgcccttgtcggcggccttacccttggcgccgcgcgccggtttttccggctgctcttcgacttcttcttcctcggcttcttcctcggcctcgtcggcctcgtcagaaagctctacttcctcggggaagcagacgacttcgaggaattccgggttggtttccgggtgcttcttcttcttcggattgaagcccgggttcggcgcctggaggaccggcagggtttcatcgtcgtcaccttcctgctccggctcgaagccgaccacgaggtagatgccgccgtcttcgaggatacgatcctcttccttggtctgctcgtcgtagccgaggaacttgactcggctgccgattacgatttcttgttccgcctgttccacgaccggggcggtcttgggcttggtctgtgacatggtgtcttccatcctgtttcgttggttttaccggcaagccggtcgttggttatcgaatttacgatgcagatcgtaggtcaaaagtatatcgcaatcaacctacagaagccaaatgtgcaagcgcttgcatctcgaaggctcccttgcctgggtgcaatgttaccttacttttcgcccgtaacgcacgttgcaaaaactccgccggtccgatcccgtgaagtaacgcatggcccaatcgatacaaagcatacgcgtctacttcatcgttattcaaactgctatctacaccgaagtgctcaatagcggcagctttcatcgcaggcttcttgctgttccccttgccagtgaataccagctttagggtagtaggaggcactaggataaccggaatcccgcgtttgtaaagttccagcttgaattgcccgcccaactctgcggattcccaacgaccaacccccttggcgccgattgcatacccttcgtatacgcaaactatccggtaatcggtcgcctcctccatcaccgtatccaacagcccacgaaactggtttaactggtgttccagcctctgcattccacgaaacttagtacagagcctacccactaccgggcgaccgtcaagctcataacagtagccggtagaccccaatgacgggtctacgcccagaacgaacaactgaccatcagacttagtacgtcgtatccgcataatcagctcctgaagcagagttcgcatacagggcagtctttagctgccttgacatcctccctggcgcaagtgcgggtcggaagcggtgccgacgttttcgcaatcatggctttacggtgcgcccgatagcgtttggcgtcctcgatcatatcgtccagacgtttcacttccttttcaggttctaccagaaactctttattgttgatctggtcaccgaacagccatccctttgtgtagtaaactatgctgcatcgatccgtcatccgctttccggcccggcgcatgagtagccaataccacactacttgcaagacatgctccggcttagggcgggacagctccttccactgttcatgcgaaatcgatttaagttcgacgacgtggtaggcagaggatcgcggaatgtagaagagaaggtccgggttaccgactacgccatactcctcgtcgaagaaactcacttcgtggtactgatccacatgcgtgtggcaatgcgggcaaacctcttcctgatcgatttcttcgtacaggcaggggtcttcgtgtttgaggtattcacatttgcacgaccacttaccccaagtgaggtatggcgccccatcgcgcgccagggatttgccaacgtcatgtatcgcatcgccctgagcgaacgtggtcaggtccgacacagtaagtcgctgctgcggtagcgacaactggtgcaactcatgcaatgcgcgggctcgaacgcaccgatgaatcaagtccgaaacgtgaaggtactcgcccgttcgcattggcttgctctggcgtgctgccgggcggatcgcatctccgtatacgtaatcggtgttgggtgcccggactacctcacccaaaccgctagacgcaggctgcactaaattgcgagcagctcgtatgcgcaaacgggtggagggcttttcctccgggctcgaccggcggcgctttagtacctcttccgttctggtggctggtggctttccggaagtagggcggcgaatagttggcatcagtttacctctactagatcgtccaaaacatagctagggcaaatgaccaactcaccttgcttgcgcccagcgccgtcgttgaactctacgactagcactggcatctcccctgacaaggtggccgccatttccagtttttctatcatctcgcgcgtgacagagaacgatttgttcttcgtcgttttgcattctatccgaaccactctctttacccgcacatcccctttcacttctcgggcgcctgacgcgggagttaaacgaccgcctactcgcttcgccacttcccgctcttgggtcttgctcctgcgatggctcttggtgagattcgatttcttttcgaaccggtcgaatactgggttgctcatactcaatccctagctctgttaaggctttagatatctcctcatcactccacgtctcgccccgtacgttagcgcggctgccgtgccatacagaagcggtatataacaaagaggagaaggaggccccctgttcatgcatctttggataccagcggagcgcctgtttaaggcgctcgtgtttggtttccgtcactacggctggcctactccgtctcaagcgattcatagaaggcaatccccggcgcgtcgtcaccgtagaaactttcgatgaagtaatctggcatccgcaattttgcagcatgcagagctattaggtggttacggaaccgccagtacaacagaggatcgccatagatgtttgcgatatgttcgtcggcactacccagcgtgatctcctcgtccggcagatacaacgtccagctacggccagcccccgcatatacccccatcttcttagcgtacgcgatcatagtcggcacattgtctaccatgccctcgcggaggtcctcgatctcgtcatggtcacgacgcattagctggtattcaccctgccgcatacctgcacagattttattcttctcaagcgtgaaggcgtgctcgttgtaatcaaggaggtcgaatccgtcactgtccttgcgcatattttccttatttttaaacctcgcgattatgaggaaagcatgctccagggctttaccgcccggcaggctaacggcttcctggcccggtggggcccatttgccaacgccggctcgctgctggttgatgcagagcactgaaacctcatgtcctcgcgatttctcaacccccaaagccgtggtaagtttccgcaccaaacgggtacagagtttggcgtggataccaacgtgctggtcctccgaagccccttcgatttccttgtggggagtaagcgtaccgatggagtccaaaactaccaaacacacctcagccacccgcaccataccctccatgaggtctaccgcatcttcgccagtatctgcggtgataacttccagacgggccaaatcgacgccacacttctcggcccacaccgagtcgaaactacgctcctgatcgacaagtacacataccttatccgggtagtgcatctgggcctgggcaattaccagataagcaaaagtcgttttgccgctagagcgtataccgtgaatctctgtcataccttgaatcggtatgccgccgcaagtggccacatcgaaggtgaacacccctgacgggaatcgcttggccgtccgtaagtcagaggcccgagttactacgtgctctccgagtttcttctcgatggctttcagggccgcagtaagctcggctccggtcctggggttgtctctggtcgcattggtcatcttacgcgaggtcttcggcgctctccgctgtacagccatttctacttaccccttttaggaaaccaaagcgcttcttcttctgccagcttttcattacagaaatcggaagcttcccgataagtatcttccagccgatcaacccggcaagggagagttacggagacttctagccgcagagagttaaagtcgcccatgttgtacgtttggcctacgcctacccgtataaacgcgggttcctccccggcggggaacgttacctcgtcgaggtcttcgcgaagttcttgctcgtccagtagggcttttcgatgtcgtaaagccgagttacttaccacggagcccttgtgggttgtagaaggtttcggtctcttggtattgatccggcgtggtagtgccataactcacctcatagtcgttcggcgcgatatgcgctaatgcgctgctgtatctggtgccatacatgcgtatgggtccggcatgcattcgcgaaggtttgcgcatgctcggacagaaccgcagcaatcacctcaagctccccacggctatagacgtggtagccggaactggtctcctccaaatacggctcagggagcatgtcctctcggagccaccgtcggaaggtactcagcgacttgcctaacgcctgcgcagcctcggcgatgctgaaggtttcgatggtggtgggcctcggcgcaccttcgaaggtgatttcccttcgcgtgcctcgccctcggaccgctgacgggagaggcgcttggcccactcgcgtatatcggtctcgtgcttttgccctttggtcttcgcgcagggtgctgtcgttagcatatttataccgtctgagttcgtttcgatcctcattgctttcagttcgtttattcggtttcgtatctcggcgaataggcgctttggttcgcattcagtaagtcccccgtcttcgccccattcgaagaggcgagagttgtttttgacatactcctgagccctccctaggaacgagttcagaagtcggtagttctgcctgtccacgatagttacccaaatgggcatatgctttccggcgacctcccgctggatacgacccatttgctgctcagaggttcctcgcggcgtggcgtcagcgcccccagccaacctcggttcatccacacctttttgaaacatcccatacgtagccaggataattttagactcccgcaagacttgcttcaggcgggcctttggcactttcttacggactcgctcgtatttcagaggagtgtactccgtatctcgcacataacctttcgggcggcgcgcaggacgtgggtccttggtaagacggaataccgggtcttccccagtataaagtcctatgcgatcttcttccacgcccatgtatttcagcaaacaaccgagttccttgagttgctctattcggtcgcccaggacaagcacatccctaccggtctcgtacaaccatactacggcgtcagccaacttgagatttcgaaaaccatcgtctgcgatttccgtcaatatccgtccaaccttcggcgacacattcccataccacgaataaacggtgtcatggtagatcatgtataccgcagactctttgtgttctttgtcagcggccacccttaccttgcccaggttatgatctagagccttctgcaaaccgtcccggcgtttcggagtagcggatatcccaatcctccatgtagccgcgaactgagtcagaacctgactaaaggtcggggctccaatagtatggacctcatccgatattaccaaaccgtggtagcggtagacctcctcatcgaattttttttgtgcaagcgtttgcaccatcgcaatagttacgggcttgccttcgtagtcacacttttttccctgaattcgccctatatcctctgggttaagaccgaagaggctagggtcagtgagccgctctacccattggtccgccaagttatcttgatcgacaataatgagggtagatacacccaaacgggcggccacgataagcgcgccgatggtcttgccccatcccgtgtgcgctcggaaaagaaaatcgtagtagttgtcacaggcttgaactatatcgtccaaggtctggatttggtattcgcgaggtaccggtactttagggaaagaaacaggtaccccggatgaggtgtggtcctcccactcaatgcctagtttattgcatagcgcaagaccatactgcctgggcacacatataaaatccccgtcgtagaaaaacgctttaacttcgatatcatcctccgcccccattggtctatagaggtaggtagtatgcttttcgacaagttttgcgttaatccgagcgacggggattttcatggcgcccgctaggatagccgggcgcatggtttagcgacgccgtagagcttcgcggcggttggcagcgccaccggagcgagcgggccgttgtgggcgctcatcaggcagggtatcttctaccgaatattcgtccctcgaacgagcagaagacgcgcgactggagcgtcgcggggtttgctgctcttcggcttcttcgtcctcgtattccggctcctccgcctcttctctcggctgccggcgaggggcggaacgtgaagcggcgcgggccgaggtacggccagccgaacgcctcggagtgcgctcttcagcctcttcagcttcctcttcttcctcgtactgctcttccggctcttcgcgggcagcgcgacgacttccagatcggctagagtcccggctggccgaccgccggccacgggtgccgctatcctcgtcctcctcctgctcctcgctggagcgacgagagcgacgaccaagatcgcggtcgatatcgtctcgattacccatggtcgaacgagagcgaccaccggctaccgctacgagttgcttctcggtccactccgggaaagcctcttcgtagtcgaacggctcatgcccgataacgtcatgctctttaccatccttgtccacataaaccgtctcgtacgcgagcaggtcatcttcgctgatatcgccaacgcactccacatcgttgccgatggcagcatcggtctcgcggtcgcgagtgacttcgatcacgagccctcgcaggctcttctcacgctcctgcaaacgaagatatttcttctgctgcgcaggcttgattaccatcaggcgcttggaccattcgatgacttcgccgttggagttctcgtacggagtggtatcgatcacggtgaggtacgcggcgaaatacgacggcttgttttcgctgttagcgcacaccgggcaattggcattctcgttgacgcacggtacgaaaaggtcgaaacggccagttgctgtgttgcgaagagcgtgctcatagcggaagaaagacagctcttcatcgacgatgatcagctcccgggtctctcccggtttcatccagaagcgacgaggctcagacgagcgctgcttctgcgcttctcggcgagcctcctgccggcgagcttcctcctgcatctggcggcggccatcttcggcgccgtaggtggagcgaaggggtttggtagtagttgccggcttacgggacgcagaggtccggctagggcgttgagtacgactcatcgattcactccgtacacttcagtcaatgtttcaagtaaggctttggccagcgatggggctaccccagctcgaaccaagtcgcccgctcctttacgtgatccagggaagatcacacagctacccctccccaggagggaaatcacaacggccctcgggtccaatccataaagctgaccaggggcaaaaagaattccagtattagtcgtccgctctaccgccaacttaccgtggcgggatttagtaacctgtaccaacttcgcagtctggccgttgcgtaacggcaaaagggtctggagtaggtagttcggttcatctacatgaccagtagtcatctgctcaccatactaggctatagccctgcgtatgtcaatagttgcgtctgcttttaagccagtttggtttatcccatagcctagcggttctcttcatctcccaaaactcttcatcggtcatttctcccgggtctttcaagaaatggccgccttctttttctgcgctgttaggtatccaaactttggggtaggtcggtcggaatacgctcacatgctggcatagctgattcgctgctatcgggatcgctttttggcccgcgctgtcgttgtcgtaaaacaaataggttggcgcgccgaaccctgccacaattgcggcttgaaacttggtcatggtcgagtgcattaccgcacagccgcaagcccccatgtcgtgaactaccgcgtagtccactaaaccctctacgactaccacgtcatcagaagggcctacgagatgggccccgagcacgcaatgcgatttggccagaccgtggtagtcccgcaccttgaggcgggcggtatccctcgtagcacgtccgctgaacccgtacagttctccccccggccctcgtaccgggaaaagtattctaggctcgccttcgctatcggaggggtcgaacaatagctccagtttacgggcggtggagttagatataccccgctgccgcaggtatgggtgcccaacggcagaatcatataggtccatgtaaatgccctcattgatgggcattagaacttcttggaggttgtccgttagccacgcatccatgctggatatggttcgtgggcccagaaactcgccctcctccagctcctcgataaggtcgtccaggtcctccccggtaaactcagcgtaccgggttagcatcacatggaacggtgccgtatttttgcaagtgaagcagttgaaaatactggtgccgtcctctactacggataccccggcagaagcgtgcgtgtccgacccgctatcatgtgtccagggcgctaacgggcatcgcatactgacccaatcgccagtgtcgatcatttccgttgaacgcccgaacacatcgcggacgacctcttttatcgttccgcgatccataccctacttcgactcctctttgtccttagcccgtgcaagcgcttgcacttctttctcagcgtaggcagcgatgatgtcggttaggctgtcgatctcgactgcggcctccgcgagcaagacgataacatcgggccggctactggcgtcacggtaacgattgaccacggtacgaatacgctcacgcaaattattcatttcctcctcccggctgccgccttcttacgctccacgaccagcttttgtccttttttagtggccggtatcgttttagctatagcatcaatttcgtcttcggtcagaaccttaccaagctcccccaaactcacttttactacagcgaagaaatccttctctgcgagcttgttgtagagcttgtgcggatcgacttcacggctggccctggtcatttcgtctttccaggctgccgatataccggcaaactcgtggtcagatttacccagcgttttcaaaagctcaccaacgcgattgcgcaaagcttccatagcatcggtcgccgcctcgaccttagctagctcaaccgcaatgagctggagcttggtgttgagttctttctcagctttcgccttgtcttcccgctcgaccgtggctgcgggacggcgcagccggcggacttgcggattgcgcatgattgccatgggtagtgttctccatttgttaggattatacctgtttagccggtaatgctgacctacaccgattacctgctcctacggatgcgcgaggttgctggtgctgtctcgctatcttccgcgaaacgcactttgaccggcgcttgacctttccggtggccctgtttctttgtgctactcacactggctccgcgtatccggcggacgcgagcgatggagaccacattctcatcctcaaactcatcaggctccgtgtacggtgaaccgaccaagtaaccatcattagcgggagcgacctgctctggaagttcgaacagtggcctaccttccttgtcttggattgtcgagaagtcataaggctcgtcgagacggaaattactcaactcgaacatttcgccctgattccagccgaaggaagcatccgcaacaatcgggatacgcatgcgtaaattgaaagtttcctccagcgggttggtctccatgtagcgcttcaaggtcttcaagccccagtcaacatgctcctccctgacataggcataaatcgcgtcatgcacaaaaccaatgagcttgatgtactctgggtccaactcctcgtccatgcgacccagggccataaccccgagggtcgagccaaactcctgcacagggctgttaatgccctggcgaatagcttctgcctggatgtactcctcatggctccacacggtagggagatggcggataaggcctgagtaagaccgaatccatccccgctcttgtacaatctgccgtactctcttgtgccatacctccagattcttgtagcggcggaaatagtccgtacgaatacgcttcgcctgctgctcagtgaataccgcgccgtactgagttttggcgtaaatgatgaacttccgccaagacatgccatagaggaaaccgaagttcaccgctttcgctttagtacgcaggtccttcttctgctcctcgggcagtctatggaattcctcttcgctaatgcgggccacgatacatgccgtggttatgtgaatgtccccattctcgttgtagatacggatgatctctgggtcacgagccatatcccccgcaatgcgcagctcagcctgcgacaagtcgcaagagatgatgacccaccccctcggcgccacaaaggacttctggtacatcttggctttcttgccgcgcttcgggaagttctgcccgttggggtcgcgagatgccgtacgtcgggtcaccgtgacatccagatagtaggtagggcgcaccataccgtctacgacgaacttttcttggaaacccaggatgttcgtctccaacatccgtttatccttcacatactgggccaactccagcgtgaacgggcatgtgtcgaagaagaacggaagatggtctttcgaactggtgcttggttcttttaggttcgggtcctccagcttgtcagtagtctttgtaaagactaccggtttgagccggaaccctttcgggtgatagaacagaatctccttcaggaaatcagttcgcccgaaagacagcgcctcctccgcagacttatttttattctgggatttgctcaagaagtcccttaccacatcccgcttcaagccacggtcaagctgcgaaagcaagctaagccgctgcctctcaacctcctcctccatgtaggcctggaactgcggcatcgcctcctccatgtccacatacataccatgggtttcgatgcgagccatggcattcaggccagggatggagacctgggtgtagtgagtccagagcccctcgtcgctcgcgatcttgtcctccaaaatatggtatagacgaaaaaccgcatcggtatccccaccgccgtaggggacaagactgtccaaggtggtctcccacatccgatctttctgcacgatttgattgaaccgatccgcgtacccggccatatccttcacgaacagcttggtcaatacgtccaagtttttctcgggtaggttttcgtccacgatagcagccatcatcgaagtatcgccgccgatgcggaagcgaatgccctcggtcatccacaaagctacgttatcgaacttgagacgctggcctaccactatccgattaggcgcacataacaactggcggagctgattacgcaggcgaggtttatcctcctccggtattgggtcttccgggtggtcccaggccagtacgtaggatttgccagcttcgacggtaaactgcatagtgagtatctggaaacgtggctgaaaccaagctttgccctcgtggagatgcttacggtagctccgtacatcaacgccacgttggtaccaacgcaatccagtagtctcagtatcgaacgacagaatctcagggtccatatcgatgaggaattgcaggtctttgaccagcgtgtactccccggtggtgaaggcgtcagcggtatcgatatctagatcggcatctacaagacggccaaaactcgcgcagtctgcggctaacaacggctcgttctgagggtagttaaccgctacggcgatagactgcatcgggaataccggtaccccaaactcctcagagtgatgcgctaggccgcgtaccttggtgatcttgacgttcttgttcatcacctgaccggtggcatctgcccccagcgtgataatcgcagccggcttggtctcgtcgatataatcttcgaggtgtcgccggcagtggcggttaatctgcttcttgaaagcagtcttgtaggaatccttgttgtacgggcaacggcacatagggactaggcggaaatccgctgtatcgaactcttcatccttcaggcacttagcgagcaatttgccctgattggcgggaagcatgcggccttttctcgcgccagcttggctaggcaaatctgtaactatcaggtagggggcatcctcgttcccgtctacgccacggtacggtgtgaactcgccctggatcgggcagccatcacatagtgtttcatcaaatgccataattacctccaagcgtatccttgatcatcttcttcggcagccgccggacccgccgacacggcctcaacctcctccgccgtcatctcgttgaggtttgcaggcttgaaacggtagtgcgtaaagaatgacccgaattccccttcccgtcctttgagaatatcaagctcacgagactcatacgggttctccgtgggtccgtgctttactgctactacaatcgagctgtgataaccaatcacatcggacatagcgatggactccagggtgccatccttaccgcccttgcccgctgcgcggttgaactgggtattgactacccaagggcggttagtgtcgatggttagctgctttatctcatcaaacacataagcggctgtttcattacggttgagcggtcggctctgcttactcggtttgagcaaataagcaccgtcgatgaatacgatatcaggacttaactcctccactgccgcctcaatctgtgatatctgcgccccggtgccgagcgacagaatccggaaacgctctcgccccagcaactcagcttcgtactgttcgatctgccgtagaagtcgagtagagaccatgccggacttcagcatcttggcatccaggccgtaatgcatcgccatccatcggcgagctatctgctccgaccccatctcggttgtaactaccaatgcggagtagccttcctcgtagacgtggttggcctgccagagaagggtcatagttttaccagtacccatacgacccacaaacgtgatgaggtccgagtcttggtaacctgcggtctctgcgttcagagtgggccatggtgttggaacccccgacatacctcccatggcttgcacccgtacaaggcgctggcggacctccgccatgccctgggcaaggtctaccagtccagcattccgctgtgtgctacgtacgcgccgcagcccggattcaagggcctgaatagccggggcgggctgccgcgtacctatcgcatcgcgtagggttgagtacgtatcgcggatcacgtcaaacgtgcgacggtcatacagattgtttcggtggaattcgacatccccccgaggagtaggcagccgtaccccggtatgctcctgcacagtcctggcgtcagggacggaccggtactgccggtagtagcggcgcacgaactcgaaagcagcctgctcctgctcccctacaaacaaatcctcctccagcctagctagggatgatcctgtctcggaggccacaacggtagcaaggaaattaagacctactgtcacatcacacctccacgattaccgatgtgtcaaagatcaatttttccagctccagggggaaacccacacctcctttgtactcagtgagtagcttgtgcgccgccgaggtcagaagaatccctccttcgcaaatgtgggaatgcaggaaacccatggcttcgttgtaagcgtgttgctggccgcccggccaactgacgacagtaggcaggaccggtatcgcgaaaaagccacgacctaccaacgagtgaatgtcctcgtcgctagcctctcgtgtttcagagagacgtaacaattggacgaggcgagagaaagttaggtacttcccgggcacgccatgcactacagcctgacgggcatgcaactccacggcctcgatagcgagcatggaggactcccggctcgctggccgaacgtagacgccaagccggctctcacgactatcgaaacgcttatacacatcttcagcccaagtgttgatatacccacggcccgtgtcgtcgcagttaatagcccagcaagagcgaggaaccccagccttgcgcagttgagaagggatattcactttcttcattttaacctccccatgccggagcttcgtactctggcagatcatcatcagaatcctgcgccattactgccctgcgcttacggaatgcctcttcttccctgcgacgctgacgttccgcttcctcgtccctgcggcgcacctcattacgacgggctatgctggcttcggattccttgcgcttggctgtttgtattttttcttgcttcgctttcagttcgctagctagttgcttctcggtatagcagcggtcttggtagaaccggaagaagtaggggaaccgatacgccagatcactgaagttaggtgccatgtgcatttccgattgcgcagccttggtagccttattacgcaactgggtcgctttggcttttcggtttgcactggcaaccaacgtccagttcgttagagcccaagagaagaaatcaactacgtcttctacctcgctttccaacatacgccgatggagtaccccgatacccttagagactaccgtgatgcgagggcacgaggtgcctacagactcccgcgctttatccaatagggcctgtagggttcgggtctcccatttcttcgtaggtaccttggtgttcaccgcccgatcattgcgcttcgccttggcggcaccaagtatctgctctgcccgttcacgggcagacatagtagtgggctctgtgcaaacgcttgcacgcctaaccctcttggccataggtagcttgaaagcccgctctacaactttttcccccgatccggatacatcttctttagaagatgttttagagatacgtagtatctctttattgtgtgccaaattttggaggggggtgtgccaaattttggaccccctcccagactcctcaaaaccgtctctatctggctgtttttccatggttttccgggacctgatgagcatacttcgaaccccttcggtatctctatcagccactattttggctgtttttacctcgtaaacacgcgatatcgtctcggttttaccgtccaaaaacgcgtgaatatcaactattcccttatctaccagcccttcgagagctgtacggagtgtgttatgtgacactcctacgccgctacaaacccgtgtaccgtctgccgcaatcactccattcagaaaatgagccttgctgatggactccgctttctttcgaaacatgagcgttcgcccaagaatgaaagtgagggttgccagctcggccatcgttaggaactcaggccagaatctcgccagtacagaagtgacttctacgtattcgctaaagtaaagatcatcccgcctagtgagatcggccactacctccgcatatctgcgggcttcgtgcgccgtgactttcacagttagctccccgagggaacggcccttacccaagatggcatacgcgccaccgatgtttgctctcgaaacatttctagaaccttctcttcgtcatacccgcaacgctgctcgaatcggaagatcagcaaagtatgcatccctacgatgttcgaagctgggccaggaagcttagaaaagtgctctgcaatacctccattgctccgagcctctgcgatgctctgctctataccgtcttgatgtaagtgactgagtagccctacagtgctatgcaactgctgaaccgcaccgagtaggaaaaacctgttgggcgttaccggccctactactacctcggacccggctgatacgaccgaacaggcgaactcagaccagcttgcggtaatttcgagagtgtcatacctcgccattttggccaggacgtggacccccaaaacatacgccgagtcctctagggccccagtaacccagaacacccgcagacgacggtacagatcgttgaaagcgctcttaggcactcttcttgcgtctttcgaccgcgccatgccaactccccacaatggttatcgacatgagcctgcccccgccgcgtagccggctgacctgctcaacctgaataaaaccattctcctttagcctctccaggcccaatgcaatcgtgtttaccgatagaccaaccttatccacacgacctcgctcgtcggagaacccgaactgaatctgccgagcactgatagtgaaagggttggagttttgccgtgcagcctcggcggtcaaccacgcgaacaggcatacgtctgctggcatggctacctccgattcagttgcacgtctacgagagagccggctaagcgcggcccccgcagcaacgtcgttgaacgtggccatggtatttctccagagtatgagactgccctattatgatactattttgaacatatgaagtcaagtaccacaaattgaagacaataaaaagcccgcgctaggcgggctttgtaggaatggtcagtatcaaccgcagacttgcttgatcgcttggtgaaggtaattaccgccggcttgactggaccattcaacgtggttaagccggcataggttgagccacgccacctccacgtcgcgagcccgctcgtaactggtgaaacctttctggagagcccagccactgacgcgagtaccaaactcgtccagaactacaatctcccaatcctgctccttctcgcggaacagaatttccgtagctcggcggatttccttcctccccaagaaacccaaatcgaatttatcgatgtgcagaccggatacttcaccgttcggagaaaaggagagcacgagagatttatgagggttttcgatatgcatgatgtagtctcttggggccgtaccgtcagttcaggaagttggactcgccggtctcggcgtagaactcgggtttggtatcgtcgtcgctacggtcacgatcagacccggtgatagccaaggcgatggcctccgtcgcagcggcgcaaccctgcccctgaaagttctcggcgtccaccttggtagtgccgtccggctgcacagtgattcgaatttgcttactcatgttggctctccttagtagccctggacgagaagttcaatggcgccgtcttggccaaccgaacgagtggtagtcatgccctgctggtgggcgtgctgctccgctgcgttcagagcatacgcctggaagatacggcccagctttgcctgctcggtggtttccgagctggaggctttcgccccgactacacgctcgacgctaccgccccagaaatcggtacgcagttcgtaaccggaaccgtccgggagggcgtagacaccaacgtcgtacttggcatcgtgcagccggatcacgtagggggccttgcccatgccttgctgattctggaagtaggcgcgcggcaccgcgtcggtggcgatgtcgatgttcatgccgctagcgcggagttcggccacggcggcggtcaaggcggagatcgattgaatcttgatgcttttgatgctgctggtgtgagacatggtatttcccttttcggttagtgcggatcaagcgacgttcggagtttgacggattctctccgatttcgctagctacagtggttttgttcctttgttccggagcggttaagcagaatacgtcccgggagccaagaacttcaacgtaaaggcttttgcttgccccgaacacccgaatctctacactatcggactcgggtctacggttaacctgtaaccagaagtgcttgaatcgttctagagattttaggccccttgccagctcgctcatactccggtcgccttttctccgagaaatttccagtagtcgcaagaacaacttccgaccgctctcggtctcggggctgcgcctacagaaagagtaggtcatagcctgcggtaggttgacgccgtacaacgaccctcccgacgacagcaactcctcagctctctttctggttagttttagagggggcgtagcctgatactcccggtcgctaacccatataccgaaggattcggcagacaggcggcggtctgtacattgcagagccggcggcatctctgcgactacccgagccggctcaacgtatgctagcaggactatacgaacggagttctcgcatgaattgcgcacgatgtttagctctaaccgttcacgacccgtcaggctgtgcaaaaactcataaaacccaagataggtagagcaaatagctaatgcctgtaggtggtcgtccgatactctatacagatactcaacaaaccagggaagccctatggcacgtagctggctatagtcccgccttgctatatcgtcagctaactgctccgggcgcaaggtcaggctatagcccctcacgcccggaaggtctatcgtgtttacggtatcgaagagagccattctctacctccttcgcggagtgcggactttaggttgggctatagctactacctccacccctttggcagttaccacggcggcgaaagaaggcttaggcagttgagaaggcactacctcccgcaccgtgaagagttcgtcaacttccgtcttattgcaaaaagtacggctatacccgttagggatgtctatcccgagtacacgtagctcggcagcggtcattacatccgcttttccacggaagacttccaagctatcgccgagtaaggtgatgcggtttaccggtaaccgcatagaaatggtgagcaacggaccggactccctaaggcgcaagaaaagacccgcctgcgcatagggcaagccccgcctagtttcgttagcttgtaggagcactaccgttccttccgggtgcccgaccgcctgaaggaagaaccctttctcagtagccgtgcgcgatgccgctagccaaactcggtcattcaacgaccggcggcccgtatcgatagccgacttgcgaacccaaggagcctctacgcgcatcgccgagagcgttacttgatgtttgtcgaggttaatcgaacccactgcgtactgggcaaaaagcatgatcatttccctccgaggatacgattggagcggctacgcggcgcccgatcacgacgaaccacggcagggctggtttcgctacgcactcgaataggctggttgactggtaccgcatttttctcggcccattcggttatacgggcgatctggtctgcatgcgattgcgacatggggacgctggagtccatggccgagagaatatgccgcatctcaagtgtcggattaccggggtcctcgttgaacgcgagaatgagagcatccttaacagcttgctctatctcagcggggacataaccgtccgagaccaactcgaattcggacagctcgctttcgctgaaatcctccaggctgcggccacggcggcgcaagtgaacttccaataccgctcggcgctcctcggcgttaggcatgccaaccgaaaatacttgatccatacgacccttacggaacagctccggcggcagtccgtcgatccggttagcggtaaccactacgaggcacccgctatcgttatcctgcatccaagtcaggaaagccccgagaacccgagagctagtgcccgagtcggcgccgccctggccgattccacccagacccttgtcgatctcatcaaccatgagtacgagttgccccatggaggagaccatattgagtgccgacctcatgcgcgactccgagtcgcctacccatttcgagaagactttaccgaagtccaaacgcaagagcggcacgccgagaaccgaagagatggcctttgcagcgaggcttttaccggtacccggaacgccaaccaaggctacagctttaggtggctgaataccgaactccctggcttcgtcagagaagcagtttacccgggcccctacccagtccttgaggcccgccatgcctgccacattctcgatgctctccggatggtagagttccagaatatcggaagacttgacgatctcggttttgccgcgtgcgataccttcgaagaacggctcggcagtgagggtatctacctcgctatccgggttgtctacaatgaccagacaaacgcaattgtcgaattcgtctttggtcatccccaggcccagccgagcgagagcggtatactgcccatcgtcgatttccagaccaccttcgtaatcgtctgctacccgctccaaagtagcccgcaggtagttctccagctcgggcagggtgggagtgggagcgttgatgacatgcaccattccagtcggaagcttgagcgagtcttcgccggtgatgaagatcatgcatgccttggtagagggcaaaactgtgtagtagagcttcaacgtctccaagaccaagtgattgttgttggtcatttcaatcgggacgttgtagatgcaatggagtagcttggggtctggctgccggagaataccgtcagagctgttcaggtcacggtgaatgaacccgatagcctcgacgtatttcatggcgtcgttgttcgtaccggccagggcatgttcgctgtagttcgccgggctgaagttgttgcgagtcccggaaacgatgtcgaactcgcggtaggcgttcgccgggtcggcggtaatgtgcttgcgcaggatttcgagggtgcgccccggctcccgggtcttgatctggatgatggagatgccggcgtctaccaaccgtaccagctcgtcgcaaagcatacgctcagactcgggggtttcggataccgccgaggcggagatattctggctcatgtcctattcctgtcgtgtagtggcggcgggtaagccgcagtcattcgagacctagtgcaaacgcttgcacgatgacgattatacggaaaaggacaagactaggcaaaggaacagagggtaagcccgccgtagcgggctattgaggggcttttgctttttggactagaggagctagtaggcggatgagagctgcatgcctcgcggtgcattgactaccaactgataccgcatcgttgtagtcatctaggaggtctccaaccgagttccccggaggcggccccaataccggcccctggcgcagcaggtcatccggtatgctcttcaaggcaatctggtattcgttgggttcctggggctgtttgttgctgcatccggcggcgaataacgccatcccaaacactactgcaactgcatacgtgcctttagccatgcttccctccccccaggcggcagtggttgagcgtagaagctcggattggcaattttctccgcctggatggttttgttgatagtggtgtttactaccttgatgttgtcaatccgatccagcagctcgctatacaccttgtcagcttgattgcggtattggtcggttagttccgtctgcttcttcaggagaccggccatcctgtcctgctctgccttcagttcgcgatccgctatccggccacggtactcccaagcaccccaggtacccaaaacgccgagaagacctacagctaccaacgctaggccccctatcacgtacatctttatcgagcccatagttcatgccctgtcgtatatcagatttttaacatcgccgagaaacaactcaacgatttggctattcctcccagtagcctcgtcgattgccgcctctattccgtttcgcggaagctcaagctcgtgcagtctagcccagttctcgacaaaagaggagttggacagggctgctcttacacatacccagaaatacgggtccttatagtgctccataccttagaacaccaaagcgttagctcgacgcaaataatccaggcgttcttttaccgtgctggtatcgccctggatgatctggctgacccgtgtgaactgatccgcatcagccagcgcattcaaccccttatccatccaaaaatacccagccgacatcgccggcccgtagaagctgtcagtattgcgcagtagctccggctgatctaccagacggtcatccttgtaaatccgcctggagatagcgcgaaagttgtccttaaacgtcagcataatgaagcctgacccgcgataccgccaaccatccccagaggcctcgtcgccattcccgaaacgattggcataggcgcggttggccagcttttcaggattgcgcagatagccttttgcgaactcaatctcgtccgcgtcggcaactccattgcggttaaggtcgaagccagacttgaagatgcttaccacccgcttagggtcggtgtagtacagactctcctccacccgggtaaagtcacgagactcgtgggcgcactgggccaggaaatagcgcagccgacgtggagtgttggcgatgccgcacgtcatagctgctaagttgagcgcttgtaccagattatctatgcgctttaccgactttacgcccggcgccaggatacgaagctgctctgcggttacttgtgccattgtggttctcctttgcaagcgtttgcactcactctaccgttattgtaatcgtcggtagtttcaatttttcttctcggaaaaagttcagcctgtatctcgcctctacctcgtagtggtaggtccccggcttgacgtaaggcggtatgtataccccatagagcgggtagccgatccggacattagcgggccgcaaatccgcatcgtcgataacaatcatgtcgcgggagtcgcgggaggacatcagataacgactactgttatctaccccgatcaattcggcgctcagctcaacctgaatggatgtattgggagggctaatcggattctctacatacatcagagttcctcttgccgccttgtagttcagttccagcccctccgagttgatagagagaggggctggctgaaggatcatccagtagacgaacatcatcagcaagcccgctgctaccagtgcgcttaccgagctgcacactacctcccaaagggatttgcctacctcgatagttttgttcatcactcagcctctctggatttttgatcggcggtaggaatccccagacgtgtcctaaccatgttctggaagtcatccccggcccgtttacccatcatacccacaatacctactatcacagtagtcatacctacacttacctcattttcgacgcagaggcagtaagccatgaatcctgtgaagccactggtaaggagtacaatcaggaccccgccgatggtctttggttccggcttgtttaggtagctgactagaccacccatagccgcaatccccaggacccataccagcgtcgtgaggggaatactcattaccccagtttgcgcgtccattgcagcccccagtcaaagcgcatgatactgagtatggcgaactgcgcgagcgcgatcaggtagcatacgaccaacaaagggatattggcacggtcggaccgcgacgtagccgtttgctgtagcccgtacaagctgttgatgatgttgcctaccagctcgtcggcctccttctcgaaactctgtgctttcgctttgtactcctcattgctaggtttcgtgttcagaaagagaacaaaggttgatgtcgatttcaaaagctgctgtttagctttttgaaagtccatgtactgcgggtcctgctggatgtacgccggtacccgcaaaggcatgttgaagtacgacgtaaaagtcgtacgacgaatctcagccgctttgcaataactactgtcccagaaccgacaatgatcttccgcacggataatcgcttgtgggttactctccggctccacatacgaggaagccactctgcccagggtctgggtaatgttgcgcgcgtccccgtcaataacgtactggtgtacgtaaaacaggacaaacagactcagtatcgttagccccaggcaaacagactggagcctgcgataagttttctcggagtatgtagcgcccatgccgttctcctagttggtgggttcagttacaaaaacggggccgaagccccgtatgtgcgcaaagtaccagggtagccaatggctatcaacacaaattgactatccccggtgcaaacgcttgcactcagtatgcacgaactacagcttcatacccttcctgagcctcgtacggcgggatgtacaaccatccatcggtgttactcgtgctgcgccagagtgcaggctcgtacggcgccatttcgttgtcggtaatcactacgggctgacctccgtattccgcaagatacagacgctcgttattcacgaaagtcatgtagctctgtttccacgaggcagggccagactctgtacccgagaaaatgttctgacgaatgaaaagagcagagtaagtcgggggagacgcggggaatacctgcgcggctactatcagggacgaaccggagagcctgaaagccgcagggttcacctcgacagtagtacggatgcggaacgggcctttccatccagcggactcgtcgtagttcataacaaaatgaatatcgttgtctgcccaaatgacgaagccgttatcgcttccttggatcgcacgaaggaagtacgggttaccgttatttagcggcagttggtaggcatactcggcccaggtctgcccgaaatccgtggttacccaaatcttgccatcgttatttattgccaagtggcggccactaccgaacgcgcaaccaataaattgaccggcctccggcggcgtggcgttgctagcctgccaacccaacacatttccgttttcgtccatgtcccgccatacgagtttcgaagtcccgtctgcgaaccccgcaagagagtgcggagcgtagttgccggaaggaagagagccgaccataaactcggtgccggattgcacatccggaaggtctatcggagtgatggagtccagagcattggcggaactgtacaagcgcccctgatcgtcgcgtaccagtaccagacgttgttgaaattcttcaacctgcacaatcgcggagttcaaacgatcacccgccgggtccaagtttttcgctacccacggcccagtatcgccctgcacacgtacccaggccatttgccgttgatcagcgaatgcatcgccgcccggatcgccatacccgaccacacaatcataatacgtagtggtcccgttggagatgtaggaaaacgatgaggcgtaggcgatcatttgtgcttgagtgccggcgttagctcccacgtctgggactttctccaacagcgcaaagttgaagagatcaccaaacgtttggttactgcgcatagctagagccggatattgcgaagacggcagaagttggccggtgggaatgaacccatcgtttaccggcggcccagggatttcgaacttggcccggatacgcagactgccgatatttccgcgcgctgctgtagtatccaaacccggcagggtgtaaggcaccaaagggataggctctggattaccagtgggtatgtggtacaggccgaacactaccgactgcatctgggtgccgcctagatagtctccgccgggcccgaccatatcgtagaacttggtatctcggtcgttcatgaggtcgccggctttccacgggaaaggcttattgttgttgatggctacccagccgtacttgctcgggtccttgtccagagagctagctacgttgagtgctacgttagtttgcgaagagcccgacagcgcaaaagattcaacgtccatcaggtactgcactggatagtctggaagtttgatctttaggtaaacgccccagatcaacgcatccggagtatttaccgggtccttgaattcgaagcggccattaccgagatcggtgaaggtggcagacacgcctagggcttgagcctcagtgacggaaagaatggcattgttgttaggtacggagttaccgctaacgccgtagaacagctcgtactcggtaactacaggttcgacgtttccgcccccgctgctgcctccagtcgcgttgatagtgatcaccccggactggtcctgaagcgtaacgttgttgcctgccttcagttgtttcgcagacagagtagtggtgttcggcgccgagagtaaggctaccccgccgctgcccgcctgctggagcgtcttgaatgccaccgaaccagtagcggaaaagtcaagggtgccctcactcaccgtaatgctaacgctaccatcggtagagaccagggttttgaactgaaggacgttaccaacggtttgtaggtaaacggcggcaccgttgcccaagttctcgccgctagcagggtcaatatcctcttgcgtgatcagtgcccaggtcccgtcattagcgagaaatcgcttcacttcagtagccggcggcgcgggtactaggcctttcaccccaggtccactagcgtcggagggaacgaaaactggctgaccgcttatcagactttgcacggtgctgacggtgggcaggttattcgcggagctgccaacattggacatcgccgcagtccccagatcgtcgatagtgagagtggcctgctcccacttagccgcacccgccgtagtgtctacgcagaagtacagctccttggtggtggtgttcaaccatttgctaagaatcgaatagccctgcgaggcatcgtccgccgaggtcgggtccgtggtggccgtgaggttatgcttcagcaacttttcgtaacgcgcatcaccctctgcgatagtgagtccccccgtggataactgttcggtccaggctcctcctttgaacccgaagattttgtattgcccgttaccatccagttggtccagaactcgaacctcccagccatttttaggggtgaaatactcccacccagcgagaacaacgctgtagcgcgctagcttgttctggttggcgtcggccccggtgtaaacgtatacgtcaccatccgccataccggaagtcggcagggaagagacaatactgcgtacccgaggctgaaccaaagcatctaggcctcgcatgaagttcatcagaggatcgtagtgttcttcacccttggcaccgccggacatcagccccaggttaccgccataaacaatagtcatatcagtctccttctccgagagcgtaacccagactcaacccgaggccaggacgaattagaatttgtggtatgtcgtaaatctgccaagactgcaacccgtccacttcggaccaaagctgcactcggaattgtccttgttcagcgtcgataccgtcctcgccgatcatcacggcagtatactcccaggacgtaccggttaatccagtaaccgagcgcaagacctgacctgacatcttcaggattcgcaaggtgtacgtaacccctgcgggcagcgtcaggctcccgtaggtgtggccaaatagttggtcagcctgagcttcgcgatttcggtgggcccaggtaaacgtgacgggacgagctaccggctccagggcgcccaaggccagtccgttagttttgaaattacctggggggtacggcctcgcatgacgcccacgcatgttcagcacatcgaccggcgcgtcagatagcgccaatagctcgcgaccggtccgggtaagcattttgacgttagctgtggtgttaggcaggtaggtggcgtccgaatccgccgtatagtcgtcatacaaccaaatacgagagccagcagcatggtaggcgggcactgtatccagagttccgcgcccgagcacaacaacgttggtggacgtgtttatcgacttgacaatgaactcctcttcgtcgatcacagccggcgagccaacctgtgcaaagtccagattctccgtaccaccgagcggcacttcaatgtcgtcagcagccatcggtatcgatatgccaatctgggcgttaggtacccaatcgccttccccgtggtcagcgaagccggaagaagcataatcgacgtacagctcgtaaccgtaggccataccgaacggtttagagcccatcccatacagataggtggtggaggcgtccagccggtcgaggtcagacggggaggtaatccgaaacaaatcgcgataccccatctcccgaactcggcggataggtacaggccgcgccgtttggtccggcggaacccactcggaaggttgaggttccacaatagaccgttgcggcaggccgtacacatcctgcactgcggtaaaggttatccgcccctctagatgggatgaggaatttacactgccgatccgcaccggcatgttctgtatgccgtgctctgggatggtgatactgattacgtccccaggcaggatatcccagccagtacgatcaaaacccagttcgaggcgccgcagataccccatactagcttcgagatcacgtaaggctacgcgaccagccagagaaggagttggcaaccccaaatactcattgttctgggctatcgaaccattcagccttaccgccgctaggttttggactggcgcggatgacagtttctcaccgttaatagggttgatgtaggtgactgttaccgtgttcgtagcggtgtagggggtaccttgggtaacatcgctggcactaatgatgccttttccgtaggtgtagtgcggcaggttcgcggggtcgtaatcggcacgcagtagcttgatggtgaacagggcagtccttggatggaagtagactacggcgccgatgtggtcaatgacagtctgcatgaaattactgacactatcggtacgcgtccacttcaggcaaagaccaaatccttcatcgaatagggtatcggcggcctgggagaagctggcttcgtcgatcagaccacggtcaaggcctttaccccactcggagttagtagcacattcgtagagtatgtgtgccgggttcatggcccggatcggctctccgtttggtccctccagatttatgacggctttcgacgggtaccacggctccccttgccatcctttcaaggctcggcgcacccgcattttccaaaccttcgggtaagggttcatagcgctgacctgtccgtcgaaaaacaaggtacagactcggcggaaagcaggtacgataccgcccaacatagccgcaaggcggccagatactggctgctcccagtcccccatcagcacctcaacggaacctacgatgccgccctcgcttttatccccgccgaagagattcgggttgttgatcgtgaaggtgccgttctccgtaatagagcctttccaggcggagcggtcaccgacctgaatctctactagctcatctatagggccacgacatacccccatgtggatgcccatgtagtatttgtagccaatcgtgacgctctttttactacccacgggccaccccctttctgatcttgtctaccattgcgatgacgtgagcatcgccagtagcttccagtttgctagccataacaccgttccttacgaaatccttgaagtcgatgccaagtatagaggcccaatgcttcatcccgggtacgcaaaagtcaggctccccgggctccgcgcgcatgtacttaacatgctcggttgtgatcaagaaatcgtccattttcccctccgtgcaaacgcttgcacttatttaccggacttcgttttgattttcgaggtgcggtaattgcccaagcccaacaccatccagtcttcggtccatacgtcgccgaacacgacagcctgcggcgtaccttcctcgaactgagggaagttccagtcggaaagagcagcaggtttcggtttatccggtttaggggcgatggccttattgatgtagtagctgatggcgatccaggccaggtagtatagagcttgcatgctgctctccttaattgaaaatggggttgccgtcgaaaggagaccgccccgggagatgcggctggccgccgtagttgacgctgttattgaacttatcgtggcaggtagcaatgacgcgattgcaccctgggtacaatgctacagcagcccccgtataaagccctgccgtgcctcctagaagaacgaaatgcctgccagtctgcctacgtaccccgcgaagctccgcgaaaccatcttgcgtagtgaaagtgatatatcctccactgaagtaatcgtcaggaaatccgaccagcaaagctacatctacactcaagccatccatgcttgctacctgatcgtccacgcgataggcctccgggttcacgcggcagtcttcgtcgtaaagtgtattcgagcatccccgctgccagcaacgacgaagacccggctgcgtcatggaagatgccaacgactgacacgatagcttgcacgtaatgctatccggccaactggccgtctccaccaaacccatccacgttacgacgaaatcatccgacccgtagtgcccatcccagatcaccagatttacgccattagacggcgggaaagcctggaaggcgcgagagatttcggagtctatgtggatcgttatctccacggtactggcatccatgccgcccgtgttctcgaaaccagggtccgctatctgggacccactcttgaactctatgttgttgagtgtgatgttgcggttcgccgaagtgtacgcaaagaccactgaacttcgagtaaaccgatagcaacggacaggctgacccagctcctcggatttctcaatatcgtcgaatgccatattacacctccatatcatcccgcaaactgcgccaagtggttttgcacgaacccaagccctcgatatcggtttcgtggttgatttcaatgaaatcttgatcgagccgacatagggccatgaagctgatccgcttgactttggttaccggcaaatcttgaccaatctgcccatccagagtcaggcgctcgaacgtttctccattttcagtaagctccgtggccgactctaccctgcgaagaattaccgagttgtccgtaagcaaaatatagagatcgcgacgcccagggcgcgagataccgaaacgggcgtaaccgccgggcagcacatccaaggtagtgtcttgggtcagcgcttggcggtacatcgttacatcatccgcgtgggtaggaagccatacggatttccaccggccacgaaggctgtagtagaagctgcgcatcgccgagtgctcttccgccccatgcatcagccaacggtgggcagtagccgtgaatcccaaattggcagtgtcccggtacgcgggcaggtccgtgctattgtcaatcatgcgaagtagacgaacgtaggtacttgtgagttcctccgactcctccggcggcaattcaaatacaggcttaccgcgatagatcaacgatggcgataccgccggccagtctgagttttcactcagttggaacttagctttcaccgagtatgcacggtccgttatccgggtgaaggtaggttgttcggagaacctcgctgacctcgcaggaaacaccttgctcttacgagcccatgcactttgaagagggcgctgcaaagttacccggttgggttccactgtagcgatcactgccacctcatagtcgaaggccgtagctccgcgaaggataaccaaaccgccgggccggaaatcccgattcgcggtaatacagggtatttctagtgatgcggctccaatgccgactgccagctcttgaacatctgggtaaataggcagtgcccaattccggccaccccaatcgaaaaccgccatgtccattagttgtcgttcgcggtctcgtg

SEQ ID NO: 4 - DNA, 58217 bp, unidentified
  source 1..58217: mol_type = genomic DNA
gactttactactgattttattatctttttacctatttaccctaagtaccctaagttaagaagaaattaagcgtgtatacagaaagaagaagtaaggtcatcacatgatgtcgtgtcgcattacgcgtatacggaatttttgcaacttagggcacttagcagatcaccccataaatgtttatattcaacaacataacgtgccctaagttgattttcggcacttagggtgatcggcaggaaaatgacgaaaacatggaaaatttggaaaatcgaatcaggaagtttttgtttcctgttaacgattatctagtattgatcattgaaaaaccggccatagcaccagtctgtttttcgcagaaaaagaaaaaccgcccgaaggcggttctttttatcgacgaaatttccaatcaaagatcgtcatcgacgtcttcgaacgcagtttcctcatttttattgtgctcagcgaagtgcaaacgcaccttttccgggtctggatttccgtcttccttcatccattttctcgggtctaacgcgtaaacatagccttcctctccatccaccctgacgcgagaccaggatttcgtcccgaccttcttatacccgaggtcggaaaggcggcggttgatagcactggtacggccgatttcccgttctgggatagcatccacgatgaactttttcaggtctggcaggaaaatgagttcgttgctgacgccgcgaatcttcccgctgtacgtcgcctctttaatcgaccgcgtgatgtcgtccactgcgtagtcgatcatgtagtcgcgagctgcagtatcccgcggagcctggccggcgtcgtgggcaaacccttcgctgaactcccattccgaaaaccacttacgcaaagccccagggtagcggcggaacgcggcatccatgcgacggaagaagttcgggtcactgcgcttgaaggcgttcaacacttcggtattcgtgaagtaggattcgacgatcagccaccgacggtcttcgttgtcgatcggcagcgccgccgggtcgttggtgaaggcgatatatgacgcggtgttcctgatctcgaaggagtccacgcctttcctttcgcacggaatgaatgggttcgtgatcttgtccttcaggtcgtttatgacgtcatatttgtggcgcggcacggagacttcctcgagcaccttgaccaaaccgccttcagtccacccggtgaacttaccctggaaaatcttgttgttcacgacgtcgacgtttgtgtggccaagcaagcatttcagcatcgaggcgatgatcgatttaccggagccttgcgtgccgcggatgaccagagcgtagtctacgcgccggccggggtttgcgatgacccaggccagccagtccatgacatagcggcgctccttgtcgtccgggaactggacgaagaagaagtccttgagcgtgttgatcgctttcagttcctgcttcgagtaggactcaggcatcttagccagggtatccgggttgaaggtgttcaggaacgtccgtcccagctcgtctttgaacaagtcggtgccaggcaggcgccagtgacgcttccattcgttgttcggcaggtcgccgtgatgcagcgggtcatagcgctggccatccacaaccggaaccgggaatattttcgtggccagatccaccggcaacgcgccgccctctttgtcggaggaaacgaaacgctggtttgccatgttgaaagcgcccggcaaaacctcgatggcgttattccggttgacgaacgcgccggcaacctggtccaggacccacggcttcagccactccggcagatcgtcggagttcgggcggtagtccagataagatttcgcctcggctttatccagcgggcggttgaatatgcgtttgaacgcctcaatggccaccgcgcgcacgtcgtttcgagcgacgccgaaaattgcctcgcgtttcaggcggtcagcggcttccagccattcttttttggacgtggccatgtcgaaaagcggcataagctcttctgcaacaacggccgcctgctctttttcgatctcgcgggcttgttttatgaacgtagccagggtgatcaggctatcgccatcgtggtggaagcccttctgccacttgtcttcgaaggcgtccatgtcgaaattgaaggcctgcgccgaccatttcagggcaatatcgtaggctaaatcctgattttcaacgcttatttgcagcgccgcaatcatctcaatccaggtctggtagtcctcataccccggaatgagcatgaccaaacgctccagttcttcgtcagaaccttcccatttgcgcaccagatcagcgtctgacaggccaaaagcgtcatcatcgccgatgctggtggccgccggccgcgattttttggccttcagctttctcacccaacccgcttcctctgccatctggtcgaaggcatcgcaaatggcgcgcgcatcctccggcgtcagcgttggaagttcatcgaccgccgtgttcgtcgggtcttttccgtcaacccaccaatacggctcgtcggtgtccgggtgaatgccgaaagccaccagctgctggccatcgccgaggatttctaccttgctggcgtgcccgcttggcgaagtccaggtgccggaggtaattttggtgaacggttcatcggttctgaacagcatcaaccgcttcggcgccatgccgaaacgaaccggcgccatgccgtagtcgaactctaccagctcctgaaccgcagaagccatggcgtcgtcgtaaacgtcaatatcaacagccggggtataggcggtattcaggcctgcgccgccatttttgaactctttttcccagcgtttcgccagttttgagtccgcttgataggtcgtccattcgtcaaatggcggccgttttgtcttgaatcgcagaggcagaaagttatagccgttctctaaaaggtcgggccatacctctgccagtttggttttgtgcagattatcgtccatcaccgccccactttattggtcgatttcattaggcagacgttcaaaatcatccctggaaatcacatccttcccgacgatcgcttcgatcgccagcggcaccgccggcggaatcgtccgtcgattgagtgcggagtataaagcattgtcactcgcgtatccgcacgcgcgacaaacagatgaaatgccgcctttttcgccgagataggctgcggctatgcgaaacataaaacggcatcttgccaggtatcgctccgatggcgataacgtcgggtcagtgacccacgccggcagcgggaagttcttcatgcactacctctctttctctcttttagcagtaatgtgaaacatcctactctagaagaaatatcaaaatcaatgtgaaaaagtatttgacaacaatttcaggttaggccattctgatttggcggcgaacgagagtcgccattttacaaaaactgttaatcatttttcggagaagaaaccatgtctgaagttaccgttaaaatcgaaattgtaggcctggctgacctgatcggcgcactgaacacccacacccaggctctggccggcggtgctgcaccgaccgaaaccaaaaccaccagcaccaaaacgactccggccaaggaaaccaagccgaaagtcaccaaacctaaagcctccaaagctgaagtgagcaccgccctgggcgaagtgaaagcggccttcggcatcgacgctgctcgcgaaatcatgtccgaaaacggcggcttcgctaaaatggcggacatcactcctgagcactacgatgctatcgttgctgcctgtgctgcaaaaatcgcggaaaacgatgacggcgccggcaccgaagaagaaggcgacggtctgtaaggcctgatctcttcatcttagcaataaacaaagcctgccatccggcgggctttgttccagaagggggtagtcttgaccgagcataagctagacctggataaacttgtagccgatcgaaacggccattccgttttcgggccatccggcagtgctatgtggtcgacctgcgccggctcgcttatccccaacttgctcgctcctgatgaaacgtcgttcgaagccgccgaaggcaccgtggcgcacgccatagctgaggaatggctgaaaacaggcatcaagcccaagcaccacctgggcacgacgcaaatagtcgaagaaaacggcctaagattcgaaattgtgatcgacgtcataatgttcgattacatcaaatcgtatgtcgactgggttcgcttccttcccggtcagcattatgtcgaaaggcgggtcgatttttcccggcttatgccgataccgaagcaaacgggtactgccgatttcatcgcgctggagcacggcaagatgtggatcagagacctgaaatacggcgctggcgtatacgttgaggtcgaaaacaacacccaggcgctgctgtatgccctcggggcgttctacgaatacgatgaaatctatgatattcaagagattgacatcggaatcggccagcctcgaatggataatttcagcacctggaccatttcacgcgaagaattgctgtcatgggaaccgtatttcagggaaactgcagcagcggcatggaaagtcaacgcgccgcgccatccttcagtgaagggttgccggtggtgcaaggtcaaagcgacctgcggcgcccaggctaaacttcaggaagagctggcaacgttggcgtttgacgacctggcggaagacaaaacggaacaggacatcgaagcgttgaaaaacaggattacggacgaccttgagccgtataaactggagttcgtctctccgagtgaccttaccgtggagcagatggccatcctcgtaccgtatcgccgcctggcggaaatgtggtggaagtcaatcgaggatgaactggaaaaacgcctgcaggaaggggcgaaagtgccaggctataaacttgtcgatgggcgaaagtcgcgagacttcatcaacgagaaaaaggcagcagaggaattggagtggctaggcctcgaaccgggtgagatttttgagcgtaaaatgatctcgccggcggaagctgaaaaacgtcttcgcaaaaaggggtatcgcatggctgacatgccgtcccttctgaaaaagatcgtttatgccaagccagggaagccaacgatggcacccgaaagcgacaaaagggaagcagtgcaagacatggccggtgacgcgttcgaaagtatggacgacgacctgtagccagaaaaccagaaaccgtgaaaatcacgaaacccgaatagaaggaatatcacatggctttagaagtcgctaagaaagtaaaaaacgccgttctgtacaccaacggctgcattcgcatcgacggcgttcgcgcttcgttccctcacctggataagccatacgcaggtgaagacggcggcacaccggcttacggtttggtgagcctgctggacaaaaccactcacaaagaggcgaaagatctcattgttgagcggattaacgaagtcctggcgcagaacaaaaatgcgaaagttgctgctgatcgcaaattcatccgcaacggcgacgactctgcccgcgatgaagagaacggccattggacagtatccgcgcgcgaaaccaagcgccctagcatccgcgataaggataagtcggtgctgtcacctgaagaagccctggataagatctacggtggctgcttcgtaaacgtgcttatccgtccgtggttccaggataataagtacggcaagcgcgttaacgcaggtctggtagcggttcagttcctccgagacggcgaggccttcggtgaaggtcgcatcgacgacgaagaggcgtgggatgatgtcgacgacggaaatgaaggcttcaccgacgatgatgaagacggcctgtaatacagggcgctgaaccgccggtctgccagccgccgtggagaagttttctccgacgaatgaccgatggaagttctattgccctcctaaccgagggcatttttatttcgaaagggggaggccatgtcccaaaccgtaatcaaccttgactacgaaagtaaatcgccggttaacctaaagacggaagggttagaccggtattccgcgcatcgagataccgaagtgatgatggccagctggtcaattaacgaaggtactgtccagcactgggattacaacaacggccgcatgccaggcgaacttcgcgaagcgcttgaagatcctgacgtggagaagtgggcattcaacgctcaattcgaacgcatcatgacccagcgcgttctgaagataaaaacgccatacgagtcttggcgctgtacgatggtgctggcctacatgcaaggtttctccggcacgcttgaacaggtaggccgcaacgtcggtctaccggatgacaagcagaaaatggcagacggcaaacgccttatccgtctattttgcacgccccaaaagcccacaaaaaaccagccgcatatctggcgcgatctgcttaccgacccagacgagtgggagcatttctgcctgtataacatccgagacaccgaaaccgagatggccatcaagcgccggctgataaagtacccagtcccggcctccgagtgggccctgtacgcgcttgaccaacagatcaatgaccgcggagttccggtcgacatggatttcgtcaacaatgcaatctggatggcggcgcgccgaaaaaaggaactcattgcagacctgaaaaagatcacgaaactcgctaacccaaactccacgcctcagctgctcccgtggttaaaagaacgcggatacccgtttgacgacatccagaaagatacggtgaaaaaggttgtttcgatccctctcggaaagctgaagcgccagctggatattcgccaaatgctcagcgtcgacgagtacctcgatgcagatgacccaggaactgttcctgttgaaaaacagcaggagatgaatcgcgcgctggacgttcggaagaatgcctcgcgcatcagcattaaaaaatacaacgccatcgaggcggcgcgcggcgaagatgaccggttccggttctctctgcagatgcacggagccagtcggacaggccgatgggccgggcgccggctgcagacgcaaaaccttccccggacgccgaagttcctggaggaagacgaggacttggaaatcgctaccgagatgattcgcgccggcgagtatgacatgttccagctctatgtcggggagccgatggaagccctggttggccttgtacgatcgtcaatccgcgcgtcagaaggccgtgaactccgggtgtgcgaccttgcctcgattgaatccgtggttatcggctggatgtgcaaatgtgcgtggctaatgggcgttctcgaccgcgggcacgatatttatcagtcgttcggtgtccatttgttcggcaagccgtatgagcgggtaacgaaggcagaacgaaacatctccaaaccaccaacactcggctgcggctaccgccttggcggcggcgagatgcgagacggaaagaaaaccggcctgtggggctacgcagaaaacatgggcgtaaacatgactcaagacgagtcgatttacgcagtaaaagtcttccgtgaactctgcccggaaatcgttcagacctggtatgacctcgaaagggctgtagagaaatgcgtcagaaccggggcaaccgttcaatgcggtgtcattcggttcgagatgaaaaaaccgtacctttgtgccgtcctgccgagcgggcgccgcctctactacttcaagccgaggatcgaaaccaagcagttcgagggtaaaaccggtccatacaccaagaagagcttcagctacgagggccaggatcagaagacaggccaatggacgcgaatttactcccacggtggtaagctggtggaaaacttcgttcaggcgatcgcccgtgacatcatcaaggccggcttgcttcgcgcagaccgcgcgggcttcgacatcgtgatgcacgttcacgatgaaatcgtcaccgagcagccgaagaacgatgacgttcgcacagtggcgcttctgtccgagtgcatgacggcaccgatcagctgggcgccaggacttccgctcaaggccaccggctgggcggggcagttttacaagaaggactgaccatgactatcagagaatcaaaggtcgaaagcgatattcggaaatatgcggaaaaacgcggctggtggcagtgcaagttcacgtctccaagcctgcgcggcgtgccagatcggatatttattcgagcaggcagacacgttttcatcgaggtgaagagacccggcaaaaaggcaacagctcagcagaaaaagcgccacgttgacatgcgtgaaaaaggtgctgaagtttacgtcatcgactgcgtggaggatgcatatgcgatactcgagtgacctttacgagacaatccggcgccggttcgctggtgttgagctgcaggaaaatcagctacacctctatcaggaggacgcgatagacttcctcgatacgaaccgttatagcgcgctgttcattgacatgggcttggggaaaacgatcatctccctgacgctggcgtcccggcttttgaacgacttcaaggccaacagagtcctggtcatagcaccactccgcgtggccaataccacctggccgaacgaaatacgcttgtgggaacacacggctgggttcgatttcacggtgctgaccggcagcgctgaagagcgaatgaccgcgctcaaggaaggcatgcaccgcaactgcccgatattcatcatcaatcgcgagcagatcgaatggctggccgacatctacggcaacaaatggccgttcgacaccgtgtttgtcgatgaaagctcgtcgctgaaggatcacacgacaaaacgttttaacgcgctgaaaaacgtgcgccggtacatccgccgtttgcacgaactcaccgcgacgccggcggccgagacatacttgcacctcttcgcccaaatttacctcctggatcagggaaagcgccttggcaaagacatgcgtaatttcactgataaatatttcacatacaatcattacaatcgcaagtataagctccgccccggcgccgaagaggaaatcgtcaacctgatttccgacatttgcctcgtcatgaagcgagaagactacctggaattccagcagccgatcttccacaacgtgatgatcgacatgtcggctaagcagcaggcgctatacaaaacgatggcaaccgagttcatcgtcgaactggataacggcgttgaggtcgaagcagaaaccgccgccgctctatcgagcaagctgctgcagatggcctccggcgtcctttacgataccgtcgatctggaagacgccaccaccggcgacgtaaagcagttgaaaaaagtctacgagctgcaccggcataaaattgacgaactggagcgaatttacgaagaggcgaacggcgaaccgatcctggtatcctaccacttcaaatcgtcactcgaccggctgaagaaggccttcccgaaagcggtggcgatggacagagaaggcaaggctgtcgcgcagtggaacaagaagaaaatcccgatgctcctggtccacccgcagagcgccgggcacggccttaacctccaaaagggaggccatcacatcgtgttctacgacatcccatggtcgctggagctgtacctgcagctgataggccgactcgaccgccaggggcagcagcacccggtaattgtccagcacctgatttgcagaggcacggtcgataacctggttattgcagcgctgatgaagaaaaacgatgcgcaggatgcgctgttcaggatgctgaagcgcgcgagagccaaactgaagaaagaccgtgcggccaaagtcgacctggccaccgaggcgtttgacgacgacgatctgtgattgacggccgcgcgccggtggcatattatcaaaccacttccccgcaccccacgcggagaggccatagataggtttcccttttaacccgcttcggcgggtttctttttgcctgcagttgccagactgaagatatgtgatatgttcattgcatatatgaggacgcctgcagtgaaccaaaccctgacccagttcgaagaccacatcgccatggggccaacattcgcagcgcgtttgctgggggtcgcctacgccacttacgcccaataccggagcggccaccgccgattgcagaaataccaccagcgccacatccaagcgctgatgctgctgagcgacgaagacctgcagaacttaataacggagcatatccacaatgacccaggcaaaaaagccagctaaaaacagcctcgacgaaacgagttcctcgatgctttatcagggctgcaatttatcgcagctcggcatcttgttccgccgagaccaccgaactattgttgagcgcatccacggcataaagccggccggcacgcgcggcggccatagcatttatcaggttcacgaagttgcgccgtacctggccgacattccgccggacatcattgaagaccgcatcacgcgcatgcaccacaatgagctgccgaagatgttgacgaaagagttctgggccggtcagcggagcaagcagctcttccagctggaggccggcgacctttggccgaccagcaagatcatcgataaagtcggcgaggcgttcaaaacctgtaagatgtcgatcatgctgctgagcgacgcggttgagcgcgagaccgaattcaccgatcggcagcgcgaaaagctacgagaattaattgacgggacgctcaccgatttacacaagtcgttggttgagcgcttaacagcggacgatggagacgacaatggcgaaagtgacgaagacgaagacctttaagacgatcggcagcatcatcgccgacctcgccgaactcttccgtccgccggagcgcctctccgtatcccaagcggcggagaaatacgtctacctgaacacaccgggcgcctacgtcggtccgtacaaaaacgaaaccgcgccgtacatggtggagccgatggacacgatgaccgcacgcgacaaaagcggcattgctttcgtcgggccggcccagtgtgccaagacgcagtgtctcctgctgaacttcgcggcatacagcatcaaggtcgaccccatggacatgatcctgtattgcccgacaagcacggcggcgcgagatttctccatgcgccgtatcgaccgcatgcaccgctacagcaaagaagtcggtgcgatgcttatgcaaaaacgcgactccgacaacacgttcgacaagcactacaccaccgggcagatcctgaccatgagctatccgtcagtgacggaaatggcggggcgcccggtaggccgtattatgctgacggatttcgaccgcatgccggaagacgtcgacggcgacggtaacccatacgacctggcatccaagcgaactaccacattcggttcattcgccatgaccgttgcagaaagttccccgagccggccgataaccgaccccaagtggattccaacgtcgctgcatgaagcaccgccgacgaccggcatcctggcgctctacaaccgcggcgaccgacggaagtggtactggccgtgtccgcattgcgacgagtatttcgaaggtaaattcaaactgctggtgtgggatgacagcctcgataataacctggataaggccgagacgactcgcatggcatgcccgcattgcggttgcctgattcaccctgacgaccgattcgaaatgcagttgtggggccgctgggtcaaagatggccaggcgatcgataaaaacggacggatttatggcccgccgattagaacggcgatcgcgtcattctggcttaacggcacggcggccgccttcaccacctggaaaaagctggtgaacgactacctgacggcgctggacgaatatcaccggacaaacagcgaagagtcgttgaagaaattcttcaacaacgacctaggcgagccgtactacccgaaaagcgacggcgacgtccgcttcccggaacacctgaagagcatggcgtttaaactgccagagcgggagataccagaaagcgttcgtttcctggtggccaatatcgacgttcagcgttctagttttatcgttcaggtgcatggcatatcgccgggcacgccgttcgacatcacggtcatcgaccgctttgaaatcaagaaatccgatcgcgtcgattcggacaacgacccgttgccgataaagccggccagttacctggaagactggcggaagatcgaagagctggtcatgaacaaaacctacccgctcagcgacggcagtggccgccggatggcgataatgatgaccagctgtgactccggtggttattcgaaaggcaaaggcgagtccgttacaaccaaggcctatgatttttatcgtctgctgaagagcgaagggaagcacggccgattcgtcctgatcaaaggcgactcgaaaaataaccaacctcgcacgcgggtgacgttccctgacgcgcagaagaaagacgacaagtcggccgcgcgcggcgacgtaccggttctgttcttcaactccaacctgctgaaggacgatctggacggccgactggattgcgtcgaaccgggtaaggggatgttcatcaccccggactggttatccgacgacttctatcgcgagctttgcgccgagacgcgaacgccgcaggggtggaaaaacatcggcggggcacggaacgaagcctgggacttggcctattattgtatagggctgtgcatttcgaaattcatccgaatcgaggttattgattggaacaacccgccgagctgggcagcgccgtgggacaataacttccatgtgtcaagtgttgacgaccctgaaacatttgcacaaaccaatgatagcggatacgatttcgctaaattagcggaaacccttgcctgacctaacggagacacaaaatgacgccagaaacctgccggtctttactggaggacgcccggaaagcgtaccacaaccttgttaccgggcagatggcgcgcgtcgtcgtcgataaggacggttctcgggttgaatttacagcggccaacaaagaggcgctctacaactacatcctccgcctcgaagcgatctgcggttgtggcggtgctggctctatgtcgccaggcggtccagcggggttcctgttctgatgaaaatcaacaaagccaatgttcagccactccaggaaaacgccatcggcggcggcctggaaggggcagaaaggacatcacgggaaacgctcctgtggaatccttcaaatcgttcggcagacgcgctgattaacccggtcaaggacatggcagataaccgtgcccgcgacatcgttcagaatgacgggtacgcgctcggtgccgtggccatccaccgggacagcattgtaggttcgcaataccggctaaacgcccaaccaaataaccgcgtcctgaacgccccagacggctgggcagaagagttccagaaggtagtcgaagcccgattcaacttggtggccgactccccggaaaactggttcgacgcccaacgaatgggcaccctgaccgacatgattcgcctggcagtcggcggcatggtcatgtctggcgaagtgctggcctctgcagaatggatcaccgacgacccccgccggccgtttaaaaccgcaatccagatgatcgccccgtctcgcctgtcaaacccggacggggtgagcgactccaaaatactgcgccgcggtgtggaatgtgatcgccgcgggcgcccgattgcgtataacttcaggagttcgcacccaggagacgtttactgggacactgaccttttcacctggaatcgcgttccggcggaaaccccatgggggcgcaaacaggtcatccacatcatcgagcggctgatgccagaccaacaccgcggtatcgcagatatggtcgccacgctcaaacagatgcgcatgaccaaatcgttccaagaggtcgttctccagaacgcggttatcaatgccagctatgctgcggccgtagaatccgaactgccgtccgaacttatctggaaaacgctcggcgctcctggcccgaatgatgctaaagacccgattaagcagatgctggggtcgtacctcgattccctggccgcgtattcccgcggcggcaacaacatcgcgattgacggctctaagattccgcatcttttcccaggcaccaagctcaacctgaagccgatcggcacacctggcggcgttggcaccgggttcgaagagtcgctgctgcgccatatcgcttcgtctctcgggctgtcctatgagcagttcagccgtgactacagcaaaacgaactacagttcggcgcgcgcctcgatgggcgaaacgtggaaatacatgcagtcgcgaaaaaaagtggtggccgaccgcctggcgagccacatttacgctttgtggctggaggaagagatttcggccggcaacgttccgctaccaccgggtgcccggcgtgacacgttcttctacaagccgatgcacaaggacgccctctgccaatgttcttggattggggcttcccgcggccagattgacgaactgaaagagacccaggcggcgctgctccgcatcaacgccggcttctctactcgcgagaaagagatcgcccgccttggcgaggatttccgcgaagtattcgaccaactggcacgcgagcagaaactggcagacgaccttggcttgacgtttgctattgacgcgcagaaaccgggcgctaatatggcgcagcagacactgaccgccaacaacaaaaacgaggacaacgcaaatgagtgaatcagcagcacgcgccgcgttatctcggatgaacatgcgacctgccgccattgcgcccgtatacaccgaactggccagtgacctgcagaaattgtcgcaagtaactccgcagcgcgcgaaagaaatgcagctcgaagtccgggctggtctggccgcgcaatacggcgatgacgaaaatgattacgaagaagaaacccagaagccgttctacttttcgtcaggcatggccatcatcccgatttccggctcactgattaaccgcttcggcggctgctatggctggatcaccggctacagctacatccagcgccagctgaatcttgctctcggcgacccggatgtgaaaggcatcatcttcgacgtcaacagctacggaggggaagcggcagggtgtttcgaactggcagatatgatttttcaagcaagatctatcaagccatctatggcggttgttgattctaactgctattctgccgcttatgcacttgcgagcgccgccggaaaggtcgttgtaacaccaacaggcggggctggcagcatcggtgtagttgctatgcacgtcgactattctaaaatgatggaagagtggggtctgaaggtcaccttcatttacgcaggcgcccacaaagtcgacggtaacccatacgaagagctgagcagcgatgtcaaagctgacatccaggccagcgtgaacaaatcgtatgagatgtttgtgaacgaggtggcacggaatcgcaacctctctgctgaggcagtgaaggccacggaagcgcgggtgtaccgcgcagatgaagccatgagcctcggactgatagacgccgttgccacgcctacccaggcgctgcaggggttctataacgagctttccggctcgacaattcaactggagaacagcaagatgcctcaagaaaatctcgaaaccaagccggccgttgtagaccaagcggcactggaccaggcgactcgcgctggcgcagtggcggaacgtgcgcgcatcggcgaaatcctcggccacgaaagtgcccaaggtaaaaccaagatggcccaattcttcgctttcaaaaccgacatgtcggtagcggatgcggtagttggcctggaagcagcagcactggaagttgctgcagcgccggcgaaggatgaaaaagcggaaaccaccccgctggagcaagccatggccaagaccaaacagccgaacgttgaagccgatggctctgacgtggatggcgaaggcaaggagaacatgtccgccgccgaccgcatcctggcgaaccaacgtgtcgccaacggcaccgctgttaaacactgatcggagagaaagagatgggatacccaactttcgcaggttctcaatcccaggcattcggtgccgcggttgagattttttctggtgactctgacgtggttaccaccgacggcccagtggccaacgacgcagttctgccgcaatactcggttatcgcgctgaacgccactggccaaatcgtcgcgctgaatccagctggtgcagacgctaccgcttctgaagcagtggcgatcggcatcacctgctacgcagcagacgccagcgcttccggcgcgcaacgcggtaaacgcatcgcctactactccggcggctcgttcaacccagctgctctggtatggccggcagcaatcgacaccctggcagagcgcaccaaggctttcgccgggactaacatcaacattcgcgaagtgctggcgtaaaggagcagtatcatggcgtttgaactctacgatatggcgaccctggtacaggttcgccgccaacaagtcgacgtgccggtgttctggttgaacttcttcacccagacactgaccttcgaaaccccgtacattgacttcgagaaagtatctcgtcgttacaagaaactggctccgctggtttcccctaacgtacagggtaaagtaatcagcagccaaggcagcaacgtaatgcgctttgcgccaggttacgttaagccgaagtcggtgatcgacccatccaaagtggtagcacgtcagccgggcgagtctgcctatcagccgatgtccctggcagccaagcgtgatgccgtgattgcggaaaccacccgcgaccacaaagcgcgtcacctgaaccgtctggagtggatggccgccaacgcggtaatcaacggcagcatcaccctggaaagcccggactatcctctgtccaccgttgatttcggacgcgacgccagcctgactatcgtgctgaccggtgcgaacaaatgggatcagactactgcgaaacctctgaccgacctgaagctcggtaagagccgtgtgtttgacctgagccagcagaagctgaaccgctacatcttcggccaaaacgcctgggatctgttcgcaactcgcatgggcttcgacaatccgcaaagcggcaacctgctcgatactaacttccgcggctctgaaaccagcatctctcgcgttctggatggttacccaggcgcagagtacgttggcaccgtgaaaggtactgccggccagggtgcgatggagtgctgggtttatcacgcgacctacgaagatgacgccggcgtggagcaagacctgctgaacaccaacgacgttgttggcctggcggacatccagggcttccgttgcttcggtgctatcatggacgcagacgcaggttacatcgccactgaagtgttcatgaagaactggcgcgaacaagacccaagcgtcgaatacctgctgagccagtcagctccgctgatggttccgggcgagccgaacggtgcattccgcatccgcgtagcttaaccgatacctgatcgcctgcttcggcaggcgttctttctatcgaccgaacgattacaagattttaatttaaggaattaagccatgccaaaacgtattgcggtacacaccctggttctcacccgcgacggtaaacgtgtgaacgttaagccgggcaccctggttgacctgactgcagaagaagtgaaagagctgaacgccctgaacgcgatccgcctgccagtcaacgaaaccggcgcgccggaagacgaagcgccaaaggcagaagataagaagtccgaggacaaagcgccgaagactgacgataagaaagccggcgccaagaaggaagaagacctgtaatggcctttgacttctccgaagcactggggaaaatgcggagagtggtgcatgacaccctctcccttcccgccacataccaggatcatactctcgctgatcctgtccctctgcgcatccgctggcacactaaactcaacctgaccggcgacctggaaaacaacggctatgcgcagttcattgagggcatcgagagtatcatctttgaccggacagagcttgcgctgaaaaacattacgatccgcgaacgcggaaaagttacgatcaccgccccgaatttctccggtcaggtgattatgattggccctaaagacccgttgtatgggccgatagaagaagtctacaaggtctcattgacatgacgataaaggtctcggtggatggtctgcttgacgtacaggaatacttcgagcgcatgcccgaggccaccaccatggccgcgagaatggccgtgaacgatgtagctcaacgcggtgccttaaaactggcacgcgacgagatgatgcaagaagttgccttccccgcgaagtacctcgatgacagccgcctcggcgtaaccaagaaagcccgaaataacgacctcgaagcgatcatcaccggccgcaagcgcgccaccagcctcgcacgattcgcccctggccaaacaccttccagcacggcgaaacgtggcgtgtcggtcaccgtgcagcgcggccggacggtcactcttcgagaaggctggctggttcgcctgaagcgcggggcgtcattgaccgaagaccagtacaacctcggccttgccgtccgcattggtccaggcgagaaaatcactggcaaacacaccagccaccagtcatggcttgttcgcgggtcggtcgccctgttgtacgggccctccgtcgaccaggtattccgtgatgtgaaagaggacattgctctgccggttctcgatatggtggccactgaattcctccgacaatttacgaggctgacaaatggctgaaatcccacgccgtctgcttatcctgcagcggttaagcgaacacctgaagggcatcaccccgctcaacgggtataactatgacctgtcccagtccgtctaccggggcaagcgcatgttcggcgcagagacgcagttgccggccgtgtcgatcatcgagtcgccaaggcctgacgtcggcgtctatactggaacaggcgacgagcagcgctccgagaactgggtattactggttcagggatgggtagaagacacggacgatcttgttaatccgacagacgccgcgtattatctggctgctgccgttgaggaaaggctcaccatgattatggcgaaaaaagcgaagggcacccggcctatgccggtttatcctgacctatatcgcctgggcggtttgattacagacctgcagctttcgcccgccgtggtcagccagcctcaagatgaaatctccagcaaagccttcttctaccttccggtaagaatcggtctcgctgcagatgtcggccaaccttacgttgacggctgatgattaatccaatattgaaggaaacaaaattatgcgtactccagtagaaccgaactacgtcattggccgcggtgaactgcacttcaataagttcgctaccggcactaaaactggcaccggcgagatgtatttcgggaacaccccggagctgtccgccgcatccagcattgatactctcgatcactataactcagaccacggtctgaaagagaaggacgagagcgtcacgctggaagactcgcagaccctgaccttctccacggatcacattgcgccagaaaacctggctctgtggttcggcggtgacctcagcaccctggccattactgccgccactgggcagacgtactccctgggtgcggtaatcctgggtcgctcctatcagatcggagcgtctgcgacaacgccatccggcgctcgcaaggtgaccaacgtcgtagttaaaaaaggtgcgacgactgttgcccccgaaggcaactacgaagtcgaccttgaactgggccgcgtctacattgagtccgatgctacagacgtcatcgaaggcgacgtgctggaagtgacgttcgatcaggaggcctacagccgccaggttatcgtcggcaaggcgaaggaaatccgcggcgccctgcgcttcatttctgacaacccagtcggcaagcaaaaggacatgttctggccatacgtcaagatcaccccgaacggcgattacgcactgaagggcgacgaatggcaggtaatgtcgttcaccgtggaagtgctgaagttgaacggcaccaccgaacgtgtctacattgacatgcgcgacgccaccgtttaaaggggattcacatgggactgatggactatcagccgccaagtgaagaagtcaagtttaaaggggggtccttcgaattgaagggcctttctactcaggacttctcattgttgctggcgcggcattcagtagacctggaaaaggtcttcgatttgtacgagaaaggcggagaagacgggacagacttcactgccgcaggactggccaagtttgtcatgacgctgataaacgacgctcccggattggtggccaacgccatcgcgttatccgccggcgaaccgaaagcggcagatcatgtcatgaaaactttcggtatcgcgctgcagctgcagtgcttaaaaatcgtgggccggctaacctttgaggaagcgggcggcgtaaaaaagttcgtcgaaagccttcgggagatctacgggaacgtccagatcccagggaagaactcaaacgagctgggcgagtagacagcgaaacacccgagccggaggaaacccggcttattcagttctatcactcgctccggcgagacgtatgcacgctgttagcggaaggacatcagcatgccatgcactatccgttgtgccacgtctggtcggaagttgagttagtcaggcaacgggtacatcagcgcatggctactgaggctgtgctacttcaacaggccattgtctctgtcctcgctggcggcaaagatttctcgaaattgctgaaggacttgaccgatggctgacaatgtgaaggatgtagaactacggatcagggcgaaggactatagccagaaagacctgaaagccctggtcaaaactatcgactcgctgacggccgcaatggccgagcagcagaccgccgcgaaaaagggggaggccagttctaaacaactggccgattcctataaacgcatggagctggcggccaacgcccttctgaaacaatccgcgttaatcgacaccttcaagcagcaggcgaaagccgttgatacagccgctcagaaaactgaggtggcgcagcgcgcttatgcttccctggcgacgacaatcgcatccctggaaacgccgaccaagaagcaaatttccgacctggccaaactggagcggactgtttccaacctgaccgtttctgaggcgcgcgccgaaaacacctggcggaacaccggaacgacgctgaaggccttgggcgtcgacatcaataacgtcgcggcttccgagcagaagatagcggcggcatcccgcactgcctcagcggcgctgaaggaacaagagaccgcgatcctcggcctcgatgcagcgcagaagcgcctggctcagcagcaggccgcggcggccgaacaagaaaggatccgcgcgcagcagtcgcaagcggcggccagcacctatgcatctttctgggtgacggcgctgaacgccaaagaggcggccgagaaaaaggcagcggcagataccgcgctgcagcaggcagttactgctcagcggcaggcgctgcagcaggccgccaatcaggctatcgctacggccaacggctaccgctcactggccagtacgttccgcgatgcggccaccggcgctaacccactagccacggcgctgcgccaaattatcgatccagctgggcaggcgagaactaacctgaacggcctggaggctcaggtgcgcgaattggccaccagcatctccgccatccgtgggccagtggataatctgaaaacctcgatgcagcagctcgacgccgctcagaaggcggccggcagtctggcgcagatgatcgatagctacaaccgacagctgcaggtgttacgccaggcgcggcaggaatactctgcatcgcgacaggcggttctacaactggccgaacaggtcaggtctgccgtatcccctaacaacgagctgaacgcatcgctggcggccgctcaggcacgcttaaaatcggcatcgcaggagatgcagaacgtcgtcaacaccacgcggcaaatgcgagcgggtttacaggacgccggcgttaatacgcgaaacctgcagggcgaagctgctcggctgacgaatacggtcaaacagaccaccaccagtgccaacgatctgacggccgccgtcaagcggtacggtatcgccgccaaggacgctgacacgaacacccgagcattcggcaacggggcgcgccaaagcctgtcctacgttcagcgcctgcgcggcgaagtgctcggcatggccacggcgttcatcggcatccagtcggcggtgaaactggcaaacgactcgctggaagcgtaccgctcaaaactggccatccagacccggttgtcggtgctggtgggcaacgatgccaaagcgatcggcaacgagtggaactatctgcaggggcaggccgaccgcctgggcttcggctttgagcaaatggccatgtcctactccaaattcgctatcgccgccaagatgtctggttcgacgctgcaggaagcgcggttcatcttcgaagggttcgccgaagcggcgcgcgcctcgaaaatgagcacggacgatttcgatggctcactgaaagcggttgagcagatgctcagcaagggcaccatccaggcagaagagctgcgccaacaactcggtgaccgactgcccggcgcattccagctggccgcccgcgcggcgaacatgagcgtcgcacaattcacgaaagccatggagaacgggcagatcagttccgacttcgtggtcaacatcgcccgcgagctgaaaaccgtttacaaagacatcctgccgacggcggtcgataccatgattgccgctgaaggacgtctgcagaccgcctactacaatttccgcctgtctatcgcagaatccggtttggtggaagcctattcgcaattcgtcgctaaacttacgaacgtgctgaagggcgatcagggtgccgcgctggcgggtcagctgtcatcgatgttcagcagcatcgtctcgctcctggggttcatcgcggatcatgctgacgtggccaagacggcgctactggcgatggtcgggattcaggtgctgatgcagttcagcggattggttatggccatcggcaacatcggcaccgcattgaagacgctgtacacggtaatcgtcacggctaccgtggccatggtgaacttccgcacgactattgcaacgatcgccgcgtcatcgttcatcgtctggatgcgcaatgcggcggcggccatgacggcagcgactgcggcagcgacgggactcagtgcggccacggcggccatcgtctggccgattgccttggcgacggcgttcgctggcgcggtttactacctggtcaccgccttccagcgcggccggaaagaggcgcttgatttcgccaacgcactggacgtttcggcagacgccctgcgacgcatgactgcagctcagcgcaccgcggctctcgataaacttggcgacgccgccgacgaaggccgtcgtcagctgaaagacctgacgagcgaagtctacgatctggataaatctctgaaggaactgaaggcgcgggacaatggcggtatcgggatgaaatcgtccatcgatgctctgcagcgcaaatacaacgaacgccgcgcccaactggatgacaccgagaaaacgctgcgggataccgaagccaaacagaaagcgctgcaggacgcgcagaaggggcagttccaatccacccctacagcggccggcacggctgacccgggcactggtggcagcactgacaacatcacggccgccgccctggaaaaagagctggcaaaagcccgcacccgagctgacaagaaactccaccagcaaagcctcacggatgcgaagcgaaactgggaagagcgcaaagcgctgattgtcgaagaatacaaagtccagcaggacatgatcgacaacctggcgaagacgcagccggagaaagccaagaagttgaacgagcagctgcagggcatcatcaacgaccgcgttaaggccgaagagatggcctacaaccgggaagaggcgaagatcggtgccaaggaagaagcgaagggcgcacgccgggttcaactggcgcaggaagtcgccaaccagatttccgctatcgaagatgacctggccaaccgcaatgccaaggtagacgggacagtctcgttcgccgaccggcgcgcggccgcgattcaggccgtcagcaattcgtatgatgcgttgtacgcgaagatccagaaactcggcggtgccgaaggcgcggcagcaaaaacgcagctcgacgccctggttaagcagcgtcaggaagtcgaaggcctgaagcaggataccgaggaaatgactcgcctgcagaacgtgctcaacgagcgcatcaaaacccggacagcgttacttgatgtgatcgagaaacgccgtgccgccggaaccatcggcgccggggatgcgatcgagcagaccaagggcgtgatggcgcagacgggtccagggatcaacgaagcggccgatgcggtgaccaagttcgctgagtcgctgaaaacgatcaccccagaccaactgacgcagatcaaggcggagatggaaggcatcaagttgtcgacggttgactcgaccaacgccatggccaacttcgccaagacggcggttgactcgctagtcggtaacgcgcagaccgcgttttcttcaatgtctgcggcgctgggcgatttcattaccggcatcaagagtggcaaagaggctatcggggagctgtgggcagcaaccaaacagttcttcgccagcttcctggccgacattgcgcaggcgatcattaagcagctcttgctcaacgccattcaaagcgctggcacggcgatgggcgggacgttcggcacggcgatttccggcgctgcagcagcggtgaaccacgcgggcggcatggcgggcggcagcggacggtcgcgcgccgtacagtccagctggttcgccaacgcgccacgcttccacgatggcgggttccctggcctgaagtcgaacgaggttcccaccatcctggagcgcggagaggaagtcctgacggccaacgacccgcgcaacgcgatgaacggtggtgcagcgccgggtgctgcttcaagcggcggaccgacagcccctccgcagaacattcgcgtgattaacgccatcgattcgggtagctttatctcggccggcctgtccaccccggaaggccagacggcgatctataactacatcaaggcgaatgcgtccgacttgaaaaccttcttcaaatgaggtgctgaatggcaggggttaacgataataaaattgtgatgaatagttccgggcttgtcgcccggagctacaacgcccccggcggcacaacagatagcggcctggtaactccgtcggacggcactagccttgaccagttctcgatcatcctgggtctgctgaacgcctatgaaacgataggagacccgcgtgcgttggttcgggctgagcaggcggttaatgccatcctgccgatccttctgcgaggcaccccagtctccgccacggcgaataacgttttgccgcattggctgttcaacgtcaaatacgagcacgacggaatcgccatggaccaggtgttcgagtcgttcggtggcatccgtccgttgaacgccggcgagataaaagccgaaatgcggatgatgttggcggcgccgcgcgcgctgaataagtttgccagcatcaccggcaaaacgatgttcgctgacatggcgcaggccatcataaagaatgcgcgagaccttgcatcaagcatcggggttatgccgcggtggatcatcctgaatacgcagacgggagagaagttgccgagcgccggataccccgtatgggcgcagctcggttcctcgtttgacatttcgatggtggacaccatagcggcgagattgcagacgatgcagacgtcatggcaaaccgcgctcggagaactcgggccattcgcgccgatgttcaaccgcgataccggccagggcgactggaacacgctgctatcatatgacgtcggcggccacgggcaatactgggcgttgaccgacctatcgcttcacgtcgcggcgtcacctaccgctcgcggcatcgtcatcgagttcctcgattggctggcgacggacgccaactggttgccgaacgccccgtcgtttaacgataaactgacggcaatgatcaaccggtcttcgctgaacgacttccagtcctatgatgaagttcaggcgacgatcgatgcagtcaccctgccgaaactcggacctccagatttctaccgggcaactggtgcagcgcatgtttcagacgacccgtgcttcgccgccatggtgatggaagccgccgtccacctggataactatcagcgcccgagcggaaccggttcgatctacctgtcagtagagaacgtcatccataaagccatggccatcatgcaggtgacctaccggaacgctggcgagatggccggcacgttcgccgatggcaacgactggacgtcctacaaacatggcgcgatgatgtcggcgatctcgtacaccctgcaatgggcacagaaaaccaaacgtccgtctattgagacgctgtgccgagagtggctgtccgggatggtgaagtgggtggatgccaatacccggcgtgccggctctccgtggggcgctcgcccgtggcctttcgagattgactgggcgtccggcatgacggagagcattgaattctccaccaacgtgatcacttctcgcagcggccgggagcagaggatttcccgtcgcaggaacttccgtcggcgcctgtcatacaattacacgatgctgacgtcgcaagagtcttccgatttccaggccattctcaagtctcgacagaactcgcctgccatggttccgcagtggcagttctccatggtggcgggacagttctcagccgcgggcacaaacgtcattcaactggccgaaactccgcctgactttgtagaggcgggggtcccgcttatcatctcgcaaggaggcctgcagcagctgacttacgtccgggagatgaccgacacgacgctgacgctggaagacgaactggatttccctgtgttccccggcggcctggcagtgccgagcgttaacgcaataattgacgcatccacttcgtcgacgaaactcagtgcatcctacatgcaggcggttgttgggttccaggttctgccgcaggaagattaccgcaccttggccaaccggccgcagcccggcaactggatttccttcatatcgaccgccggtttcctgtgggcgaagtacatcaacaatgcatcactgctggaagtaaaattgccgctgtattggccggagccgggtcggttcgatttccccatcccgacgttcaccgttgggtcagaaacgcgcgagttaatcgaccgccggcccaactgggcatccgctgtttccgttgacgacacctggtcctatgacctggtttcctatgacgccggcccgataaccccaatgggcggggaatcattcgggcgccggacagtgaaggcgacatggacgttcttcaccaaagaagaccgggaagacttcgagtcactgctgaaacgcctgcgcggtatgcaggttgcgtgttggatcccatcctggacccgtgatttcgagatgcaggcggacatgaccctgggcaaccgcttggttgttcagaagaacgccttcattgacgaagggctgttcaacgcggcttacactgggatctgcatcatgacaaagggcggtggaatgctttgcgcccgcgtggcatcatccgtgataaatggcgaccaggcgacgttaactctcgatagagacctcggcttcctggtcaaaatggatgacgtggcgaaaatcagcgcgctgtatcgagttcgccaggcgtccgacacggcagaaatccattaccacaccaaggccgtggcagagtgttcaatctcgtttgtcacagtaaacgacgggccggtggcataaagccggcttaggagacctccgaatgtcctacgattctatcgagaacagcattgacatcagcaagccggtatttctttacgaggtggtctacgggccgtctccgcaggacgtttaccgctacatcgccgaagaacgcgatcagaccgccatgggcgccgtatggaaagcgcagagcataaaacacggcgacatcaccagcgcgggcaccctggataaacaatcgctgaccctgaccgcgcgccgggatattgacgtgtgggacctgtaccgcatcgggccgccgagttatgtcgttcagctgcggatataccgcggccaactggacatcaatgagttctccacggaatgggtagggcggattttactggcgcaacgccgcgattccgaagtagagttcagctgcgagccgatctccacgtctttgcgaaatcccggtttgcggcggaaatatcagtacggatgccctcacgttctctacgacgatcagagctgtaaggccgatgtcaacttacacacggccgccggttcaatcatcgcgattgagggggtcacaacgcccgtggttcagttcgccggcgaccagcatggcgtaaccgccgaaaacatggtggccggtatctttcgcacctacgactccaaaggcctgatgatcaggcgcgcggtggtttctgcagaagacctgggaaatcggcagttccgcctgcacctgatgacctacgcaccggacatggccgtcggacgcatagccaacgtgtctaaaggttgcccgcacaccttcgccgcgtgccgggacacattcaataacgtgggaaatttcggcggatgtcctaatataccaacaaagaatccgtttacttcgaaccagttctaaggagaaacacgatgccagtatgggcggttcaggtcatcattgccgtcgttatgatggtggccagttatcttctcaccccgcggcctaaaacgccggagaacaaaccgcaggtgttagaggcaccgacggtttctgccggccgggctatcccggttccgtttggccgcgtcagggtaaagtcgccgaacgtgctgtatttcggcggccagcgaaacgaggaaatcaaacgatgacaatttacgttaaaccacaagacctgcctaacgcgggtctctgcgtaactggggcgaaggactggttccgccagtacaacctggatttccgcgaatttatcttcggcgaaggttgcccggtagaggtgatgcgcgctgcggcgcctaatgaccctctcggcgaacgtgcctgtcaggaagccgaagcgcggcatgcccgcgaacagggagctgaataatgggtggtaagggcagcaaaaaggccgtcgtcggctacaaatacttcataacgatgcatgtggctatctgccatggtgagatcgacgccattcgcggcctctggtggtcagataaaaaagcgtgggaaggaaacctcggtaagacatccgacggcaccagcgcgactttctggcagacgaacaccgggctgttcggcggtgatacgtcggaaggcggcgttgacggactggtggaagtcgggttcggcggcggtaatcagcagatgatcggtatcctgccgaacggcacctacgacccagcggtaatgccgacgctcggcggacggttcgggtcatactggcgtccagcgtggccggaaatggcatgccagtatcgcggcctcgccgttcttttcatgcacggaaattacatcggcaacaacgcctacatgaaagagataagtgtcgatgtagagcgtttctggcgcggctggttcccggaggtcgcgacggtgaatggccatgcgaatgcggcgcacatcatctatgaatcgctgacgaaccgtgtctggggattggggtacgacgtcgactccctggatgacgcgacattccgagcggcggcgcaacggctttacgacgagggcgtcggcctatcgctgctatgggacagcgagcaagacctgcaggccttcattgacgacatcaagtcgtgtatcgacgcctcgttctatctgaaccgccgaaccggcaagtgggaactgaagctgaaccgtcccggcgacccggtgaaaatgacgatcaccccggacaacgccaagctggtatcgttcacccggaagtcgctgggggaaaccttcaatgagatttcggtgaaatataccaatcccgaaaccgaggattacgtcagcgtcaccgtgcaggataccgcgaacatcgagtcgcaaggccgcgtcgtatcaacgaccaaggagtatatcggcgtccgggacgaaggcttggcggttcagttcggccagcgcgacctgcaggtatcctcggccacgttggctacggcggaagtgctgtgtaaccgggaagcgtatgacatgtaccccggcgacgtcgggcacttctcctggcccgaggacggaatcatcaaccttcgcatgcgcgccgtcgaagttaccgttaaggcgggcggcgacgaccagatctccatgaagctggtggaagacgtcttcggccaggcgaatgggtcgttcaatggcatcagcccgcctggctgggttgacccgcgccaaaaagccaccctgttcgacgttgtgcagccgtgggaattcccgttctggttcgttgtccagggttcggggatctccgttggttcgctgccgacactggccgcatacggaacatcgtttgccgtcggaaccaacccagacgcgcgggcagtgcgcctgttcggccgccagaataccccggatggcgctgcatgggtgctggagaacatcgggccgcaaacgcctaccgcgacaacggccgccgtgctgccacccgcgccgacctctacgttctcaatcgaccccggaacagcgcgcctgttcaacaatattgagacgaccagcttcctggtgatgatcggcggcggacgccaagaaatcatgttgatcactgcggtatcgtcagacctgcagacgttcaccgtgatgcgcggcttgatggatacgcacccgtacaactggccggccggcacccgtattttcttcatgggcgagaactcattcgtttccgattacacacagcgctcgctggcagaaaccgtgcagtaccgtccggccatgcaaacgtcgattgaccagatggacgtcaacagcgtgccatcggcgacgatcacaatgcgcggtcgatacgagctgccttactcagtggccaacgtccgcattgaggggcaatactggccggcaagcattaacgcggccgacgtttcattcgaggtttcgtgggcgagccgtaaccgcttgctgcaggacgccgccacgcaggttccgtgggacgccggaagcatcgcgccggaagaagggacgctgacgcaccttgacctcctggacgccgggggaaccgcggtagtggagcttcggaacctcaccggattgacggcggccgtgccgactgacaatctgacgtctggttcttacaccgtggtagtgtggactacgcgagacggccgcatgaactaccaggcattccgccacacgtttaccattgacatcccaacccgcgacaccgggtatggcaacagctacggcttcgattacggcgaataacaggagagcatcatggcagcatcacaaggccctaacctcggcattaactatggctggaatctcggagagaacggctggaaacccggcatggatgccaacctgaaaacggttgatgcgctcctgcacttggctgttaacggggtggtcaattcaccgagcgtcacgacgaacggcactcgttatctggtatcgacgtctccctctggcgacttcgcgggccaggcggggaaaattgcagtgcgagttgccggtgcgtgggagttctacgctccgaaagccgggatgatagcgcaggtgataaaccaggaaacagagtacctgtatacaggtggcctgtggaaacttcggttcggtgagcagacgcgcctcgatgtttctgacccggctgggttcctgctgccggcatccaacttcgccaagatccccctgaataccgttaacgtcgacaaccgttctggctgggacgccggctcttcctcttacacgccgccgcaagacgggctatatttgctaacaggcatcgcgcgcccacggagaacgtcaaccggagccatccccgatagcattcctttcgctctcggcgtcggcgggactgctgctgactcggaaaatgtggtatataactcgggtccagtgggtggcggcatctttactgtatcggtttcccgcgttgcccgattgaccaccagcagcactgttttcctgtttggacggcattaccactcggcgtcggtggcagttgactcggcgaacctcacagtggtcaggctctccgcttaatcaccagaacttattggaaggacttcaacatggcagaccaagattacccgcagctatatcagcaaatgaaggactcggcgcaggccatgtccgatgctgccgtggccaacaagcagatgatgacggcggatgaaaacaccgacgtgacgattccggggtatgggccgaagccttcctactccaaacagatcctggcgatgacgcagagtgtcatatccaacgcgccgtattcctttgcgggcaaggtatacgacaccccggctcaaggcgtcacgcaaccgggtggcgtgggcgaagggaagttctacaatgcgcgcgtgccgggtaacgaagacgtctacatcggggagtaccagaacatcggcggcgtggccactccggttctgaactcttccggccagcccatcatctacccgactgacgtgttcatcctgaagaagttcaatcaggtcgtcgacatggtgaacatgctgctgccgaacaacggcattttcaacgtcgccggcgagaatgcgatggagatcgccggcagcaacaagacaacggccgcgtcgatcgacaaactcggcatcctaaatctgttcggcgggttgaacgtgctggatcttcaggttcttccgctcaaccccaacagcgaatactgccatgcgtgggggtctaaaactcaaaccatccttggtatcggtaaggcaggatggattgagttctttggcatgttccgtctgcagatcgtagaaggcccgaacattattgaaatcgttgacatcaacaacaagatgaccgcaggcctggataaggacggaacggcgtttaccaagggcgtcgacgagacaccggaaggcgatgccttccttgacttcgttgagcgcatgcacctgatcatctacgggcagtcgctgtcaaacggtgctgttggcgttccggtattgaatacgccggtttccgacgccctgatgttcaataccggggttcggagctatcagaaaaatccggcgtcattagtcgacctgaaagaaaatcaagaagggactaacggcgaaacgatcgcttccgggcttgtccatggttttgtcgctaagtgcggcgggatgtacgggcgcaaacttatcctcaacgctggcggcgttaatggtgcgacaattcagcagatctcgaagggcacggcgtcctacaaccaagtcatcaccaacgtcacctggacggcgggtctgatggctacggaaggccgtcagtacagcccggacttcatgccttatctgcagggcgaggccaacatgaacatcgggacgccgaaggagacctataagacgttgctatccacactctacagcgacttcaacaacgatctgcgtcctgttcgtggttccggcctggagatggtgatgctgacctatcaaacatcatcccacggctactacaacggcgcgccggggttcccgccggaggatattgcacaggggcagttggaattagcgctgactcactctcacattgatatgtgggggccgacctatatgtgcaagaaaggcgactcgacaacgcccggccacgccaacgttcaccacaacaaccacggctaccgactgcagggtctgtatgctcagaaagtcatccgccaccgcctgcgcacccgcagcgccgaaaacccagaaggcgagaagtacattccggtgcatgccaagtcggccaagaagataaacgatacaaccgtgctggtggacgttcacacctaccacccgccattggtgattgatgcatctctggtcaccgaattgccggacggcaatcacggggttgagcttcacgattccaccggccgtttaacgatcgccagcgtttctattgtcggcggtactaaaatcaaggtagtctctcaaacagcaatcggctcaggcgcgttcgtggcgtttgcctggacgccggataaccgcggcccgcttgacgaaaacaataaataccctgaatggttcttcgggagagaaactggggtgagaaccacgatccacgattctgacccggagaccactgatctggtcaacgaaaacggcgcgaactatccgctgttcaattacatggccattcaaaaactggagatcgtataaatgaccttacaaatcgttgttaaacagccgttcaccaatccgctgttccctccgtctgacgcggtatcgcttatttaccgagattccgctcaggggatttacgaaatgctttcgtcctatgaccgcagcggcaaaggaaaccacctgaaggtgaacggccatacgttcgggccgcaaggtctcatttccacgccgaacaccgcgcattacgccgacacgggtatcatgctgccgaacacgttttccatgatcttctcgcactacattgctgcggcgccgacggctaccgggaacatctattctaacctgaacccaggggcgaatccgttcaccggtactcgaatggcagtttcgtctgcgggcggtgccggtagctttgcgatcggccaacaaggcggatcggcgttcttgttaaatattggcggcattgtctccggctggacgacttattgtttctccgtgaatggcggccactaccgactcacccgtcaaaacggacagacgttcgagggtgatattccgggaacagtagcgccgggcattgttccggttcgcctggctggtgggtatatcccaaccgtgaacattccgatggacgggattttgggtctggccgggatttacttcgaagatctgacggtagctcagcagcttgacctaatcacaaaggcacggcaaatattagctcggcgaggtattgcttaaaccaatcagaaaagcgctctacacgggcgcttttttataattataattagtccgaatcttataacacagcatgggcagggtgatcataatgtcagaaccgatttctgcaggaacggccacggccgtagtcggtggtgtagcaaccgccggctggtatgccggtatcgacgccggcgtggttgtttgtgcattcgcgggcgccgtgttttacgtcacccaggcaaaggatatttcgaagtgggcacggttaggtttcttcctggtgagcttcgtagcaggcatcgccgccagcagaccggccgctgaactaatctcagccgttgttccgacaaaaagccagatcccggaagccgtcggcgcgttggccgcagctgcctccgccgtgaaccttctgctggctctgaatgtcaaaaccattggcagcaagattttaggtttcgtcggctcggccatcgccaacggcatgaataagggcggagggaacgatggtaagtgaaatcctcgtaacaggaaccacggtcgacgtggttcttatcgcatacaacgcgattgtgtgctgcatcacctccctggcgctgatgttcttcgagcgcaatggggcgacgcaccgccgctgggcatcgtggattgcatgggcgctctgcgtgtcctacatgtcattcccgatccgattcctgtttggccaggttctcgctatagactggtcaactctcgtcatcaacaccgtcttctgtgctgcgatcatctgcctccggggcaacgtagcgcagctattcaaaaattctggagccacgcatggcactaacgcaaaaagattatgagcaggccgccggccgcctttcaattcaggtcgccgcgctgcgggctttcgccgaagtggaaagcaacggcagtgggtttattgacggcgtgcggccgaaagtccagtacgaaccgcacgtcatgtatcgacgcctggtcctgaataaagatcgcgcgttcgccaattacgccctgggcaagtggccggatctggtctccactaagcccggcagctatcagtcgctgaacaaggaagacgaggacatggggcgcgccgccaagttcatcgaccgtcgaagcgcgctggaggcatcgagctggggcgcattccagatcatgggttaccactggaaaacctgcggctacgaaaacatccagcagttcgtcaacgcgcaatacagcgacgctggccagctcgattctctggtgcgcttcctgttggccaacccaggcatcctgaaagccatccgcgagaagaactgggcgcgcgccgccgagctgtacaacggtcctgggtacaaggccaataagtacgataccaaactggacgccgcgttcaggaaatacgggggtgtttgatgcagatagttaactggaagggctacctggccgccagtgcagcgacagcggtcatcacggccgctgtttgcttttggctcgccgggcggcattatcagccgacgatcgataacctgaaccgcgagctgggaacctgcagcaccgcgcgccagtctgccgagaacaccgtcgcttatcagaacggcacgatcgagcgaatgaaggccgacgcggacaaacggaagcaggacggcgataagaagcaggattccgcggcctcagaagcccgtggagactatgcaaaggctcaagaggtaatgacggaagccgtggacatggcgaacgagtgtagaagcgctcaggaagcctttgcgaacgaactgaaacaggagcgcgccagatgatcagaattgcagccgttgtggccatcgctctatcgatcgccgcgtgcagcaccaagccggcgacgccgacgttcgttgaagtgaaagtcccggtgacggtgccgtgccaggctaagccggttgagcagcccgtgttcgcggttgacctgctgaagagtgatgccagtatctcggcgatgatgaaggctttgagggcggagcgccaccagcgcatcgggtatgagaggcagctggtggcagcgatcgaggcgtgtcgttaactcagtcttccgcggttgtgctttcatcgaaatccgatagggacttttcatacccggagtcccaatctctcgccttttggtgttccttttctctgctatacggattaatgctgatcttacccggactcgctctgtcgaaaagaaaagcgttgtaaccttcgatccattcgctcgaattaaccggcgcctctcccttatccatgtagaaatcagaacgggctttttcatacccggagtcccaatctctggccttttggtgttctatcttatttttaaccctgctgtatgggttaacgtttattgtagtctcgctccgactatcgagaaggaaggcattatagccttcgctccattctcgcgaattacccggcgaaacgaacacacgtctttcgttggcttctatggccgatcttatggtggcacggcgttgcgcgtcgaaggagtggcggacggattccatcgtcaattctatcttcgggccagccggcatcttgacgaacgtgttgtacacatggttgatgtccacgttcgggaacggcagttccatagtgtcgttggccagcaccatgaaccagccatcttctaaacgggtgatcgtctgccacgtttcccgaaccgcgtcgctcgggtcttggaagaggtcttcgtactgctgcagagtgatgtcagtgtctttgaagaacagatatttcatgatttagctccagttgaaaagtcctgccaggcaccccagcagttgacaatggaaacgccttgtttcatggcgtagtccacacagtttgatgtgccgccttttgacccgttgtgcagcgccagcagcatcccgcaatggtcgaccatccactggttgcgaatagacatcttccctggcgcataggcccctctgctgacgacaacggtctgtgccgctacctgaagcagttgacggtatcccacctggtcgccattattccacatggcttcctgcccgatgaacggtactgcggcgatgaacggtatcttcagcagcacgcatgcttcggcgacagccgtgtcccagccgagcgccatgccggaaatgaccttctcgatgggatgaaccttgcccatggactgcaggcaatccacggcgaattcgcgtagcttttcacggctttctgcagagtagccgccgagcttttccggccggtggccagtggctgcgattatcattccccgcccccttgatagctgtcatcgtgcagcccggactgctggcgctcgatgatgatgtgatccaggcggtcggtcatcgctttgttcttcagcgcgcgccagtgggataccatggctgacttctcactacaggtcacccactgttcccagtggaaggactcctgacgcccggactggaagaagcgtttctcacgccggtatttcttgccttccagctccacggtatacatggggtgcatcttcccttcggtgccgcgccagccggttaccgttacgatgaccgggtggaactcttcagtcagcgtctccgggtggagtatcggccgctcgatcttggccaagtgggcgacgacctcattgtcatacggcgacacccggcgcagagagtagccgacctctgcctttccgcgcagcgtgaacgaggagtgctccacccatttgcgtttacggatgcagaagtgactgcatacggcacgcgtcttcgtgatgcgtgtaattctcaggaccaggtacatggtgccgaacgcgcatggcccgaagaacatcaggtcgccgatcttcggttcgccatgggcgtcccagcgtttctggaaatctccgatcataattcggtatcctctaaaaagttgattaacccgcattcacggcacaccacttctcgcggcaggccgttccagtaactgctgataacctcgcagttgtagtggctgcaggcttcctggcggcgctgctccgcctcccgctctgcgtagtccagcgcctcttgctgccggcggcgctcctgccgcattctctggctattgctcatggccatcgtcagacccccagtctacgaaggcacagccgtcctccgttctgacttctgccgtgcggctgcacgcaggacattgaacttcgtcaccttcgaacagcaggaaggcgtttcctttcgtcgtaaaggcgtgcaaaacaccaaatccgcagctcttgcatggcccgagccagctaagcttgaattcgtcacgccgaggttccggcagcggtgcccatttgataggggcgatttggctggtgcagaaggtatctttgttcccgcaccactcccgccacctgccgtcgacaaaccgacattcgacccaggcattcccgttgcgaaccataccgttaaccgaacggtcgtattcgaaggtgtgggtcaggatcagcacgcaggtgccatcagttggcgcggcggccatgtcttgaacgttgctcatagctcttcatcctcttgccagtcattgacctcataaccggcttcatatgctttacggtagccctgaaaatcaatcggctctacgagctgaccggattccaggatttcatcgacacgcgcttcgtcgtgctctttccatgcgcattcgctttcatggtgtatcaggtagaacgtggccattacgcgtaccccgagcctatgccgtttccatgccgacggtggccgacgtggaatttcatgcagaatgggcactggtagacgtgcagctgaccctggtgcccgcggctcctgacgagcgatatgatcgcccgcgccgccttctctgcggtttcgtgttttatcttcccttcgcacgttcgccggcgaagtcgtcttttgctggccatggcgcgctcctacagcagattcgaatcatcgaggatgtgccaccactctgttcgctttttatgcccccatgtcttttctacatggcgaacgtaggcgccgttgatgtccttggccttctcgatcacctgcagctcgttggccagttgggcatcgtcgtggatgttcgaatacggcgagatgactaccgggtgattgaacatcgcctgcccgtcggtgattccagcgtgtcggcgctccagcgcgcgctgaaggacgaaccggcgcagcagcaggtgataatcgtcatactccgtcgtcaggccggtggagtacggcgcgaccgagtagatgacgaactgcagctgatctatcggccattcgtccatcaggtcgttctcgctcacaggtttccggcgcgagcggaggattccgtcgaagatgcagccgtcttcccagacgtcccggtcatcggtcttgctgatcatccaggtatctacgcaagacggcatgctgttggccatggccatcaggtgctcggcgacggtcgataccgcgtagttgttcacttccggcgcccggccggcagtaccgttcagccacataatgaaatcgtcaatgtcgatatgcgatttcacgatcgcctgggccggcgtgccgattaaagccggcatcatgatcgccggatactccagaccagtgccttcgaacggcagcggctcctgcaggtttatcctcatggcagatttccttcagtggtcagcaggcaccccagcgcttccagcatgtcgaaatactttttcttctgctggtggcactgcttgttgatctgctgcacctgaagctcggtcatccggtcttgctcctggcggcgcgggacataaacgggaacggtcctggacccgtattccggcaccagcaacagacagatgttcctgctgtggcgcgacattcctacccggagcgagaatttcatgccgctcgggtaggtgcttatcacttctttcaggcgttcaggggtcatagttcgtccctcaagtgctcccaggtttgttgttcaccccggtgcgcgggcgtcagtgtcgcgccgcatgccgggcattcgcaaaggagaacgtcatcgagccggccgctcaggtcgctgtgctccagggctttcagatgacaggcttccacttcaaaacgggtattgcatgacgagcagattttcatagcatctcctacttcgaccgcttggtttttccccggctaccgcgctggcgcttctgctggttgtcctgcagctgcatccagaaaggcttctgcttaaccggctcctgctgattgtcggtcaggtatgtttcaaggtcggataacgttaaaggacggccgcagcgggcaacatcagctttcaaaatcagaaagtctgtttgcgctacacgccgaaatgcttctcgccacttggccataaggacgcccaggtcgtccggcaagtgggcggttatcttcgtctcagcgaacccgtaggcgaccgtttggaatcgtcttatgtgcttcacatcgatggctctcggcatctccttcagctgaggatagcgatgcgtcggcgaaatcccgcggacaattcgaggacagctagcgtagatctctgcctggcttttgccgccgtgcagacgctcttccagctcttcgtggagttcttcgcccagggctttcatttttgagcggtagtcgtcatccggcaggccgttatgttcgtagatgaactcgtcgtggatgacgcgggggattttgatagtcacgatatttctccgattatctgtagaaatgaaggtcaagagatatgatcgtctttaccggtatattatcgatgtgctgtttctcagaacaggtcatgtagtccgcttctttccaggtgccgtatgtttcctggtaggcatagctgaccatcatttttcggcgttcataaagacgggtgtccattgcgcggcgaccgaatccgttaaaccggcgaaacccgtggataaggccgttgatggagtgcgaccttttattttctgaagttatggcctgcagggcttctttcagcgccgaatattgccggaagaagtattcgcctttctctttggcgacgattgcttctgcggtagtggtcaatgttttcatggccgtcttttctctttgaagagcgccctgtccgggcgccgggatttcaggttagcgaacgtcaatgtccgggatgatgaccgacggcttgaacgtgacacggtagaaatacttgctggccttggcgccgtcgacctgctccatgaaccaggtcacgttgtcactcagccccagcatgtgctttttgtaggtcgccggcccggtctggcagacgacaccaagcgtggtcgccgtgctgctgttgtccttcgagcacaagccctcgatggtcagcagatactcgccggtgatcccgttgtagaagacaaagcggcgattgacttcgaagttgtcgctggcagtgctcaggttgcgggaaacgacgtcggcgtcgttgacgtcgcagccggccagggctaacagagacagggcaatcagggatttcttcatggtgttcctcgtttgttgcgggttatcgccggtgtggcgggttgatttataatttagatgatacaaaacttgtttgcaaggttttattgaaattaatttaggcaaaataaaaggcctcttatcgaggccttctgttatccgagaagccagactgcccacttcaggaacaggccgacaatcgggatgaagatgacgatggccagtaccgtggccagcgccgttccgaagtcaggtttttgcttttccatgtgccaatcgtctccgtgctgccgtcagcgcagatcgcactgggtgaagccggcgccggttctcttccgcctggcggtcgatggcctgctgaaaagtttcaacgcactccggcgggtcgggatcgttgacatcccaagtcttgccgcacgcgctacaggatacctgatcgctatactggcgcggctggcggtgtgggttcttcatcgttggtgtctccgttttgctgcatgtgataccacgctacgaggttctccaagtgagggaacaccgggatgccgcagtaatgcgcatgatacatttcattccgcgtgcctacgcttttatcataggcatgcggctccgttagcagcactgcatcgcatttccgcatcatctccagcgtgcccttcaggaaatactcgtcgcttacctccggcacgtccagctccatgtgggcagtgctcatgtgcgggactaccgggaacatgttcagttgcaactggatcacctgcaggccggtgcgccgggcggccagaatattctgctcgacagtcttcccgccttcgacgtgccggtatgggccggcgatgtagatcagcatcatgcttcgtctcccaggttcaggtgggtgcagcctcgcatgccttcccggacgatgcgccataacacgacggcctgatggcgggcatcatcgatgccgatgtgcggcgtgccgtcgaatggggtgttggatcggaagccggggaacagggaattcagcgttctgaagcaccggtgaccccaccatggccacggcgtaccgaggtcatgtcgtgcataggcattggccagccataacgcatctgccagttcgtcgttactccacacctcaacctgactgctatccacgccgtgcttagcagagaggcgctctatccaggcggtcagtgcaagcagagcttctatctccgggacagcattgcgttgatgatgcgtgatatgcaggcgcgaagcgtcaccctgcctcatccaccacaacacggtttgcgggtcgacatggccgccggaaaacgcgctgtctatcagaatatggctgtcaaacgttgagatgataaggtcatgccggtttccggcactaaggtcgaacgcgacggccgagatagacagcagggcagcattggccttggtgctgaggctttcgcagtccagcataattcgcagcgggggaatcatagttcatcatcctcttcaggtgctggcgcagcagcaggttttacggcgggcgggaatgcccaggcatgcaggtctgccagcagctccatgccttctttttcgtccatcatcggcagcgccagcaggcggctctgcagcgaatccggcagcttcggttcaacgttctcagcgtccttggccttctgccatgcgcctttctgcgttaccggcggcttgacgctggccgggaatacagcttcgacgccgtgcagtgccgcctgggtggcgaggtagttgaacgacccttggcacttctcggcggtctcttccagcaaccactgcatttcctgcaggtcgcacgccgtggactccagcagctcgttctgcagcatgccggtgaccaggtggcggatcgtcgggtagaagcccaccagcgcgcgggtctcttcgccagggttcttcgactccaggcctttctctttcagctggtggaggatgaactggtatttgtcgctggtgatgacgtaggcatcggcgacgcggatttcgattgacatatggattcccctttatgagatgtcaccggaaagcataatgcaataaaaacgtgtttgcaacgatttccggtgactaattatcagaacttcttgccgccttctttttcccggttctccggcttgtggtcggcgcggtcgacgttgtatccccacttctcgatcagcgcaccgccgacgtcataaccatgtgccgccgcgtaatccaggatgcggataatggcgtcggccagttctacctccggcatcttgcggtgaggaagatggtcatccatcaggcctttccgctcaccttcgaacgcttcgacgatttcgctgacgatcagcatcagcgtctcacctttgttgcgcagcaccagctgcccggactcgtcctttttccaccagccggcattgatgttttgctgcaggatgcgtttctgcagccattcgaaagtggacgccacttcgatcaggtcaaccggttgagcttccaggttctctacttgtgacaggcggcgagccgcccggtaaacattggtttcatttttcattggttttctccgttaaagctcgtcagcggccagttcagcgtcgaacacggcggtcaggatacgaacgccgatccagcgcatcacgttaaccgccatgctgtttcccaccgccttatagcgtgacgcgtcagacgtgtggcctttcttgtagggtatcttcgtccagccgtcgggcatgccctgcagacgctcgcactccaccggcatcaggcgccgaacggaaaggtcgcagtcttcctgccgcacggccatggaaaacaccgcatggtttcctgccgtatcaatcgttgggcatgggtcagtcgggcgcccaatgccaagaccgttggcgctgccatctccgttccgcgttggcccgccacctttgtaccgggtggccttgtcatggatcggaatgcagtaggcgatgcatggcgcggcgtccccttttcctgtctcgccggcggtggccgttaacgcgttcacgatgtcgcccatatccccgcggccattgcgtgcgatacgcggctggaacgcatggacgatggccggcgtctggcagcaattcagcgcgccggagatgtcatccagtccgagcgtcgacgatgtattgccggccgcctggtggttgaacgccagcacgacggccaggtcagtagcggatttgtggtcgcgcgactgaaccgtggacgagcgctcgtcatcctcgtacttcccgaacttcgacagcctgaaggccttctggcctataaggtgtcctgctcgggcttggttgtcgtcagcaccaccgactccaacgccatttgaagtaagggcggcaacgatctctcccgtttcgcggctcggctcaagatcccttcgcacgccgtcgaactcaaataatacttgggcgggatcgatgtccttacgagcacttgcgacaacgaacacacgtcggcgtcgttgggccagtccgaaatattgggcatcgaggatccgccacgcgactgccctttggggtccatagacacaaccagcgtttgcccacttcggaacgtgcttaccggttttcgcgtcccagcgccaatgggtagaactccgtcccgattcaggtcgtggtgatggttcgagttcatcatctgcgccagccagtccggcaagcaggcatccgaatgcgttggttttgtcactgagtacccccggtacgttttcccatacactgatcacctccggctttccgtcttccaggcgtatgtgatcgatctcattggccagtttgacgtaggtcagggtgagctgtccgcgcgggtcattcaggccttcgcgcagaccggcgatcgaataggcctgacatggcgttccgccgacaaggatggccggcgccggaatctcccggttggccaccataaacggcaggcaggtcatatcgccgtagttcggcacatcgggaaaccgttcggccaggatggccgacggtatttcctcgatctcgctgagccacgccgcccggaatccgagcgggtgccatgccaaactggcagcttcgatgccactgcatacgctgccaaagtcgatcataaatcgtcttcctcttcgtcggcgttgcggtaaacccgcgtgccaggcagaccgtagatctcgtcctcttctaccgcgaggatggaaatcttgatgttcgcgctggccgccagcggccgcaggaattgcagttccttcggcgctacgccttcaatgaaaaacgattcgcccggctctaggcgcttcatgccgtcgcgatagcggcggttacgctcggacatctggcgcccgtttccgggacgcgatgctgggggccggccgacccctttttcgattttcacgtccttcttgctcatagctcgtcatcctccgggtcgacctggtgctggccgaacgtggttttcaggtcagtggggaacggatcgtcgccgcacagcgccgggttgctgttcgggcaaccatcatcgtctggttctgcgtccgcttcgccgcccatggcgtccacgacatcgttgagcaggctgaccagctcggccgacacgatcgtgaagtaggagtcaaaactctcccggcgttcgtcagcgtcgccgcccccgctcttcaggttctcggtcacgatgtccgggaacttgatgcgcttgaacacgaatttatcgttgataaagaactcgctctgcacctcgccgcgcgtgaagtggcgcagctgcacctcgcggacaacgtagtcgttatcgaggtagctggtcacctgctcgctggtcaggtcttcgtccttcaggcgcgcgttaccgccgtcgtggccggccggcgcatgcagagatgccgccgtgcccagcagcaggttcagttcttcgtcgccaccggagctgaccaggcgtgtcaggacatcgaccgccagcttgttggtggtgatgggggtcactggcaaactgccgagcgcggcgcgcagttcgtgcaggcaatcctccgccactttgacgctgctggtcagcaccaggatcaggtcgtcgatcagcagcatgaagatggtcttgcgtttgataaacgcggacttcagcagcgcggcttccgccaggtctttcagctgagcgtactcgtttttataggccttgcggccttcccggcgctccagctcggcgacgcgcttaccgacttcctcgcgcagcaccgtgcccggcaggtcacgcaggttgaactgcgccttgatcagcgtagcgccgcgatagccgcggtaaacgaaacgctcttcagccgcccactcgatcggcagcacgataccgaagccgccggagcgccaggcactggccgccgggtcttggacgtgacgggcttcgagcagcacttccagctcgtcttcgtccttcagcttctcgaccgtctccgggtcggtaatgcggtatgcaatgaaattcttgaaatcagccatcagaggctccttatagcgatttgccggtggtagtgacctgcaggtgtgcgaacgcctctaagaagcgctcacgggcggcctgcaggtgctgctgttgaagagtgagcagcgcttcttccgagatgatcgggtctttgcgcttatcctcttcgcaggcagcgtagaacgcctgcagcgccatctcttcgacgcgcgtcagcacatcggatacggcgttgcgcacttcggcgatgttcgagttcctgtccaggagagccgcgaggccggcatccgtgccgtaccacttattacggtcgttcaggctcatgcatcgagactcgcgaggccgagcgggtcgccgcggtactgcagcatcatggcgtaggcaatcacgttgagatgatcgccggtctcgtccaggtggcgctgcagtgcggcgcgcagtacgtcggcactgcagtcgtttttatcctgccagccgcccttcccttccatccgcttctcggccaggcgttgcttcagcagctgcgccaggcggtctaccacggaattgtcgaactggccggcgtcagaggcatagccgttggcaacacgctgttgctcgatggcttcgtctttgtgcgtcagggtgtacatcaacgcgtcgccgttcccgctgcgcttctccatgttgatgtcgaactggtttatcaacatggccaaatctgcggtggagacttccagctgacgcatacccatgttcatcatggccatgccgacggcgaatttcgagatgtgctcgatctgctcgttgtgcttgttctcagcattggccaccagcgtttccgcttctaccggcgtcatcagtgacaggtgagcagtgtcatcttccatcgctgacttgatgcggtcgatggtctgcagctgcttctcccagtaggacatgtcgcctgggtcaagggaaacggcgccgtgtggattgcaacagtccatggcgatcagcaccgctttgcggaaatcgtcaagatgggtaagcaggtcttccggcaggtagcgccattcgttggtcgggattgctttcggttcagtcatttctttttcctatcgtagatagtgtcgaatgcccaggtggctaacgccgtagggacggtgattggccaggcgagcgcgaacacccagatttcccgttcgaaattacgggtggagatccgcaccagaaaatcggatttggtcgcccagcgatgcaggatggtcagcaccacgtttatgatgactgccaccaccagccaaagaataactatcgttggcatcacaggctccctttcgttatcagcatgaagtcttcccggtacgctgccttgagcgtgccatcggacatcaaggatggcagttcgtgggtgaacactgggcgcccgagacgcttttcaacgtctgcgtggaacgcgctgaactcgcacatcatgatgccggtgaatccggtcaggataatgccttgtgcctggcttatccgtggctcttttgttggcatgatgcagctcctttcatcaggtcatcgaccaggttcatgcgctgcaggatctcctgcatccgtgcccgctcatgactgccttccggcgtgtctttgtagcacagctcagtaacgttccgcgcggtattgatcagcgacgtcagaatgccgaggcgaaccatgcgcgtttggctgttagcgaacagggtactgaagtctttgttcagctcgacgccgcgctcttccggtgacggcttcgactcgaacttcggctggcaccccttgatccaatagcggatgttcgggttgcggccggtcttcacgatgctgccgtcgcgcgccaggttggacaacgtgttcgaaacgttggcaccgttcacacggccggccagtgcgcgccggacgtcgcctccatggcaccctgggttctcagcgataaagttcagaaccagcgggcggttcggtgatgggggtttgctcatggttactcttcctcttcgtcaaaggggactccgccgtatggcgctgccattgcaggcgacatagatgtcgcgcggggtagcgcagccgggctttcgacctgtacatctactcggcctgttttggtgctggggccttcagtccatccgccctgtttcagttccgccttcagagcacatccctggtcgatcatgattgccgacagctccgcctgcagcatcagcagtttgtctcggccgcgagccgcaactttgcgcaggaacgccgacatcaggtcgcggttttcttcggcgcccagcaggtcaatgaagcggtgatggcaatcgtattgaccgattggctcatagcgcaggcctgcttcgttatcgccggcgttgatcttcagcgtgccgggcttggcgttttcggtggccttctgcaggaagtcaaggtcgccttgcagcttctccacttcggccattttcttctgtagttctttcatcttaggtctccgacaagggtggtaacggttttgcgaagggtcgatcccctacgcaggtatctggcggccggcgctgctcttctggcagacatcccggcgcgccggatcactcaacgcggatgacaacgatggacgtcatcgcgcggtactgggacgacacgaaagagccggtctccacggtgaacttgagttcgggattcgccttggtcaggcggctggtggcgttgttgatgatgcggcggcgggtcagcacctcgcccgtcatccagtctcgcatgccggcagatgacaggccttccggggcaatcatcgtttcgctgtatgcctggccgacgcccagctggctgataagacgattgcgggaggtggagtagcggtcggccttgctgacgccttcttctggcgacatcatgctggcttccagctcggtatccagggcagttttggtctcggtcatcgggttctctctttccgtgttcctgttaaacgtgtttgaagattagctgctacaagtttttaatgcaatctctttatgaaactatttattgttcatccgccatcctggaatttttcagaacgggctgggtctgtgaagtaagcctcattgaccggccagggtgctgaagtaagccagggcgcctggtcctgggtcattgcggcatcgaggacaaccggccagggttctgaagtaagcgacgccagccggccagggttctgaagtaagccagggcgcccggcaccatccaggcgcatctcaactttcatttcaaaataatcgggaaaatttttggaacgggctgggtctctgaagtaagcggcgggggttggcattggcgcctgccggtttttctataccgaaagccggggtgaagtaagccgggaactaccgcatttcatggcgcctgcctcgcgtgcgtataaaggatcacgcgcgcgtttcactatccggccgggcggattggtcatttacaaaagatttacggaaaggcttgcaataaagtttttaatgccctacattagtcacatcaccgcagaacgcggacacttagaaagagggttttatcatggttaaatttacccgcagtcagtacatgaaaggcgaatgcacgcatcaagattactatatgcaattcgccaccccgcagatcgtcgcccgtgtgaaacgcttttgtgatttaaaagtgctgcgatcatccactgacgaacatcttaatgatgtttaccgtcttacccactgggataatgtgatcggcgctttccccgccgacgtcgccgcaatgatgcgtgaagcgggcgattatccaacgcttgccggttcggtcagtctggcgaaagccgttgcccgttcactgatttcggaataaggggtttatcatgcaatccatcattactaaatatcgctctggttcacgtcgcatttccgctaaatgctggaacggtcaaaccacgcttgattatgatttggcgttgaacagtgaagataatcacaaggccgccgctgaaaagctggtttcaattttgaataaaggccgcaatgtttcgtggggtatcgtttcctccgcgcctttgattgacggcgggcaatggattttcattatcaattatgtgccggacgtcccaccgatgcatatgtcgatcacggttaagtttctgccgtgtaataattccaaaccggcgcgaatgaaagttttttcgtggctgtatgaaaaaggtttcgaggtcgcatattcgcctagcattgctgatgcctcggatatgtatcaatgtgcgctgtatgcagcaaacaaaatgctggaagaagtgaacggcgtggcgtttgaacataattgcgagtatcgcattaaagattatgtgcatactttcgacggtaaccgcattttcaccttggagttaatataatggctacggtgattactttatctgaagcgtgcccgattatatacgggcacgaattgccggaaaacgtgcgggaagattatgattatatttccgacgatgaactaccggaaacctcattctttaaaattgagggcgtttatcattgtatatctgatttcgttaaatcggatacctacgcggcgcaaggtttccacggcatagcgggcgaaactgcgtttagttcaattcttatcggcgtcattgatggcgatagtgatcatttaaaggtaggccgcgtatgctcataatcgcaatccttggctatctggtcatcgcgctttacatcgcgcacgatgtccgcgcagttatcaaacgttgccgcccggagttgcgccaatgtttcggccgcaaatcggtttacctgcgtgaacgtaaaacaggccgcctgatcgcctgttccaataacccttttactttattgctggtggcttaatatgaaactttcttccgttaataaagagcaacgcctttatgttatccgcgctggcgcaggttattcctgctacgggttcgactcactcaaccgccaagcggctggcgttttaaattggcttaaagagcaaggccgcacggctgaattattgttaggcgcgcataaagtggatgtgaaaagcctttctatcccggaacgcgtcggcaccaaaaagcattttgaggcgtgcaattcaatattgaacgctggtgctcaattttcgcgtgattccggcatccgttgctttgccgaattgcatccgcagttattgggcatggaaggaaagcgggtaaagattattgatgccgcaggaaacccgcgcagctttattgtagggcgatcttctggttggttgccgaatcatctggaaatatcacgccgcgattcatccggtggcgtgccggtttcgcgtgaatcttttcagtcggtttcgctggtgtaatcgctatggaaaaatacaaagttgttgagaagaaaaacccgttagcggtgcattgcttaacgtggtctaaacaacgggcgcaggaatggatcgagaagtacggcgattctaaaatgttcactgacaaaacgttaaatcgggatagttttacgattgtttgtcctgattcagataaaaattgattgggggtttaccatggataaattaaatatgtcctacgcgcaggccgtttcgtttttcacaatgggcatgcttagcaacccggaaagattcggccgcgcggttgatttgtacgacgccgggcaaattgaattcatggaatcggtattgccgttagccgttgcgattgagcgcgtttatgatgcgattgaagagcatcacgataattgttccgttgtttggggctacgatattttggaaccattcggcgcgtgggtagttgaatttatgtcagaccctgaaaattgtggttgcgtgcccggacaagaggcgatcgactatttagcgccgcgcttgttggaggcgttcaacgtaagcaattacccggacaaattcgctaaacaggttgccgaggcattccgccacggtgtaggggctatctgatggcacgcaccccggagcaacagcgcagcgaggccgcccggtggcgcggctactggaaagctaagggcttttccatgggtggcgaccgtgatcgcgtcgtactgacgcgcccggactgccggatcgaatacaagcgaggcggctggcacgttgccactgctgaaggcgttaacagcatgcgggcgataggctgcgcggaaatgctggtcggcgcgttaaaactagttcactgaggggctaccatgaaaactgtaaacgttaccgccgagatcgtcggcactggcgcgaatctgtataacgtcgaattaaccccggatggctgggattcgaacgcgctttatctggccgcaggcgataccccggagcaggccgccgccgccgcgctggctgatctgaatagtgacggcgcaattgatggatgcgacgttgaacaggccgtgcatattatcgggactttttgccatgggtaaacgcatcgttttaaacccgcttgtcgtgcgcctggctaaccgggaagtaaaattccagctggtgcggggatccgtgccgggtgagtacgatttagagcgctgcgatagcctggccggttgcgaggttgtcggacgggttagcttttgcagtggggcgatggaaccttgttactttgtcaaaccgtgggggcggtggaagtccgcgccccgttacattgaatcaacgttggcgggcattttggcaaaatccgctgaaaaagaggtgaaaggctatgatcaaatttaaaatcatgatgatcgtccggctgctgttgtctggcgtcgtggcggtcgctggtctggtcatcgctggccgctattggctaatcgcggatgaccttgtgaacgcggcgatcgtcgtcattatcgcgggcgtcgtcgtctcaatctgcgccccggatgccgctgatctggaagacttgcagcggttaattgaaaaatagcgcattaaaaacttgtatccctcgttatctagtgttagatttgttttaacagatttacattactagataacaggggttcaaaatggacattatcacttcagaatcaatcatcagccttttcctttcttacgcgtcgaaagtccatcatggcaagtcgacgataaaaaatgcccgtgaacgtatcgcggccgagatggctgcatatcaaacgcaagtcatttgccagtatacccgccgccgcattgcgttttatgcggatgccgtggcaaacgccaaggcagcacggaaaaacacgtttttcacgttgctggtggatctgtcaaacgacgtgcgggaagaggcgcgcctgatggcggttgcccatagtgaagcgctgatcgtcaatgagtcatacgacgaaacgccagaatgcccgaacggtaaccgcctgatccacgccgtggcgcgcgtattcggtcgcgctgaatcactctcatgggttagtaccgctggtgtggttaatgtccgctttgatggcgatgacggcgtgatctacagctacgacgaagattcaaacggcagcggcacgatcgccgagtttgacggcgcggcgtggcatttcgtggggtggctggaagtcaaaaacccgactgtagaaaacctgcgcgccgcgctgccgaaatccgctattgaaccatgggggcggtttaccttgcagatcggcaatcacaattacacggggttcacgtctgccgctgcggcgcgtgatgcctatgaccaaatgcgggacgaatccaacaagggcgcgcgtgattggtgggtgcattgctcaatcactgacggcaaggaacgcgtttacctgtcttacaacgggcgcgcgtggcgtgacaaggcgcacactaaagaggttttcttaacgctgaataccgggggtttttgattatgtgcgtgattaattatctgaatcgtttttctaatcgcggcgtgtggttgcacctgatcgccttttgtctggtgtgccatgctgcggcggccgctatcgttatcaatctgctctaaggggttatcatggatcttaaaaatattcccgtccgtttcgcgcttgaggtcgccgcgtttgctggcgctggggcgttgtccgcgaaagaggcgatcgaaaaagcgctacgcgcggccgatcgtgattacaacatgtacgacgcaacaggctacggtgaccgggaaaccgtttctctgattgtccccgccgtggtttgggcttgtgattccctgtacggtaaccaacgcttcaatcctccggctgatacgctgatgtgctacgactacgtagaaccttgcgccggatgtttagccgcgcggctgttgcctaccccggaaaccggagatcgtgattttgttgatttcatccggtcttatgcgctggggtggatggaagaccttgctcgtcagcgcggcgacctcaaagaggaaaacccggatgaaatcgcgcccggtctcggcgtgtttaagaccgtgtgcgcatcaaccgatcacctgcctttcgccgattatcagctgttgaaggcgcacgcgtaccgggaagacgtggaagcggccaccgcgccgtattggattatcaatcaccccgtgggcgtccagctgcagccggttaccgcgccattgtgggcggatgaattgcgggaattcggcgttagtctggcaaccgtgaacatgatccgcgacgtgctgcggcttggctatggcgcggtgctgttccacgaagacgccgacgtcttggagggcgtgcccgtggcgcgggataacgatggcgcttgattgggtattggccgcgctgggtttcgagacgctcggcggcctgatttcgtatgtgtctattttcgtcgccgtcggctttatcgcgggcaagattttcaggagaaagtgatgaaacaattttatgcaaatattccgggcgtggcgcacgcgcttaaagttttcgggcgtgacgaaaaagaagcgcgccaacgcctgcgagatcgtgaaggatatggcgaccgcctgcctgcggggactaaattgtatccggttgaaggggtcaaatgatgatcgttcaaaagcacatttgcgatgtccgtccgggtgacgttgtgcgccacgcaggcgcagatcgcacagtcgggaatgccgacctgaagtccgggtttatgggtgtaacgttattcggcgattcgtaccgcctcggtcgccagctggtagacgtggtagtcattgaaagcgccatgcctaacgccgcaggggcgcaatcatgaatggcattttgttatccgccatggtgctgttgtttctgatttgggcggatctctttgtgctggatagtgcccgcaaaaagtggcaacgcggcattatccaccgcgatcagtttcgacgttttgagatagcgattctggcaaagggtttagttatttacccgacgtttgttttccttttcatccaactggttttagcgaggtaatcatgattattcaatgggcaatcgttttgactatttgcacggctggcacgccggataagccggaaagctgcgatgactgggtgcgcgatatgcgcccgacttattccgaatgcgttaaggagttgcccaaatatgcaggggaaccggggttagccgtgggtgcatgtgaacgcgctattccgatgcctgaaggcacgccacccgcctgatcgtttgtccaaatcgccactaatcaagcccgccacccatgcgggctttttgtttgtgatctgcaatccgcatttcttcaacgcctggccgcctcgatttaaggggtgtacagcctacctacacgacgcgatcgcccctataggcaagccgtcatatagctaacccagaataacgccgtctcggccttctcagaacgttttaagcgaacatatacccgcccgctaccctggccgcctggtttgtgggggtgtgggctggccgtggtgcgcatgcatggcttgttgtctggctgttgcgtggctgggtgtgctgcatggcgtggtgacggtctggcatggtgcgcatgcgtggcttgttgcctgttgctctgatgttcacggggtggctacggctacatggtgcgctgtatgccctgctatctgcgtatcaatcaactaacaacgatttaaatttatttgtctaggctggctggtcacccggtagcggtcaaaaggaatcacatcgtaatcggtcaaaaggtactcctaagcccttgtttctgcggggcgggggcgcggagcagatcggaagagc

SEQ ID NO: 5 - DNA, 42609 bp, unidentified; identical to SEQ ID NO: 1
  source 1..42609: mol_type = genomic DNA